(19) United States
(12) Patent Application Publication
(10) Pub. No.: US 2019/0169593 A1
(43) Pub. Date: Jun. 6, 2019

(54) PRODUCTS AND METHODS FOR ASSESSING AND INCREASING KLOTHO PROTEIN LEVELS

(71) Applicant: KLOTHO THERAPEUTICS, INC., Las Vegas, NV (US)

(72) Inventors: Joseph F. Tarsio, Manlius, NY (US); Dinesh Raturi, Eastvale, CA (US); William Ian Ramage, Del Mar, CA (US); James R. Plante, Las Vegas, NV (US)

(21) Appl. No.: 16/208,552

(22) Filed: Dec. 3, 2018

Related U.S. Application Data
(63) Continuation-in-part of application No. PCT/US2017/035755, filed on Jun. 2, 2017.
Application No. PCT/US2017/035755 is a continuation-in-part of application No. PCT/US2017/063149, filed on Nov. 22, 2017.
(60) Provisional application No. 62/344,743, filed on Jun. 2, 2016.
(60) Provisional application No. 62/375,046, filed on Aug. 15, 2016.
(60) Provisional application No. 62/401,600, filed on Sep. 29, 2016.
(60) Provisional application No. 62/425,237, filed on Nov. 22, 2016.
(60) Provisional application No. 62/456,318, filed on Feb. 8, 2017.
(60) Provisional application No. 62/425,237, filed on Nov. 22, 2016.
(60) Provisional application No. 62/456,318, filed on Feb. 8, 2017.
(60) Provisional application No. 62/595,567, filed on Dec. 6, 2017.
(60) Provisional application No. 62/666,868, filed on May 4, 2018.
(60) Provisional application No. 62/692,195, filed on Jun. 29, 2018.

(51) Int. Cl. C12N 9/96; G01N 33/573; C12N 9/24; C07K 14/765; A23L 33/15; A23L 33/155; A23L 33/13; A23L 33/135; B01D 15/38; B01D 15/34; B01D 15/18

(52) U.S. Cl. CPC C12N 9/96; G01N 33/573; C12N 9/2402; C12Y 302/01031; C07K 14/765; A23L 33/15; A61K 38/00; A23L 33/13; A23L 33/135; B01D 15/3804; B01D 15/34; B01D 15/1871; A23L 33/155

(57) ABSTRACT

Disclosed are products and methods for monitoring Klotho protein levels and for stabilizing Klotho protein in a mammalian blood sample, especially at room temperature or without freezing, for a period of time. Methods of detecting and quantifying Klotho protein levels, particularly endogenous and/or exogenous soluble alpha Klotho protein levels, methods of diagnosing Klotho protein deficiency, and methods of increasing Klotho protein levels or production, particularly endogenous and/or exogenous soluble alpha Klotho protein level(s), expression, or production, in a mammalian subject, and products useful in performing the same, including diagnostic kits and compositions for treating Klotho protein deficiency, are disclosed. Compositions are configured or formulated to augment natural soluble alpha Klotho protein production, attenuate Klotho protein damage or degradation, and/or supplement Klotho protein levels with exogenous, recombinant protein. Treatment methods and uses include administration of the compositions to human or non-human mammalian subjects.

### CROSS-REFERENCE TO RELATED APPLICATIONS

The present application is a Continuation-in-part of International Application No. PCT/US2017/35755, filed on Jun. 2, 2017, which claims the benefit of and priority to: (i) U.S. Provisional Application No. 62/344,743, filed on Jun. 2, 2016; (ii) U.S. Provisional Application No. 62/375,046, filed on Aug. 15, 2016; (iii) U.S. Provisional Application No. 62/401,600, filed on Sep. 29, 2016; (iv) U.S. Provisional Application No. 62/425,237, filed on Nov. 22, 2016; and (v) U.S. Provisional Application No. 62/456,318, filed on Feb. 8, 2017. The present application is also a Continuation-in-part of International Application No. PCT/US17/63149, filed on Nov. 22, 2017, which claims the benefit of and priority to: (i) U.S. Provisional Application No. 62/425,237, filed on Nov. 22, 2016; and (ii) U.S. Provisional Application No. 62/456,318, filed on Feb. 8, 2017. The present application also claims the benefit of and priority to: (i) U.S. Provisional Application No. 62/595,567, filed on Dec. 6, 2017; (ii) U.S. Provisional Application No. 62/666,868, filed on May 4, 2018; and (iii) U.S. Provisional Application No. 62/692,195, filed on Jun. 29, 2018. The entirety of each of the foregoing applications is incorporated herein by specific reference.

### BACKGROUND

### 1. Technical Field

The present disclosure relates to assessing and increasing Klotho protein levels in a patient. In particular, the present disclosure relates to products and methods for monitoring, detecting, and/or qualifying the level of endogenous and/or exogenous Klotho protein and for diagnosing Klotho protein deficiency in a human or non-human animal (e.g., mammalian) subject, and to the production, purification, and administration of pharmaceutical or nutraceutical compositions for increasing Klotho protein levels in the subject.

### 2. Related Technology

Klotho (or alpha-Klotho, α-Klotho, etc.) is a recently characterized protein encoded by the KL (or klotho) gene, located on human chromosome 13. Two transcripts that arise from a single klotho gene through alternative RNA splicing have been identified. See FIGS. 1 and 2. The first transcript is predicted to encode Klotho isoform 1—a full-length, 1,012 amino acid, single-pass transmembrane-membrane protein, with a short cytoplasmic tail (human residues 1003-1012), a transmembrane (TM) domain (human residues 982-1002), and extracellular region or domain (human residues 1-981) comprising two (largely) homologous (internal repeat) domains (termed KL1 (human residues 56-506, which is 450 residues long) and KL2 (human residues 515-953, which is 438 residues long), which each share 20%-40% amino acid sequence homology to β-glucosidases, but may lack similar levels of glucosidase catalytic activity), and a signal sequence (SS) domain (human residues 1-33). The SS, KL1, and KL2 domain-containing extracellular region (human residues 1-981) may be enzymatically cleaved by α/β-secretases, and released into the circulatory stream as a 130 kDa circulating protein, termed soluble klotho (or sKlotho, s-Klotho, alpha soluble Klotho, etc.). The extracellular region can also be cleaved into separate 68 kDa protein (KL1+SS) and 64 kDa protein (KL2).

The second transcript, a splicing variant of alpha-klotho mRNA, encodes a second isoform of Klotho protein corresponding mainly to the KL1 domain. The internal splice donor site is thought to be located in exon 3 of the klotho gene. The resultant alternatively spliced transcript contains a 50 bp insertion after exon 3 (FIG. 1; gray), with an in-frame translation stop codon at the end thereof. The expressed protein product is secreted into the circulation and is termed secreted Klotho (or Klotho isoform 2), which differs from the canonical sequence of isoform 1 at amino acid residues 535-549: DTTLSQFTDLNVYLW→SQLTKPISSLTKPYH, and with amino acid residues 550-1012 missing.

Accordingly, there may be a number of different Klotho proteins in the circulation at any given time, depending on gene expression, RNA splicing, and enzymatic cleavage. Despite the existence of various forms of alpha-Klotho protein, only the full length, membrane-bound, isoform 1 is known to form a complex with fibroblast growth factor (FGF) receptors and functions as an obligatory co-receptor for FGF23→a bone-derived hormone that induces phosphate excretion into urine and which has a regulatory role on Pᵢ and vitamin D metabolism.

Klotho is highly expressed in the kidney, brain, and to a lesser extent in other organs, and may also be found in the blood, cerebrospinal fluid and urine of mammals. Circulating levels of soluble Klotho proteins in mammals, including humans, are thought to decrease with age. In addition, Klotho-deficient mice exhibit accelerated aging phenotypes, whereas over-expression of klotho in mice has been shown to extend lifespan. In addition, Klotho has been implicated in a number of cellular processes related to aging. In light of the foregoing, a developing hypothesis states that soluble Klotho may function as an anti-aging compound in the human body.

Aging is an inevitable and progressive biological process resulting in dysfunction and destruction of almost all tissues and organs, ultimately resulting in death. The aging of the human body, for instance, is associated with the decline of cellular function, which can lead to the development of a variety of diseases. Aging is thought to be driven by a tightly regulated and complex interplay between genetic and acquired factors and is typically characterized by an increase in senescence, a quantitative and qualitative decrease in stem cells, and abnormal structure at tissue levels.

As the so-called “baby boomers” generation continues to advance in age, the population of aging individuals (e.g., age 60-65) is rapidly increasing globally. The increased demand for health care for this aging population places significant financial burden on any healthcare system. Recombinant klotho proteins may provide promising therapeutic agents to counter age-related health conditions. To date, all relevant treatment data related to Klotho is pre-clinical, research trials in animal models. Developing strategies and health intervention products and methods based on (i) the production and/or purification (e.g., to substantial homogeneity) of recombinant s-Klotho proteins or protein variants and (ii) the administration of recombinant s-Klotho proteins or protein variants and/or s-Klotho protein level-increasing health supplements to subjects, especially humans and/or within an increasing aging population, may help to ameliorate this situation.

Currently, there is not a product or method for providing an exogenous form of human Klotho protein, such as recombinant soluble human alpha-Klotho protein or protein variant, suitable for human use, especially protein that is Current Good Manufacturing Practice (cGMP) regulation compliant, as determined and enforced by the U.S. Food and Drug Administration (FDA), whether alone or in combination with one or more additional active components.

Likewise, there is not currently a product or method for (naturally) increasing endogenous Klotho protein levels or protein production, in a safe and effective manner, especially a product that complies with the Dietary Supplement Health and Education Act of 1994 (DSHEA) or that is Current Good Manufacturing Practice (cGMP) regulation compliant (e.g., as determined and enforced by the U.S. Food and Drug Administration (FDA)), whether alone or in combination with one or more additional active components, drugs, pharmaceuticals, or nutraceuticals.

Moreover, there is not currently a product (e.g., kit or system) or method for efficiently, reliably, reproducibly, practically, and/or (commercially or economically) viably monitoring Klotho protein levels, particularly a product or method for on-demand and/or real-time monitoring and/or quantification of endogenous and/or exogenous Klotho protein levels, and more particularly endogenous and/or exogenous soluble alpha Klotho protein or protein variant levels, or for diagnosing Klotho protein deficiency, particularly serum soluble Klotho protein deficiency, in human and non-human animals.

There is also not currently a suitable, large scale method for efficiently, reliably, reproducibly, practically, and/or (commercially or economically) viably producing and purifying recombinant Klotho proteins, particularly, pharmaceutical grade recombinant Klotho proteins, including recombinant Klotho protein fragments and recombinant Klotho fusion proteins.

### BRIEF SUMMARY

Embodiments of the present disclosure solve one or more of the foregoing or other problems in the art with products and methods for assessing and increasing Klotho protein levels in a patient. Some Embodiments include (1) compositions and methods for stabilizing Klotho protein in human or non-human animal (e.g., mammalian) blood or serum, (2) compositions and methods for monitoring, detecting, and/or quantifying Klotho protein level(s), particularly endogenous and/or exogenous soluble alpha Klotho protein level(s), and/or for diagnosing Klotho protein deficiency in a human or non-human animal (e.g., mammalian) subject, and to products useful in performing the same, (3) pharmaceutical or nutraceutical compositions for increasing Klotho protein levels in the subject. Some embodiments include Klotho protein(s), or fragment(s) or variant(s) thereof, as a therapeutic agent, and more particularly, cGMP-grade, human (or non-human) recombinant soluble alpha-Klotho protein(s), or fragment(s) or variant(s) thereof, or a (pharmaceutical) composition comprising the same. Some embodiments include products and/or methods for manufacturing and administering (recombinant) Klotho protein(s), or fragment(s) or variant(s) thereof, to a human or non-human subject, and particularly for increasing serum soluble Klotho protein levels in the subject. Some embodiments include a nutraceutical or health supplement composition for increasing serum soluble Klotho protein levels in the subject. In particular, some embodiments include products that, when administered to the subject, cause an increase in Klotho protein (particularly endogenous soluble alpha Klotho protein) level(s), expression, or production, in the subject, and (4) methods of purifying recombinant Klotho proteins, particularly pharmaceutical grade recombinant Klotho proteins, recombinant Klotho protein fragments, and recombinant Klotho fusion proteins.

Various embodiments include a composition. The composition can comprise (i) one or more recombinant human Klotho proteins, protein fragments, and/or protein variants, expression nucleic acid constructs and/or vectors, cell lines and/or cell suspension cultures, and/or (ii) one or more ingredients that, when administered to the subject, cause or induce an increase Klotho protein level(s), expression, or production, particularly endogenous soluble alpha Klotho protein level(s), expression, or production, in the subject. Certain embodiments include methods of manufacturing, purifying, and administering a composition of the present disclosure to the (human or non-human animal) subjects. Some embodiments include methods for increasing Klotho protein (particularly (exogenous and/or endogenous) soluble alpha Klotho protein) level(s), expression, or production, in the subject. Some embodiments include products (e.g., diagnostic kits) and/or methods for monitoring, detecting, and/or quantifying Klotho protein level(s), particularly (endogenous and/or exogenous) soluble alpha Klotho protein level(s), and/or for diagnosing Klotho protein deficiency in the subject.

Some embodiments of the present disclosure can include one or more of: a recombinant human alpha soluble Klotho protein, protein fragment, and/or protein variant (e.g., cGMP-grade human recombinant soluble alpha-Klotho protein, protein fragment, and/or protein variant); a composition (e.g., therapeutic composition, pharmaceutical composition, medicament, formulation, etc.) comprising a recombinant human alpha soluble Klotho protein, protein fragment, and/or protein variant; a composition comprising a recombinant human alpha soluble Klotho protein and a (pharmaceutically-acceptable) vehicle (e.g., a carrier or excipient); a composition comprising a recombinant human alpha soluble Klotho protein and at least one additional (active) ingredient; a nucleic acid construct or vector that encodes a recombinant human alpha soluble Klotho protein; a cell line that (i) contains a nucleic acid construct or vector that encodes a recombinant human alpha soluble Klotho protein and/or (ii) expresses a recombinant human alpha soluble Klotho protein; a cell suspension culture comprising one or more cells that (each) (i) contain a nucleic acid construct or vector that encodes a recombinant human alpha soluble Klotho protein and/or (ii) express a recombinant human alpha soluble Klotho protein; a method of manufacturing, and optionally purifying, a recombinant human alpha soluble Klotho protein; a method of manufacturing a medicament (or therapeutic composition—i.e., formulation) of recombinant human alpha soluble Klotho protein; a method of administering a recombinant human alpha soluble Klotho protein to a (human or non-human animal) subject; a diagnostic method for determining Klotho protein deficiency in a subject; a method of diagnosing Klotho protein deficiency in a subject; a method of diagnosing a subject as being in need of receiving a recombinant human alpha soluble Klotho protein by administration; a method for evaluating the efficacy and/or determining an effective dosage of the protein to a subject in need thereof; a recombinant human alpha soluble Klotho protein for use in treating a specific medical or other condition in a human or non-human animal (e.g., a non-human mammal); use of a recombinant human alpha soluble Klotho protein for the treatment of a specific medical or other condition in a human or non-human animal; use of a composition comprising a recombinant human alpha soluble Klotho protein for the treatment of a specific medical or other condition in a human or non-human animal; and/or use of a recombinant human alpha soluble Klotho protein in the manufacture of a medicament for the treatment of a specific medical or other condition in a human or non-human animal.

Some embodiments can include a recombinant Klotho protein, wherein at least a portion of the protein has at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152. Some embodiments can include recombinant protein comprising a Klotho protein sequence having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, preferably one of SEQ ID NO: 52, SEQ ID NO: 54, SEQ ID NO: 66, SEQ ID NO: 125 through SEQ ID NO: 128, SEQ ID NO: 133, and SEQ ID NO: 152 and, optionally at least a portion of an immunoglobulin Fc domain sequence, human serum albumin sequence, His (e.g., 6His) tag sequence, and/or a Twin-Strep tag sequence, with or without an optional linker sequence therebetween. Some embodiments can include two or more recombinant Klotho proteins.

Some embodiments can include a pharmaceutical composition, comprising a pharmaceutically effective amount of the recombinant Klotho protein as described herein and a pharmaceutically-acceptable carrier. Some embodiments can include a pharmaceutical composition, comprising a pharmaceutically effective amount of a recombinant soluble Klotho protein, at least a portion of the protein having at least 80% amino acid sequence identity to at least a subset of amino acid residues 1-981, 29-981, 34-981, 36-981, 131-981, 1-549, 29-549, 34-549, 36-549, or 131-549 of human alpha Klotho isoform 1 or isoform 2, or at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, and a pharmaceutically-acceptable carrier. Some embodiments can include a pharmaceutical composition, comprising a pharmaceutically-acceptable carrier or excipient and an effective amount of a recombinant protein comprising a Klotho protein sequence having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, preferably one of SEQ ID NO: 52, SEQ ID NO: 54, SEQ ID NO: 66, SEQ ID NO: 125 through SEQ ID NO: 128, SEQ ID NO: 133, and SEQ ID NO: 152 and, optionally at least a portion of an immunoglobulin Fc domain sequence, human serum albumin sequence, His (e.g., 6His) tag sequence, and/or a Twin-Strep tag sequence, with or without an optional linker sequence therebetween. Some embodiments can include a composition, comprising a pharmaceutically effective amount of two or more recombinant Klotho proteins.

Some embodiments can include compositions that comprise a therapeutic Klotho protein and at least one other active component, such as a drug, antibody, hormone, human cell, tissue, cellular or tissue-based product (HCT/Ps), etc., and/or methods of administering the same to human or non-human subjects. Combinatorial compositions and methods can be useful for treating subjects having an age-related disorder or condition, a clinical (e.g., metabolic) disorder, a chronic disease, an acute injury, and so forth. The prophylactic administration of combination treatments to subjects with no apparent condition or disorder can also be useful to delay or prevent certain conditions or disorders described herein.

Some embodiments can include a nucleic acid or nucleic acid construct. For instance, embodiments can include an expression vector or nucleic acid. The nucleic acid can encode a recombinant human alpha soluble Klotho protein, protein fragment, or protein variant. The nucleic acid can encode a native or non-native signaling sequence. For instance, the nucleic acid can encode a non-native signaling sequence upstream of (or N-terminal to) an encoded Klotho protein sequence. Some embodiments can include a nucleic acid construct, comprising a nucleic acid sequence encoding a recombinant protein, the recombinant protein comprising a Klotho protein sequence having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, preferably one of SEQ ID NO: 52, SEQ ID NO: 54, SEQ ID NO: 66, SEQ ID NO: 125 through SEQ ID NO: 128, SEQ ID NO: 133, and SEQ ID NO: 152; and, optionally at least a portion of an immunoglobulin Fc domain sequence, human serum albumin sequence, His (e.g., 6His) tag sequence, and/or a Twin-Strep tag sequence, with or without an optional linker sequence therebetween.

Some embodiments can include a cell line. The cell line can comprise (a plurality of) Chinese hamster ovary (CHO) cell(s), HEK-293 cells, or other suitable (protein expression) cell line. In some embodiments, the cells can be dihydrofolate reductase (DHFK)-deficient CHO cells, such as CHO-S cells, or glutamine synthetase (GS)-deficient CHO cells, such as GS−/−CHO cells. The cells can contain one or more (copies of an) exogenous nucleic acid (comprising a transgene or cDNA) that encodes a polypeptide with at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152. The polypeptide can comprise a human recombinant alpha soluble Klotho protein. The exogenous nucleic acid can include a transgene or cDNA, preferably having at least 80% nucleic acid sequence identity to one of SEQ ID NO: 76 through SEQ ID NO: 106 or SEQ ID NO: 121 through SEQ ID NO: 124. In some embodiments, the nucleic acid can (also) include or encode a promoter (associated with the transgene) and/or an optional (exogenous) enzyme, such as a (functional) dihydrofolate reductase (DHFR) enzyme, (functional) glutamine synthetase (GS) enzyme, etc.

At least one embodiment includes a suspension cell culture comprising the cell line growing in a liquid medium, preferably comprising a carbon source, a nitrogen source, and/or one or more vitamins, minerals, salts, amino acids, supplements, or additives, such that the cells express the polypeptide encoded by the nucleic acid. The liquid medium can be (human, bovine, fetal bovine, or other) serum-free and/or animal (or animal-derived) protein (component)-free. For instance, the liquid medium can be free of bovine serum albumin, human serum albumin, etc. In some embodiments, the suspension cell culture can comprise a liquid medium, preferably a serum-free and/or animal protein component-free liquid medium, wherein the liquid medium preferably comprises a carbon source, a nitrogen source, and one or more vitamins, minerals, salts, amino acids, supplements, or additives, more preferably wherein the liquid medium lacks hypoxanthine, thymidine, and/or glutamine, and the cell line growing in the liquid medium such that the cells express the polypeptide encoded by the nucleic acid, the polypeptide comprising a recombinant Klotho protein.

Some embodiments include an extract of or from the cells, the liquid medium, or both of the suspension cell culture, the extract containing a recombinant protein having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152. Certain embodiments include a human recombinant alpha soluble Klotho protein-containing extract of or from cells, liquid medium, or both (e.g., of the suspension cell culture). At least one embodiment includes an isolated recombinant protein having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152.

Some embodiments can include a method of manufacturing recombinant Klotho protein (e.g., a recombinant human alpha soluble Klotho protein). Illustrative methods can comprise producing a recombinant Klotho protein in, for example, Chinese hamster ovary (CHO) cells or HEK-293 cells, preferably in dihydrofolate reductase (DHFR)-deficient CHO cells, more preferably in CHO-S cells, or preferably in glutamine synthetase (GS)-deficient CHO cells, more preferably in GS−/−CHO cells, the protein preferably having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152.

In some embodiments, the manufacturing method can include growing the cells in a liquid medium, producing the recombinant soluble Klotho protein in the cells, and/or purifying a recombinant soluble Klotho protein-containing extract from the cells, liquid medium, or both. The extract can include at least about 90%, preferably at least about 92%, 93%, 94%, 95%, 96%, 97%, 98%, or 99% dry weight recombinant soluble Klotho protein and/or less than about 1-100 ppm host cell proteins (HCP). The cells can be HEK-293 cells or dihydrofolate reductase (DHFR)-deficient CHO cells, such as CHO-S cells, or glutamine synthetase (GS)-deficient CHO cells, such as GS−/−CHO cells. The produced (expressed) protein can be released (e.g., secreted) from the cells into the liquid medium and/or can have one or more glycans attached thereto.

The cells can contain one or more exogenous nucleic acids that encode the protein and, optionally, a functional enzyme, such as dihydrofolate reductase enzyme, glutamine synthetase (GS) enzyme, etc. The exogenous nucleic acid can include a promoter (e.g., a strong promoter, weak promoter, etc.), such as a promoter customary or typical for use for expression of exogenous protein in cell. The exogenous nucleic acid can include a transgene or cDNA (e.g., under control of the promoter), preferably having at least 80% nucleic acid sequence identity to one of SEQ ID NO: 76 through SEQ ID NO: 106 or SEQ ID NO: 121 through SEQ ID NO: 124, or any other suitable nucleic acid sequence encoding a Klotho protein as described herein (e.g., S-Klotho variants).

The method can include introducing, such as by transfection, the exogenous nucleic acid into the cells. The method can include growing the cells in a liquid medium, such as a (human, bovine, fetal bovine, or other) serum-free and/or animal (or animal-derived) protein (component)-free medium. The medium preferably comprises a carbon source, a nitrogen source, and/or one or more vitamins, minerals, salts, amino acids, supplements, or additives, preferably in a bioreactor. Depending on the particular cell line, the method can include introducing an effective amount of methotrexate (MTX), methionine sulphoximine (MSX), or other agent into the liquid medium and/or selecting (e.g., by cell sub-cloning, limited dilution, fluorescence activated cell sorting (FACS), etc.) a suspension culture of viable cells growing in the liquid medium.

In some embodiments, selection and/or gene amplification can be performed by culturing the transfected cells in a selection medium, such as a medium lacking hypoxanthine and/or thymidine (e.g., -HT medium), glutamine, etc. In at least one embodiment, a low concentration(s) of MTX can be added or used to amplify the transfected nucleic acid (or gene(s) thereof) and, thereby, select for increased protein expression (e.g., in DHFR-deficient cells transfected with a DHFR transgene). Alternatively (or in addition), selection and/or gene amplification can be performed by adding MSX (an inhibitor of (endogenous) glutamine synthetase (GS)) to suspension cultures of cells having at least one (exogenous) glutamine synthetase (GS) transgene.

The method can include sub-culturing surviving cells or cultures (e.g., MTX-resistant and/or MSX-resistant cells or cultures). The selected suspension culture and/or selected cells can have or exhibit an increased production of the protein (e.g., by the cell), an increased concentration of the protein (e.g., in the liquid medium), and/or an increased copy number of the exogenous nucleic acid (e.g., per cell) (e.g., as compared to a non-selected suspension culture or cell).

The liquid medium can also include an effective amount of MTX and/or MSX in some embodiments. The suspension culture (or cells thereof) can (be selected to): exhibit an increased production of the protein (e.g., by the cells); exhibit an increased concentration of the protein (e.g., in the liquid medium); secrete the protein (e.g., into the liquid medium); and/or have an increased copy number of the exogenous nucleic acid (e.g., per cell), preferably as compared to a non-selected suspension culture. The protein can have one or more glycans attached thereto.

Some embodiments of the present disclosure relate to compositions configured or formulated to augment, improve, or increase natural soluble alpha Klotho protein production. Some embodiments include a composition or method for increasing (endogenous) Klotho protein levels in a subject. Some embodiments include a (nutraceutical or health supplement) composition for increasing Klotho protein levels, particularly serum soluble Klotho protein levels, in a subject. Some embodiments include products that, when administered to the subject, cause an increase in Klotho protein level(s), expression, or production, particularly endogenous soluble alpha Klotho protein level(s), expression, or production, in the subject.

Some embodiments of the present disclosure relate to compositions configured or formulated to attenuate Klotho protein damage or degradation. Some embodiments of the present disclosure relate to methods of manufacturing compositions of the present disclosure. Some embodiments of the present disclosure relate to use of compositions of the present disclosure or methods of using the same to treat human or non-human animal subjects. Some embodiments of the present disclosure relate to treatment methods involving compositions of the present disclosure, including administration thereof to human or non-human animal subjects. Such uses and treatment methods can increase endogenous Klotho protein levels, such as by augmenting natural soluble alpha Klotho protein production and/or attenuating Klotho protein damage or degradation.

Certain embodiments include compositions of matter that comprise a health supplement that, when administered to human or non-human animal (e.g., mammalian) subjects, increases the levels of endogenous, soluble alpha-Klotho protein in the human or non-human animal subjects. The composition or health supplement formulation can comprise (synthetic) chemical and/or natural components or ingredients. In other words, components or ingredients of the composition or health supplement can be or originate from (synthetic) chemical and/or natural sources. An exemplary composition can be adapted (or chemical configured), or include a plurality of components adapted (or chemical configured), to raise serum soluble Klotho protein levels by increasing or enhancing endogenous production of soluble Klotho protein by the mammalian subject.

An exemplary composition can comprise one or more components, preferably selected from the group consisting of, for example, Vitamin D3, Vitamin E, Vitamin C, Vitamin K, 3,5,4′-trihydroxy-trans-stilbene (Resveratrol), Pterostilbene, N-acetylcysteine (NAC), Troglitazone, Rosiglitazone, Notopterygium incisum Ting, Gentian (Root; Extract), Cordyceps (Cordyceps Sinensis (CS) Mycelium), Isoflavones (Soy), Genistein, Daidzein, Quercetin (dihydrate; dill, bay leaf, oregano), Docosahexaenoic acid (DHA), Astaxanthin, Sesamin (Semen Sesamin Nigrum (Black)), Sesame (Seed Extract), Rosmarinic acid (RA; (Marjoram)), Tetradecylthioacetic Acid (TTA), Dicalcium Phosphate (Anhydrous), Mircrocrystalline cellulose (MCC), Croscarmellose Sodium (Primellose), Stearing Acid, Magnesium Stearate, Alpha Lipoic Acid, Conjugate (alpha) Linoleic Acid (CLA), Probiotics, Activated Charcoal, and others as known in the art.

An exemplary composition can comprise one or more components, preferably in suitable nutraceutical concentrations, and preferably selected from the group consisting of, for example, vitamin C, vitamin D3, vitamin E, N-acetylcysteine (NAC), quercetin (dihydrate), rosmarinic acid (RA), pterostilbene, docosahexaenoic acid (DHA), nicotinamide riboside (NR), nicotinamide adenine dinucleotide (NAD+), nicotinamide mononucleotide (NMN), and one or more probiotic. The one or more probiotic comprises an effective amount of the Bifidobacterium species and/or strains Bifidobacterium lactis BL-04, Bifidobacterium bifidum/lactis BB-02, and Bifidobacterium longum BL-05, and the Lactobacillus species and/or strains Lactobacillus acidophilus LA-14, Lactobacillus rhamnosus LR-32, and Lactobacillus paracasei LPC-37.

Certain ingredients or components can have positive benefits and very low side effects in treating or affecting one or more conditions that have been associated with cellular and tissue dysfunctions that occur with aging. Certain ingredients or components can (directly or indirectly) increase the circulating plasma level of soluble alpha Klotho (s-Klotho) levels in human or non-human animal subjects following administration thereof.

In at least one embodiment, a composition of the present disclosure can be or comprise a Dietary Supplement Health and Education Act of 1994 (DSHEA)-compliant nutritional supplement, which may be sold OTC, marketed and/or sold direct-to-consumer (DTC), and/or purchased without a prescription/physician order. In some embodiments, a composition of the present disclosure can be or comprise an approved Food and Drug Administration (FDA) pharmaceutical.

In some embodiments, ingredients can be co-administered, preferably via at least some co-formulation. In some embodiments, ingredients can be co-administered in separate formulations. For example, in some embodiments, ingredients can be co-administered in a multi-pill, -capsule, or dosage pack. Some ingredients can be provided in solid, granular, powdered, liquid, or other form.

Some embodiments can include one or more (additional) ingredients or components, as described herein. For instance, some embodiments can include one or more recombinant (e.g., human or mammalian) Klotho proteins, protein fragments, and/or protein variants, as described herein. Certain embodiments can include expression nucleic acid constructs and/or vectors, cell lines and/or cell suspension cultures, and methods of manufacturing, purifying, and administering the one or more recombinant (e.g., human or mammalian) Klotho proteins, protein fragments, and/or protein variants to (human or non-human animal) subjects. Some embodiments can include one or more active ingredients, such as a pharmaceutical or prescription medications (e.g., ARBs (e.g., Losartan, Valsartan), testosterone, Vit. D receptor agonists (e.g., calcitriol, paricalcitol), PPAR (gamma) agonists (e.g., thiazolidinediones, troglitazone, rosiglitazone), and/or others described in the patent references incorporated above).

Some embodiments can include a composition, comprising a pharmaceutically effective amount of one or more recombinant Klotho protein and an effective amount of a composition (e.g., health supplement) formulated to increase endogenous Klotho protein levels or production (e.g., in mammalian subjects). In some embodiments, the recombinant Klotho protein can be co-administered with the composition. In some embodiments, the recombinant Klotho protein can be combined with a pharmaceutically-acceptable carrier. In some embodiments, the administered recombinant Klotho protein can raise serum soluble Klotho protein levels by providing exogenous Klotho protein, while the composition can raise serum soluble Klotho protein levels by increasing or enhancing (natural) production of Klotho protein by the patient or subject.

Some embodiments can include a treatment method. The method can comprise administering to a subject in need thereof a (pharmaceutically) effective amount of a composition of the present disclosure. The composition can include (i) a pharmaceutically effective amount of one or more recombinant soluble Klotho protein or protein variant of the present disclosure and/or (ii) a pharmaceutically effective amount of a composition (e.g., health supplement) formulated to increase endogenous Klotho protein levels or production (e.g., in mammalian subjects).

Some embodiments can include a method of treating Klotho deficiency (e.g., in a human or non-human animal (e.g., mammalian) patient or subject). The method can comprise administering a composition to the mammalian subject, optionally or preferably after diagnosing the mammalian subject with Klotho deficiency. The composition can comprise, for example, one or more of: a pharmaceutical composition comprising a pharmaceutically-acceptable carrier or excipient and a pharmaceutically effective amount of a recombinant Klotho protein, recombinant Klotho protein fragment, or recombinant Klotho fusion protein disposed in the carrier or excipient; and a nutraceutical composition comprising a plurality of components adapted to raise serum soluble Klotho protein levels by increasing or enhancing endogenous production of soluble Klotho protein by the mammalian subject.

Some embodiments can include a method of treating an aging-related or other condition, disease, or disorder. Some embodiments can include a method of treating and/or preventing acute kidney injury (AKI), chronic kidney disease (CKD), or other condition. The subject to whom the composition is administered can be suffering from or at risk for a variety of conditions (e.g., disorders, diseases, injuries, illnesses, etc.). For example, some embodiments include a method of treating one or more chronic diseases and/or aging-related condition, such as a physical, mental, neurological, or other condition associated with (human) aging. Some embodiments can promote healing, recovery, longevity, and/or other beneficial outcome through one or more mechanisms or action. Administration of the inventive composition(s) can have a positive therapeutic effect on the course and outcome of the condition, including chronic and/or age-related disease and longevity in human subjects, and characterization of the same.

The pharmaceutically effective amount of the composition(s) can be sufficient to raise or increase the serum soluble Klotho protein concentration of the subject to a predetermined level, such as greater than, equal to, or between about 50 to 3000 picograms of soluble Klotho protein per milliliter of serum. The amount can also or alternatively be sufficient to maintain the serum soluble Klotho protein concentration of the subject at or above a predetermined threshold for a predetermined period of time. Embodiments can also include administering the composition(s) to a subject in need thereof so as to maintain the serum soluble Klotho protein concentration of the subject at or above a predetermined threshold for a predetermined period of time.

Embodiments can also include determining a serum soluble Klotho protein concentration of the subject. Embodiments can also include calculating a pharmaceutically effective amount of a composition of the present disclosure. Embodiments can also include determining a rate of soluble Klotho protein decline in the serum of the subject. Embodiments can also include calculating a subsequent dosage time at which the serum soluble Klotho protein concentration of the subject will be at or below a second predetermined level based on the determined rate. Embodiments can also include calculating a subsequent dosage time and/or amount of the composition sufficient to raise the serum soluble Klotho protein concentration of the subject from the second predetermined level to the first predetermined level. Embodiments can also include administering the subsequent dosage amount of the composition to the subject. Certain embodiments can include prescribing a regular (e.g., daily) dose or dosage form of the composition to the subject (e.g., based on determined levels of serum soluble Klotho protein concentration of the subject).

In certain embodiments, the composition (e.g., protein or supplement) or raised soluble Klotho protein levels can (be effective to) modulate the IGF-1 and/or Wnt signaling pathways, exhibit β-glucuronidase and/or sialidase activity, suppress the p53/p21 signaling pathway, and/or reduce H₂O₂-induced cell senescence and apoptosis, preferably through suppression of the p53/p21 signaling pathway. The protein or raised protein levels can function or be functional as a humoral factor, preferably exhibiting pleiotropic activity and/or preferably in the regulation of oxidative stress, growth factor signaling, ion homeostasis, and/or regulation of activity of glycoproteins on the cell surface, such as one or more ion channel proteins and/or growth factor receptors, such as Insulin/Insulin-Like Growth Factor-1 receptor.

In certain embodiments, the composition (e.g., protein or supplement) or raised soluble Klotho protein levels can (be effective to) treat one or more aging-related condition (or condition associated with (human) aging), such as frailty, bone density loss or bone mineral density loss, weight loss, muscular atrophy or degeneration, decline in muscle mass, decline in muscle strength, hand strength, leg strength, or physical fitness, decline in movement, freedom of movement, quality of life assessment, ejection fraction, or exercise capacity, decline in learning, learning capacity, memory, or intellectual quotient, cognitive deterioration or forgetfulness, decline in cognitive capacity or function, decline in synaptic plasticity or synaptic function, and cellular senescence.

In certain embodiments, the composition (e.g., protein or supplement) or raised soluble Klotho protein levels can (be effective to) treat one or more aging-related condition (or condition associated with (human) aging), such as Alzheimer's disease, Parkinson's disease, dementia or vascular dementia, amyotrophic lateral sclerosis (ALS) or motor neuron disease (MND), atrial fibrillation, chronic obstructive pulmonary disease (COPD), fibromyalgia, adult onset diabetes, arthritis or rheumatoid arthritis, osteoarthritis, osteoporosis, glaucoma, cataracts, macular degeneration and other eye diseases/disorders, multiple sclerosis (MS), lupus, and/or ulcerative colitis.

In certain embodiments, the composition (e.g., protein or supplement) or raised soluble Klotho protein levels can (be effective to) treat one or more other diseases or conditions. For instance, some embodiments of the present disclosure can be useful in one or more of treating cancer, lowering serum phosphate levels in a patient, treating diabetes or a diabetes-related condition (e.g., Type 1 diabetes mellitus, etc.) in a subject in need of such treatment, treating a heart condition (e.g., cardiovascular disease, left ventricular hypertrophy (LVH), pathological LVH and/or congestive heart failure, etc.) in a subject, treating acute lung injury in a subject (e.g., using nanoparticles), protecting the lung of a patient against oxidant injury, detecting early acute kidney injury in critically ill patients, attenuating vascular calcification in a subject, improving cognition, treating renal and/or liver ischemia, modulating stress response in (human) senescent endothelial cells, prophylactically and/or therapeutically treating, preventing, attenuating, arresting, and/or reversing acute and/or chronic kidney injury, disease, or disease progression and/or uremic cardiomyopathy, reversing or attenuating age-related therapy resistance in melanoma or other cancers, targeting apoptosis of senescent cells, preferably restoring tissue homeostasis thereby, and a variety of other indications.

Accordingly, embodiments can also include a composition for use in treating one or more aging-related or other conditions. An exemplary method of treating an aging-related or other condition, disease, or disorder, the method comprising administering to a subject in need thereof an effective amount of a composition of the present disclosure. The composition can include (i) a supplement, as described herein, and/or (ii) a recombinant soluble Klotho protein (e.g., having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152). Optionally, the composition can include a pharmaceutically-acceptable carrier or excipient.

Some embodiments include a method of detecting and/or quantifying endogenous and/or exogenous Klotho protein, and more particularly endogenous and/or exogenous soluble alpha Klotho protein. Some embodiments include a method of diagnosing Klotho protein deficiency in a subject. Some embodiments include a product (e.g., a system or kit) for detecting and quantifying Klotho protein levels, and more particularly endogenous and/or exogenous soluble alpha Klotho protein levels. Some embodiments include methods of diagnosing Klotho protein deficiency in a subject. Some embodiments can include a method of treating Klotho deficiency in a subject.

Certain embodiments can include or utilize a blood or serum sample of the (mammalian) subject, preferably capillary blood and/or blood obtained from a finger-prick or other non-phlebotomic (non-venesectic) blood draw technique. It will be appreciated, however, that phlebotomic (venesectic) and other suitable blood draw techniques are also contemplated herein.

Some embodiments include a kit. Embodiments can include, for example, a sample collection container. The container can have an opening configured for receiving capillary blood. The capillary blood can contain, or be suspected of containing, an amount of soluble Klotho protein in some embodiments. Embodiments can include a sample preservative or anti-coagulant, preferably comprising one or more of EDTA and Heparin. The sample preservative or anti-coagulant can be disposed in a sample collection compartment of the container in some embodiments. The solution can be configured to stabilize soluble Klotho protein for at least 24 hours, and up to 7 days or more, at room temperature or without freezing. Embodiments can optionally include a capillary blood sampling device, preferably comprising a lancing device or lancet.

Some embodiments include a method. For example, some embodiments can include a method of stabilizing Klotho protein in a mammalian blood sample. Embodiments can include, for example, drawing or obtaining capillary blood from a mammalian subject and storing the capillary blood in a container for at least 24 hours at room temperature or without freezing. The container can have a preservative or anti-coagulant disposed therein, such that the preservative or anti-coagulant is mixed with the capillary blood in the container. The preservative or anti-coagulant can stabilize the soluble Klotho protein for at least 24 hours at room temperature or without freezing. The preservative or anti-coagulant can be or comprise one or more of heparin, lithium heparin, EDTA, and K₂ EDTA.

Embodiments can also include collecting the capillary blood in a container, and/or mixing the capillary blood with a preservative or anti-coagulant to form a mixture. In some embodiments, drawing capillary blood can include lancing skin of the mammal with a lancet. In some embodiments, the step of collecting the capillary blood in the container can include collecting at least or about 10-1000 ul, preferably about 50-200 ul of the capillary blood in the container Some embodiments can include storing the mixture or allowing the mixture to be stored for at least 3 days at room temperature or without freezing, the preservative or anti-coagulant stabilizing the soluble Klotho protein for the at least 3 days at room temperature or without freezing.

Some embodiments can optionally include assaying the mixture for the presence of the soluble Klotho protein. The step of assaying the mixture for the presence of the soluble Klotho protein can include detecting the soluble Klotho protein and/or quantifying the amount of soluble Klotho protein. Detecting the soluble Klotho protein and/or quantifying the amount of soluble Klotho protein can include contacting the mixture with an antibody configured to bind the soluble Klotho protein. Detecting the soluble Klotho protein and/or quantifying the amount of soluble Klotho protein can include performing ELISA on the mixture using an antibody configured to bind the soluble Klotho protein. Other detection methods may include mass spectrometry or Multi-Analyte Profiling (xMAP), such as Luminex technology (e g , miniaturized liquid array bioassay, small lasers, light emitting diodes (LEDs), digital signal processors, photo detectors, and/or charge-coupled device imaging), etc.

Some embodiments include a method of diagnosing Klotho deficiency in a mammalian subject or a method of treating Klotho deficiency in a mammalian subject. Embodiments can include, for example, providing or obtaining a fluid biological sample. The sample can include mammalian serum, preferably in the form of capillary blood. The method can optionally include mixing the mammalian serum with a preservative or anti-coagulant to form a mixture. The sample can also or alternatively include a mixture comprising mammalian serum, preferably in the form of capillary blood, and a preservative or anti-coagulant. The method can include assaying the mixture for the presence of soluble Klotho protein. The step of assaying the mixture for the presence of the soluble Klotho protein can include detecting the presence of the soluble Klotho protein, if any, in the mixture and/or quantifying the amount of the soluble Klotho protein, if any, in the mixture, optionally after storing the Klotho-stabilized mixture for a period of time at room temperature or without freezing. The method can include diagnosing the mammalian subject with Klotho deficiency when no soluble Klotho protein is detected in the mixture or when the amount of quantified soluble Klotho protein in the mixture is less than a predetermined amount. In some embodiments, diagnosing the mammalian subject with Klotho deficiency can include displaying the Klotho deficiency determination or diagnosis on a user interface of the computer system and/or producing a file or report, in physical or electronic form, that displays the Klotho deficiency determination or diagnosis.

In some embodiments, providing or obtaining a fluid biological sample can include drawing capillary blood from the mammalian subject and/or collecting capillary blood in a container. In some embodiments, the step of assaying the mixture for the presence of soluble Klotho protein can include performing ELISA on the mixture using an antibody configured to bind the soluble Klotho protein. Some embodiments can include administering a composition to the mammalian subject when no soluble Klotho protein is detected in the mixture or when the amount of quantified soluble Klotho protein in the mixture is less than a predetermined amount. Mass spectrometry can also or alternatively be performed.

The disclosed strategies, products, and/or health intervention methods specifically configured to increase circulating and/or endogenous levels of soluble Klotho may help to ameliorate the situation and problems associated with decreased Klotho levels in patients.

Some embodiments may include any of the features, options, and/or possibilities set out elsewhere in the present disclosure, including in other aspects or embodiments of the present disclosure. It is also noted that each of the foregoing, following, and/or other features described herein represent a distinct embodiment of the present disclosure. Moreover, combinations of any two or more of such features represent distinct embodiments of the present disclosure. Such features or embodiments can also be combined in any suitable combination and/or order without departing from the scope of this disclosure. Thus, each of the features described herein can be combinable with any one or more other features described herein in any suitable combination and/or order. Accordingly, the present disclosure is not limited to the specific combinations of exemplary embodiments described in detail herein.

Additional features and advantages of exemplary embodiments of the present disclosure will be set forth in the description that follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above-recited and other advantages and features of the present disclosure can be obtained, a more particular description of the embodiments briefly described above will be rendered by reference to specific embodiments thereof which are illustrated in the appended drawings. For better understanding, the like elements have been designated by like reference numbers throughout the figure(s). Understanding that these drawings depict only typical embodiments of the disclosure and are not therefore to be considered to be limiting of its scope, the disclosure will be described and explained with additional specificity and detail through the use of the accompanying drawing(s) in which:

FIG. 1 depicts a schematic illustrating cellular production of various Klotho proteins according to an embodiment of the present disclosure;

FIG. 2 (A-D) depicts: A) schematic structures of isoform 1 and isoform 2 of human α-Klotho, and the location of the epitope for the antibody binding used in generating C-D (residues 800 to 900); B) The full-length α-Klotho protein sequence of 1012 amino acids, with KL1 and KL2 shown in red and green, respectively, and TM highlighted (black); C) and D) Western blot analysis of human cell lysates (C) and human tissues (D);

FIG. 3 illustrates stability of Klotho protein in human serum at various temperatures over a time.

FIG. 4 is a 20% SDS-PAGE of Klotho proteins.

FIG. 5A is a dose-response curve for 6xHis tagged Klotho.

FIG. 5B is a dose-response curve for Fc-tagged Klotho.

FIG. 6 is a dose-response curve comparing Klotho and Klotho-Fc.

FIGS. 7A-7B SDS-PAGE (reducing and non-reducing) illustrating purity and integrity of purified antibody.

FIGS. 8A-8B illustrate raw absorbance data at 450 nm and corresponding antibody titration curve.

FIGS. 9A and 9B illustrate ELISA assay results for HRP and Biotin-anti-Klotho IgY.

FIG. 10 is a graphical representation of the (daily) change in circulating Klotho protein levels in a human subject.

FIGS. 11A-11B illustrate results of quantitative ELISA.

FIG. 12 illustrates Klotho level at different incubation time at RT for donors A-D and donor A, spiked.

FIG. 13 illustrates an example report graph or chart.

FIG. 14 illustrates a chromatograph of Klotho-Fc protein.

FIG. 15A illustrates stable pool 291645 expressing the FL-ECD (34-981)-Fc Klotho derivative (SEQ ID NO: 52)

FIG. 15B illustrates Klotho protein under native and non-reducing conditions.

FIGS. 16A-16B illustrate Western blots of Klotho protein FL-ECD (34-981)-TEV-Twin-Strep expressed by stable pool 291647 (SEQ ID NO: 66) under native and non-reducing conditions.

FIG. 17 is a gel of the various indicated recombinant Klotho protein constructs.

FIG. 18 is a series of gels for the respective indicated recombinant Klotho protein constructs.

FIGS. 19-26 illustrate density, viability and production of various cell lines.

FIG. 27 illustrates copy number stability in various cell line clones.

FIGS. 28-32 illustrate stability of various cell line clones.

FIG. 33 illustrates an ELISA gel for purified Klotho proteins.

FIGS. 34A-34C illustrate Klotho protein concentration measured and spike recovery observed in the respective samples.

FIGS. 35A-35B illustrate preliminary global glycan analysis.

FIGS. 36A-36C illustrate growth, viability, and titer curves, respectively, for various cell lines.

FIGS. 37A-37C illustrate growth, viability, and titer curves, respectively, for isolated cell lines.

FIGS. 38A-38C illustrate growth, viability, and titer curves, respectively, for a chosen cell line.

FIGS. 39A-39B illustrate Western blot analysis was performed on expressed protein pools.

FIG. 40A illustrates Protein A Sepharose 4 Fast Flow purification of Klotho proteins.

FIG. 40B illustrates Superdex 200 purification of Klotho proteins

FIGS. 41A and 41B illustrate Superdex 200 fractionation for klotho-Fc protein.

FIG. 42 illustrates results of a protein binding bioassay for various Klotho proteins.

FIGS. 43A-43E illustrate proliferation of stimulated cell in a dose-dependent manner.

FIG. 44 illustrates, graphically, the average (mean) body weight (+/−standard error of the mean; SEM bars) of rats in each group over the course of the 12-day study.

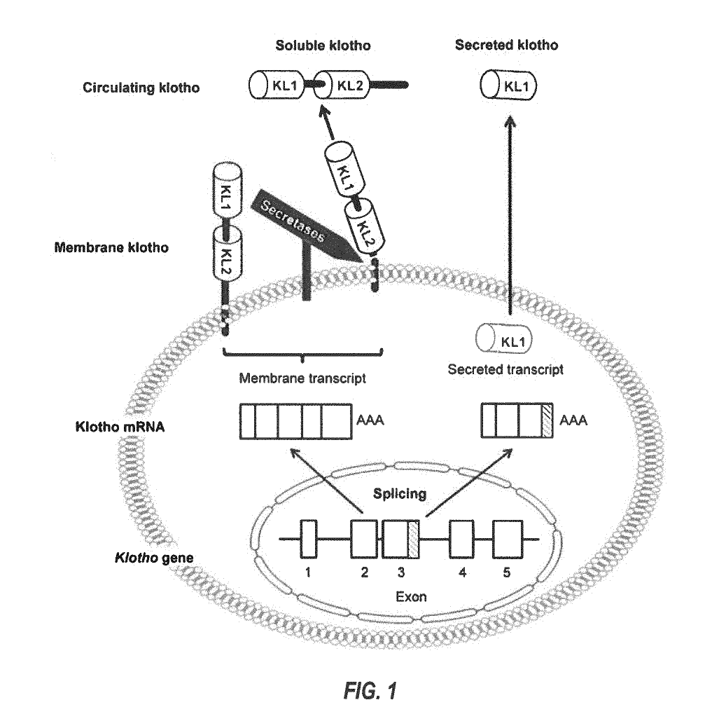

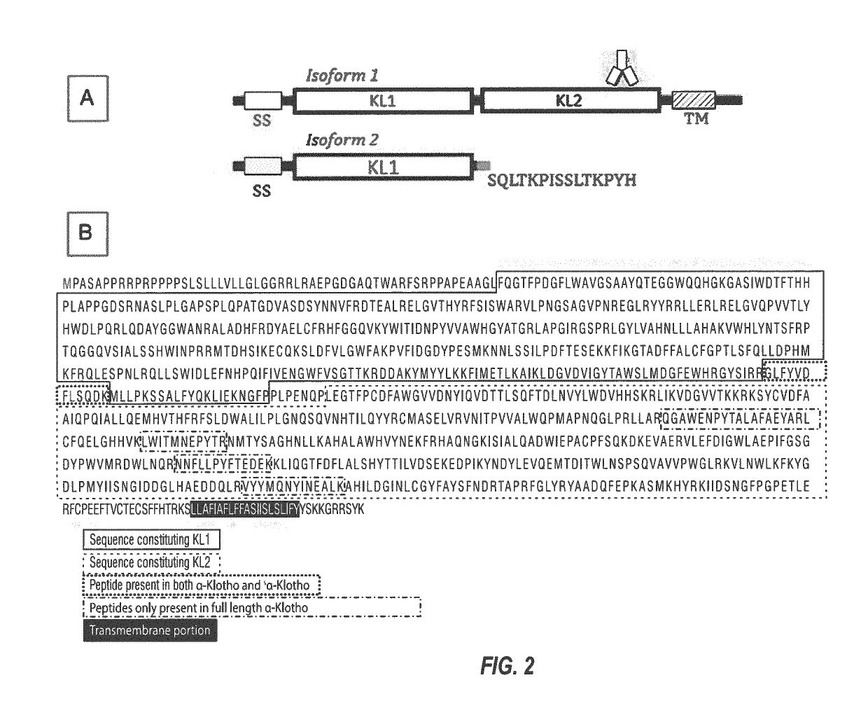

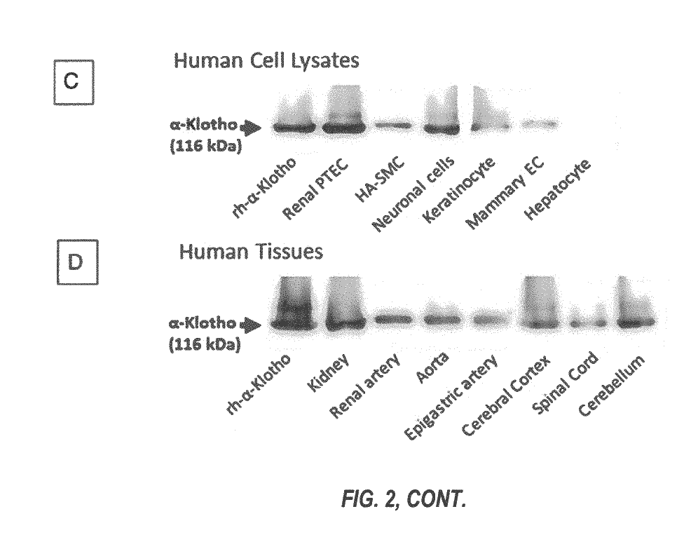

### DETAILED DESCRIPTION

Embodiments of the present disclosure relate to compositions and methods for increasing serum soluble Klotho protein levels in a human or non-human animal (or mammal) subject and to compositions and methods for monitoring, detecting, and/or quantifying Klotho protein level(s), particularly endogenous and/or exogenous soluble alpha Klotho protein level(s), and/or for diagnosing and/or treating Klotho protein deficiency in the subject. Some embodiments include one or more compositions, comprising (i) recombinant human Klotho proteins, protein fragments, and/or protein variants, expression nucleic acid constructs and/or vectors, cell lines and/or cell suspension cultures, and/or (ii) products (e.g., supplements) that, when administered to the subject, cause an increase Klotho protein level(s), expression, or production, particularly endogenous soluble alpha Klotho protein level(s), expression, or production, in the subject. Certain embodiments include methods of manufacturing, purifying, and administering the compositions of the present disclosure to the (human or non-human animal) subjects and/or methods for increasing Klotho protein level(s), expression, or production, particularly (exogenous and/or endogenous) soluble alpha Klotho protein level(s), expression, or production, in the subject. Some embodiments include methods for monitoring, detecting, and/or quantifying Klotho protein level(s), particularly (endogenous and/or exogenous) soluble alpha Klotho protein level(s), and/or for diagnosing Klotho protein deficiency in the subject.

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein. By way of illustrative example, disclosure of “up to 1,000 mg” of a particular ingredient or component includes a specific disclosure of: (i) any value greater than zero and less than or equal to 1,000 milligrams, including but not limited to 0.01 mg, 1 mg, 5 mg, 10 mg, 50 mg, 100 mg, 500 mg, 750 mg, 990 mg, and 1,000 mg; and/or (ii) any range of values from or between greater than zero and less than or equal to 1,000 milligrams, including but not limited to 0.01-1,000 mg, 1 mg −990 mg, 5 mg −750 mg, 10 mg −500 mg, and 50 mg −100 mg. Similarly, disclosure of “at least 80% amino acid sequence identity” or “80%-100% amino acid sequence identity” includes a specific disclosure of: (i) any whole percentage value between 80% and 100%, including 81%, 82%, 83%, 84%, 85%, 86%, 87%, 88%, 89%, 90%, 91%, 92%, 93%, 94%, 95%, 96%, 97%, 98%, 99%, and 100%, as well as any fraction of a percent value therebetween; and/or (ii) any range of percentage values between 80% and 100%, including, by way of non-limiting example, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, and 99%-100%.

### Abbreviated List of Defined Terms

To assist in understanding the scope and content of the foregoing and forthcoming written description and appended claims, a select few terms are defined directly below.

Unless defined otherwise, all technical and scientific terms used herein have the same meaning as commonly understood by one of ordinary skill in the art to which the present disclosure pertains.

Various aspects of the present disclosure, including systems, methods, and/or products may be illustrated with reference to one or more embodiments, which are exemplary in nature. As used herein, the terms “embodiment” mean “serving as an example, instance, or illustration,” and should not necessarily be construed as preferred or advantageous over other aspects disclosed herein. In addition, reference to an “ embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

Except in the examples, or where otherwise expressly indicated, all numerical quantities in this description indicating amounts of material or conditions of reaction and/or use are to be understood as modified by the word “about”. As used herein, the term “about,” with regard to a value, means +/−10% of the stated value or amount represented thereby. For instance, throughout the present disclosure, the term “about” is used in connection with a percent concentration or composition of a component or ingredient (e.g., in a mixture, such as a fluid or liquid mixture, aqueous mixture, solution, etc., optionally or preferably measured as a w/w percent, w/v percent, v/v percent, etc.). In such instance, the term “about” and/or the term “+/−10%” implies and/or includes +/−10% of the stated numeric value, as opposed to +/−10 percentage points of the recited percent. By way of example, where 20% w/w of a component or ingredient reflects 20 g of the component or ingredient per 100 mL of total mixture, the term “about” and/or the term “+/−10%” implies and/or includes a recited range from 18 g to 22 g (i.e., from 18% w/w to 22% w/w), not a range of 10% w/w to 30% w/w. Alternatives for so-called “about” values and/or +/−10% include +/−1%, +/−2%, +/−3%, +/−4%, +/−5%, +/−6%, +/−7%, +/−8%, or +/−9% of the stated value, each of which is contemplated as a suitable alternative to or substitute for the term “about” or the use of +/−10% herein.

As used herein, the terms “approximately” and “substantially” represent or imply an (or any) amount close to the stated amount (e.g., that still performs a desired function or achieves a (desired or expected) result). For example, the terms “approximately” and “substantially” may refer to an amount that is within, or less than, 10%, 5%, 1%, 0.1%, 0.01%, or other percent of a stated amount. As used herein, the term “substantially devoid” means (1) an undetectable or unquantifiable amount, (2) less than or below an amount generally considered by those skilled in the art to reflect a detectable or quantifiable amount, and/or (3) less than or below an amount generally considered by those skilled in the art to be functional or able to achieve a (desired or expected) result (e.g., less than 10%, 5%, 1%, 0.1%, 0.01%, or other percent).

By “Quantum satis” (also referred to as “q.s.” or “qs”) is meant the amount that is enough. Accordingly, a component or ingredient “qs 100%,” “provided at qs 100%,” or “qs to 100%” indicates that the component or ingredient is provided or included in an amount sufficient to complete the composition or to bring the total (of all components, whether recited or not) to 100%. It is noted, however, that a (final) component or ingredient “qs 100%,” “provided at qs 100%,” or “qs to 100%” does not indicate that the mixture consists of, consists essentially of, or only contains the components listed or recited immediately before the “qs 100%” component. In other words, “qs 100%,” and similar terms, is meant to be an open-ended expression indicating the source of the remainder, whatever that remainder may be.

As used herein, the term “product(s)” includes compositions, formulations, and mixtures, as well as devices, apparatus, assemblies, kits, systems, and so forth. Similarly, the term “method” includes processes, procedures, steps, and so forth.

As used herein, a “feature” or “aspect” of the present disclosure or embodiment disclosed herein refers to a property, attribute, component, ingredient, element, member, part, portion, (method) step, or other facet of the subject matter at hand.

The word “or” as used herein generally means any one member of a particular list, but also includes any combination of two or more members of said list. Similarly, the term “and” can be interchangeable with the terms “or,” either of which can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “plurality” and “at least two” are used interchangeably.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

The terms “medical professional” as used herein, generally refers to any individual or entity that is responsible for or participates in providing health care to an animal, including human and non-human animals, such as non-human mammals, with particular emphasis on licensed health care providers or unlicensed providers, such as assistants, technicians, and/or volunteers, particularly those covered under the (blanket) license or insurance of a health care provider. This term may, when contextually appropriate, include an oncologist, a surgeon, a physician's assistant, a nurse, a phlebotomist, a veterinarian, etc.

The term “cancer” refers to an abnormal, typically uncontrolled, growth of cells. A “cancerous cell” as used herein comprises a malignant cell having an abnormal, typically uncontrolled, growth. As such, the term cancer is an umbrella term encompassing a plurality of different distinctive diseases characterized by malignant cells growing in a typically uncontrolled manner.

The term “diagnosis,” or diagnosing,” and similar terms, as used herein, is not intended to convey or require (in all cases) a certified, regulatory-compliant, medical diagnosis, as required or regulated by the U.S. Food and Drug Administration (FDA) or any other government or non-government entity, organization, or agency. Rather, “diagnosis,” or diagnosing,” and similar terms, as used herein, relates to providing information that may be indicative of a condition and/or helpful in making a determination related to the condition.

The term “co-administration” and similar terms refer to concurrent, sequential, and/or combined administration of two or more components. For instance, two components can be co-administered by administering each component in a separate dosage concurrently, simultaneously, or sequentially (e.g., distinct administrations separated by a period of time). The period of time can be very small (e.g., substantially, immediately following a first administration) or longer (e.g., 1-60 seconds, 1-60 minutes, 1-24 hours, 1-7 days, 1-4 weeks, 1-12 months, and so forth, or any value or range of values therebetween). Concurrent or simultaneous administration can include overlapping administration timeframes for the two or more components or administration of a combination product comprising a mixture of the two or more components.

As used herein, “room temperature” may refer to any temperature above freezing (0° C., or other (equivalent) freezing temperature, depending on the presence of freezing point adjusting components) and below normal human body temperature (37° C., or other (equivalent) boiling temperature, depending on the presence of boiling point adjusting components), preferably above about 4° C. and below about 35° C., more preferably between about 5° C. and about 32° C., between about 8° C. and about 30° C., between about 10° C. and about 30° C., between about 10° C. and about 25° C., between about 10° C. and about 20° C., between about 10° C. and about 15° C., between about 15° C. and about 30° C., between about 15° C. and about 25° C., between about 15° C. and about 20° C., between about 20° C. and about 30° C., between about 20° C. and about 25° C., between about 20° C. and about 22° C., or any value or range of values therebetween.

As used herein, “refrigeration” may refer to any temperature above freezing (0° C.) and below about 32° C., preferably between about 1° C. and about 30° C., between about 1° C. and about 25° C., between about 1° C. and about 20° C., between about 1° C. and about 15° C., between about 2° C. and about 20° C., between about 2° C. and about 15° C., between about 3° C. and about 20° C., between about 3° C. and about 15° C., between about 4° C. and about 20° C., between about 4° C. and about 15° C., or any value or range of values therebetween.

As used herein, “nucleic acid,” and similar terms, refer to a naturally occurring or synthetic oligonucleotide or polynucleotide, whether DNA or RNA or DNA-RNA hybrid, single-stranded or double-stranded, sense or antisense. In particular, nucleic acids can include, without limitation, DNA, RNA, cDNA, gDNA, ssDNA, dsDNA or any combination thereof. Nucleic acids of the disclosure can also include nucleotide or nucleic acid analogs (e.g., BrdU), and non-phosphodiester (internucleoside) linkages or backbones (e.g., peptide nucleic acid (PNA) or thiodiester linkages), known in the art.

The terms “peptide” or “protein” as used herein refers to any peptide, oligopeptide, polypeptide, gene product, expression product, or protein. A peptide is comprised of consecutive amino acids. The terms “peptide” or “protein” encompass naturally occurring or synthetic molecules.

As used herein, the term “standard amino acid” includes: alanine—ala—A; arginine—arg13 R; asparagine—asn—N; aspartic acid—asp—D; cysteine—cys—C; glutamine—gln—Q; glutamic acid—glu—E; glycine—gly—G; histidine—his—H; isoleucine—ile—I; leucine—leu—L; lysine—lys—K; methionine—met—M; phenylalanine—phe—F; proline—pro—P; serine—ser—S; threonine—thr—T; tryptophan—trp—W; tyrosine—tyr—Y; and valine—val—V.

As used herein, “codon optimized” or “codon optimization” refers to the process of modifying or changing codons in a nucleotide sequence to codons that are preferred or more closely match the pattern of codon usage in the organism used for expression of the molecule. Thus, codons can be optimized for usage in a particular organism in which expression is desired based on known codon usage in the organism in order to enhance the effectiveness of expression of the nucleic acid, e.g., to achieve faster translation rates and high accuracy. The codon usage in a particular organism is known.

The encoding nucleic acid molecule can be a modified wild-type or a codon optimized sequence, where the codons are optimized for expression in a particular host cell, such as mammalian cells, e.g. , CHO cells or 293 cells, or in a yeast, or a plant cell, eukaryotic cells.

In particular examples, the nucleic acid sequence can be codon optimized, for example, to increase expression levels of the encoded sequence. The particular codon usage is dependent on the host organism in which the modified polypeptide is expressed. One of skill in the art is familiar with optimal codons for expression in mammalian or human cells, bacteria or yeast, including for example E. coli or Saccharomyces cerevisiae. For example, codon usage information is available from the Codon Usage Database available at kazusa.or.jp.codon (see e.g., Richmond (2000) Genome Biology, 1:241 for a description of the database. See also, Forsburg (2004) Yeast, 10:1045-1047; Brown et al. (1991) Nucleic Acids Research, 19:4298; Sharp et al. (1988) Nucleic Acids Res., 12:8207-8211; Sharp et al. (1991) Yeast, 657-78).

The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

### Illustrative Embodiments

Illustrative embodiments of the present disclosure include, but are not limited to, compositions, compositions of matter, products, articles of manufacture, formulations, combination products, co-administrations, co-formulations, dosages, process, methods, treatment protocols, diagnostic methods, kits, and so forth.

### Products and Methods for Stabilizing Klotho Protein in Blood

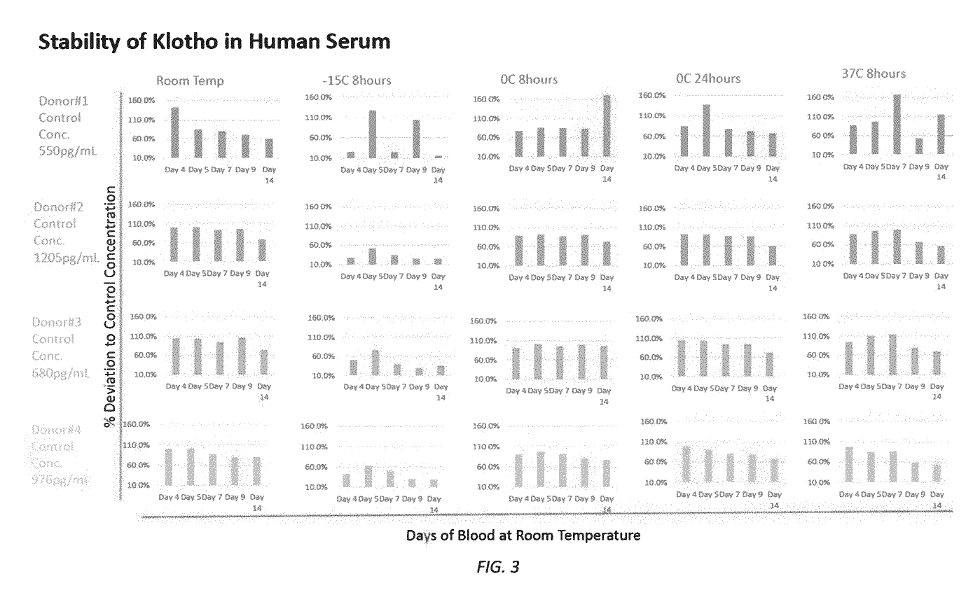

The stability of Klotho proteins (e.g., soluble Klotho) in blood or serum, especially at room temperature, and more especially for (extended) periods of time, has not been reported previously. FIG. 3 illustrates the surprising and unexpected stability of Klotho protein in human serum at room temperature, as well as other temperatures, over extended periods of time.

Without being bound to any theory, typical blood or serum protein diagnostics require a venous blood draw to collect a suitable amount of blood for monitoring, detecting, and/or quantifying protein (levels). Moreover, the drawn blood samples are typically processed soon after sampling (e.g., same day, within minutes or hours of sampling, etc.) or refrigerated (below room temperature) to preserve protein and/or sample stability. The Inventors have found, however, that Klotho protein in blood can be stable for up to 7 days, 9 days, 14 days, or more, at room temperature, in some embodiments of the present disclosure. In some embodiments, for instance, adding EDTA and/or Heparin to the sample can stabilize Klotho protein for at least 3 days, 4 days, 5 days, 6 days, 7 days, 8 days, 9 days, 10 days, 11 days, 12 days, 13 days, 14 days, 15 days, 16 days, 17 days, 18 day, 19 days, 20 days, 21 days, 22 days, 23 days, 24 days, 25 days, 26 days, 27 days, 28 days, 29 days, or 30 days at room temperature (data not shown). By way of non-limiting example, levels of Klotho in blood samples collected from four donors were relatively constant over a 7-day period (and over a 14-day period), at room temperature, with added EDTA and/or Heparin (see FIG. 3). Additionally, both tested anticoagulants, EDTA and Heparin, showed no significant difference in terms of Klotho concentration. This was surprising and unexpected to the inventors.

Room temperature storage may be important for commercial diagnostic applications, in which refrigeration of hundreds, thousands, tens of thousands, etc., of samples may not be commercially practical (logistically and/or monetarily). With a suitable room temperature stabilizing method, as described here, commercial diagnostic testing of Klotho blood or serum levels can be accomplished.

In light of the foregoing, an embodiment of the present disclosure can include a method and/or product that combines EDTA and/or Heparin to a biological sample (e.g., blood or serum) that contains Klotho protein. Some illustrative products can include a sample collection kit comprising a sample collection container and a sample preservation composition.

Some embodiments include a method of stabilizing Klotho protein in a mammalian blood sample. The method can include obtaining capillary blood from a mammalian subject. The capillary blood can contain (or be suspected of containing) an amount of soluble Klotho protein. The method can include storing the capillary blood (e.g., in a container) for at least 24 hours (or, preferably, at least 2, 3, 4, 5, 6, 7, 8, 9, 10, 11, 12, 13, 14, 18, 21, 28, or 30 days, or more), preferably at room temperature. The capillary blood sample can be mixed with a preservative or anti-coagulant. For example, the container can have a preservative or anti-coagulant disposed therein or added thereto, such that the preservative or anti-coagulant is mixed with the capillary blood in the container. The preservative or anti-coagulant can be or comprise one or more of heparin, lithium heparin, EDTA, and K₂ EDTA. The preservative or anti-coagulant can stabilize the soluble Klotho protein for the at least 24 hours (or longer), preferably at room temperature. It will be appreciated, however, that refrigeration and/or freezing are also contemplated herein.

In addition, the Inventors have found that measured Klotho protein levels from finger stick (about 10-1000 ul, preferably about 50-200 ul of blood) are consistent with Klotho protein levels measured from venous blood draw in the same subject. Thus, Klotho protein can be accurately, reproducibly, and reliably measured in relatively small, commercially practical volumes of blood. This was surprising and unexpected to the inventors. In particular, the Inventors had expected that the quantity of blood collected in a standard vial may be required in order to accurately, reproducibly, and reliably measure Klotho protein in blood.

Without being bound to any theory, venous blood draws (e.g., for FDA-approved or other diagnostics) typically require a professional phlebotomist. Venous blood draws are also inconvenient and painful to the patient. A finger stick method, on the other hand, is convenient and low cost. Commercially, direct-to-consumer applications (e.g., diagnostic kits) may require at-home blood sampling. In such cases, a venous blood draw may be undesirable and/or impractical. Embodiments of the present disclosure can permit capillary blood sampling with a simple and convenient finger prick (or other means of sampling capillary blood). The amount of capillary blood sampled can be, for example, between about 10-1000 ul, preferably about 20-800 ul, more preferably about 30-600 ul, still more preferably about 40-400 ul, still more preferably about 50-200 ul of capillary blood.

In light of the foregoing, some embodiments of the present disclosure can include products and methods for capillary (non-venous, non-phlebotomic, etc.) sampling of blood or serum. Some embodiments can include a capillary blood sampling device. Illustratively, the capillary blood sampling device can be or comprise a lancing device and/or comprise a (blood) lancet). In some embodiments, the blood sampling or lancing device can be or comprise a finger stick apparatus, such as a (spring-loaded) finger pricker. It will be appreciated, however, that the present disclosure is not limited to fingers and quick-prick devices. In some embodiments, the lancing device or lancet thereof can be disposable and/or replaceable. Embodiments can also include written instructions for using the kit to collect a blood sample.

An illustrative method of stabilizing Klotho protein in a mammalian blood sample can comprise obtaining capillary blood from a mammalian subject, the capillary blood containing an amount of soluble Klotho protein, and storing the capillary blood in a container for at least 24 hours at room temperature, the container having a preservative or anti-coagulant disposed therein, the preservative or anti-coagulant being mixed with the capillary blood in the container, the preservative or anti-coagulant stabilizing the soluble Klotho protein for at least 24 hours at room temperature, the preservative or anti-coagulant comprising one or more of heparin, lithium heparin, EDTA, and K₂ EDTA. In some embodiments, storing the capillary blood mixed with the preservative or anti-coagulant for at least 2, 3, 4, 5, 6, 7, 8, 9, 10, 11, 12, 13, 14, 18, 21, 28, or 30 days at room temperature, the preservative or anti-coagulant stabilizing the soluble Klotho protein for at least 2, 3, 4, 5, 6, 7, 8, 9, 10, 11, 12, 13, 14, 18, 21, 28, or 30 days at room temperature. The sample of mixture may also be refrigerated in certain embodiments.

In certain embodiments, a small amount of blood (1-4 drops, about 10-1000 ul, preferably about 50-200 ul can be collected (e.g., at home) and sent via unfrozen, unrefrigerated, or uncooled (e.g., ambient or room temperature), normal shipping methods to a diagnostic facility. This provides a substantial benefit over existing methods and products. Accordingly, personal, at-home, or other non-clinic-based testing or diagnostics (e.g., lateral flow with quantification/measurement indicator, home ELISA kits, etc.) are contemplated herein. It will also be appreciated, however, that clinical or clinic-based testing or diagnostics are contemplated herein.

### Products and Methods for Measuring Klotho Protein Levels

Some embodiments include a method of detecting and/or quantifying endogenous and/or exogenous Klotho protein, and more particularly endogenous and/or exogenous soluble alpha Klotho protein. Some embodiments include a method of diagnosing Klotho protein deficiency in a subject. Some embodiments include a product (e.g., a system or kit) for detecting and quantifying Klotho protein levels, and more particularly endogenous and/or exogenous soluble alpha Klotho protein levels. Some embodiments include methods of diagnosing Klotho protein deficiency in a subject.

It has been postulated that levels of circulating, soluble Klotho protein in the serum of mammals decrease with increasing age. Prior to the present disclosure, however, reliable, reproducible data as to what age this postulated decrease in serum Klotho levels may begin to take place, what the normal level (or range) of naturally-occurring, circulating soluble Klotho protein is in human and non-human animals, what the rate of decline in circulating Klotho is in human and non-human animals, and how low the level (or range) of naturally-occurring, circulating soluble Klotho protein gets in human and non-human animals, was not available or widely accepted. Commercially-available antibodies may not be suitable for reliable, reproducible, and/or cost-effective detection of (both endogenous and exogenous) Klotho protein in a (crude and/or substantially unpurified) mammalian blood or serum sample. Accordingly, suitable antibodies (e.g., for blood sample ELISA detection) were developed in accordance with the following procedure.

Phase-1: Klotho Protein Expression: (mouse and human) alpha Klotho proteins of various sizes (e.g., amino acid residues 34-981, 34-549, etc. for human) with a (C-terminal) 6xHis tag, (C-terminal) Fc tag, or (N- or C-terminal) HAS tag were manufactured. Gene constructs corresponding to the above proteins were synthesized and cloned into a commercially-available expression vector, which were then transfected into HEK-293 cells or CHO cells for Klotho protein production. Expressed Klotho protein was purified by methods, known and described in the present disclosure. Illustratively, IMAC and IE protein chromatography were used, yielding 0.78 mg/mL protein sample (measured by UV absorbance at 280 nm) in 330 mM NaCl, 20 mM Na-Phosphate buffer, pH 8.0. Similarly,

Protein A chromatography followed by size exclusion chromatography, as described herein, yielded active protein in monomeric and multimeric (e.g., dimer and tetramer) form.

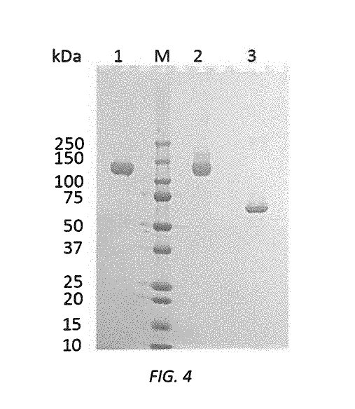

Quality control: protein purity and integrity of purified protein was analyzed in 4-20% SDS -PAGE (reducing and non-reducing) stained with GelCode Blue reagent. See e.g., FIG. 4, in which:

Lane 1: Klotho ECD, Lot# 170918A, 2 ug/lane, reduced.

Lane M: Protein molecular weight marker.

Lane 2: Klotho ECD, Lot# 170918A, 2 ug/lane, non-reduced.

Lane 3: BSA standard, 2 ug/lane, reduced

The theoretical, calculated molecular weight of the expressed protein was 109.97 kDa.

Phase-2: Chicken Polyclonal (IgY) and Mouse Monoclonal (hybridoma) development: Expressed protein was used as an antigen to immunize mice and chickens for development of mono- and polyclonal antibodies. Mouse monoclonal is preferred in some embodiments for ELISA antibodies. After the fusion process (splenocytes with myeloma cells), two rounds of hybridoma selection were performed, around 3,000 clones were initially screened and finally narrowed to 10 clones, based on activity. These 10 clones were banked and also cultured and purified to produce ˜5 mg of purified antibody from each.

The tables below present illustrative mouse sera IgG titer evaluations using target antigens, by indirect ELISA (Standard ELISA protocol. TMB 5 mM)

Antigen 1—6xHis tagged Klotho 0.5 ug/mL in 50 mM Na-bicarbonate buffer.

Antigen 2—Klotho-Fc 0.5 ug/mL in 50 mM Na-bicarbonate buffer.

Summary of Table 1: Mice M3 and M5 were selected for spleen collection.

Primary screening of Klotho hybridoma: Splenocytes from mice #M3 and #M5 collected and fused with myeloma cells according to standard protocol.

Hybridoma supernatants were evaluated by indirect ELISA using target antigens (standard indirect ELISA protocol. TMB 10 mM.).

Antigen, Klotho protein (Lot#170918A, 0.78 mg/mL) 0.5 ug/mL in 50 mM Na-Bicarbonate, 100 ul/well, +4° C., overnight.

Anti-mouse HRP, Peroxidase AffiniPure Goat Anti-Mouse IgG (subclasses 1+2a+2b+3), Fcγ Fragment Specific, 1:4000 (Jackson Lab., Cat#115-035-164) in SEDB, 100 uL/well, 1 hour, RT.

Selection cut-off OD450 nm>0.5.

The tables below present primary Klotho hybridoma screening. Clones with OD450 nm between 0.5 and <2 are highlighted in yellow (light grey). Clones with OD450 nm>2 are highlighted in orange (dark grey).

Summary of Table 2: 2,976 clones isolated and analyzed. 66 target specific clones expanded for confirmatory screening.

Secondary screening of Klotho hybridoma: Hybridoma supernatants were evaluated by indirect ELISA using target antigens (standard indirect ELISA protocol. TMB 10 min.).

Antigens, (1) Klotho-6xHis protein (Lot#170918A, 0.78 mg/mL) 0.5 ug/mL in 50 mM Na-Bicarbonate, 100 ul/well, +4° C., overnight. (2) Klotho-Fc protein (Vista, 0.1 mg/mL) 0.5 ug/mL in 50 mM Na-Bicarbonate, 100 ul/well, +4° C., overnight.

Detection Ab: Anti-mouse HRP, Peroxidase AffiniPure Goat Anti-Mouse IgG (subclasses 1+2a+26+3), Fcy Fragment Specific, 1:5000 (Jackson Lab., Cat#115-035-164) in SEDB, 100 uL/well, 1 hour, RT.

Summary of Table 3: 28 Klotho-6xHis specific clones were confirmed (OD450 cut-off: >0.5, Klotho-6xHis plate)—1F6, 2E1, 2E12, 3D2, 4H4, 4H5, 5D12, 7A12, 7F9, 8G11, 10A8, 10D1, 10G3, 11C9, 11F7, 11H3, 12G4, 12H3, 14B2, 14B7, 14B12, 15E9, 16H1, 18C2, 18G5, 21G12, 26G3, 28F11.

1st arrays of 9 Klotho-6xHis specific clones recommended for native (untagged) Klotho ELISA development (OD450>2, Klotho-6xHis plate)—2E12, 4H4, 4H5, 5D12, 11C9, 11F7, 14B12, 26G3, 28F11.

2nd array of 11 Klotho-6xHis specific clones recommended for native (untagged) Klotho ELISA development (OD450>1 and <2, Klotho-6xHis plate) - 1F6, 2E1, 3D2, 7A12, 7F9, 8G11, 10G3, 11H3, 14B7, 18C2, 18G5.

1st array of 10 Klotho-6xHis and Klotho-Fc specific clones recommended for the development of ELISA capable to measure both, untagged and Klotho-Fc proteins (OD450 >1.7, Klotho-6xHis and Klotho-Fc plates)-1F6, 2E12, 4H4, 4H5, 8G11, 11C9, 11F7, 14B12, 26G3, 28F11.

2nd array of 2 Klotho-6xHis and Klotho-Fc specific clones recommended for the development of ELISA capable to measure both, untagged and Klotho-Fc proteins (OD450>1.2 and <1.7, Klotho-6xHis and Klotho-Fc plates)—5D12, 14B7.

mIgG isotyping: mIgG isotyping of Klotho hybridoma selected clones: Antibody isotyping from selected hybridoma clone culture supernatants.

Samples of hybridoma culture media were used to determine antibody iso-types using Rapid ELISA Mouse mAb Isotyping kit (ThermoFisher, Cat#37503) following manufacturer's instructions.

The table below presents the results of mIgG isotyping of Klotho hybridoma selected clones.

Summary of Table 4: mIgG isotype determination:

1F6, 2E12, 4H4, 4H5, 8G11 and 11F7: IgG1

11C9, 14B12, 26G3 and 28F11: IgG2b

Quality Control: Indirect ELISA was performed for quality control: An ELISA plate was coated with unconjugated 6xHis tagged Klotho protein or Klotho-Fc protein at 0.5 ug/ml overnight at +4° C. in 50 mM Na-bicarbonate buffer. Purified 1F6, 2E12, 4H4, 4H5, 8G11, 11C9, 11F7, 14B12, 26G3, and 28F11 antibodies were applied in 5-fold serial dilutions starting at 500 ng/mL and the secondary antibody, goat anti-mouse HRP, was applied at 0.16 pg/ml. Reaction was developed for 7 minutes. Raw absorbance data at 450 nm and corresponding antibody titration curve.

TABLE 5
1. 6xHis tagged Klotho protein

| [Ab], | 1F6, | | 2E12, | | 4H4, | | 4H5, | | 8G11, | | 11C9, | | |
|---|---|---|---|---|---|---|---|---|---|---|---|---|---|
| ng/mL | Lot#180410A | | Lot#180410B | | Lot#180410C | | Lot#180410D | | Lot#180410E | | Lot#180410F | | |
| 500 | 0.051 | 0.048 | 0.047 | 0.047 | 0.048 | 0.048 | 0.048 | 0.045 | 0.046 | 0.046 | 0.048 | 0.048 | No antigen |
| 500 | 1.782 | 1.793 | 2.533 | 2.531 | 1.822 | 1.662 | 1.759 | 1.726 | 1.861 | 1.9 | 2.977 | 2.993 | |
| 100 | 0.827 | 0.852 | 1.398 | 1.327 | 0.681 | 0.684 | 0.712 | 0.67 | 0.695 | 0.789 | 1.994 | 2.207 | |
| 20 | 0.266 | 0.271 | 0.452 | 0.426 | 0.209 | 0.194 | 0.199 | 0.217 | 0.259 | 0.223 | 0.862 | 0.946 | |
| 4 | 0.096 | 0.102 | 0.152 | 0.143 | 0.09 | 0.08 | 0.082 | 0.086 | 0.091 | 0.087 | 0.251 | 0.32 | |
| 0.8 | 0.064 | 0.061 | 0.065 | 0.074 | 0.058 | 0.061 | 0.06 | 0.06 | 0.062 | 0.06 | 0.105 | 0.124 | |
| 0.16 | 0.056 | 0.055 | 0.062 | 0.058 | 0.052 | 0.059 | 0.056 | 0.052 | 0.055 | 0.062 | 0.064 | 0.067 | |
| 0 | 0.051 | 0.049 | 0.052 | 0.05 | 0.051 | 0.049 | 0.05 | 0.052 | 0.052 | 0.052 | 0.054 | 0.061 | |

| [Ab], | 11C9, | | 11F7, | | 14B12, | | 26G3, | | 28F11, | | |
|---|---|---|---|---|---|---|---|---|---|---|---|
| ng/mL | Lot#180410F | | Lot#180410G | | Lot#180410H | | Lot#180410I | | Lot#180410J | | |
| 500 | 0.071 | 0.067 | 0.059 | 0.058 | 0.06 | 0.062 | 0.08 | 0.072 | 0.076 | 0.073 | No antigen |
| 500 | 3.023 | 2.876 | 1.754 | 1.779 | 1.863 | 1.856 | 2.975 | 2.92 | 2.572 | 2.586 | |
| 100 | 2.171 | 1.859 | 0.472 | 0.506 | 0.88 | 0.857 | 2.103 | 2.145 | 1.44 | 1.565 | |
| 20 | 0.988 | 0.864 | 0.154 | 0.137 | 0.29 | 0.253 | 0.994 | 1.001 | 0.513 | 0.586 | |
| 4 | 0.28 | 0.282 | 0.075 | 0.072 | 0.122 | 0.102 | 0.291 | 0.341 | 0.175 | 0.191 | |
| 0.8 | 0.114 | 0.104 | 0.059 | 0.06 | 0.066 | 0.06 | 0.107 | 0.126 | 0.082 | 0.081 | |
| 0.16 | 0.051 | 0.068 | 0.057 | 0.054 | 0.053 | 0.055 | 0.075 | 0.065 | 0.057 | 0.059 | |
| 0 | 0.072 | 0.057 | 0.06 | 0.06 | 0.057 | 0.059 | 0.06 | 0.063 | 0.068 | 0.062 | |

2. Klotho-Fc protein

| [Ab], | 1F6, | | 2E12, | | 4H4, | | 4H5, | | 8G11, | | 11C9, | | |
|---|---|---|---|---|---|---|---|---|---|---|---|---|---|
| ng/mL | Lot#180410A | | Lot#180410B | | Lot#180410C | | Lot#180410D | | Lot#180410E | | Lot#180410F | | |
| 500 | 0.065 | 0.058 | 0.056 | 0.059 | 0.058 | 0.058 | 0.059 | 0.069 | 0.058 | 0.056 | 0.06 | 0.065 | No antigen |
| 500 | 1.328 | 1.307 | 1.78 | 1.838 | 1.333 | 1.37 | 1.366 | 1.25 | 1.325 | 1.352 | 2.74 | 2.756 | |
| 100 | 0.505 | 0.462 | 0.749 | 0.765 | 0.466 | 0.44 | 0.412 | 0.423 | 0.43 | 0.455 | 1.801 | 1.787 | |
| 20 | 0.176 | 0.146 | 0.241 | 0.223 | 0.151 | 0.147 | 0.14 | 0.135 | 0.15 | 0.144 | 0.742 | 0.732 | |
| 4 | 0.087 | 0.088 | 0.094 | 0.093 | 0.075 | 0.076 | 0.076 | 0.078 | 0.079 | 0.072 | 0.231 | 0.322 | |
| 0.8 | 0.073 | 0.065 | 0.062 | 0.063 | 0.057 | 0.057 | 0.058 | 0.061 | 0.06 | 0.058 | 0.101 | 0.127 | |
| 0.16 | 0.068 | 0.065 | 0.061 | 0.058 | 0.056 | 0.057 | 0.057 | 0.058 | 0.055 | 0.055 | 0.067 | 0.148 | |
| 0 | 0.07 | 0.07 | 0.065 | 0.057 | 0.07 | 0.059 | 0.056 | 0.075 | 0.06 | 0.063 | 0.096 | 0.112 | |

| [Ab], | 11C9, | | 11F7, | | 14B12, | | 26G3, | | 28F11, | | |
|---|---|---|---|---|---|---|---|---|---|---|---|
| ng/mL | Lot#180410F | | Lot#180410G | | Lot#180410H | | Lot#180410I | | Lot#180410J | | |
| 500 | 0.072 | 0.065 | 0.058 | 0.057 | 0.06 | 0.06 | 0.066 | 0.063 | 0.063 | 0.065 | No antigen |
| 500 | 2.623 | 2.613 | 0.798 | 0.82 | 2.044 | 2.085 | 2.641 | 2.607 | 2.417 | 2.325 | |
| 100 | 1.886 | 1.686 | 0.291 | 0.288 | 1.047 | 1.012 | 1.822 | 1.784 | 1.337 | 1.427 | |
| 20 | 0.816 | 0.717 | 0.104 | 0.108 | 0.33 | 0.316 | 0.828 | 0.787 | 0.54 | 0.558 | |
| 4 | 0.23 | 0.24 | 0.067 | 0.066 | 0.125 | 0.135 | 0.265 | 0.267 | 0.165 | 0.171 | |
| 0.8 | 0.183 | 0.101 | 0.056 | 0.061 | 0.071 | 0.069 | 0.106 | 0.104 | 0.083 | 0.083 | |
| 0.16 | 0.078 | 0.068 | 0.059 | 0.058 | 0.057 | 0.058 | 0.063 | 0.058 | 0.059 | 0.059 | |
| 0 | 0.076 | 0.066 | 0.066 | 0.061 | 0.057 | 0.06 | 0.057 | 0.057 | 0.059 | 0.066 | |

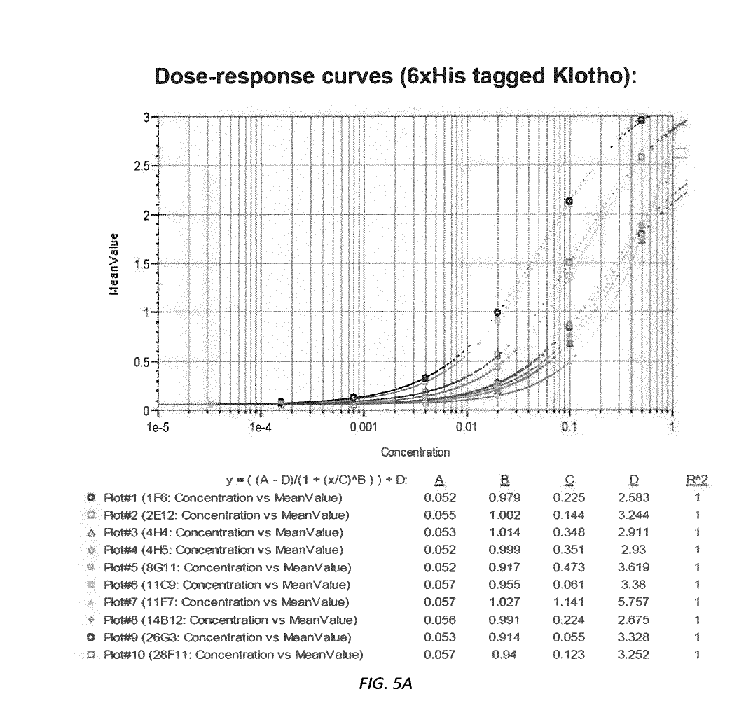

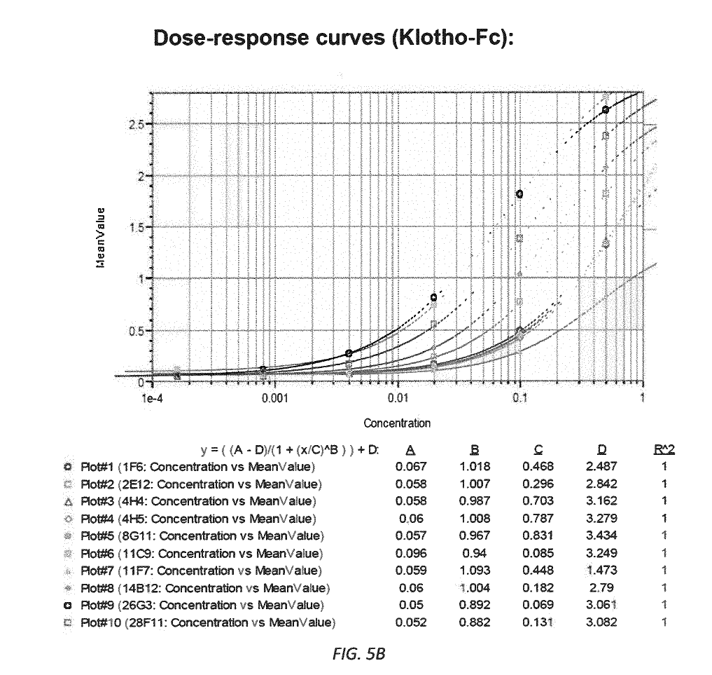

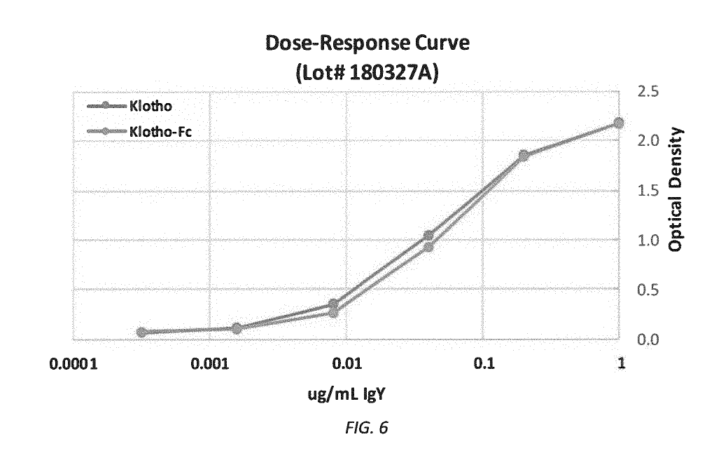

See also, dose responsive curve illustrated in FIGS. 5A, 5B, and 6 respectively.

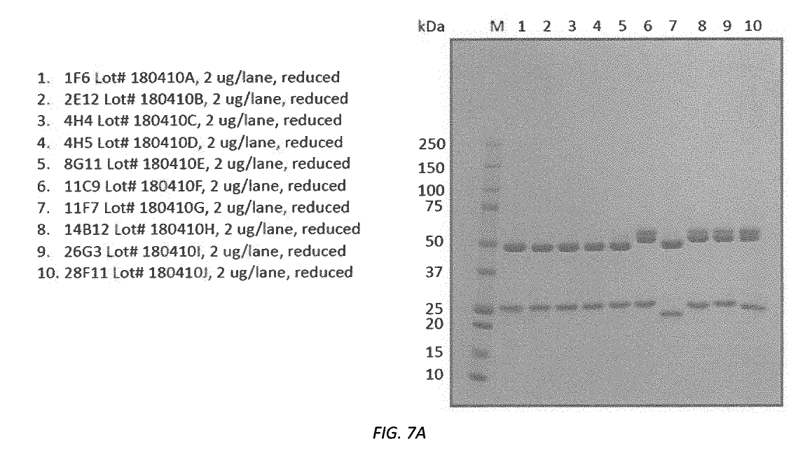

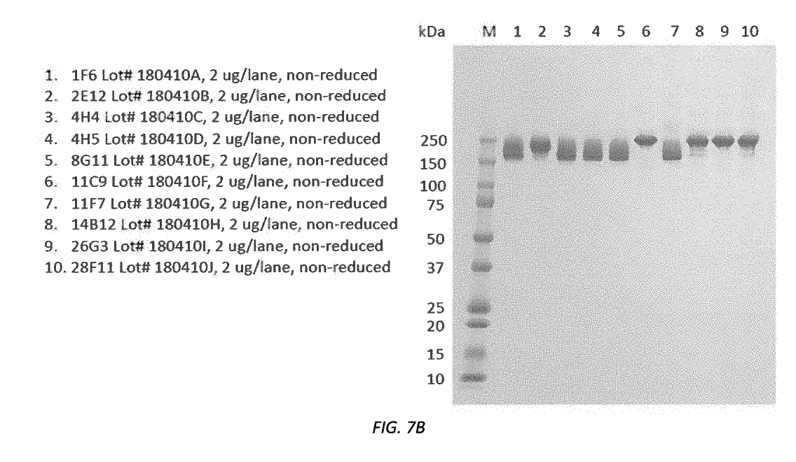

Quality control: Purity and integrity of purified antibody were analyzed in 4-20% SDS-PAGE (reducing and non-reducing) stained with GelCode Blue reagent. See FIGS. 7A and 7B, respectively.

Polyclonal IgY antibodies: Polyclonal IgY antibodies were also developed. The above design was adapted as necessary for production of polyclonal IgY antibodies in chickens.

Chicken serum IgY titer evaluation using target antigens, by indirect ELISA (TMB: 5 mM). Read at 450 nm

Antigen: Klotho 1 ug/mL (0.78 mg/mL, Lot# 170918A) in 50 mM Na-bicarbonate.

Samples: Chicken #1, #2, RT, 1 hr (5% milk in PBS).

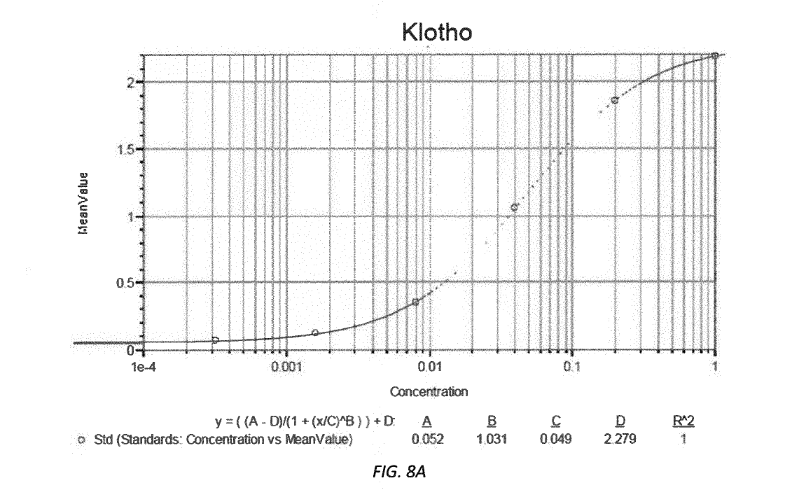

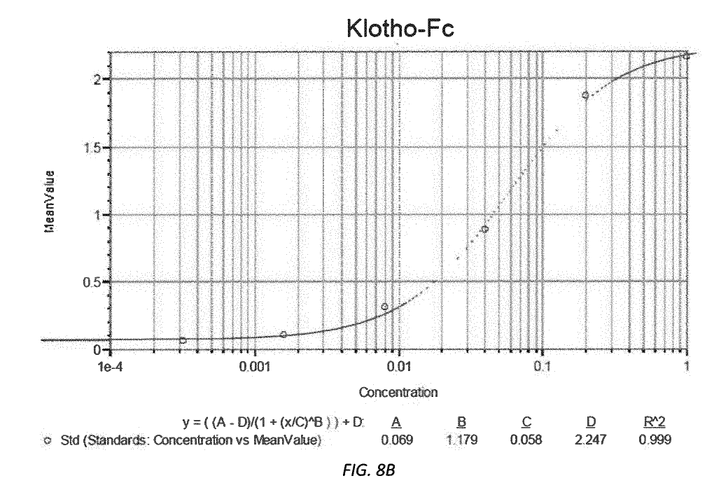

An ELISA plate was coated with 6xHis tagged Klotho protein or Klotho-Fc protein at 0.5 ug/ml overnight at +4° C. in 50 mM Na-bicarbonate buffer. Purified anti-Klotho IgY antibody (lot# 180327A) was applied in 5-fold serial dilutions starting at 1000 ng/mL and the secondary antibody, goat anti-chicken HRP, was applied at 0.16 μg/ml. Reaction was developed for 5 minutes. Raw absorbance data at 450 nm and corresponding antibody titration curve. See also, FIGS. 8A-8B.

TABLE 7

| [IgY], | Antigen | |
|---|---|---|
| ng/mL | Klotho | Klotho-Fc |

| | | | | | |
|---|---|---|---|---|---|
| 1000 | 0.047 | 0.046 | 0.046 | 0.054 | No Antigen |
| 1000 | 2.192 | 2.179 | 2.137 | 2.171 | |
| 200 | 1.916 | 1.795 | 1.797 | 1.942 | |
| 40 | 1.076 | 1.032 | 0.865 | 0.9 | |
| 8 | 0.354 | 0.337 | 0.328 | 0.293 | |
| 1.6 | 0.123 | 0.118 | 0.106 | 0.109 | |
| 0.32 | 0.063 | 0.073 | 0.058 | 0.062 | |
| 0 | 0.046 | 0.047 | 0.047 | 0.045 | |

Summary—Both HRP and Biotin-anti-Klotho IgY show significant signal in indirect ELISA. Bio-IgY has stronger signal than HRP-IgY.

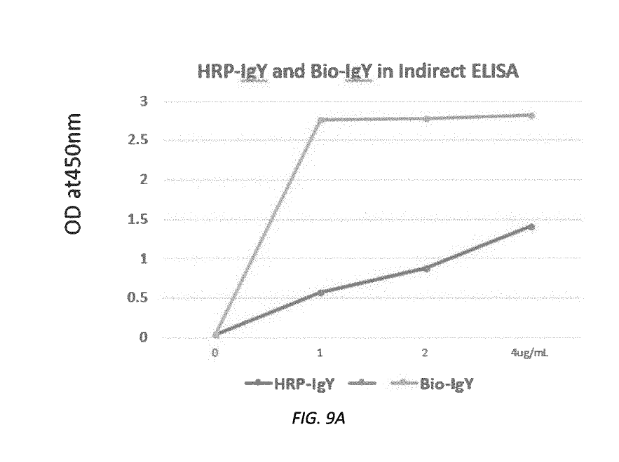

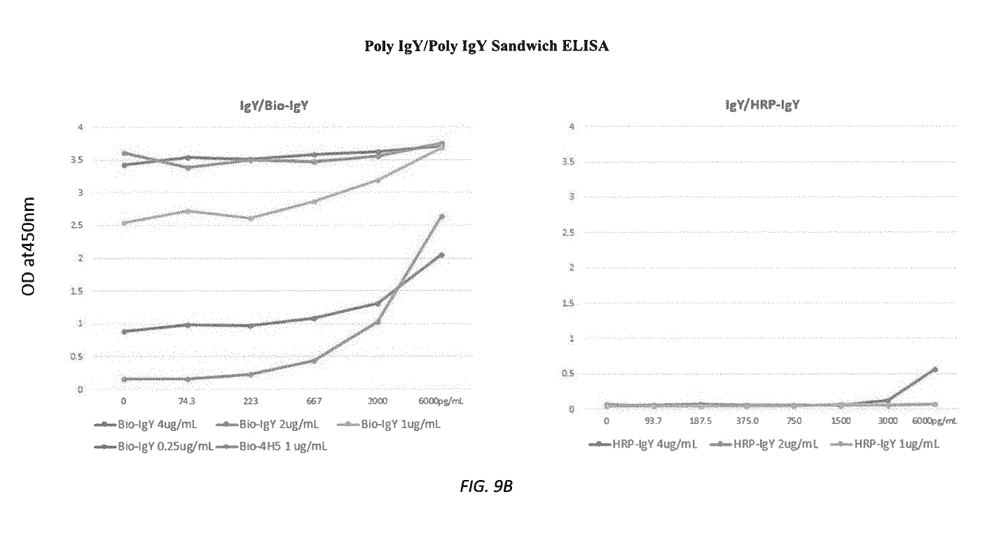

As illustrated in FIGS. 9A and 9B, there is a relatively weak signal in IgY/HRP-IgY pair. IgY/Bio-IgY shows relatively high background, even at low concentration (0.25 μg/mL). IgY/Bio-4H5 pair appears to work better than IgY/labled-IgY pair.

It has been postulated that levels of circulating, soluble Klotho protein in the serum of mammals decrease with increasing age. Baseline measurement for naturally-occurring, circulating soluble Klotho protein in a human subject has been reported in various publications. A thorough literature review yielded the following average range(s) for circulating soluble Klotho protein in the indicated human subject (see Tables 8-9).

TABLE 8

| Age | Sex | |
|---|---|---|
| group | Healthy male | Healthy female |
| 0-15 | 1692 (372, 5694) | 2487 (964, 5866) |
| 15-35 | 459.4 (335.1-617.2)-508 (448-661) | 459.4 (335.1-617.2)-508 (448-661) |
| 35-50 | 476.9 (416.9-545.5)-692 (618-866) | 476.9 (416.9-545.5)-692 (618-866) |
| 50-65 | 141.8 (94.7-189.2)-377.17 (359.46-394.90) | 141.8 (94.7-189.2)-377.17 (359.46-394.90) |
| >65 | 354 (264-410)-999.5 (887.7-1186.4) | 354 (264-410)-999.5 (887.7-1186.4) |
| Age | Disease | |
| group | GH deficiency | CKD all |
| 0-15 | 340 (433, 7826) | 1746 (671-3438) |
| 15-35 | | |
| 35-50 | | 396.3 (306.3-558.4)-478 (348-658) |
| 50-65 | | 613.0 (504.0 to 811.0)-809 (674-981) |
| >65 | | Men 596.0 (464.3 to 790.3); Women 614.0 |
| | | (558.0 to 811.0) |
| Age | | |
| group | CKD stage 1-2 | CKD stage 3 |
| 0-15 | 1418 (501-3314)-1736.5 (1272-2737) | 1418 (501-3314)-1736.5 (1272-2737) |
| 15-35 | | |
| 35-50 | 540.5 (371.3-824.8)-482.7 (360.2-683.1) | 386.4 (319.2-456.7) |
| 50-65 | 388 (254-532) | 316 (199-464)-337 (217-489) |
| >65 | | |
| Age | | |
| group | CKD stage 4 | CKD stage 5 |
| 0-15 | 1019 (502-1592)-1418 (501-3314) | 1019 (502-1592)-1418 (501-3314) |
| 15-35 | | |
| 35-50 | 337.8 (251.0-444.2) | 295.6 (207.7-387.5) |
| 50-65 | 215 (135-331) | 249 (100-452) |
| >65 | Mean ± SEM 473.9 ± 121.4 | Mean ± SEM 546 ± 164.3 |
| Age | | |
| group | Kidney transplant | T1D |
| 0-15 | 1698 (1110-2123) | |
| 15-35 | | 454 (420-492) |
| 35-50 | | 659.3 (525.3, 827.6)-787.7 (629.5, 1007) |
| 50-65 | 550 (304-651)-605 (506-784) | |
| >65 | | |
| Age | | |
| group | T2D | CAD |
| 0-15 | | |
| 15-35 | | |
| 35-50 | 572.4 (541.9-604.6) | |
| 50-65 | Men 67.3 (52.8-69.3); Women 65.7 (51.6-71.6) | 490.5 (349.0-549.4) |
| >65 | 493.7 (287.8-777.7) | Men 45.1 (33.7-54.1); Women 40.7 (31-56.4) |
| Age | | |
| group | COPD | MS |
| 0-15 | | |
| 15-35 | | new cases Mean ± SEM 585.56 ± 153.99; |
| | | chronic cases Mean ± SEM 696.94 ± 170.52 |
| 35-50 | | |
| 50-65 | | |
| >65 | 250 (168-330) | |

TABLE 9

| | KL-VS genotype | | |
|---|---|---|---|
| Age group | Non carrier | Heterozygous | Homozygous |
| >65 | Mean ± SEM | Mean ± SEM | Mean ± SEM |
| | 780.9 ± 19.6 | 891.7 ± 45.3 | 599.1 ± 75.7 |

In the table, above, CKD: chronic kidney disease; GH: growth hormone; T1D: type 1 diabetes; T2D: type 2 diabetes; CAD: coronary artery disease; COPD: chronic obstructive pulmonary disease; MS: multiple sclerosis.

In light of the variability observed in the literature, a more robust, more consistent, and/or more accurate means of measuring serum Klotho levels is needed. Embodiments of the present disclosure include a Klotho diagnostic kit and method of using the same, as described in further detail, below.

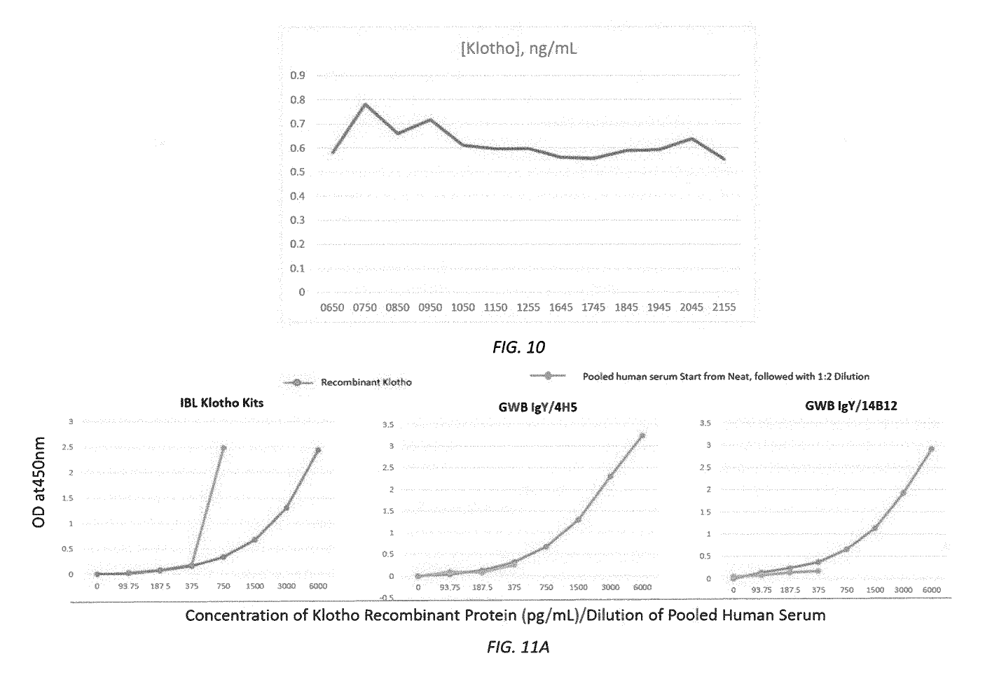

Table 10 presents a baseline measurement for naturally-occurring, circulating soluble Klotho protein in a human subject (prior to receiving any treatment, e.g., health supplement, pharmaceutical, therapeutic protein, etc.). FIG. 10 is a graphical representation of the (daily) change in circulating Klotho protein levels in a human subject

TABLE 10

| Time Collected | [Klotho] (ng/mL) |
|---|---|
| 0650 | 0.579 |
| 0750 | 0.782 |
| 0850 | 0.66 |
| 0950 | 0.717 |
| 1050 | 0.611 |
| 1150 | 0.596 |
| 1255 | 0.597 |
| 1645 | 0.56 |
| 1745 | 0.556 |
| 1845 | 0.588 |
| 1945 | 0.592 |
| 2045 | 0.638 |
| 2155 | 0.551 |

Once per hour, beginning at 0650 hours, a blood sample was taken from the human subject and Klotho protein levels were measured by ELISA.

Quantitative ELISA according to an embodiment of the present disclosure is able to detect (1) Native (e.g., endogenous) Klotho, (2) recombinant Klotho-Fc fusion, and (3) recombinant 6xHis Klotho in human plasma samples.

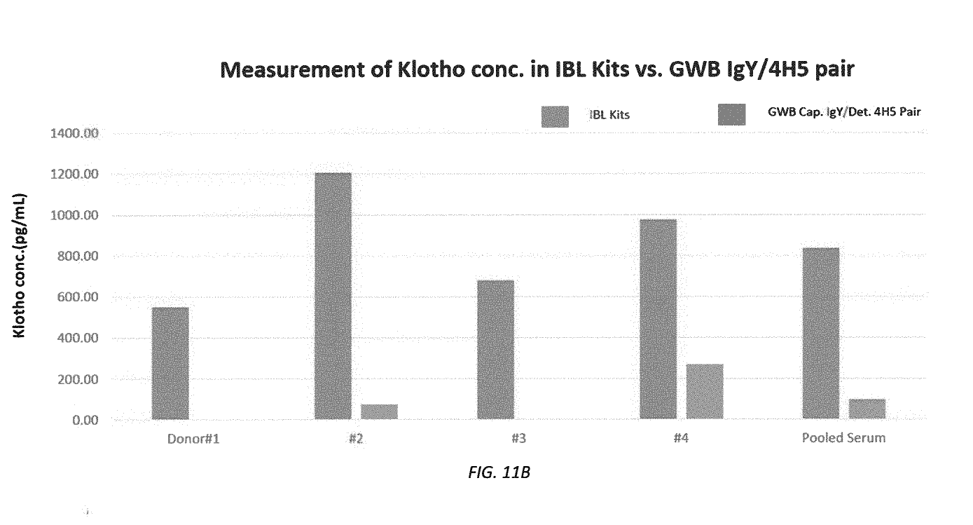

Further measurements of Klotho protein are illustrated and presented in FIGS. 11A-11B and Table 11, below.

TABLE 11

| | Adjusted Calculated consentration of Klotho (pg/mL) | | | |
|---|---|---|---|---|
| | | | IgY/4H5 with | |
| Pooled Human | IBL | IgY/ | mouse IgG in | IgY/ |
| Serum Dilution | Kits | 4H5 | Assay Diluent | 14B12 |
| Neat | 6125.652* | 331.86 | 99.732 | 233.301 |
| 1:02 | 838.748 | 263.299 | 92.946 | 394.267 |
| 1:04 | 859.529 | 585.664 | 21.05 | 511.614 |
| 1:08 | 813.752 | Out of Range | 580.529 | 700.209 |

*Kits manual recommend sample dilution at 1:2 or 1:4

### Klotho Stability in Human Plasma Using ELISA Kit

Samples of venous and capillary (“finger”) blood were collected from four donors (A, B, C, D) and stored in (i) EDTA or (ii) heparin containing tubes. 1.5 ng/mL of Klotho (lot# 170918A—human soluble alpha Klotho) was spiked into a set of venous and capillary samples collected from donor A. Collected blood (blood samples) were stored at room temperature for 0, 1, 4, 16, 24, 48, 72, 96, or 168 h, then spun-down. Plasma from each sample was stored at −80° C. prior to ELISA test. ELISA plate maps, raw data, standard curve data, and concentrations are presented in Tables 12-21, below.
ELISA plate map 1:

TABLE 12

| | | | | | |
|---|---|---|---|---|---|
| STD, 6 ng/mL | A (0 h) EDTA | A (1 h) EDTA | A (4 h) EDTA | A (16 h) EDTA | A (24 h) EDTA |
| STD, 3 ng/mL | A (48 h) EDTA | A (72 h) EDTA | A (96 h) EDTA | A (168 h) EDTA | A (Finger) EDTA |
| STD, 1.5 ng/mL | A (0 h) Heparin | A (1 h) Heparin | A (4 h) Heparin | A (16 h) Heparin | A (24 h) Heparin |
| STD, 0.75 ng/mL | A (48 h) Heparin | A (72 h) Heparin | A (96 h) Heparin | A (168 h) Heparin | A (Finger) Heparin |
| STD, 0.375 ng/mL | B (0 h) EDTA | B (1 h) EDTA | B (4 h) EDTA | B (16 h) EDTA | B (24 h) EDTA |
| STD, 0.188 ng/mL | B (48 h) EDTA | B (72 h) EDTA | B (96 h) EDTA | B (168 h) EDTA | B (Finger) EDTA |
| STD, 0.094 ng/mL | B (0 h) Heparin | B (1 h) Heparin | B (4 h) Heparin | B (16 h) Heparin | B (24 h) Heparin |
| STD, 0 ng/mL | B (48 h) Heparin | B (72 h) Heparin | B (96 h) Heparin | B (168 h) Heparin | B (Finger) Heparin |

ELISA plate map 2:

TABLE 13

| | | | | | |
|---|---|---|---|---|---|
| STD, 6 ng/mL | C (0 h) EDTA | C (1 h) EDTA | C (4 h) EDTA | C (16 h) EDTA | C (24 h) EDTA |
| STD, 3 ng/mL | C (48 h) EDTA | C (72 h) EDTA | C (96 h) EDTA | C (168 h) EDTA | C (Finger) EDTA |
| STD, 1.5 ng/mL | C (0 h) Heparin | C (1 h) Heparin | C (4 h) Heparin | C (16 h) Heparin | C (24 h) Heparin |
| STD, 0.75 ng/mL | C (48 h) Heparin | C (72 h) Heparin | C (96 h) Heparin | C (168 h) Heparin | C (Finger) Heparin |
| STD, 0.375 ng/mL | D (0 h) EDTA | D (1 h) EDTA | D (4 h) EDTA | D (16 h) EDTA | D (24 h) EDTA |
| STD, 0.188 ng/mL | D (48 h) EDTA | D (72 h) EDTA | D (96 h) EDTA | D (168 h) EDTA | D (Finger) EDTA |
| STD, 0.094 ng/mL | D (0 h) Heparin | D (1 h) Heparin | D (4 h) Heparin | D (16 h) Heparin | D (24 h) Heparin |
| STD, 0 ng/mL | D (48 h) Heparin | D (72 h) Heparin | D (96 h) Heparin | D (168 h) Heparin | D (Finger) Heparin |

ELISA plate map 3:

TABLE 14

| | | | |
|---|---|---|---|
| STD, 6 ng/mL | Spike (0 h) EDTA | Spike (168 h) EDTA | Spike (72 h) Heparin |
| STD, 3 ng/mL | Spike (1 h) EDTA | Spike (Finger) EDTA | Spike (96 h) Heparin |
| STD, 1.5 ng/mL | Spike (4 h) EDTA | Spike (0 h) Heparin | Spike (168 h) EDTA |
| STD, 0.75 ng/mL | Spike (16 h) EDTA | Spike (1 h) Heparin | Spike (Finger) Heparin |
| STD, 0.375 ng/mL | Spike (24 h) EDTA | Spike (4 h) Heparin | |
| STD, 0.188 ng/mL | Spike (48 h) EDTA | Spike (16 h) Heparin | |
| STD, 0.094 ng/mL | Spike (72 h) EDTA | Spike (24 h) Heparin | |
| STD, 0 ng/mL | Spike (96 h) EDTA | Spike (48 h) Heparin | |

ELISA 450 nm absorbance raw data (plate 1):

TABLE 15

| | | | | | | | | | | | |
|---|---|---|---|---|---|---|---|---|---|---|---|
| 3.44 | 3.39 | 0.238 | 0.231 | 0.231 | 0.247 | 0.235 | 0.233 | 0.248 | 0.244 | 0.261 | 0.276 |
| 2.03 | 2.005 | 0.235 | 0.235 | 0.242 | 0.235 | 0.247 | 0.248 | 0.254 | 0.264 | 0.283 | 0.282 |
| 1.117 | 1.077 | 0.239 | 0.241 | 0.229 | 0.236 | 0.236 | 0.248 | 0.253 | 0.264 | 0.256 | 0.283 |
| 0.593 | 0.573 | 0.237 | 0.23 | 0.241 | 0.248 | 0.239 | 0.26 | 0.252 | 0.248 | 0.254 | 0.258 |
| 0.31 | 0.328 | 0.162 | 0.157 | 0.16 | 0.163 | 0.162 | 0.161 | 0.173 | 0.173 | 0.184 | 0.181 |
| 0.206 | 0.202 | 0.167 | 0.178 | 0.17 | 0.172 | 0.173 | 0.177 | 0.18 | 0.174 | 0.182 | 0.199 |
| 0.133 | 0.131 | 0.162 | 0.173 | 0.175 | 0.172 | 0.174 | 0.172 | 0.173 | 0.176 | 0.176 | 0.185 |
| 0.059 | 0.059 | 0.177 | 0.18 | 0.181 | 0.182 | 0.176 | 0.163 | 0.165 | 0.185 | 0.182 | 0.19 |

ELISA 450 nm absorbance raw data (plate 2):

TABLE 16

| | | | | | | | | | | | |
|---|---|---|---|---|---|---|---|---|---|---|---|
| 3.409 | 3.302 | 0.228 | 0.223 | 0.237 | 0.237 | 0.232 | 0.233 | 0.24 | 0.233 | 0.26 | 0.275 |
| 1.986 | 1.904 | 0.246 | 0.239 | 0.242 | 0.247 | 0.244 | 0.242 | 0.251 | 0.256 | 0.227 | 0.251 |
| 1.066 | 1.052 | 0.229 | 0.226 | 0.222 | 0.228 | 0.226 | 0.236 | 0.234 | 0.246 | 0.244 | 0.251 |
| 0.589 | 0.56 | 0.225 | 0.233 | 0.242 | 0.238 | 0.266 | 0.239 | 0.25 | 0.251 | 0.244 | 0.23 |
| 0.329 | 0.327 | 0.192 | 0.181 | 0.165 | 0.174 | 0.187 | 0.174 | 0.196 | 0.168 | 0.174 | 0.163 |
| 0.195 | 0.195 | 0.174 | 0.176 | 0.184 | 0.188 | 0.196 | 0.184 | 0.205 | 0.172 | 0.169 | 0.17 |
| 0.136 | 0.129 | 0.136 | 0.165 | 0.177 | 0.165 | 0.163 | 0.171 | 0.191 | 0.168 | 0.177 | 0.186 |
| 0.064 | 0.072 | 0.19 | 0.177 | 0.184 | 0.177 | 0.198 | 0.188 | 0.197 | 0.18 | 0.17 | 0.167 |

ELISA 450 nm absorbance raw data (plate 3):

TABLE 17

| | | | | | | | |
|---|---|---|---|---|---|---|---|
| 3.482 | 3.393 | 0.449 | 0.442 | 0.553 | 0.558 | 0.51 | 0.506 |
| 2.04 | 1.976 | 0.438 | 0.429 | 0.587 | 0.603 | 0.501 | 0.509 |
| 1.11 | 1.087 | 0.426 | 0.427 | 0.506 | 0.498 | 0.481 | 0.503 |
| 0.6 | 0.577 | 0.442 | 0.441 | 0.514 | 0.518 | 0.568 | 0.524 |
| 0.341 | 0.331 | 0.471 | 0.453 | 0.506 | 0.522 | | |
| 0.202 | 0.201 | 0.47 | 0.462 | 0.537 | 0.548 | | |
| 0.137 | 0.137 | 0.514 | 0.517 | 0.54 | 0.537 | | |
| 0.062 | 0.064 | 0.558 | 0.539 | 0.564 | 0.57 | | |

Calculation: Standard curve 1:

TABLE 18

| | | | Back | | | | |
|---|---|---|---|---|---|---|---|
| | Concentration | | Conc. | | Mean | Std. | CV |
| Sample | (ng/mL) | Wells | Calc. | Values | Value | Dev. | % |
| St01 | 6 | A1 | 6.062 | 3.44 | 3.415 | 0.035 | 1 |
| | | A2 | 5.934 | 3.39 | | | |
| St02 | 3 | B1 | 3.03 | 2.03 | 2.018 | 0.018 | 0.9 |
| | | B2 | 2.985 | 2.005 | | | |
| St03 | 1.5 | C1 | 1.525 | 1.117 | 1.097 | 0.028 | 2.6 |
| | | C2 | 1.464 | 1.077 | | | |
| St04 | 0.75 | D1 | 0.758 | 0.593 | 0.583 | 0.014 | 2.4 |
| | | D2 | 0.73 | 0.573 | | | |
| St05 | 0.375 | E1 | 0.358 | 0.31 | 0.319 | 0.013 | 4 |
| | | E2 | 0.384 | 0.328 | | | |
| St06 | 0.188 | F1 | 0.21 | 0.206 | 0.204 | 0.003 | 1.4 |
| | | F2 | 0.204 | 0.202 | | | |
| St07 | 0.094 | G1 | 0.102 | 0.133 | 0.132 | 0.001 | 1.1 |
| | | G2 | 0.099 | 0.131 | | | |
| St08 | 0 | H1 | Range | 0.059 | 0.059 | 0 | 0 |
| | | H2 | Range | 0.059 | | | |

| | | | | | |
|---|---|---|---|---|---|
| Concentration: 1y = ((A − D)/ | A | B | C | D | R\hat{ }2 |
| (1 + (x/C)\hat{ }B)) + D: | | | | | |
| Std (Standards: Concentration | 0.069 | 1.0781 | 0.627 | 9.614 | 1 |
| vs MeanValue) | | | | | |

Calculation: Standard curve 2:

TABLE 19

| | Concen- | | Back | | | | |
|---|---|---|---|---|---|---|---|
| | tration | | Conc. | | Mean | Std. | CV |
| Sample | (ng/mL) | Wells | Calc. | Values | Value | Dev. | % |
| St01 | 6 | A1 | 6.13 | 3.409 | 3.356 | 0.076 | 2.3 |
| | | A2 | 5.868 | 3.302 | | | |
| St02 | 3 | B1 | 3.082 | 1.986 | 1.945 | 0.058 | 3 |
| | | B2 | 2.931 | 1.904 | | | |
| St03 | 1.5 | C1 | 1.504 | 1.066 | 1.059 | 0.01 | 0.9 |
| | | C2 | 1.482 | 1.052 | | | |
| St04 | 0.75 | D1 | 0.769 | 0.589 | 0.575 | 0.021 | 3.6 |
| | | D2 | 0.726 | 0.56 | | | |
| St05 | 0.375 | E1 | 0.385 | 0.329 | 0.328 | 0.001 | 0.4 |
| | | E2 | 0.382 | 0.327 | | | |
| St06 | 0.188 | F1 | 0.188 | 0.195 | 0.195 | 0 | 0 |
| | | F2 | 0.188 | 0.195 | | | |
| St07 | 0.094 | G1 | 0.1 | 0.136 | 0.133 | 0.005 | 3.7 |
| | | G2 | 0.09 | 0.129 | | | |
| St08 | 0 | H1 | Range | 0.064 | 0.068 | 0.006 | 8.3 |
| | | H2 | 0.002 | 0.072 | | | |

| | | | | | |
|---|---|---|---|---|---|
| Concentration: 1y = ((A − D)/ | A | B | C | D | R\hat{ }2 |
| (1 + (x/C)\hat{ }B)) + D: | | | | | |
| Std (Standards: Concentration | 0.071 | 1.039 | 14.67 | 11.673 | 1 |
| vs MeanValue) | | | | | |

Calculation: Standard curve 3:

TABLE 20

| | Concen- | | Back | | | | |
|---|---|---|---|---|---|---|---|
| | tration | | Conc. | | Mean | Std. | CV |
| Sample | (ng/mL) | Wells | Calc. | Values | Value | Dev. | % |
| St01 | 6 | A1 | 6.108 | 3.482 | 3.438 | 0.063 | 1.8 |
| | | A2 | 5.89 | 3.393 | | | |
| St02 | 3 | B1 | 3.063 | 2.04 | 2.008 | 0.045 | 2.3 |
| | | B2 | 2.948 | 1.976 | | | |
| St03 | 1.5 | C1 | 1.514 | 1.11 | 1.099 | 0.016 | 1.5 |
| | | C2 | 1.479 | 1.087 | | | |
| St04 | 0.75 | D1 | 0.757 | 0.6 | 0.589 | 0.016 | 2.8 |
| | | D2 | 0.724 | 0.577 | | | |
| St05 | 0.375 | E1 | 0.389 | 0.341 | 0.336 | 0.007 | 2.1 |
| | | E2 | 0.375 | 0.331 | | | |
| St06 | 0.188 | F1 | 0.193 | 0.202 | 0.202 | 0.001 | 0.4 |
| | | F2 | 0.192 | 0.201 | | | |
| St07 | 0.094 | G1 | 0.1 | 0.137 | 0.137 | 0 | 0 |
| | | G2 | 0.1 | 0.137 | | | |
| St08 | 0 | H1 | Range | 0.062 | 0.063 | 0.001 | 2.2 |
| | | H2 | Range | 0.064 | | | |

| | | | | | |
|---|---|---|---|---|---|
| Concentration: 1y = ((A − D)/ | A | B | C | D | R\hat{ }2 |
| (1 + (x/C)\hat{ }B)) + D: | | | | | |
| Std (Standards: Concentration | 0.07 | 1.043 | 13.427 | 11.242 | 1 |
| vs MeanValue) | | | | | |

Calculation: human plasma samples:

TABLE 21

| | | | | Mean | | | | Klotho |
|---|---|---|---|---|---|---|---|---|
| Sample | Wells | Values | Result | Result | Std. Dev. | CV % | Dilution | (ng/mL) |
| A (0 h) | A3 | 0.238 | 0.256 | 0.251 | 0.007 | 2.8 | 4 | 1.003 |
| EDTA | A4 | 0.231 | 0.246 | | | | | |
| A (1 h) | A5 | 0.231 | 0.246 | 0.257 | 0.016 | 6.3 | 4 | 1.028 |
| EDTA | A6 | 0.247 | 0.269 | | | | | |
| A (4 h) | A7 | 0.235 | 0.251 | 0.25 | 0.002 | 0.8 | 4 | 1 |
| EDTA | A8 | 0.233 | 0.249 | | | | | |
| A (16 h) | A9 | 0.248 | 0.27 | 0.267 | 0.004 | 1.5 | 4 | 1.068 |
| EDTA | A10 | 0.244 | 0.264 | | | | | |
| A (24 h) | A11 | 0.261 | 0.289 | 0.299 | 0.015 | 5 | 4 | 1.197 |
| EDTA | A12 | 0.276 | 0.31 | | | | | |
| A (48 h) | B3 | 0.235 | 0.251 | 0.251 | 0 | 0 | 4 | 1.006 |
| EDTA | B4 | 0.235 | 0.251 | | | | | |
| A (72 h) | B5 | 0.242 | 0.261 | 0.256 | 0.007 | 2.8 | 4 | 1.026 |
| EDTA | B6 | 0.235 | 0.251 | | | | | |
| A (96 h) | B7 | 0.247 | 0.269 | 0.269 | 0.001 | 0.4 | 4 | 1.077 |
| EDTA | B8 | 0.248 | 0.27 | | | | | |
| A (168 h) | B9 | 0.254 | 0.279 | 0.286 | 0.01 | 3.5 | 4 | 1.143 |
| EDTA | B10 | 0.264 | 0.293 | | | | | |
| A (Finger) | B11 | 0.283 | 0.32 | 0.319 | 0.001 | 0.3 | 4 | 1.276 |
| EDTA | B12 | 0.282 | 0.318 | | | | | |
| A (0 h) | C3 | 0.239 | 0.257 | 0.259 | 0.002 | 0.8 | 4 | 1.034 |
| Heparin | C4 | 0.241 | 0.26 | | | | | |
| A (1 h) | C5 | 0.229 | 0.243 | 0.248 | 0.007 | 2.9 | 4 | 0.991 |
| Heparin | C6 | 0.236 | 0.253 | | | | | |
| A (4 h) | C7 | 0.236 | 0.253 | 0.261 | 0.012 | 4.6 | 4 | 1.046 |
| Heparin | C8 | 0.248 | 0.27 | | | | | |
| A (16 h) | C9 | 0.253 | 0.277 | 0.285 | 0.011 | 3.9 | 4 | 1.14 |
| Heparin | C10 | 0.264 | 0.293 | | | | | |
| A (24 h) | C11 | 0.256 | 0.281 | 0.301 | 0.027 | 9 | 4 | 1.202 |
| Heparin | C12 | 0.283 | 0.32 | | | | | |
| A (48 h) | D3 | 0.237 | 0.254 | 0.249 | 0.007 | 2.8 | 4 | 0.997 |
| Heparin | D4 | 0.23 | 0.244 | | | | | |
| A (72 h) | D5 | 0.241 | 0.26 | 0.265 | 0.007 | 2.7 | 4 | 1.06 |
| Heparin | D6 | 0.248 | 0.27 | | | | | |
| A (96 h) | D7 | 0.239 | 0.257 | 0.272 | 0.021 | 7.8 | 4 | 1.088 |
| Heparin | D8 | 0.26 | 0.287 | | | | | |
| A (168 h) | D9 | 0.252 | 0.276 | 0.273 | 0.004 | 1.5 | 4 | 1.091 |
| Heparin | D10 | 0.248 | 0.27 | | | | | |
| A (Finger) | D11 | 0.254 | 0.279 | 0.279 | 0 | 0 | 4 | 1.114 |
| Heparin | D12 | 0.066 | Range | | | | | |
| B (0 h) | E3 | 0.162 | 0.146 | 0.142 | 0.005 | 3.7 | 4 | 0.568 |
| EDTA | E4 | 0.157 | 0.138 | | | | | |
| B (1 h) | E5 | 0.16 | 0.143 | 0.145 | 0.003 | 2.2 | 4 | 0.579 |
| EDTA | E6 | 0.163 | 0.147 | | | | | |
| B (4 h) | E7 | 0.162 | 0.146 | 0.145 | 0.001 | 0.7 | 4 | 0.579 |
| EDTA | E8 | 0.161 | 0.144 | | | | | |
| B (16 h) | E9 | 0.173 | 0.162 | 0.162 | 0 | 0 | 4 | 0.647 |
| EDTA | E10 | 0.173 | 0.162 | | | | | |
| B (24 h) | E11 | 0.184 | 0.178 | 0.176 | 0.003 | 1.8 | 4 | 0.702 |
| EDTA | E12 | 0.181 | 0.173 | | | | | |
| B (48 h) | F3 | 0.167 | 0.153 | 0.161 | 0.011 | 7.1 | 4 | 0.644 |
| EDTA | F4 | 0.178 | 0.169 | | | | | |
| B (72 h) | F5 | 0.17 | 0.157 | 0.159 | 0.002 | 1.3 | 4 | 0.635 |
| EDTA | F6 | 0.172 | 0.16 | | | | | |
| B (96 h) | F7 | 0.173 | 0.162 | 0.165 | 0.004 | 2.5 | 4 | 0.659 |
| EDTA | F8 | 0.177 | 0.168 | | | | | |
| B (168 h) | F9 | 0.18 | 0.172 | 0.168 | 0.006 | 3.7 | 4 | 0.67 |
| EDTA | F10 | 0.174 | 0.163 | | | | | |
| B (Finger) | F11 | 0.182 | 0.175 | 0.187 | 0.017 | 9.3 | 4 | 0.749 |
| EDTA | F12 | 0.199 | 0.2 | | | | | |
| B (0 h) | G3 | 0.162 | 0.146 | 0.154 | 0.011 | 7.4 | 4 | 0.615 |
| Heparin | G4 | 0.173 | 0.162 | | | | | |
| B (1 h) | G5 | 0.175 | 0.165 | 0.162 | 0.003 | 1.9 | 4 | 0.65 |
| Heparin | G6 | 0.172 | 0.16 | | | | | |
| B (4 h) | G7 | 0.174 | 0.163 | 0.162 | 0.002 | 1.3 | 4 | 0.647 |
| Heparin | G8 | 0.172 | 0.16 | | | | | |
| B (16 h) | G9 | 0.173 | 0.162 | 0.164 | 0.003 | 1.9 | 4 | 0.656 |
| Heparin | G10 | 0.176 | 0.166 | | | | | |
| B (24 h) | G11 | 0.176 | 0.166 | 0.173 | 0.009 | 5.4 | 4 | 0.691 |
| Heparin | G12 | 0.185 | 0.179 | | | | | |
| B (48 h) | H3 | 0.177 | 0.168 | 0.17 | 0.003 | 1.8 | 4 | 0.679 |
| Heparin | H4 | 0.18 | 0.172 | | | | | |
| B (72 h) | H5 | 0.181 | 0.173 | 0.174 | 0.001 | 0.6 | 4 | 0.697 |
| Heparin | H6 | 0.182 | 0.175 | | | | | |
| B (96 h) | H7 | 0.176 | 0.166 | 0.157 | 0.013 | 8.6 | 4 | 0.626 |
| Heparin | H8 | 0.163 | 0.147 | | | | | |
| B (168 h) | H9 | 0.165 | 0.15 | 0.165 | 0.021 | 12.6 | 4 | 0.658 |
| Heparin | H10 | 0.185 | 0.179 | | | | | |
| B (Finger) | H11 | 0.182 | 0.175 | 0.181 | 0.008 | 4.6 | 4 | 0.723 |
| Heparin | H12 | 0.19 | 0.187 | | | | | |
| C (0 h) | A3 | 0.228 | 0.236 | 0.233 | 0.005 | 2.2 | 4 | 0.931 |
| EDTA | A4 | 0.223 | 0.229 | | | | | |
| C (1 h) | A5 | 0.237 | 0.25 | 0.25 | 0 | 0 | 4 | 0.998 |
| EDTA | A6 | 0.237 | 0.25 | | | | | |
| C (4 h) | A7 | 0.232 | 0.242 | 0.243 | 0.001 | 0.4 | 4 | 0.972 |
| EDTA | A8 | 0.233 | 0.244 | | | | | |
| C (16 h) | A9 | 0.24 | 0.254 | 0.249 | 0.007 | 2.9 | 4 | 0.996 |
| EDTA | A10 | 0.233 | 0.244 | | | | | |
| C (24 h) | A11 | 0.26 | 0.283 | 0.294 | 0.016 | 5.3 | 4 | 1.177 |
| EDTA | A12 | 0.275 | 0.305 | | | | | |
| C (48 h) | B3 | 0.246 | 0.263 | 0.258 | 0.007 | 2.8 | 4 | 1.031 |
| EDTA | B4 | 0.239 | 0.253 | | | | | |
| C (72 h) | B5 | 0.242 | 0.257 | 0.261 | 0.005 | 2 | 4 | 1.042 |
| EDTA | B6 | 0.247 | 0.264 | | | | | |
| C (96 h) | B7 | 0.244 | 0.26 | 0.258 | 0.002 | 0.8 | 4 | 1.034 |
| EDTA | B8 | 0.242 | 0.257 | | | | | |
| C (168 h) | B9 | 0.251 | 0.27 | 0.274 | 0.005 | 1.9 | 4 | 1.095 |
| EDTA | B10 | 0.256 | 0.277 | | | | | |
| C (Finger) | B11 | 0.227 | 0.235 | 0.253 | 0.025 | 9.9 | 4 | 1.01 |
| EDTA | B12 | 0.251 | 0.27 | | | | | |
| C (0 h) | C3 | 0.229 | 0.238 | 0.236 | 0.003 | 1.3 | 4 | 0.943 |
| Heparin | C4 | 0.226 | 0.233 | | | | | |
| C (1 h) | C5 | 0.222 | 0.228 | 0.232 | 0.006 | 2.7 | 4 | 0.928 |
| Heparin | C6 | 0.228 | 0.236 | | | | | |
| C (4 h) | C7 | 0.226 | 0.233 | 0.241 | 0.01 | 4.3 | 4 | 0.963 |
| Heparin | C8 | 0.236 | 0.248 | | | | | |
| C (16 h) | C9 | 0.234 | 0.245 | 0.254 | 0.012 | 4.9 | 4 | 1.016 |
| Heparin | C10 | 0.246 | 0.263 | | | | | |
| C (24 h) | C11 | 0.244 | 0.26 | 0.265 | 0.007 | 2.7 | 4 | 1.06 |
| Heparin | C12 | 0.251 | 0.27 | | | | | |
| C (48 h) | D3 | 0.225 | 0.232 | 0.238 | 0.008 | 3.5 | 4 | 0.951 |
| Heparin | D4 | 0.233 | 0.244 | | | | | |
| C (72 h) | D5 | 0.242 | 0.257 | 0.254 | 0.004 | 1.6 | 4 | 1.016 |
| Heparin | D6 | 0.238 | 0.251 | | | | | |
| C (96 h) | D7 | 0.266 | 0.292 | 0.272 | 0.028 | 10.3 | 4 | 1.089 |
| Heparin | D8 | 0.239 | 0.253 | | | | | |
| C (168 h) | D9 | 0.25 | 0.269 | 0.269 | 0.001 | 0.4 | 4 | 1.078 |
| Heparin | D10 | 0.251 | 0.27 | | | | | |
| C (Finger) | D11 | 0.244 | 0.26 | 0.25 | 0.015 | 5.8 | 4 | 0.998 |
| Heparin | D12 | 0.23 | 0.239 | | | | | |
| D (0 h) | E3 | 0.192 | 0.183 | 0.175 | 0.011 | 6.5 | 4 | 0.701 |
| EDTA | E4 | 0.181 | 0.167 | | | | | |
| D (1 h) | E5 | 0.165 | 0.144 | 0.15 | 0.009 | 6.3 | 4 | 0.601 |
| EDTA | E6 | 0.174 | 0.157 | | | | | |
| D (4 h) | E7 | 0.187 | 0.176 | 0.166 | 0.014 | 8.2 | 4 | 0.666 |
| EDTA | E8 | 0.174 | 0.157 | | | | | |
| D (16 h) | E9 | 0.196 | 0.189 | 0.169 | 0.029 | 17.3 | 4 | 0.675 |
| EDTA | E10 | 0.168 | 0.148 | | | | | |
| D (24 h) | E11 | 0.174 | 0.157 | 0.149 | 0.012 | 7.7 | 4 | 0.595 |
| EDTA | E12 | 0.163 | 0.141 | | | | | |
| D (48 h) | F3 | 0.174 | 0.157 | 0.158 | 0.002 | 1.3 | 4 | 0.633 |
| EDTA | F4 | 0.176 | 0.16 | | | | | |
| D (72 h) | F5 | 0.184 | 0.172 | 0.175 | 0.004 | 2.4 | 4 | 0.698 |
| EDTA | F6 | 0.188 | 0.178 | | | | | |
| D (96 h) | F7 | 0.196 | 0.189 | 0.18 | 0.013 | 6.9 | 4 | 0.722 |
| EDTA | F8 | 0.184 | 0.172 | | | | | |
| D (168 h) | F9 | 0.205 | 0.203 | 0.178 | 0.034 | 19.3 | 4 | 0.713 |
| EDTA | F10 | 0.172 | 0.154 | | | | | |
| D (Finger) | F11 | 0.169 | 0.149 | 0.15 | 0.001 | 0.7 | 4 | 0.601 |
| EDTA | F12 | 0.17 | 0.151 | | | | | |
| D (0 h) | G3 | 0.136 | 0.1 | 0.122 | 0.03 | 25 | 4 | 0.488 |
| Heparin | G4 | 0.165 | 0.144 | | | | | |
| D (1 h) | G5 | 0.177 | 0.161 | 0.152 | 0.013 | 8.2 | 4 | 0.61 |
| Heparin | G6 | 0.165 | 0.144 | | | | | |
| D (4 h) | G7 | 0.163 | 0.141 | 0.147 | 0.008 | 5.7 | 4 | 0.586 |
| Heparin | G8 | 0.171 | 0.152 | | | | | |
| D (16 h) | G9 | 0.191 | 0.182 | 0.165 | 0.024 | 14.6 | 4 | 0.66 |
| Heparin | G10 | 0.168 | 0.148 | | | | | |
| D (24 h) | G11 | 0.177 | 0.161 | 0.168 | 0.009 | 5.6 | 4 | 0.672 |
| Heparin | G12 | 0.186 | 0.175 | | | | | |
| D (48 h) | H3 | 0.19 | 0.181 | 0.171 | 0.014 | 7.9 | 4 | 0.684 |
| Heparin | H4 | 0.177 | 0.161 | | | | | |
| D (72 h) | H5 | 0.184 | 0.172 | 0.166 | 0.007 | 4.4 | 4 | 0.666 |
| Heparin | H6 | 0.177 | 0.161 | | | | | |
| D (96 h) | H7 | 0.198 | 0.192 | 0.185 | 0.01 | 5.6 | 4 | 0.74 |
| Heparin | H8 | 0.188 | 0.178 | | | | | |
| D (168 h) | H9 | 0.197 | 0.191 | 0.178 | 0.018 | 9.9 | 4 | 0.713 |
| Heparin | H10 | 0.18 | 0.166 | | | | | |
| D (Finger) | H11 | 0.17 | 0.151 | 0.149 | 0.003 | 2.1 | 4 | 0.595 |
| Heparin | H12 | 0.167 | 0.147 | | | | | |
| Spike (0 h) | A3 | 0.449 | 0.542 | 0.537 | 0.007 | 1.3 | 4 | 2.147 |
| EDTA | A4 | 0.442 | 0.532 | | | | | |
| Spike (1 h) | B3 | 0.438 | 0.526 | 0.52 | 0.009 | 1.7 | 4 | 2.079 |
| EDTA | B4 | 0.429 | 0.513 | | | | | |
| Spike (4 h) | C3 | 0.426 | 0.509 | 0.51 | 0.001 | 0.2 | 4 | 2.039 |
| EDTA | C4 | 0.427 | 0.511 | | | | | |
| Spike (16 h) | D3 | 0.442 | 0.532 | 0.531 | 0.001 | 0.2 | 4 | 2.124 |
| EDTA | D4 | 0.441 | 0.53 | | | | | |
| Spike (24 h) | E3 | 0.471 | 0.573 | 0.56 | 0.018 | 3.2 | 4 | 2.24 |
| EDTA | E4 | 0.453 | 0.547 | | | | | |
| Spike (48 h) | F3 | 0.47 | 0.571 | 0.566 | 0.008 | 1.4 | 4 | 2.263 |
| EDTA | F4 | 0.462 | 0.56 | | | | | |
| Spike (72 h) | G3 | 0.514 | 0.634 | 0.636 | 0.003 | 0.5 | 4 | 2.545 |
| EDTA | G4 | 0.517 | 0.638 | | | | | |
| Spike (96 h) | H3 | 0.558 | 0.697 | 0.683 | 0.019 | 2.8 | 4 | 2.733 |
| EDTA | H4 | 0.539 | 0.67 | | | | | |
| Spike (168 h) | A5 | 0.553 | 0.69 | 0.693 | 0.005 | 0.7 | 4 | 2.773 |
| EDTA | A6 | 0.558 | 0.697 | | | | | |
| Spike (Finger) | B5 | 0.587 | 0.738 | 0.75 | 0.016 | 2.2 | 4 | 3 |
| EDTA | B6 | 0.603 | 0.761 | | | | | |
| Spike (0 h) | C5 | 0.506 | 0.623 | 0.617 | 0.008 | 1.3 | 4 | 2.468 |
| Heparin | C6 | 0.498 | 0.611 | | | | | |
| Spike (1 h) | D5 | 0.514 | 0.634 | 0.637 | 0.004 | 0.6 | 4 | 2.548 |
| Heparin | D6 | 0.518 | 0.64 | | | | | |
| Spike (4 h) | E5 | 0.506 | 0.623 | 0.634 | 0.016 | 2.5 | 4 | 2.536 |
| Heparin | E6 | 0.522 | 0.645 | | | | | |
| Spike (16 h) | F5 | 0.537 | 0.667 | 0.675 | 0.011 | 1.6 | 4 | 2.699 |
| Heparin | F6 | 0.548 | 0.683 | | | | | |
| Spike (24 h) | G5 | 0.54 | 0.671 | 0.669 | 0.003 | 0.5 | 4 | 2.676 |
| Heparin | G6 | 0.537 | 0.667 | | | | | |
| Spike (48 h) | H5 | 0.564 | 0.705 | 0.71 | 0.006 | 0.9 | 4 | 2.839 |
| Heparin | H6 | 0.57 | 0.714 | | | | | |
| Spike (72 h) | A7 | 0.51 | 0.628 | 0.625 | 0.004 | 0.6 | 4 | 2.502 |
| Heparin | A8 | 0.506 | 0.623 | | | | | |
| Spike (96 h) | B7 | 0.501 | 0.616 | 0.621 | 0.008 | 1.3 | 4 | 2.485 |
| Heparin | B8 | 0.509 | 0.627 | | | | | |
| Spike (168 h) | C7 | 0.481 | 0.587 | 0.603 | 0.022 | 3.7 | 4 | 2.411 |
| Heparin | C8 | 0.503 | 0.618 | | | | | |
| Spike (Finger) | D7 | 0.568 | 0.711 | 0.68 | 0.044 | 6.5 | 4 | 2.719 |
| Heparin | D8 | 0.524 | 0.648 | | | | | |

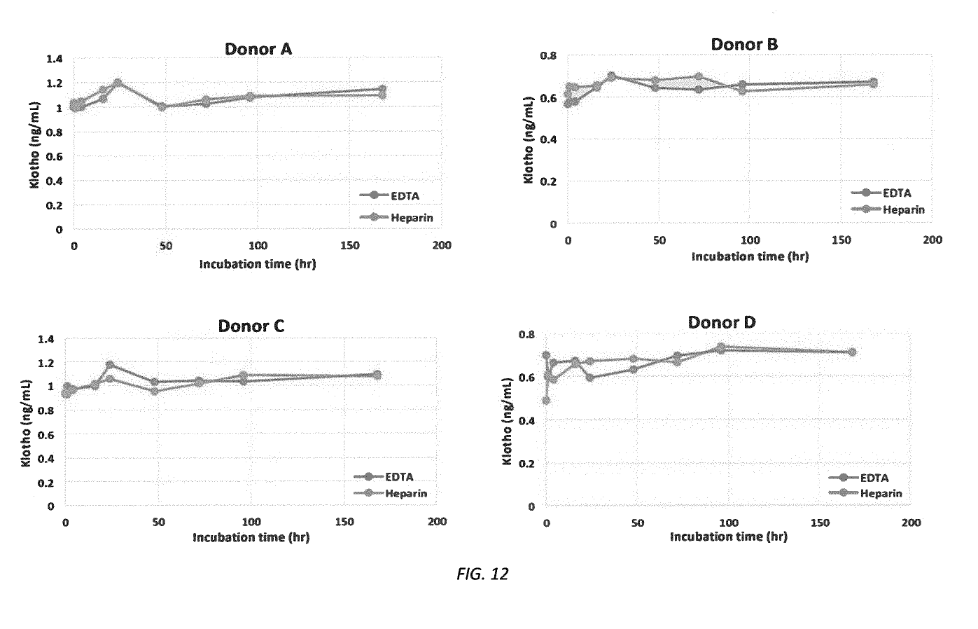

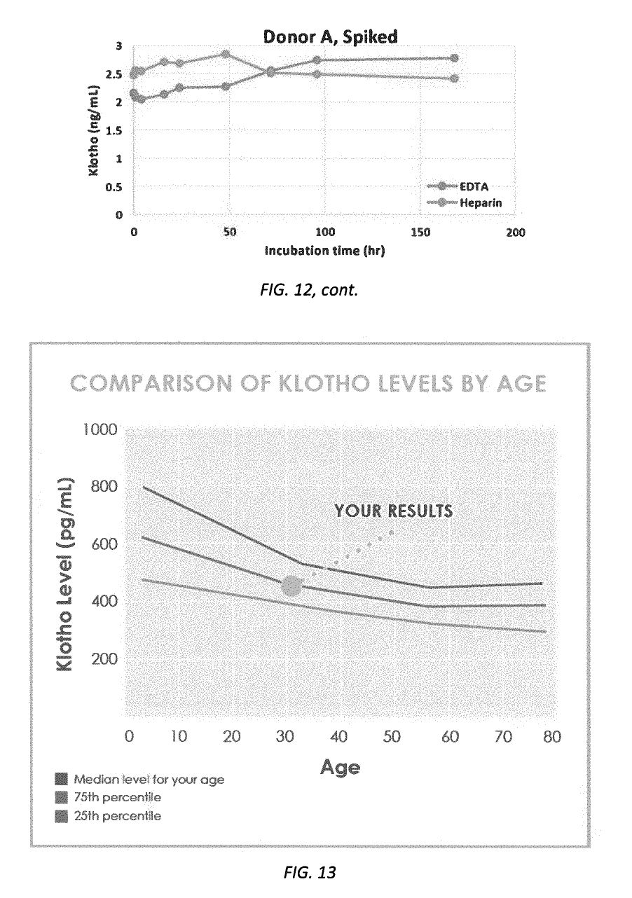

FIG. 12 illustrates Klotho level at different incubation time at RT.

Table 22, below presents average Klotho protein level (ng/mL) in plasma:

TABLE 22

| Blood sample | |
|---|---|
| Venous | Capillary (finger) |

| Anticoagulant | EDIA | Heparin | EDIA | Heparin |
|---|---|---|---|---|
| A | 1.06 | 1.07 | 1.28 | 1.11 |
| B | 0.63 | 0.66 | 0.75 | 0.72 |
| C | 1.03 | 1.08 | 1.01 | 1.00 |
| D | 0.67 | 0.65 | 0.60 | 0.60 |
| A, Spiked | 2.33 | 2.57 | 3.00 | 2.72 |

Table 23, below presents spiked Klotho recovery rate (%)

TABLE 23

| Blood sample | |
|---|---|
| Venous | Capillary (finger) |

| Anticoagulant | EDIA | Heparin | EDIA | Heparin |
|---|---|---|---|---|
| A, Spiked | 84% | 100% | 115% | 107% |

### Klotho Stability Testing at Various Temperatures Emulating Shipping Stress

Phlebotomist(s) collected >12 mL blood (sample) from each of four donors into heparinized blood tubes. Technician(s) immediately dispensed 300-400 uL of blood from the collected sample of each donor into separate heparinized Micro Tubes (from BD). Tubes were stored and processed according to the study design (see table below). Blood was processed at each timepoint or centrifuged and stored (plasma) at −80° C. All samples were tested at approximately day 17.

For the study, 120 data points were collected, representing 6 conditions, over 14 days (see below).

TABLE 24

| | Donor 1 | | | | | Donor 2 | | | | |
|---|---|---|---|---|---|---|---|---|---|---|
| | 0 | 4 | 7 | 9 | 14 | 0 | 4 | 7 | 9 | 14 |
| Temperature Group | Hr | Day | Day | Day | Day | Hr | Day | Day | Day | Day |
| 20° C. the enire time | X | X | X | X | X | X | X | X | X | X |
| 8 hrs @ 0 C., 20° C. | X | X | X | X | X | X | X | X | X | X |
| remaining time | | | | | | | | | | |
| 24 hrs @ 0 C., 20° C. | X | X | X | X | X | X | X | X | X | X |
| remaining time | | | | | | | | | | |
| 8 hrs @−150 C., 20° C. | X | X | X | X | X | X | X | X | X | X |
| remaining time | | | | | | | | | | |
| 24 hrs @−15 C., 20° C. | X | X | X | X | X | X | X | X | X | X |
| remaining time | | | | | | | | | | |
| 8 hrs @37 C., 20° C. | X | X | X | X | X | X | X | X | X | X |
| remaining time | | | | | | | | | | |
| 24 hrs @37 C., 20° C. | X | X | X | X | X | X | X | X | X | X |
| remaining time | | | | | | | | | | |
| | Donor 3 | | | | | Donor 4 | | | | |
| | 0 | 4 | 7 | 9 | 14 | 0 | 4 | 7 | 9 | 14 |
| Temperature Group | Hr | Day | Day | Day | Day | Hr | Day | Day | Day | Day |
| 20° C. the enire time | X | X | X | X | X | X | X | X | X | X |
| 8 hrs @ 0 C., 20° C. | X | X | X | X | X | X | X | X | X | X |
| remaining time | | | | | | | | | | |
| 24 hrs @ 0 C., 20° C. | X | X | X | X | X | X | X | X | X | X |
| remaining time | | | | | | | | | | |
| 8 hrs @−150 C., 20° C. | X | X | X | X | X | X | X | X | X | X |
| remaining time | | | | | | | | | | |
| 24 hrs @−15 C., 20° C. | X | X | X | X | X | X | X | X | X | X |
| remaining time | | | | | | | | | | |
| 8 hrs @37 C., 20° C. | X | X | X | X | X | X | X | X | X | X |
| remaining time | | | | | | | | | | |
| 24 hrs @37 C., 20° C. | X | X | X | X | X | X | X | X | X | X |
| remaining time | | | | | | | | | | |

The plate layout (see Table 25) was the same for all four donors (see Tables 26-29, below).

TABLE 25

| | | | | | |
|---|---|---|---|---|---|
| A | STD, 6 ng/mL | O h | @−15 C., 8 hr, Day 7 | @−0 C., 24 hr, Day 4 | @37 C., 8 hr, Day 9 |
| B | STD, 3 ng/mL | @RT Day 4 | @−15 C., 8 hr, Day 9 | @−0 C., 24 hr, Day 5 | @37 C., 8 hr, Day 14 |
| C | STD, 1.5 ng/mL | @RT Day 5 | @−15 C., 8 hr, Day 14 | @−0 C., 24 hr, Day 7 | @37 C., 24 hr, Day 4 |
| D | STD, 0.75 ng/mL | @RT day 7 | @0 C., 8 hr, Day 4 | @−0 C., 24 hr, Day 9 | @37 C., 24 hr, Day 5 |
| E | STD, 0.375 ng/mL | @RT Day 9 | @0 C., 8 hr, Day 5 | @−0 C., 24 hr, Day 14 | @37 C., 24 hr, Day 7 |
| F | STD, 0.188 ng/mL | @RT day 14 | @0 C., 8 hr, Day 7 | @37 C., 8 hr, Day 4 | @37 C., 24 hr, Day 9 |
| G | STD, 0.094 ng/mL | @−15 C., 8 hr, Day 4 | @−0 C., 8 hr, Day 9 | @37 C., 8 hr, Day 5 | @37 C., 24 hr, Day 14 |
| H | STD, 0 ng/mL | @−15 C., 8 hr, Day 5 | @−0 C., 8 hr, Day 14 | @37 C., 8 hr, Day 6 | |

ELISA protocol was conducted according to instructions of soluble Klotho ELISA kit (human), IBL, Cat# 27998). Raw data are given below in Tables 26-29:
Plate 1, Donor 1:

TABLE 26

| | | | | | | | | | | |
|---|---|---|---|---|---|---|---|---|---|---|
| 2.689 | 2.577 | 0.138 | 0.141 | 0.09 | 0.088 | 0.133 | 0.125 | 0.107 | 0.109 | 0.047 |
| 1.481 | 1.423 | 0.174 | 0.162 | 0.107 | 0.177 | 0.169 | 0.161 | 0.142 | 0.159 | 0.053 |
| 0.801 | 0.833 | 0.128 | 0.131 | 0.081 | 0.085 | 0.125 | 0.124 | 0.114 | 0.11 | 0.128 |
| 0.443 | 0.414 | 0.129 | 0.123 | 0.123 | 0.126 | 0.121 | 0.12 | 0.111 | 0.111 | 0.065 |
| 0.26 | 0.253 | 0.111 | 0.128 | 0.132 | 0.129 | 0.116 | 0.118 | 0.111 | 0.115 | 0.06 |
| 0.162 | 0.154 | 0.109 | 0.116 | 0.128 | 0.131 | 0.135 | 0.127 | 0.113 | 0.111 | 0.062 |
| 0.15 | 0.114 | 0.087 | 0.092 | 0.126 | 0.131 | 0.137 | 0.137 | 0.099 | 0.124 | 0.062 |
| 0.148 | 0.058 | 0.219 | 0.098 | 0.115 | 0.277 | 0.243 | 0.128 | 0.052 | 0.056 | 0.087 |

Plate 2, Donor 2:

TABLE 27

| | | | | | | | | | | |
|---|---|---|---|---|---|---|---|---|---|---|
| 2.799 | 2.887 | 0.218 | 0.223 | 0.124 | 0.122 | 0.217 | 0.201 | 0.174 | 0.167 | 0.088 |
| 1.594 | 1.596 | 0.222 | 0.218 | 0.112 | 0.105 | 0.208 | 0.208 | 0.161 | 0.153 | 0.092 |
| 0.91 | 0.898 | 0.223 | 0.219 | 0.108 | 0.109 | 0.202 | 0.202 | 0.172 | 0.168 | 0.154 |
| 0.49 | 0.475 | 0.207 | 0.21 | 0.208 | 0.201 | 0.199 | 0.203 | 0.17 | 0.175 | 0.067 |
| 0.288 | 0.284 | 0.217 | 0.212 | 0.21 | 0.208 | 0.162 | 0.165 | 0.163 | 0.167 | 0.086 |
| 0.175 | 0.162 | 0.174 | 0.173 | 0.203 | 0.201 | 0.203 | 0.202 | 0.161 | 0.157 | 0.105 |
| 0.114 | 0.114 | 0.116 | 0.11 | 0.208 | 0.206 | 0.211 | 0.219 | 0.173 | 0.147 | 0.486 |
| 0.08 | 0.064 | 0.175 | 0.123 | 0.175 | 0.186 | 0.237 | 0.203 | 0.058 | 0.051 | 0.082 |

Plate 3, Donor 3:

TABLE 28

| | | | | | | | | | | |
|---|---|---|---|---|---|---|---|---|---|---|
| 2.768 | 2.8 | 0.155 | 0.156 | 0.108 | 0.102 | 0.161 | 0.159 | 0.14 | 0.137 | 0.132 |
| 1.629 | 1.642 | 0.161 | 0.158 | 0.1 | 0.093 | 0.156 | 0.16 | 0.131 | 0.13 | 0.057 |
| 0.926 | 0.911 | 0.159 | 0.159 | 0.099 | 0.105 | 0.15 | 0.152 | 0.139 | 0.145 | 0.053 |
| 0.51 | 0.484 | 0.15 | 0.151 | 0.15 | 0.146 | 0.154 | 0.149 | 0.137 | 0.138 | 0.057 |
| 0.288 | 0.284 | 0.16 | 0.152 | 0.156 | 0.157 | 0.132 | 0.133 | 0.134 | 0.134 | 0.111 |
| 0.188 | 0.165 | 0.131 | 0.136 | 0.151 | 0.152 | 0.15 | 0.152 | 0.133 | 0.129 | 0.068 |
| 0.119 | 0.121 | 0.114 | 0.115 | 0.154 | 0.156 | 0.164 | 0.164 | 0.122 | 0.123 | 0.066 |
| 0.092 | 0.071 | 0.158 | 0.113 | 0.149 | 0.155 | 0.17 | 0.164 | 0.059 | 0.061 | 0.078 |

Plate 4, Donor 4:

TABLE 29

| | | | | | | | | | | |
|---|---|---|---|---|---|---|---|---|---|---|
| 2.787 | 2.699 | 0.177 | 0.207 | 0.137 | 0.128 | 0.196 | 0.185 | 0.144 | 0.14 | 0.132 |
| 1.614 | 1.622 | 0.199 | 0.186 | 0.112 | 0.104 | 0.18 | 0.178 | 0.139 | 0.133 | 0.057 |
| 0.911 | 0.884 | 0.199 | 0.187 | 0.109 | 0.104 | 0.168 | 0.17 | 0.139 | 0.135 | 0.053 |
| 0.494 | 0.475 | 0.177 | 0.173 | 0.182 | 0.178 | 0.166 | 0.168 | 0.147 | 0.148 | 0.058 |
| 0.302 | 0.287 | 0.164 | 0.17 | 0.19 | 0.188 | 0.15 | 0.156 | 0.14 | 0.137 | 0.111 |
| 0.179 | 0.175 | 0.164 | 0.169 | 0.183 | 0.179 | 0.187 | 0.191 | 0.144 | 0.14 | 0.068 |
| 0.122 | 0.122 | 0.125 | 0.122 | 0.171 | 0.164 | 0.174 | 0.172 | 0.133 | 0.133 | 0.067 |
| 0.083 | 0.062 | 0.165 | 0.128 | 0.165 | 0.162 | 0.176 | 0.173 | 0.049 | 0.048 | 0.079 |

Data analysis is given below in Tables 30-41:
Plate 1, Donor 1:
Standard Calculations

TABLE 30

| Sam- | Concen- | | | Val- | MeanVal- | Std. | CV |
|---|---|---|---|---|---|---|---|
| ple | tration | Wells | BackConcCalc | ues | ue | Dev. | % |
| St01 | 6 | A1 | 5.965 | 2.611 | 2.555 | 0.079 | 3.1 |
| | | A2 | 5.7 | 2.499 | | | |
| St02 | 3 | B1 | 3.133 | 1.403 | 1.374 | 0.041 | 3 |
| | | B2 | 2.998 | 1.345 | | | |
| St03 | 1.5 | C1 | 1.575 | 0.723 | 0.739 | 0.023 | 3.1 |
| | | C2 | 1.647 | 0.755 | | | |
| St04 | 0.75 | D1 | 0.775 | 0.365 | 0.35 | 0.021 | 5.9 |
| | | D2 | 0.711 | 0.336 | | | |
| St05 | 0.375 | E1 | 0.376 | 0.182 | 0.178 | 0.005 | 2.8 |
| | | E2 | 0.361 | 0.175 | | | |
| St06 | 0.188 | F1 | 0.168 | 0.084 | 0.08 | 0.006 | 7.1 |
| | | F2 | 0.151 | 0.076 | | | |
| St07 | 0.094 | G1 | 0.143 | 0.072 | 0.054 | 0.025 | 47.6 |
| | | G2 | 0.069 | 0.036 | | | |

Sample Calculations

TABLE 31

| | | | | | | | | | Difference |
|---|---|---|---|---|---|---|---|---|---|
| Sample | Wells | Values | Result | MeanResult | Std. Dev. | CV % | Dilution | Adj. Result | to 0 h |
| 0 h | A3 | 0.06 | 0.118 | 0.121 | 0.004 | 3.6 | 4 | 0.486 | 1.00 |
| | A4 | 0.063 | 0.124 | | | | | | |
| RT Day 4 | B3 | 0.096 | 0.193 | 0.181 | 0.018 | 9.8 | 4 | 0.723 | 1.49 |
| | B4 | 0.084 | 0.168 | | | | | | |
| RT Day 5 | C3 | 0.05 | 0.098 | 0.101 | 0.004 | 4.3 | 4 | 0.403 | 0.83 |
| | C4 | 0.053 | 0.104 | | | | | | |
| RT Day 7 | D3 | 0.051 | 0.1 | 0.094 | 0.009 | 9.3 | 4 | 0.375 | 0.77 |
| | D4 | 0.045 | 0.088 | | | | | | |
| RT Day 9 | E3 | 0.033 | 0.063 | 0.08 | 0.024 | 30.4 | 4 | 0.322 | 0.66 |
| | E4 | 0.05 | 0.098 | | | | | | |
| RT Day 14 | F3 | 0.031 | 0.059 | 0.066 | 0.01 | 15.1 | 4 | 0.265 | 0.55 |
| | F4 | 0.038 | 0.073 | | | | | | |
| −15 C., 8 hr | G3 | 0.009 | 0.016 | 0.021 | 0.007 | 33.3 | 4 | 0.082 | 0.17 |
| Day 4 | G4 | 0.014 | 0.025 | | | | | | |
| −15 C., 8 hr | H3 | 0.141 | 0.288 | 0.163 | 0.178 | 109.1 | 4 | 0.651 | 1.34 |
| Day 5 | H4 | 0.02 | 0.037 | | | | | | |
| −15 C., 8 hr | A5 | 0.012 | 0.022 | 0.02 | 0.003 | 14 | 4 | 0.078 | 0.16 |
| Day 7 | A6 | 0.01 | 0.018 | | | | | | |
| −15 C., 8 hr | B5 | 0.029 | 0.055 | 0.127 | 0.102 | 80.2 | 4 | 0.509 | 1.05 |
| Day 9 | B6 | 0.099 | 0.2 | | | | | | |
| −15 C., 8 hr | C5 | 0.003 | 0.004 | 0.008 | 0.005 | 64.8 | 4 | 0.033 | 0.07 |
| Day 14 | C6 | 0.007 | 0.012 | | | | | | |
| 0 C., 8 hr, | D5 | 0.045 | 0.088 | 0.091 | 0.004 | 4.8 | 4 | 0.362 | 0.74 |
| Day 4 | D6 | 0.048 | 0.094 | | | | | | |
| 0 C., 8 hr, | E5 | 0.054 | 0.106 | 0.103 | 0.004 | 4.2 | 4 | 0.412 | 0.85 |
| Day 5 | E6 | 0.051 | 0.1 | | | | | | |
| 0 C., 8 hr, | F5 | 0.05 | 0.098 | 0.101 | 0.004 | 4.3 | 4 | 0.403 | 0.83 |
| Day 7 | F6 | 0.053 | 0.104 | | | | | | |
| 0 C., 8 hr, | G5 | 0.048 | 0.094 | 0.099 | 0.007 | 7.3 | 4 | 0.395 | 0.81 |
| Day 9 | G6 | 0.053 | 0.104 | | | | | | |
| 0 C., 8 hr, | H5 | 0.037 | 0.071 | 0.242 | 0.241 | 99.8 | 4 | 0.968 | 1.99 |
| Day 14 | H6 | 0.199 | 0.413 | | | | | | |
| 0 C., 24 hr, | A7 | 0.055 | 0.108 | 0.1 | 0.012 | 11.6 | 4 | 0.399 | 0.82 |
| Day 4 | A8 | 0.047 | 0.092 | | | | | | |
| 0 C., 24 hr, | B7 | 0.0914 | 0.183 | 0.174 | 0.012 | 6.8 | 4 | 0.698 | 1.44 |
| Day 5 | B8 | 0.083 | 0.166 | | | | | | |
| 0 C., 24 hr, | C7 | 0.047 | 0.092 | 0.091 | 0.001 | 1.6 | 4 | 0.362 | 0.74 |
| Day 7 | C8 | 0.046 | 0.09 | | | | | | |
| 0 C., 24 hr, | D7 | 0.043 | 0.083 | 0.082 | 0.001 | 1.7 | 4 | 0.33 | 0.68 |
| Day 9 | D8 | 0.042 | 0.081 | | | | | | |
| 0 C., 24 hr, | E7 | 0.038 | 0.073 | 0.075 | 0.003 | 3.8 | 4 | 0.301 | 0.62 |
| Day 14 | E8 | 0.04 | 0.077 | | | | | | |
| 37 C., 8 hr, | F7 | 0.057 | 0.112 | 0.104 | 0.012 | 11.2 | 4 | 0.416 | 0.86 |
| Day 4 | F8 | 0.049 | 0.096 | | | | | | |
| 37 C., 8 hr, | G7 | 0.059 | 0.116 | 0.116 | 0 | 0 | 4 | 0.465 | 0.96 |
| Day 5 | G8 | 0.059 | 0.116 | | | | | | |
| 37 C., 8 hr, | H7 | 0.165 | 0.34 | 0.219 | 0.171 | 78.2 | 4 | 0.875 | 1.80 |
| Day 7 | H8 | 0.05 | 0.098 | | | | | | |
| 37 C., 8 hr, | A9 | 0.029 | 0.055 | 0.057 | 0.003 | 5 | 4 | 0.229 | 0.47 |
| Day 9 | A10 | 0.031 | 0.059 | | | | | | |
| 37 C., 8 hr, | B9 | 0.064 | 0.127 | 0.144 | 0.025 | 17.3 | 4 | 0.577 | 1.19 |
| Day 14 | B10 | 0.081 | 0.162 | | | | | | |
| 37 C., 24 hr, | C9 | 0.036 | 0.069 | 0.065 | 0.006 | 8.8 | 4 | 0.261 | 0.54 |
| Day 4 | C10 | 0.032 | 0.061 | | | | | | |
| 37 C., 24 hr, | D9 | 0.033 | 0.063 | 0.063 | 0 | 0 | 4 | 0.253 | 0.52 |
| Day 5 | D10 | 0.033 | 0.063 | | | | | | |
| 37 C., 24 hr, | E9 | 0.033 | 0.063 | 0.067 | 0.006 | 8.5 | 4 | 0.269 | 0.55 |
| Day 7 | E10 | 0.037 | 0.071 | | | | | | |
| 37 C., 24 hr, | F9 | 0.035 | 0.067 | 0.065 | 0.003 | 4.4 | 4 | 0.261 | 0.54 |
| Day 9 | F10 | 0.033 | 0.063 | | | | | | |
| 37 C., 24 hr, | G9 | 0.021 | 0.039 | 0.064 | 0.036 | 55.3 | 4 | 0.258 | 0.53 |
| Day 14 | G10 | 0.046 | 0.09 | | | | | | |

Klotho Concentration Changes in Time Course

TABLE 32

| | Room | −15° C. | 0° C. | 0° C. | 37° C. | 37° C. |
|---|---|---|---|---|---|---|
| | Temp | 8 hr | 8 hr | 24 hr | 8 hr | 24 hr |
| Day 4 | 149% | 17% | 74% | 82% | 86% | 54% |
| Day 5 | 83% | 134% | 85% | 144% | 96% | 52% |
| Day 7 | 77% | 16% | 83% | 74% | 180% | 55% |
| Day 9 | 66% | 105% | 81% | 68% | 47% | 54% |
| Day 14 | 55% | 7% | 199% | 62% | 119% | 53% |

Plate 2, Donor 2:
Standard Calculations

TABLE 33

| Sam- | Concen- | | | Val- | MeanVal- | Std. | CV |
|---|---|---|---|---|---|---|---|
| ple | tration | Wells | BackConcCalc | ues | ue | Dev. | % |
| St01 | 6 | A1 | 5.451 | 2.736 | 2.78 | 0.062 | 2.2 |
| | | A2 | 5.633 | 2.824 | | | |
| St02 | 3 | B1 | 2.983 | 1.531 | 1.532 | 0.001 | 0.1 |
| | | B2 | 2.987 | 1.533 | | | |
| St03 | 1.5 | C1 | 1.613 | 0.847 | 0.841 | 0.008 | 1 |
| | | C2 | 1.589 | 0.835 | | | |
| St04 | 0.75 | D1 | 0.792 | 0.427 | 0.419 | 0.011 | 2.5 |
| | | D2 | 0.763 | 0.412 | | | |
| St05 | 0.375 | E1 | 0.407 | 0.225 | 0.223 | 0.003 | 1.3 |
| | | E2 | 0.399 | 0.221 | | | |
| St06 | 0.188 | F1 | 0.197 | 0.112 | 0.105 | 0.009 | 8.7 |
| | | F2 | 0.173 | 0.099 | | | |
| St07 | 0.094 | G1 | 0.087 | 0.051 | 0.051 | 0 | 0 |
| | | G2 | 0.087 | 0.051 | | | |

Sample Calculations

TABLE 34

| | | | | | | | | | Difference |
|---|---|---|---|---|---|---|---|---|---|
| Sample | Wells | Values | Result | MeanResult | Std. Dev. | CV % | Dilution | Adj. Result | to 0 h |
| 0 h | A3 | 0.155 | 0.278 | 0.281 | 0.007 | 2.3 | 4 | 1.124 | 1.00 |
| | A4 | 0.16 | 0.286 | | | | | | |
| RT Day 4 | B3 | 0.159 | 0.284 | 0.28 | 0.005 | 1.9 | 4 | 1.12 | 1.00 |
| | B4 | 0.155 | 276 | | | | | | |
| RT Day 5 | C3 | 0.16 | 0.286 | 0.282 | 0.005 | 1.9 | 4 | 1.127 | 1.00 |
| | C4 | 0.156 | 0.278 | | | | | | |
| RT Day 7 | D3 | 0.144 | 2256 | 0.259 | 0.004 | 1.5 | 4 | 1.035 | 0.92 |
| | D4 | 0.147 | 0.261 | | | | | | |
| RT Day 9 | E3 | 0.154 | 0.274 | 0.27 | 0.007 | 2.4 | 4 | 1.079 | 0.96 |
| | E4 | 0.149 | 0.265 | | | | | | |
| RT Day 14 | F3 | 0.111 | 0.195 | 0.194 | 0.001 | 0.7 | 4 | 0.777 | 0.69 |
| | F4 | 0.11 | 0.193 | | | | | | |
| −15 C., 8 hr | G3 | 0.053 | 0.09 | 0.085 | 0.008 | 8.9 | 4 | 0.34 | 0.3 |
| Day 4 | G4 | 0.047 | 0.08 | | | | | | |
| −15 C., 8 hr | H3 | 0.112 | 0.197 | 0.15 | 0.067 | 44.4 | 4 | 0.6 | 0.53 |
| Day 5 | H4 | 0.06 | 0.103 | | | | | | |
| −15 C., 8 hr | A5 | 0.061 | 0.105 | 0.103 | 0.003 | 2.5 | 4 | 0.411 | 0.37 |
| Day 7 | A6 | 0.059 | 0.101 | | | | | | |
| −15 C., 8 hr | B5 | 0.049 | 0.083 | 0.077 | 0.009 | 11.4 | 4 | 0.308 | 0.27 |
| Day 9 | B6 | 0.042 | 0.071 | | | | | | |
| −15 C., 8 hr | C5 | 0.045 | 0.076 | 0.077 | 0.001 | 1.6 | 4 | 0.308 | 0.27 |
| Day 14 | C6 | 0.046 | 0.078 | | | | | | |
| 0 C., 8 hr, | D5 | 0.145 | 0.258 | 0.251 | 0.009 | 3.6 | 4 | 1.005 | 0.89 |
| Day 4 | D6 | 0.138 | 0.245 | | | | | | |
| 0 C., 8 hr, | E5 | 0.147 | 0.261 | 0.26 | 0.003 | 1 | 4 | 1.038 | 0.92 |
| Day 5 | E6 | 0.145 | 0.258 | | | | | | |
| 0 C., 8 hr, | F5 | 0.14 | 0.249 | 0.247 | 0.003 | 1.1 | 4 | 0.987 | 0.88 |
| Day 7 | F6 | 0.138 | 0.245 | | | | | | |
| 0 C., 8 hr, | G5 | 0.145 | 0.258 | 0.256 | 0.003 | 1 | 4 | 1.024 | 0.91 |
| Day 9 | G6 | 0.143 | 0.254 | | | | | | |
| 0 C., 8 hr, | H5 | 0.112 | 0.197 | 0.207 | 0.014 | 6.9 | 4 | 0.828 | 0.74 |
| Day 14 | H6 | 0.123 | 0.217 | | | | | | |
| 0 C., 24 hr, | A7 | 0.154 | 0.274 | 0.26 | 0.021 | 8.1 | 4 | 1.038 | 0.92 |
| Day 4 | A8 | 0.138 | 0.245 | | | | | | |
| 0 C., 24 hr, | B7 | 0.145 | 0.258 | 0.258 | 0 | 0 | 4 | 1.031 | 0.92 |
| Day 5 | B8 | 0.145 | 0.258 | | | | | | |
| 0 C., 24 hr, | C7 | 0.139 | 0.247 | 0.247 | 0 | 0 | 4 | 0.987 | 0.88 |
| Day 7 | C8 | 0.139 | 0.247 | | | | | | |
| 0 C., 24 hr, | D7 | 0.136 | 0.241 | 0.245 | 0.005 | 2.1 | 4 | 0.979 | 0.87 |
| Day 9 | D8 | 0.14 | 0.249 | | | | | | |
| 0 C., 24 hr, | E7 | 0.099 | 0.173 | 0.176 | 0.004 | 202 | 4 | 0.704 | 0.63 |
| Day 14 | E8 | 0.102 | 0.179 | | | | | | |
| 37 C., 8 hr, | F7 | 0.14 | 0.249 | 0.0248 | 0.001 | 0.5 | 4 | 0.99 | 0.88 |
| Day 4 | F8 | 0.139 | 0.247 | | | | | | |
| 37 C., 8 hr, | G7 | 0.148 | 0.263 | 0.271 | 0.01 | 3.9 | 4 | 1.083 | 0.96 |
| Day 5 | G8 | 0.156 | 0.278 | | | | | | |
| 37 C., 8 hr, | H7 | 0.174 | 0.312 | 0.28 | 0.045 | 15.9 | 4 | 1.12 | 1.00 |
| Day 7 | H8 | 0.014 | 0.249 | | | | | | |
| 37 C., 8 hr, | A9 | 0.111 | 0.195 | 0.159 | 0.009 | 4.8 | 4 | 0.755 | 0.67 |
| Day 9 | A10 | 0.104 | 0.182 | | | | | | |
| 37 C., 8 hr, | B9 | 0.098 | 0.171 | 0.164 | 0.01 | 6.3 | 4 | 0.657 | 0.58 |
| Day 14 | B10 | 0.09 | 0.157 | | | | | | |
| 37 C., 24 hr, | C9 | 0.109 | 0.192 | 0.188 | 0.005 | 2.8 | 4 | 0.752 | 0.67 |
| Day 4 | C10 | 0.105 | 0.187 | | | | | | |
| 37 C., 24 hr, | D9 | 0.107 | 0.188 | 0.192 | 0.006 | 3.4 | 4 | 0.77 | 0.69 |
| Day 5 | D10 | 0.112 | 0.197 | | | | | | |
| 37 C., 24 hr, | E9 | 0.1 | 0.175 | 0.179 | 0.005 | 2.9 | 4 | 0.715 | 0.64 |
| Day 7 | E10 | 0.104 | 0.182 | | | | | | |
| 37 C., 24 hr, | F9 | 0.098 | 0.171 | 0.168 | 0.005 | 3.1 | 4 | 0.671 | 0.6 |
| Day 9 | F10 | 0.094 | 0.164 | | | | | | |
| 37 C., 24 hr, | G9 | 0.11 | 0.193 | 0.17 | 0.033 | 19.7 | 4 | 0.679 | 0.6 |
| Day 14 | G10 | 0.084 | 0.146 | | | | | | |

Klotho Concentration Changes in Time Course

TABLE 35

| | Room | −15° C. | 0° C. | 0° C. | 37° C. | 37° C. |
|---|---|---|---|---|---|---|
| | Temp | 8 hr | 8 hr | 24 hr | 8 hr | 24 hr |
| Day 4 | 100% | 30% | 89% | 92% | 88% | 67% |
| Day 5 | 100% | 53% | 92% | 92% | 96% | 69% |
| Day 7 | 92% | 37% | 88% | 88% | 100% | 64% |
| Day 9 | 96% | 27% | 91% | 87% | 67% | 60% |
| Day 14 | 69% | 27% | 74% | 63% | 58% | 60% |

Plate 3, Donor 3:
Standard Calculations

TABLE 36

| Sam- | Concen- | | | Val- | MeanVal- | Std. | CV |
|---|---|---|---|---|---|---|---|
| ple | tration | Wells | BackConcCalc | ues | ue | Dev. | % |
| St01 | 6 | A1 | 6.24 | 2.768 | 2.784 | 0.023 | 0.8 |
| | | A2 | 6.333 | 2.8 | | | |
| St02 | 3 | B1 | 3.15 | 1.629 | 1.636 | 0.009 | 0.6 |
| | | B2 | 3.182 | 1.642 | | | |
| St03 | 1.5 | C1 | 1.52 | 0.926 | 0.919 | 0.011 | 1.2 |
| | | C2 | 1.489 | 0.911 | | | |
| St04 | 0.75 | D1 | 0.705 | 0.51 | 0.497 | 0.018 | 3.7 |
| | | D2 | 0.659 | 0.484 | | | |
| St05 | 0.375 | E1 | 0.337 | 0.288 | 0.286 | 0.003 | 1 |
| | | E2 | 0.331 | 0.284 | | | |
| St06 | 0.188 | F1 | 0.195 | 0.188 | 0.177 | 0.016 | 9.2 |
| | | F2 | 0.164 | 0.165 | | | |
| St07 | 0.094 | G1 | 0.108 | 0.119 | 0.12 | 0.001 | 1.2 |
| | | G2 | 0.11 | 0.121 | | | |

Sample Calculations

TABLE 37

| | | | | | | | | | Difference |
|---|---|---|---|---|---|---|---|---|---|
| Sample | Wells | Values | Result | MeanResult | Std. Dev. | CV % | Dilution | Adj. Result | to 0 h |
| 0 h | A3 | 0.155 | 0.152 | 0.152 | 0.001 | 0.6 | 4 | 0.609 | 1.00 |
| | A4 | 0.156 | 0.153 | | | | | | |
| RT | B3 | 0.161 | 0.159 | 0.157 | 0.003 | 1.7 | 4 | 0.63 | 1.03 |
| Day 4 | B4 | 0.158 | 0.156 | | | | | | |
| RT | C3 | 0.159 | 0.157 | 0.157 | 0 | 0 | 4 | 0.627 | 1.03 |
| Day 5 | C4 | 0.159 | 0.157 | | | | | | |
| RT | D3 | 0.15 | 0.145 | 0.146 | 0.001 | 0.6 | 4 | 0.584 | 0.96 |
| Day 7 | D4 | 0.151 | 0.147 | | | | | | |
| RT | E3 | 0.16 | 0.158 | 0.159 | 0.002 | 1.1 | 4 | 0.637 | 1.05 |
| Day 9 | E4 | 0.162 | 0.161 | | | | | | |
| RT | F3 | 0.131 | 0.122 | 0.125 | 0.004 | 3.4 | 4 | 0.501 | 0.82 |
| Day 14 | F4 | 0.136 | 0.128 | | | | | | |
| −15 C., | G3 | 0.114 | 0.102 | 0.103 | 0.001 | 0.8 | 4 | 0.411 | 0.67 |
| 8 hr | G4 | 0.115 | 0.103 | | | | | | |
| Day 4 | | | | | | | | | |
| −15 C., | H3 | 0.158 | 0.156 | 0.128 | 0.039 | 30.1 | 4 | 0.513 | 0.84 |
| 8 hr | H4 | 0.113 | 0.101 | | | | | | |
| Day 5 | | | | | | | | | |
| −15 C., | A5 | 0.108 | 0.095 | 0.092 | 0.005 | 5.2 | 4 | 0.367 | 0.60 |
| 8 hr | A6 | 0.102 | 0.08 | | | | | | |
| Day 7 | | | | | | | | | |
| −15 C., | B5 | 0.1 | 0.086 | 0.082 | 0.005 | 6.6 | 4 | 0.329 | |
| 8 hr | B6 | 0.093 | 0.079 | | | | | | 0.54 |
| Day 9 | | | | | | | | | |
| −15 C., | C5 | 0.099 | 0.085 | 0.088 | 0.005 | 5.4 | 4 | 0.354 | |
| 8 hr | C6 | 0.105 | 0.092 | | | | | | 0.58 |
| Day 14 | | | | | | | | | |
| 0 C., | D5 | 0.15 | 0.145 | 0.143 | 0.004 | 2.5 | 4 | 0.572 | |
| 8 hr, | D6 | 0.146 | 0.14 | | | | | | 0.94 |
| Day 4 | | | | | | | | | |
| 0 C., | E5 | 0.156 | 0.153 | 0.154 | 0.001 | 0.6 | 4 | 0.614 | |
| 8 hr, | E6 | 0.157 | 0.154 | | | | | | |
| Day 5 | | | | | | | | | |
| 0 C., | F5 | 0.151 | 0.147 | 0.147 | 0.001 | 0.6 | 4 | 0.589 | |
| 8 hr, | F6 | 0.152 | 0.148 | | | | | | 0.97 |
| Day 7 | | | | | | | | | |
| 0 C., | G5 | 0.154 | 0.15 | 0.152 | 0.002 | 1.2 | 4 | 0.607 | |
| 8 hr, | G6 | 0.156 | 0.153 | | | | | | 1.00 |
| Day 9 | | | | | | | | | |
| 0 C., | H5 | 0.149 | 0.144 | 0.148 | 0.005 | 3.6 | 4 | 0.592 | |
| 8 hr, | H6 | 0.155 | 0.152 | | | | | | 0.97 |
| Day 14 | | | | | | | | | |
| 0 C., | A7 | 0.161 | 0.159 | 0.158 | 0.002 | 1.1 | 4 | 0.632 | |
| 24 hr, | A8 | 0.159 | 0.157 | | | | | | 1.04 |
| Day 4 | | | | | | | | | |
| 0 C., | B7 | 0.156 | 0.153 | 0.156 | 0.004 | 2.3 | 4 | 0.622 | |
| 24 hr, | B8 | 0.16 | 0.158 | | | | | | 1.02 |
| Day 5 | | | | | | | | | |
| 0 C., | C7 | 0.15 | 0.145 | 0.147 | 0.002 | 1.2 | 4 | 0.587 | |
| 24 hr, | C8 | 0.152 | 0.148 | | | | | | 0.96 |
| Day 7 | | | | | | | | | |
| 0 C., | D7 | 0.154 | 0.15 | 0.147 | 0.004 | 3 | 4 | 0.589 | |
| 24 hr, | D8 | 0.149 | 0.144 | | | | | | 0.97 |
| Day 9 | | | | | | | | | |
| 0 C., | E7 | 0.132 | 0.123 | 0.124 | 0.001 | 0.7 | 4 | 0.496 | |
| 24 hr, | E8 | 0.113 | 0.125 | | | | | | 0.81 |
| Day 14 | | | | | | | | | |
| 37 C., | F7 | 0.15 | 0.145 | 0.147 | 0.002 | 1.2 | 4 | 0.587 | |
| 8 hr, | F8 | 0.152 | 0.148 | | | | | | 0.96 |
| Day 4 | | | | | | | | | |
| 37 C., | G7 | 0.164 | 0.163 | 0.163 | 0 | 0 | 4 | 0.653 | |
| 8 hr, | G8 | 0.164 | 0.163 | | | | | | 1.07 |
| Day 5 | | | | | | | | | |
| 37 C., | H7 | 0.17 | 0.171 | 0.167 | 0.005 | 3.3 | 4 | 0.668 | |
| 8 hr, | H8 | 0.164 | 0.163 | | | | | | 1.10 |
| Day 7 | | | | | | | | | |
| 37 C., | A9 | 0.14 | 0.133 | 0.131 | 0.333 | 2 | 4 | 0.525 | |
| 8 hr, | A10 | 0.137 | 0.129 | | | | | | 0.86 |
| Day 9 | | | | | | | | | |
| 37 C., | B9 | 0.131 | 0.122 | 0.122 | 0.001 | 0.7 | 4 | 0.486 | |
| 8 hr, | B10 | 0.13 | 0.121 | | | | | | 0.80 |
| Day 14 | | | | | | | | | |
| 37 C., | C9 | 0.139 | 0.132 | 0.136 | 0.005 | 3.9 | 4 | 0.0542 | |
| 24 hr, | C10 | 0.145 | 0.139 | | | | | | 0.89 |
| Day 4 | | | | | | | | | |
| 37 C., | D9 | 0.137 | 0.129 | 0.13 | 0.001 | 0.7 | 4 | 0.52 | |
| 24 hr, | D10 | 0.138 | 0.131 | | | | | | 0.85 |
| Day 5 | | | | | | | | | |
| 37 C., | E9 | 0.134 | 0.126 | 0.126 | 0 | 0 | 4 | 0.503 | |
| 24 hr, | E10 | 0.134 | 0.126 | | | | | | 0.83 |
| Day 7 | | | | | | | | | |
| 37 C., | F9 | 0.133 | 0.125 | 0.122 | 0.003 | 2.8 | 4 | 0.489 | |
| 24 hr, | F10 | 0.129 | 0.12 | | | | | | 0.8 |
| Day 9 | | | | | | | | | |
| 37 C., | G9 | 0.122 | 0.111 | 0.112 | 0.001 | 0.7 | 4 | 0.448 | |
| 24 hr, | G10 | 0.123 | 0.113 | | | | | | 0.74 |
| Day 14 | | | | | | | | | |

Klotho Concentration Changes in Time Course

TABLE 38

| | Room | −15° C. | 0° C. | 0° C. | 37° C. | 37° C. |
|---|---|---|---|---|---|---|
| | Temp | 8 hr | 8 hr | 24 hr | 8 hr | 24 hr |
| Day 4 | 100% | 67% | 94% | 104% | 96% | 89% |
| Day 5 | 103% | 84% | 101% | 102% | 107% | 85% |
| Day 7 | 103% | 60% | 97% | 96% | 110% | 83% |
| Day 9 | 96% | 54% | 100% | 97% | 86% | 80% |
| Day 14 | 105% | 58% | 97% | 81% | 80% | 74% |

Plate 4, Donor 4:
Standard Calculations

TABLE 39

| Sample | Concentration | Wells | BackConcCalc | Values | MeanValue | Std. Dev. | CV % |
|---|---|---|---|---|---|---|---|
| St01 | 6 | A1 | 5.507 | 2.715 | 2.671 | 0.062 | 2.3 |
| | | A2 | 5.321 | 2.627 | | | |
| St02 | 3 | B1 | 3.058 | 1.542 | 1.546 | 0.006 | 0.4 |
| | | B2 | 3.074 | 1.55 | | | |
| St03 | 1.5 | C1 | 1.623 | 0.839 | 0.825 | 0.019 | 2.3 |
| | | C2 | 1.569 | 0.812 | | | |
| St04 | 0.75 | D1 | 0.794 | 0.422 | 0.412 | 0.013 | 3.3 |
| | | D2 | 0.757 | 0.403 | | | |
| St05 | 0.375 | E1 | 0.422 | 0.23 | 0.222 | 0.011 | 4.8 |
| | | E2 | 0.393 | 0.215 | | | |
| St06 | 0.188 | F1 | 0.19 | 0.107 | 0.105 | 0.003 | 2.7 |
| | | F2 | 0.182 | 0.103 | | | |
| St07 | 0.094 | G1 | 0.086 | 0.05 | 0.05 | 0 | 0 |
| | | G2 | 0.086 | 0.05 | | | |

Sample Calculations

TABLE 40

| | | | | | | | | | Difference |
|---|---|---|---|---|---|---|---|---|---|
| Sample | Wells | Values | Result | MeanResult | Std. Dev. | CV % | Dilution | Adj. Result | to 0 h |
| 0 h | A3 | 0.105 | 0.186 | 0.214 | 0.04 | 18.5 | 4 | 0.857 | 1.00 |
| | A4 | 0.135 | 0.242 | | | | | | |
| RT | B3 | 0.127 | 0.227 | 0.215 | 0.017 | 8 | 4 | 0.86 | 1.00 |
| Day 4 | B4 | 0.114 | 0.203 | | | | | | |
| RT | C3 | 0.127 | 0.227 | 0.216 | 0.016 | 7.3 | 4 | 0.864 | 1.01 |
| Day 5 | C4 | 0.115 | 0.205 | | | | | | |
| RT | D3 | 0.105 | 0.186 | 0.183 | 0.005 | 2.9 | 4 | 0.73 | 0.85 |
| Day 7 | D4 | 0.101 | 0.179 | | | | | | |
| RT | E3 | 0.092 | 0.162 | 0.168 | 0.008 | 4.7 | 4 | 0.671 | 0.78 |
| Day 9 | E4 | 0.098 | 0.173 | | | | | | |
| RT | F3 | 0.092 | 0.162 | 0.167 | 0.007 | 3.9 | 4 | 0.667 | 0.78 |
| Day 14 | F4 | 0.097 | 0.171 | | | | | | |
| −15 C., | G3 | 0.053 | 0.091 | 0.088 | 0.004 | 4.3 | 4 | 0.535 | 0.41 |
| 8 hr | G4 | 0.05 | 0.086 | | | | | | |
| Day 4 | | | | | | | | | |
| −15 C., | H3 | 0.093 | 0.164 | 0.13 | 0.048 | 36.7 | 4 | 0.521 | 0.61 |
| 8 hr | H4 | 0.056 | 0.096 | | | | | | |
| Day 5 | | | | | | | | | |
| −15 C., | A5 | 0.065 | 0.113 | 0.105 | 0.012 | 11 | 4 | 0.418 | 0.49 |
| 8 hr | A6 | 0.056 | 0.096 | | | | | | |
| Day 7 | | | | | | | | | |
| −15 C., | B5 | 0.04 | 0.068 | 0.061 | 0.01 | 16.6 | 4 | 0.242 | 0.28 |
| 8 hr | B6 | 0.032 | 0.054 | | | | | | |
| Day 9 | | | | | | | | | |
| −15 C., | C5 | 0.037 | 0.062 | 0.058 | 0.006 | 10.8 | 4 | 0.232 | 0.27 |
| 8 hr | C6 | 0.032 | 0.054 | | | | | | |
| Day 14 | | | | | | | | | |
| 0 C., | D5 | 0.11 | 0.195 | 0.192 | 0.005 | 2.7 | 4 | 0.767 | 0.89 |
| 8 hr, | D6 | 0.106 | 0.188 | | | | | | |
| Day 4 | | | | | | | | | |
| 0 C., | E5 | 0.118 | 0.21 | 0.208 | 0.003 | 1.3 | 4 | 0.834 | 0.97 |
| 8 hr, | E6 | 0.116 | 0.207 | | | | | | |
| Day 5 | | | | | | | | | |
| 0 C., | F5 | 0.111 | 0.197 | 0.194 | 0.005 | 2.7 | 4 | 0.774 | 0.9 |
| 8 hr, | F6 | 0.107 | 0.19 | | | | | | |
| Day 7 | | | | | | | | | |
| 0 C., | G5 | 0.199 | 0.175 | 0.169 | 0.009 | 5.4 | 4 | 0.675 | 0.79 |
| 8 hr, | G6 | 0.092 | 0.162 | | | | | | |
| Day 9 | | | | | | | | | |
| 0 C., | H5 | 0.093 | 0.164 | 0.161 | 0.004 | 2.4 | 4 | 0.645 | 0.75 |
| 8 hr, | H6 | 0.09 | 0.158 | | | | | | |
| Day 14 | | | | | | | | | |
| 0 C., | A7 | 0.124 | 0.222 | 0.211 | 0.014 | 6.9 | 4 | 0.845 | 0.99 |
| 24 hr, | A8 | 0.113 | 0.201 | | | | | | |
| Day 4 | | | | | | | | | |
| 0 C., | B7 | 0.108 | 0.192 | 0.19 | 0.003 | 1.4 | 4 | 0.76 | 0.89 |
| 24 hr, | B8 | 0.106 | 0.188 | | | | | | |
| Day 5 | | | | | | | | | |
| 0 C., | C7 | 0.096 | 0.17 | 0.171 | 0.003 | 1.5 | 4 | 0.686 | 0.8 |
| 24 hr, | C8 | 0.098 | 0.173 | | | | | | |
| Day 7 | | | | | | | | | |
| 0 C., | D7 | 0.094 | 0.166 | 0.168 | 0.003 | 1.6 | 4 | 0.671 | 0.78 |
| 24 hr, | D8 | 0.096 | 0.17 | | | | | | |
| Day 9 | | | | | | | | | |
| 0 C., | E7 | 0.078 | 0.136 | 0.142 | 0.008 | 5.5 | 4 | 0.568 | 0.66 |
| 24 hr, | E8 | 0.084 | 0.147 | | | | | | |
| Day 14 | | | | | | | | | |
| 37 C., | F7 | 0.115 | 0.205 | 0.208 | 0.005 | 2.5 | 4 | 0.834 | 0.97 |
| 8 hr, | F8 | 0.119 | 0.212 | | | | | | |
| Day 4 | | | | | | | | | |
| 37 C., | G7 | 0.102 | 0.181 | 0.179 | 0.003 | 1.5 | 4 | 0.715 | 0.83 |
| 8 hr, | G8 | 0.1 | 0.177 | | | | | | |
| Day 5 | | | | | | | | | |
| 37 C., | H7 | 0.104 | 0.184 | 0.182 | 0.004 | 2.2 | 4 | 0.726 | 0.85 |
| 8 hr, | H8 | 0.101 | 0.179 | | | | | | |
| Day 7 | | | | | | | | | |
| 37 C., | A9 | 0.072 | 0.125 | 0.122 | 0.005 | 4.2 | 4 | 0.487 | 0.57 |
| 8 hr, | A10 | 0.068 | 0.118 | | | | | | |
| Day 9 | | | | | | | | | |
| 37 C., | B9 | 0.067 | 0.116 | 0.111 | 0.008 | 6.9 | 4 | 0.444 | 0.52 |
| 8 hr, | B10 | 0.061 | 0.105 | | | | | | |
| Day 14 | | | | | | | | | |
| 37 C., | C9 | 0.067 | 0.116 | 0.113 | 0.005 | 4.6 | 4 | 0.451 | 0.53 |
| 24 hr, | C10 | 0.063 | 0.109 | | | | | | |
| Day 4 | | | | | | | | | |
| 37 C., | D9 | 0.075 | 0.131 | 0.132 | 0.001 | 1 | 4 | 0.528 | 0.62 |
| 24 hr, | D10 | 0.076 | 0.133 | | | | | | |
| Day 5 | | | | | | | | | |
| 37 C., | E9 | 0.068 | 0.118 | 0.115 | 0.004 | 3.3 | 4 | 0.462 | 0.54 |
| 24 hr, | E10 | 0.065 | 0.113 | | | | | | |
| Day 7 | | | | | | | | | |
| 37 C., | F9 | 0.072 | 0.125 | 0.122 | 0.005 | 4.2 | 4 | 0.487 | 0.57 |
| 24 hr, | F10 | 0.068 | 0.118 | | | | | | |
| Day 9 | | | | | | | | | |
| 37 C., | G9 | 0.061 | 0.105 | 0.105 | 0 | 0 | 4 | 0.422 | 0.49 |
| 24 hr, | G10 | 0.061 | 0.105 | | | | | | |
| Day 14 | | | | | | | | | |

Klotho Concentration Changes in Time Course

TABLE 41

| | Room | −15° C. | 0° C. | 0° C. | 37° C. | 37° C. |
|---|---|---|---|---|---|---|
| | Temp | 8 hr | 8 hr | 24 hr | 8 hr | 24 hr |
| Day 4 | 100% | 41% | 89% | 99% | 97% | 53% |
| Day 5 | 101% | 61% | 97% | 89% | 83% | 62% |
| Day 7 | 85% | 49% | 90% | 80% | 85% | 54% |
| Day 9 | 78% | 28% | 79% | 78% | 57% | 57% |
| Day 14 | 78% | 27% | 75% | 66% | 52% | 49% |

In light of the foregoing, at least one embodiment includes a Klotho detection kit and/or assay. The inventive kit and/or assay may be suitable for CLIA lab testing of clinical samples. The kit can be configured (specifically) for use in monitoring, detecting, and/or quantifying, or diagnosing deficiency of one or more proteins selected from (1) Native (e.g., endogenous) Klotho, (2) recombinant Klotho-Fc fusion, and (3) recombinant 6xHis Klotho. The Klotho protein can be detected in a biological sample. The sample can be (substantially) unpurified in some embodiments. For example, the sample can be or comprise mammalian (e.g., human) blood and/or plasma.

In at least one embodiment, a kit can comprise one or more lancets adapted, configured, or suitable for a finger prick, a sample collection container adapted, configured, or suitable for collecting blood from a finger prick, one or more antiseptic elements, such as disposable (e.g., alcohol) wipe(s), one or more adhesive bandages, a foil envelope or other biopackaging, and a shipping container, such as an envelope.

Some embodiments can include a method of diagnosing Klotho deficiency in a mammalian subject. The method can include obtaining a fluid sample mixture selected from the group consisting of: (i) mammalian serum, optionally in the form of capillary blood, mixed with a preservative or anti-coagulant comprising one or more of heparin, lithium heparin, EDTA, and K2 EDTA, the mammalian serum having an amount of soluble Klotho protein; and (ii) mammalian serum, optionally in the form of capillary blood, the mammalian serum having an amount of soluble Klotho protein, the method further comprising mixing the mammalian serum with a preservative or anti-coagulant comprising one or more of heparin, lithium heparin, EDTA, and K2 EDTA to form the mixture. The method can also include storing the mixture for at least 24 hours, or more, at room temperature, the preservative or anti-coagulant stabilizing the soluble Klotho protein for the at least 24 hours, or more, at room temperature. After the storing step, the method can include quantifying the amount of the soluble Klotho protein in the mixture. The method can also include diagnosing the mammalian subject with Klotho deficiency when the quantified amount of soluble Klotho protein in the mixture is less than a predetermined threshold amount.

As described above, the fluid sample mixture comprises about 10-1000 ul, preferably about 50-200 ul of capillary blood from the mammalian subject, or wherein the step of obtaining the fluid sample mixture comprises obtaining about 10-1000ul, preferably about 50-200 ul of capillary blood from the mammalian subject. The step of quantifying the amount of the soluble Klotho protein in the mixture comprises detecting the soluble Klotho protein using a first antibody that binds to a portion of the soluble Klotho protein, and optionally, detecting the soluble Klotho protein using a detection antibody that binds to a portion of the first antibody or performing an enzyme-linked immunosorbent assay (ELISA) to detect and optionally quantify the soluble Klotho protein. Mass spectrometry can also be used to detect proteins.

In some embodiments, a diagnostic or informational report can be prepared. The report can include an indication or display of measured Klotho level(s) for one or more Klotho proteins. In some embodiments, the report can display a comparison chart or graph depicting the measured level overlaid on an age-based graph illustrating average or median Klotho levels for various ages and, optionally, indication of the 25^{th} percentile and/or 75^{th} percentile for the various ages. FIG. 13 illustrates an example report graph or chart. Thus, in some embodiments, diagnosing the mammalian subject with Klotho deficiency comprises displaying a Klotho deficiency determination, indication, or diagnosis on a user interface of a computer system and/or producing a file or report, in physical or electronic form, that displays the Klotho deficiency determination, indication, or diagnosis, the Klotho deficiency determination, indication, or diagnosis optionally including the quantified amount of the soluble Klotho protein and/or the predetermined threshold amount.

It will be appreciated that the term “diagnosis,” or diagnosing,” and similar terms, as used herein, is not intended to convey or require (in all cases) a certified, regulatory-compliant, medical diagnosis, as required or regulated by the U.S. Food and Drug Administration (FDA) or any other government or non-government entity, organization, or agency. Rather, “diagnosis,” or diagnosing,” and similar terms relates to providing information that may be indicative of a condition and/or helpful in making a determination related to the condition.

In some embodiments, one or more additional analytes can be monitored, detected, and/or quantified. Illustratively, the additional analytes can be related to Kidney disease, fibrotic diseases, and/or aging. In some embodiments, the one or more additional analytes can be selected from the group consisting of FGF, PTH, non-oxidized PTH, Vit D, Vit E, KIM-1, NGAL, Interleukins and other inflammatory bio-markers, testosterone, estrogen, etc.

### Therapeutic Proteins

Embodiments of the present disclosure can include one or more therapeutic and/or recombinant human alpha soluble Klotho proteins, protein fragments, and/or protein variants.

The protein can comprise all or a subset of amino acid residues 1-1012, 1-981, 29-981, 34-981, 36-981, 131-981, 1-549, 29-549, 34-549, 36-549, or 131-549 of human alpha Klotho isoform 1 or 2. The protein can have at least and/or about 80% amino acid sequence identity to all or a subset of amino acid residues 1-1012, 1-981, 29-981, 34-981, 36-981, 131-981, 1-549, 29-549, 34-549, 36-549, or 131-549 of human alpha Klotho isoform 1 or 2. For instance, at least a portion of the protein can have at least 80%, etc amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, or a combination of two or more thereof. Other portions or fragments of the protein sequences described in the present application are also contemplated herein. For instance, some embodiments can include a protein having at least a portion with at least 80%, etc amino acid sequence identity to any suitable portion of one of SEQ ID NOS: 1-75, or a combination of two or more thereof.

Some embodiments can include a protein having one or more amino acid variations as compared to human alpha Klotho isoform 1. Illustratively, the protein can comprise a human C370 variant. For instance, the protein can include a C370S alteration, thereby comprising S370. The protein can include human F352 or other than F352V in some embodiments. In at least one embodiment, the protein can include the C370S alteration, thereby comprising S370, without a F352V variant, preferably with F352. The protein can include H193 or other than the H193R variant. All other standard amino acid substitutions at amino acid residue (or position) 193, 352, and/or 370 of human alpha Klotho isoform 1 are contemplated and explicitly disclosed herein.

Some embodiments can include a variation at amino acid residue 45 of human alpha Klotho isoform 1. At position 45, the residue can be a valine (Val; V), a phenylalanine (Phe; F), or another amino acid.

The protein can also include one or more glycans (attached thereto). For instance, native human alpha Klotho isoform 1 can have glycans attached (via glycosylation) at amino acids 106, 159, 283, 344, 604, 612, and/or 694. Accordingly, the proteins of the present disclosure, or Klotho protein sequences thereof, can have one or more of the same (or similar) glycans attached (via glycosylation) thereto (e.g., at the same amino acid position(s)). In a preferred embodiment, the protein includes all of the same or similar (native-type) glycans attached thereto, at the same amino acid position(s).

In some embodiments, the protein can include a signal peptide or signaling sequence. For example, the protein can include a native Klotho signaling sequence. The protein can include a non-native or synthetic signaling sequence. In some embodiments, the signaling sequence can be an N-terminal signaling sequence and/or upstream of (or N-terminal to) a Klotho protein sequence. In other embodiments, the signaling sequence can be C-terminal or otherwise disposed. Preferably, the signaling sequence can be, comprise, or have at least 80% amino acid sequence identity to native human alpha Klotho isoform 1 signaling sequence, native human alpha Klotho isoform 2 signaling sequence, SEQ ID NO: 71 or SEQ ID NO: 72.

In some embodiments, the protein can include an amino acid tag. The tag can be a C-terminal tag and/or downstream of (or C-terminal to) a Klotho protein sequence. In other embodiments, the tag can be N-terminal or otherwise disposed. The tag can be or comprise an Fc-peptide (or Fc-fusion tag, Fc domain, etc.). For instance, the tag can be or comprise an IgG1-Fc protein sequence. Preferably, the tag can be, comprise, or have at least 80% amino acid sequence identity to SEQ ID NO: 74.

The tag can also, or alternatively, be or comprise a peptide comprising a Twin-Strep protein sequence, such as a Twin-Strep tag or protein sequence (e.g., as known in the art). Preferably, the signaling sequence can be, comprise, or have at least 80% amino acid sequence identity to SEQ ID NO: 75, SEQ ID NO: 102, SEQ ID NO: 103, SEQ ID NO: 104.

The tag can also, or alternatively, be or comprise a polysialic acid (PSA). In at least one embodiment, the PSA tag can render the conjugated (Klotho) protein resistant to one or more proteases. In some embodiments, maintaining an amount (e.g., even a relatively small amount) of sialic acid in the protein conjugate can prevent protease attack and subsequent targeting, breakdown, and clearance of the molecule (e.g., through the liver).

In at least one embodiment, the tag can be cleaved from the protein. In other embodiments, the tag can be retained as part of the protein. In some embodiments, the tag can enhance solubility and/or (serum) half-life of the protein. In some embodiments, the tag can be utilized during protein purification (e.g., as part of a purification mechanism).

In some embodiments, the protein can include a linker (e.g., amino acid linker) disposed between a Klotho protein sequence and an amino acid tag. Illustratively, the linker can comprise between 1 and 40 amino acids, preferably between 5 and 20 amino acids, more preferably between 6 and 12 amino acids, most preferably between about 8 to 10 amino acids. The linker can be or comprise a GS linker (e.g., a (G₄S)₂ linker) or other linker (e.g., a GGENLYFQ linker) in some embodiments. Preferably, the linker can be, comprise, or have at least 80% amino acid sequence identity to SEQ ID NO: 73 or SEQ ID NO: 105.

In at least one embodiment, the protein can be cGMP regulation compliant, as determined and enforced by the U.S. Food and Drug Administration (FDA). For instance, the Klotho protein can be at least 90%, 92%, 95%, 96%, 97%, 98%, or 99% pure, dry weight. In some embodiments, the Klotho protein sample can include less than about 1-100 parts per million (ppm), less than about 100-1000 parts per billion (ppb), or less than about 1-100 ppb CHO host cell proteins (HCP), nucleic acid, and/or other cellular components, or any value or range of values disposed therebetween.

### Klotho Protein Variants

Therapeutic S-Klotho proteins of various lengths (e.g., S-Klotho, isoform 1 or 2, 1-981, 29-981, 34-981, 36-981, 131-981, 1-549, 29-549, 34-549, 36-549, 131-549, and so forth) can be modified in a variety of ways to achieve various beneficial effects and/or results not exhibited in native Klotho proteins. Illustratively, the QuickChange XL Site-Directed Mutagenesis Kit (Stratagene) can be used to alter the nucleic acid sequence of various S-Klotho constructs. Other mutagenesis methods and kits as known in the art can also be used. For instance, various sub-cloning methods and kits are known in the art and commercially available. Amino acid changes may affect (e.g., improve, enhance, decrease, etc.) protein expression, protein stability, protein solubility, protein binding, protein specificity, protein activity, protein function, immunogenicity, toxicity, etc.

In at least one embodiment of the present disclosure, a protein is modified with one or more C-terminal tags and/or N-terminal tags. Such tags can function to extend the serum and/or soluble half-life of the protein (in one or more therapeutic or other environments). Tags can also be useful as markers for the presence or diagnostic localization of the protein, isolation or removal of the protein, delivery or transport of the protein, binding of the protein to one or more targets (e.g., protein, nucleic acid, organelle, cellular structural component, etc.), enzymatic processing or cleavage, and so forth. In at least one embodiment, the C-terminus of the protein can be tagged with a TEV-Twin-Strep tag or sequence, an immunoglobulin (IgG1) Fc domain tag or sequence, or other tag or sequence as known in the art and/or described further herein. Additional description can be found in the articles “Fusion Proteins for Half-Life Extension of Biologics as a Strategy to Make Biobetters,” “What is the future of PEGylated therapies?” and “Strategies for extended serum half-life of protein therapeutics,” the entirety of each of which is incorporated herein by specific reference. In certain embodiments, a linker or linker peptide can be inserted and/or disposed between the (native or variant) Klotho protein sequence and the tag.

In some embodiments, the modified Klotho protein can include an alternative (e.g., native, non-native, and/or synthetic) signal peptide. For instance, in some embodiments, the native signal peptide sequences can be replaced and/or supplemented with an alternative signal peptide or signaling sequence (SS). In some embodiments, a native Methionine residue of a Klotho protein can be removed and a Methionine residue at the N-terminus of the SS can be included. Additional description can be found in the thesis “Generation of high expressing CHO cell lines for the production of recombinant antibodies using optimized signal peptides and a novel ER stress based selection system,” the entirety of which is incorporated herein by specific reference. In certain embodiments, a linker or linker peptide can be inserted and/or disposed between the (native or variant) Klotho protein sequence and the alternative SS.

Some embodiments can include one or more amino acid variants. It will be appreciated that the present disclosure contemplates variation of any one or more of the native amino acids in any of the disclosed Klotho proteins to any other amino acid, whether naturally-occurring, synthetic, or otherwise configured.

### S-Klotho V45F Protein Variant

Substituting a Phenylalanine in the place of the Valine at (native) position 45 of human alpha Klotho (V45F) has been found by the Inventors to have surprising and unexpected benefits. For example, V45F soluble Klotho, and fragments or fusion proteins thereof, express at higher levels in CHO and HEK-293 cells than does the native, wild type V45 protein. Commercially, achieving higher levels of soluble protein expression can be desirable. Accordingly, some embodiments of the present disclosure include a Klotho protein having the V45F substitution.

### S-Klotho C370S Protein Variant

In humans, the Klotho gene maps on chromosome 13q12. A variant, known as KL-VS, is present in approximately 15-25% of Caucasians. The variant is composed of six single nucleotide polymorphisms (SNPs), two of which cause amino acid substitutions (i.e. F352V and C370S—Phenylalanine 352 changed to Valine and Cysteine 370 changed to Serine). In vitro transfection assays have shown that secreted levels of klotho are reduced 6-fold for the V352 variant, while they are increased almost 3-fold for the S370 form. However, these two variants in the human KLOTHO gene segregate together and form the KL-VS haplotype that increases klotho secretion in the range of 1.6-fold. For instance, it has been reported that in a screening of over 300 individuals taken from the geographically and/or ethnically distinct cohorts, not a single individual was found to harbor only one of the V352 variant or the S370 variant.

An embodiment of the present disclosure includes a recombinant S-Klotho protein having the C370S homovariant (i.e., without the presence (or with deletion) of the F352V variant). The C370S variant can be produced and/or expressed in the context of any protein construct described herein. For instance, the C370S variant can be produced or expressed in the context of S-Klotho, isoform 1 or 2, 1-981, 29-981, 34-981, 36-981, 131-981, 1-549, 29-549, 34-549, 36-549, 131-549, and so forth, with or without a tag (e.g., (IgG) Fc tag, TEV-Twin-Strep tag, TEV sequence, etc.). Accordingly, the nucleic acid construct or cDNA from which the protein is expressed can be of corresponding length.

Embodiments can include producing a S370 heterozygous or homozygous variant construct, transferring (e.g., via transfection) the resulting construct, which encodes the S-Klotho C370S protein, into an appropriate expression system (e.g., CHO cells), and/or transiently expressing the S-Klotho S370 protein. The S370 Klotho protein may be expressed at higher levels than the F352V/C370S protein and/or wild-type F352/C370 protein. Embodiments can include purifying (and optionally quality-control testing) the expressed protein for therapeutic administration. Embodiments can include administering a therapeutic or therapeutically-effective amount of the S-Klotho C370S protein to a subject in need thereof. The subject may, for example, harbor or express the KL-VS variant. Alternatively, the subject may be of wild-type of other mutant or variant type. Administration of the recombinant S-Klotho C370S protein can lead to a beneficial increase in blood S-Klotho levels. Accordingly, the circulating concentration of S-Klotho in subjects that receive the therapeutic, recombinant, S-Klotho C370S protein may not be subjected to the dilutive effects that are observed when the F352V variant is present.

### Nucleic Acids and Expression Vectors

Some embodiments can include a nucleic acid or nucleic acid construct. For instance, embodiments can include an expression vector or nucleic acid construct. The nucleic acid can encode a recombinant human alpha soluble Klotho protein, protein fragment, or protein variant, as described herein. In at least one embodiment, the nucleic acid can encode a Klotho protein sequence, an optional (native or non-native) signaling sequence (e.g., at the N-terminus or N-terminal of the Klotho protein sequence), an optional linker sequence (e.g., GS linker), and/or an amino acid tag (e.g., IgG1-Fc or TEV-Twin-Strep), as described herein.

In some embodiments, the nucleic acid can express a protein that includes all or a subset of amino acid residues 1-1012, 1-981, 29-981, 34-981, 36-981, 131-981, 1-549, 29-549, 34-549, 36-549, or 131-549 of human alpha Klotho isoform 1 or 2. At least a portion of the protein can have at least or about 80% amino acid sequence identity to all or a subset of amino acid residues 1-1012, 1-981, 29-981, 34-981, 36-981, 131-981, 1-549, 29-549, 34-549, 36-549, or 131-549 of human alpha Klotho (isoform 1 or 2). For instance, at least a portion of the protein can have at least and/or about 80% amino acid sequence identity to all or a portion of one of SEQ ID NO: 1 through SEQ ID NO: 75, or a combination of two or more thereof. In a preferred embodiment, the protein can have at least and/or about 80% amino acid sequence identity to all or a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152.

In some embodiments, at least a portion of the nucleic acid can have at least and/or about 80% (e.g., at least about 82%, 85%, 86%, 87%, 88%, 89%, 90%, 91%, 92%, 93%, 94%, 95%, 96%, 97%, 98%, or 99%, or 100% nucleotide sequence identity to one of SEQ ID NO: 76 through SEQ ID NO: 106 or SEQ ID NO: 121 through SEQ ID NO: 124, or a combination of two or more thereof. In a preferred embodiment, the nucleic acid can have at least and/or about 80% nucleotide sequence identity to one of SEQ ID NO: 76 through SEQ ID NO: 106 or SEQ ID NO: 121 through SEQ ID NO: 124.

Nucleic acid sequences of the present disclosure can also include a stop codon, as known in the art (e.g., TGA, TAG, TAA).

### Cell Lines and Methods of Manufacture

An embodiment of the present disclosure can include a cell line. The cell line can comprise any suitable cell type, such as CHO cells, HEK cells, HL-60 cells, or other cell line as known in the art. Illustratively, the cell line can comprise CHO cells (e.g., a plurality of CHO cells). For the sake of brevity, reference to CHO cells contemplates a specific reference to HEK cells (e.g., HEK-293 cells), HL-60 cells, and/or any other (protein expression) cell(s) or cell line(s) known in the art. In some embodiments, the cells can (each) contain (one or more copies of) an exogenous nucleic acid. The nucleic acid can encode a polypeptide with at least and/or about 80% amino acid sequence identity to at least a portion of one of SEQ ID NO: 1 through SEQ ID NO: 75, or a combination of two or more thereof, preferably to one of SEQ ID NO: 2 through SEQ ID NO: 70.

The nucleic acid can comprise at least one transgene or cDNA. In some embodiments, at least a portion of the nucleic acid can have at least and/or about 80% nucleic acid sequence identity to one of SEQ ID NO: 76 through SEQ ID NO: 106 or SEQ ID NO: 121 through SEQ ID NO: 124, or a combination of two or more thereof, preferably one of SEQ ID NO: 76 through SEQ ID NO: 106 or SEQ ID NO: 121 through SEQ ID NO: 124. The nucleic acid can be or comprise a plasmid or other (structural) form of nucleic acid.

In some embodiments, the exogenous nucleic acid can encode a functional enzyme, such as dihydrofolate reductase (DHFR) and/or glutamine synthetase (GS). In at least one embodiment, the cells can be or comprise dihydrofolate reductase (DHFR)-deficient CHO cells, such as CHO-S cells or CHO-M cells. The nucleic acid can include a promoter (e.g., a weak up to a strong promoter, as understood by those skilled in the art). For instance, in some embodiments, the nucleic acid can include a (strong) promoter associated with the transgene having at least 80%, etc. nucleic acid sequence identity to one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152. Thus, the transgene can be under the control of a promoter.

For convenience, CHO cells and/or cell lines are referred to throughout the present disclosure. It is noted, however, that other cells, cell lines and/or host cells (besides CHO cells) are also contemplated within the scope of the present disclosure. Accordingly, a reference to CHO cells and/or cell lines also contemplates reference to and/or use of other known cells, cell lines and/or host cells (e.g., HEK cells, such as HEK-293 cells, HL-60 cells, and so forth).

### Transfection

A method of manufacturing the cell line can include introducing the exogenous nucleic acid into the cells, preferably via transfection, or other technique, as known in the art. In at least one embodiment, a serum-free growth optimized, cell-suspension of a cell line was used as the host cell line for insertion of a nucleic acid (plasmid) containing a promoter, a human alpha S-klotho transgene encoding a polypeptide with at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152, and a selectable (enzyme) marker. The transgenes, respectively, encode amino acids 1 through 981, 29 through 981, or 34 through 981 of human alpha soluble Klotho. In particular embodiments, the transgenes had sequence portion(s) corresponding to one or more of SEQ ID NO: 76 through SEQ ID NO: 106 or SEQ ID NO: 121 through SEQ ID NO: 124 (or had at least 80% nucleic acid sequence identity to one of SEQ ID NO: 76 through SEQ ID NO: 106 or SEQ ID NO: 121 through SEQ ID NO: 124). For DHFR-deficient CHO cell lines (such as the CHO-S cell line), the selectable (enzyme) marker was exogenous DHFR. For other cell lines, the selectable (enzyme) marker was exogenous GS.

Growth, Selection and/or Gene Amplification

Some embodiments can include growing the cells (e.g., transfected cells) on a solid medium and/or in a liquid medium (e.g., in suspension cell culture), preferably in a serum-free and/or animal (or animal-derived) protein (component) free medium. For instance, cells can be plated on solid growth media for a period of time. The cells can also or alternatively be grown in suspension culture and/or in liquid medium. The liquid medium preferably comprises a carbon source, a nitrogen source, and one or more vitamins, minerals, salts, amino acids, supplements, or additives. In some embodiments, the medium can also lack hypoxanthine and thymidine (HT), glutamine, etc.

In at least one embodiment, after a certain period of time (e.g., 48 hours post-transfection), the cells can be gathered (e.g., detached), optionally centrifuged (e.g., at 100×g for 5 min.), and/or seeded (e.g., at approximately 2000 cells/well), such as into a 96-well adherent culture plate (e.g., containing serum supplemented, -HT and/or -glutamine media). The medium can also include MTX and/or MSX in certain embodiments. Non-transfected cells may die within 7-14 days after selection (e.g., after exposure to MTX and/or MSX in -HT and/or -glutamine media.

In certain embodiments, the cells can (be selected to) contain at least about 2 to 10 copies, at least about 10 to 20 copies, at least about 20 to 30 copies, or at least about 30 to 50 copies of the exogenous nucleic acid (e.g., per cell). Accordingly, the method can include selecting cells that contain at least about 2 to 10 copies, at least about 10 to 20 copies, at least about 20 to 30 copies, or at least about 30 to 50 copies of the exogenous nucleic acid (e.g., per cell). For instance, (successively increasing levels of) MTX and/or MSX can be administered to the cells (e.g., at a concentration of about 1 nM-1μM, about 10-100 nM, etc.).

Dihydrofolate reductase (DHFR) gene amplification in DHFR-deficient CHO cells (such as the CHO-S cell line) transfected with exogenous DHFR, was accomplished by successively increasing levels of methotrexate (MTX) in growth medium. Because the plasmid contains DHFR, this allows for the amplification of the S-klotho gene (fragment) within the host cell upon exposure to MTX (10-100 nM). The GS gene expression system was also used to amplify cells transfected with exogenous GS (e.g., upon exposure to MSX). Alternatively, GS−/−host cell lines were also used, which removed the need for MSX. These steps resulted in the production of numerous copies of the S-klotho gene (e.g., between 10 to 30 copies of the gene per cell) and resultant high levels of expression of the S-klotho protein in the transgenic cell line.

In suspension culture, the protein can be secreted from the cells into the liquid medium in some embodiments. For instance, certain cells and/or cell lines of the present disclosure can secrete (or be selected to secrete) up to a concentration of 200-500 mg of protein per liter of liquid medium, 500-2000 mg of protein per liter of liquid medium, 2000-5000 mg of protein per liter of liquid medium, or any value or range of values therebetween (without concentrating the protein). In at least one embodiment, high producing cell lines (or suspension cultures) can be selected, such that the concentration of human recombinant alpha soluble Klotho protein in the medium (of the selected suspension culture or suspension cultures of the selected cell lines) is at least 200 mg/L, preferably at least 500 mg/L, more preferably at least 1000 mg/L, even more preferably at least 2000 mg/L, still more preferably at least 5000 mg/L, without concentrating the protein.

Sub-cloning of the resultant high S-klotho producing, transgene-containing colonies by limited cell dilution was conducted to further produce S-klotho-secreting cell lines that secrete in the range of 500 to 2000 mg/L S-klotho into the cells conditioned media. All cell constructs were restriction digested and sequence verified.

In some embodiments, the cells can be grown in a bioreactor having a volume or working volume of at least 10 liters, preferably at least 25 liters, more preferably at least 50 liters, even more preferably at least 100 liters, still more preferably at least 250 liters, still more preferably at least 500 liters, still more preferably at least 1,000 liters, still more preferably at least 2,000 liters, still more preferably at least 2,500 liters, still more preferably at least 5,000 liters, still more preferably at least 10,000 liters.

### Cell Line Maintenance

For the amplified high-producing S-Klotho cell lines (e.g., produced by DHFR/MTX or GS/MSX systems), optionally followed by cell subcloning, judicious use of concentrated media feeds administered to the production cell lines during scale-up and up to the final bioreactor run were done in serum-free and animal protein component-free basal media.

Scale-up of high producer cell lines was done by cell inoculum expansion in cell suspension in shake flasks or in the Wave Bag systems followed by the successive inoculation of the cells produced in 100 L and then 500 L capacity bioreactors. Cell viability was maintained at greater than 85% viable cells throughout the growth cycle in shake flasks, Wave Bags, or bioreactors; and then at 80% or greater viable cell counts in bioreactors during the plateau phase of CHO S cellular growth with the concomitant production of up to 1-3 g/L of S-klotho produced (designated as the “high producers”).

### Protein Production

Certain embodiments can employ recombinant DNA strategies that utilize strong promoter sequences and/or high copy number plasmids for production of therapeutic amounts of the Klotho protein in mammalian (e.g., CHO) cells. In at least one embodiment, for example, dihydrofolate reductase (DHFR) gene amplification in DHFR-deficient CHO cells can include providing and/or the use of (successively increasing levels of) methotrexate (MTX). Similarly, exogenous glutamine synthetase (GS) gene-containing cells can be treated with methionine sulphoximine (MSX).

The Klotho protein can also include one or more glycans (attached thereto). For instance, the native human alpha Klotho isoform 1 (SEQ ID NO: 1) can have glycans attached (via glycosylation) at amino acids 106, 159, 283, 344, 604, 612, and/or 694. The Klotho protein of the present disclosure can have one or more of the same (or similar) glycans attached (via glycosylation) thereto (e.g., at the same amino acid(s)).

In at least one embodiment, the protein can be cGMP regulation compliant, as determined and enforced by the U.S. Food and Drug Administration (FDA). For instance, the Klotho protein can be at least 90%, 92%, 95%, 96%, 97%, 98%, or 99% pure, dry weight. In some embodiments, the Klotho protein sample can include less than about 1-100 parts per million (ppm), less than about 100-1000 parts per billion (ppb), or less than about 1-100 ppb CHO host cell proteins (HCP), nucleic acid, and/or other cellular components, or any value or range of values disposed therebetween.

Glycan structures present on the S-Klotho proteins produced can be similar or identical in comparison to the structures on native S-klotho proteins isolated from human fluids (i.e., blood, serum, urine, cerebrospinal fluid). In at least one embodiment, confirmation of native-like glycans can ensure that the right native post-translation modifications (PTMs) are produced and stably maintained in the cell-produced S-Klotho protein.

Solubility and/or Half-Life Extension of Klotho Proteins

Disclosed are methods and compositions for extending the half-life and increasing the solubility of the human S-Klotho protein. Also, the purification and characterization of the protein constructs so produced to achieve these results are also the subject matter of the present disclosure. Relevant information regarding nucleic acid changes made in the sequence of the klotho gene or nucleic acid construct (see SEQ ID NOS: 76-96) and/or of changes, or chemical alterations in the amino acid sequence of a Klotho protein (see SEQ ID NOS: 1-70), and/or additions or subtraction of chemical groups, peptides, or proteins to the amino acid sequence of a Klotho protein is taught in the present disclosure in order to obtain resultant human Klotho variant proteins (novel compositions) with increased biological half-life or increased solubility in biological matrices (such as in blood, cerebrospinal fluid, urine, or various human tissues) than that of native Klotho molecules. These novel compositions can be made through the methods described herein for the modification of the S-Klotho protein.

Illustratively, fusion protein constructs can be produced by combining the S-Klotho protein with the Fc domains of an antibody (IgG), an albumin, such as human serum albumin (HSA), a transferrin, such as human transferrin (TF), and/or a proprietary recombinant polypeptide such as XTEN®. The novel proteins can also be produced by conjugating (or modifying) the S-Klotho protein with a posttranslational modification, such as polysialic acid (PSA) or pegylation. Moreover, a novel S-Klotho protein can be produced through pegylation. The foregoing and other (half-life extension) methodologies can improve the performance of the S-Klotho protein by one or more of: increasing the S-Klotho dosing interval; providing superior patient convenience and likely compliance; reducing dosing frequency requirement, resulting in lower drug use in the aggregate; reducing the cost of the drug; lowering drug quantities at the same dosing interval as the parent protein; simplifying dosage formulation and enabling subcutaneous formulation; providing higher drug levels using the same dose and dosing interval as the parent protein resulting in longer drug exposure and potentially better efficacy; and decreasing the immunogenicity of S-Klotho. Production of Fc domain fusion protein constructs of S-Klotho

Antibody Fc domain, human serum albumin (HSA), and polysialic acid (PSA) were tested for effectiveness in extending the half-life and increasing the solubility of the human S-Klotho protein. Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism through binding of the extended protein to the neonatal Fc receptor, FcRn. After binding of the extended protein to the FcRn receptor, degradation of the fusion protein in the cells' endosome is prevented. Fusions based on the addition of Fc or albumin can result in biological half-lives in the range of 3-16 days, much longer than which has been reported for typical pegylated or lipidated peptides. For a review that describes the use of protein fusion technologies such as Fc fusion proteins, fusion to human serum albumin, fusion to carboxy-terminal peptide, and other polypeptide fusion approaches to make biobetter drugs with more desirable pharmacokinetic profiles see Strohl WR. Fusion Proteins for Half-Life Extension of Biologics as a Strategy to Make Biobetters. Biodrugs. 2015;29(4):215-239, the entirety of which is incorporated herein by specific reference.

Fc domains were thusly added to the parent protein (S-Klotho) to increase binding affinity to the Fc receptor (FcRn). FcRn is present inside lysosomes in endothelial cells lining the blood vessels and functions to rescue antibodies from the degradation that makes most proteins short-lived in circulation. As a result of interactions with FcRn, proteins have half-lives ranging from a few days to a few weeks, allowing for less frequent dosing of the extended form of the protein drugs than biologics that do not have this newly-produced composition-of-matter.

The dimeric nature of Fc versus the monomeric structure of HSA can lead to the presentation of a Fc fused peptide as a dimer or a monomer in contrast to HSA. The dimeric nature of a peptide Fc fusion can produce an avidity effect if the target receptors for S-Klotho are spaced closely enough together or are themselves dimers in particular human target organs. This may be desirable or not depending on the target. Fusion of the S-Klotho protein to antibody Fc is also taught in the present disclosure to improve the solubility and stability of S-Klotho. Surprisingly, Fc-fusion Klotho proteins appear to have improved binding, activity, and/or immunogenicity. The addition of Fc domains to S-Klotho will also allow the fusion protein to be less immunogenic upon administration in human subjects.

Conjugation of the S-Klotho protein with human serum albumin (HSA)

The 66.5 kDa protein HSA, similar to human IgGs, has a long average half-life in the 19-day range. At a concentration of ˜50 mg/mL (˜600 uM), HSA is the most abundant protein in human plasma, where it has several functions, including maintenance of plasma pH, metabolite and fatty acid transport, and a role in maintaining blood pressure. HSA, which is at the upper limit of size for glomerular filtration of proteins by the kidney, is also strongly anionic, which helps even more to retard its filtration via the kidney. Like IgGs, HSA also binds FcRn in a pH-dependent manner, albeit at a site different from—and via a mechanism distinct from—that of IgG binding, and is recycled similarly to IgGs, resulting in its extended half-life. HSA also tends to accumulate in tumors and in inflamed tissues, which suggests that fusion or binding to albumin may potentially help to target proteins or peptides to those sites.

The fusion of peptides or proteins with inherently short-half-life properties to HSA for prolongation of the serum half-life of these molecules has been investigated broadly since the early 1990s. Since then, dozens of different peptides and small proteins have been fused to HSA as both innovative and potential biobetter molecules. The first HSA-peptide or protein fusion product to be approved for marketing was Tanzeum® (marketed as Eperzan® in the European Union) a DPP-4-resistant GLP-1-HSA fusion protein discovered at Human Genome Sciences and developed and marketed by GlaxoSmithKline. Tanzeum® (albiglutide) was approved by the European Medicines Agency (EMA) and the FDA in March and April of 2014, respectively. HSA thus improved the half-life of pharmacologically active GLP-1 from 1-2 min for native GLP-1 to 4-7 days, which allows for once weekly dosing. Seven other known HSA fusion protein product candidates are either now in development or recently have been in development. Also, Novozyme, has been developing modified versions of recombinant HSA with improved FcRn binding for construction of “next-generation” HSA-protein fusions that may possess even longer half-life properties. This was based on the use of a K573P mutant of HSA, which was found to possess 12-fold greater affinity for FcRn, conferred a longer half-life on HSA than the wild type molecule in both mice and cynomolgus monkeys. The expectation is that these longer-half-life mutants of HSA may be of further use as fusion proteins to improve the half-life of fusion proteins.

The Inventors herein therefore disclose the fusion of Klotho proteins to wild type HSA or to a mutant form of HSA to produce a Klotho fusion molecule(s) with significantly prolonged half-life in human blood, cerebral spinal fluid, and other human biological matrices in order to bring about a strategic therapeutic benefit such as superior patient convenience and likely compliance, reduced dosing frequency resulting in lower drug use in aggregate, and/or reduced cost of goods. Also, lower drug quantities at the same dosing interval as the parent protein may simplify dosage formulation and enable subcutaneous formulation or decrease in the immunogenicity of S-Klotho. Surprisingly, HAS-fusion Klotho proteins appear to have improved binding, activity, and/or immunogenicity.

Conjugation of the S-Klotho Protein with Human Transferrin (TF)

Transferrin is a highly abundant serum glycoprotein, found in serum at 3-4 mg/mL, which binds iron tightly but reversibly and functions to carry iron to tissues. Transferrin has 679 amino acid residues, is about 80 kDa in size, and possesses two high-affinity Fe3+−binding sites, one in the N-terminal domain and the other in the C-terminal domain. Human transferrin has a half-life reported to be 7-10 days or 10-12 days. The aglycosylated form of human transferrin, which makes up about 2-8% of the total transferrin pool, has a slightly longer half-life of 14-17 days. The extended persistence of transferrin in human serum is due to a clathrin-dependent transferrin receptor-mediated mechanism, which recycles receptor-bound transferrin back into the circulation.

Fusions of peptide and proteins have been made to human transferrin to the N- and C-termini, as well as to the centrally located hinge region that links the two major lobes of transferrin together. The N terminus of transferrin is free and can be fused directly. The C terminus is more buried and is constrained by a nearby disulfide bond, so flexible linkers are typically used when proteins are fused to the C terminus. This capability was extended by making libraries of peptides against specific targets and then fusing binders from those libraries into aglyco-transferrin (N-terminal, C-terminal, loops, or linker region) to be developed into therapeutic fusion proteins with extended half-lives.

The biotechnology company BioRexis Technologies, Inc., was founded in 2002 to develop the transferrin fusion protein platform, which they termed the “Trans Body” platform, as a therapeutic platform. Their lead molecule, BRX-0585, was a transferrin-GLP-1 fusion protein for treatment of type 2 diabetes mellitus (T2DM). Fusion of GLP-1 to transferrin was demonstrated to significantly enhance the half-life of GLP-1. BioRexis was acquired by Pfizer in March 2007. As far as can be determined, no BioRexis-derived fusion proteins are currently in the clinic. Klotho protein can be fused to human transferrin to produce a Klotho fusion molecule, which can be administered clinically to significantly prolong half-life or stability in human biological matrices in vivo.

Conjugation of the S-Klotho Protein with XTEN from Amunix

XTEN® is a proprietary recombinant polypeptide that extends the in vivo half-life of therapeutic payloads. XTEN consists of naturally-occurring hydrophilic amino acids and is biodegradable. Pharmaceuticals such as proteins, peptides, and synthetic compounds can be XTENylated via chemical conjugation or genetic fusion. XTEN proteins lack secondary and tertiary structure and their solution behavior resembles chemically prepared polymers with very large hydrodynamic radii. By size exclusion chromatography, XTEN protein polymers appear much larger than typical globular proteins of similar molecular weight. The bulking effect of XTEN greatly reduces renal clearance of attached molecules, thus greatly increasing their in vivo half-lives. In the current invention, the length of XTEN polymers added to a Klotho protein will be customized to optimize the pharmacokinetics as well as the bio-distribution of the attached Klotho protein payloads.

XTEN thusly can be recombinantly-fused to S-Klotho protein(s) to increase the molecules in vivo half-life. One benefit is that with the genetic S-Klotho-XTEN fusion constructs, a molecule is produced that has the convenience of expression, purification and characterization of a single molecule which includes both the therapeutic and bulking moieties. Recombinant fusion allows the attaching of multiple XTEN chains per protein in precisely-defined locations and has been successfully used by therapeutic drug manufacturers, resulting in best-in-class pharmacokinetics as exemplified by XTENylated growth hormone (Somavaratan, from the company, Versartis) and FVIII-XTEN (from Biogen). For example, pharmacokinetic studies conducted in children receiving different doses of Somavaratan) have shown that XTENylation resulted in best-in-class half-life due to reduced receptor-mediated elimination in addition to kidney clearance.

XTEN protein polymers can be produced as free intermediates for chemical conjugation to peptides, peptidomimetics, and other synthetic molecules. Reactive groups (thiol, amine) are inserted in precisely-defined positions via introduction of cysteine or lysine residues into XTEN-encoding genes. Amunix has developed XTENs containing 1 to 9 thiol groups with various spacing which can be provided to partners. Thus, in the present invention, orthogonal conjugation to amino and thiol groups in XTEN facilitates the production of Klotho-XTEN molecules.

### Purification of Proteins

The Klotho protein can be extracted from cell suspension culture of cells (e.g., of a CHO cell line). The cells can produce and optionally secrete the Klotho protein (e.g., into liquid medium). Secretion of S-klotho into the spent media was also observed.

Purification of the recombinant proteins of the present disclosure can be carried out by any suitable method known in the art or described herein, for example, any conventional procedures involving extraction, precipitation, chromatography and/or electrophoresis. A further purification procedure that can be used for purifying proteins includes affinity chromatography using monoclonal antibodies which bind to target protein(s). Some embodiments can include an IgG-tagged protein, Fc-tagged protein, His-tagged protein, HSA-tagged protein, GST-tagged protein, etc., that can be purified by affinity chromatography. For instance, some embodiments can include at least a portion of an Fc domain of an IgG, such as IgG1. Generally, crude preparations containing a recombinant protein are passed through a column on which a suitable monoclonal antibody is immobilized. For other tags, a corresponding, tag-binding affinity entity can be disposed in the column. After washing the column, the protein is eluted from the gel by changing pH or ionic strength. For example, the spent media from the CHO-S high producer cell lines were concentrated via tangential flow filtration; and S-klotho protein was purified by affinity chromatography followed by ion exchange cartridge or column chromatography. Size exclusion chromatography can also be used to purify the proteins. It will be appreciated that various ordering of chromatography types can be applied. Any suitable combination of chromatography steps or columns is contemplated herein.

In alternative protocols, one or more pre- or post-affinity purification steps were performed. Such steps can include, for example, (ultra) centrifugation, dialysis, membrane and/or tangential flow filtration, chromatographic separation, such as ion exchange, liquid-liquid extraction, such as (aqueous) two-phase extraction, or other known purification steps. One or more post purification processing steps were performed in certain embodiments. Such post purification processing steps can include, for example, tandem anion/cation flow-through chromatography (as opposed to bind-and-elute chromatography), viral and/or bacterial removal by membrane filtration (e.g., 0.2 micron, 0.1 micron, etc.) or by other means known to one of ordinary skill in the art.

Certain embodiments include a method of purifying a recombinant Klotho protein. The method can include expressing the recombinant Klotho protein in suspension cell culture. The method can include harvesting the Klotho protein, such as by collecting suspension culture medium and/or cells. In at least one embodiment, the method can include a first purification step. It will be appreciated that the “first” purification step need not occur first or prior to any other purification step(s), or after any other purification step(s). The first purification step can comprise, involve, or use chromatography, such as High Performance Liquid Chromatography (HPLC), Fast Protein Liquid Chromatography (FPLC), ion exchange chromatography, affinity chromatography, etc. The first purification step can comprise, involve, or use affinity chromatography. The affinity chromatography can be based on a tag or other protein fusion entity of the Klotho protein. For example, a His-tagged protein can be purified by His-tag affininty chromatography (e.g., HisTrap or nickel column chromatography). Similarly, a GST-conjugated protein can be purified by GST-affinity chromatography. Depending on the specific tag(s), a corresponding, tag-binding affinity entity can be disposed in a chromatography column for use in protein purification.

In at least one embodiment, the first purification step can comprise, involve, or use affinity chromatography using, for example, Protein A or Protein A-conjugated particles or beads (e.g., Protein A Sepharose 4 Fast Flow column (0.75 ml) (0.5 cm×3 cm) for purification of Ig-tagged or Fc-fusion Klotho proteins. The column can be equilibrated in an appropriate solution. The solution can include a buffering agent at a suitable concentration (e.g., 10 mM Tris-HC1, pH 8.0). A suitable amount of protein-containing sample (e.g., suspension cell culture supernatant, optionally adjusted to pH 8.0, with 1.5 M Tris-HC1, pH 8.0) can be loaded on to the column (e.g., at a flow rate of 0.75 ml/min), optionally washed with a suitable wash buffer (e.g., 10 mM Tris/Arginine-HC1, pH 8.0), and eluted from the column with a suitable elution buffer (e.g., 4 M MgCl₂, pH 3 (i.e., low pH, high salt)), at a suitable flow rate (e.g., about 0.50-0.25 ml/min, or equivalent), and/or to produce a concentrated sample.

In at least one embodiment, the elution buffer can have a pH of about 3. In some embodiments, the elution buffer can have a pH between about pH 2 and about pH 6, preferably between about pH 2 and about pH 5, more preferably between about pH 2 and about pH 4, still more preferably between about pH 2.5 and about pH 3.5.

In at least one embodiment, the elution buffer can include at least one salt. Illustratively, the salt can be or comprise MgCl₂, preferably at a concentration of about 4 M. In some embodiments, the elution buffer can have a salt concentration between about 1 M and about 6 M, preferably between about 2 M and about 5 M, more preferably between about 3 M and about 5 M, still more preferably between about 3.5 M and about 4.5 M.

Additional purification step(s) can comprise, involve, or use chromatography, such as High Performance Liquid Chromatography (HPLC), Fast Protein Liquid Chromatography (FPLC), ion exchange chromatography, affinity chromatography, size exclusion (or gel filtration) chromatography, etc. In at least one embodiment, the method can include a second purification step. It will be appreciated that the “second” purification step need not occur second, prior to, or before any other purification step(s). The second purification step can comprise, involve, or use size exclusion chromatography (and a suitable mobile phase buffer) to fractionate, resolve, clarify, or purify the protein-containing sample(s). For example, in an illustrative second purification step, protein-containing elution sample(s) (from the first purification step) can be optionally buffer exchanged, and purified using size exclusion chromatography (e.g., Superdex 200 (1 cm×30 cm) column) in a suitable mobile phase buffer. In at least one embodiment, the mobile phase buffer can include a suitable buffering agent (e.g., 100 mM Tris-HCl) and one or more optional reducing agent (e.g., 5 mM L-Methionine and/or 0.6% Sodium Thioglycolate), at a suitable pH (e.g., about pH 8). In at least one embodiment, the buffering agent can be other than a phosphate-containing buffer (e.g., Tris, Citrate, etc.). In at least one embodiment, the reducing agent can be or comprise L-Methionine and/or Sodium Thioglycolate, preferably at effective reducing concentration(s). Alternative reducing agents may include an acetylcysteine (i.e., N-acetylcysteine (NAC), including N-acetyl-L-cysteine, N-acetyl-D-cysteine, and racemic N-acetylcysteine or a (racemic) mixture of N-acetyl-L-cysteine and N-acetyl-D-cysteine), ascorbic acid, dithionite, erythiorbate, cysteine, glutathione, dithiothreitol, 2-mercaptoethanol, dierythritol, a resin-supported thiol, a resin-supported phosphine, vitamin E, and/or trolox, or salts thereof, sodium citrate, potassium citrate, potassium iodide, ammonium chloride, guaiphenesin (or guaifenesin), Tolu balsam, Vasaka, ambroxol, carbocisteine, erdosteine, mecysteine, dornase alfa, and so forth.

In at least one embodiment, the mobile phase buffer can have a pH of about 8. In some embodiments, the elution buffer can have a pH between about pH 6 and about pH 10, preferably between about pH 7 and about pH 9, more preferably between about pH 7.2 and about pH 8.8, still more preferably between about pH 7.5 and about pH 8.5.

In at least one embodiment, the eluted protein can be or include monomeric Klotho protein. In at least one embodiment, the eluted protein can be or include multimeric Klotho protein. In some embodiments, the eluted protein can be or include dimeric Klotho protein. In some embodiments, the eluted protein can be or include tetrameric Klotho protein. In some embodiments, the eluted protein can be or include hexameric Klotho protein. In some embodiments, the eluted protein can be or include octameric Klotho protein. In at least one embodiment, the eluted protein can be or include both monomeric and multimeric Klotho proteins. In at least one embodiment, the eluted protein can be or include a single species of either monomeric or multimeric Klotho protein. In at least one embodiment, the eluted protein can be or include greater than or equal to about 80% of the single species of either monomeric or multimeric Klotho protein. In at least one embodiment, the eluted protein can be or include greater than or equal to about 82%, 85%, 88%, 90%, 92%, 95%, 96%, 97%, 98%, or 99% of the single species of either monomeric or multimeric Klotho protein.

Additional purification step(s) can comprise, involve, or use chromatography, such as High Performance Liquid Chromatography (HPLC), Fast Protein Liquid Chromatography (FPLC), ion exchange chromatography, affinity chromatography, size exclusion (or gel filtration) chromatography, etc.

Analysis of Klotho proteins

Protein purity can be demonstrated by SDS-PAGE or other assays or means known in the art. For instance, in at least one embodiment, Klotho protein samples (50 μg) were fractionated on precast SDS-PAGE gels (4-15%, 10 wells; catalog no. 456-1083; BioRad) and stained with Coomassie blue stain. All samples were run with either an empty lane in between or on separate gels to avoid sample-to-sample contamination. S-klotho of greater than 98% was shown to be isolated from the CHO S conditioned media as determined by Coomaise blue stain and densitometry tracing, or by silver stain visualization, or by HPLC or RP-HPLC. In order to obtain sequence information, the proteins (after reduction and S-carboxymethylation) can be cleaved with cyanogen bromide, trypsin, and/or proteinase K and the peptides separated by HPLC according to known methods of protein chemistry. Thus—prepared samples were then sequenced in an automatic gas phase microsequencing apparatus (Applied Biosystems Model 470A, ABI, Foster City, Calif., USA) with an on-line automatic HPLC PTH amino acid analyzer (Applied Biosystems Model 120, ABI see above) connected to the outlet.

Proteins were also analyzed through mass spectroscopic methods. Concerning sample preparation for mass spectrometry, in order to restrict analyses to the correct S-klotho protein, only the gel band between 75 and 150 kDa was resected for analysis. Gel fractions were macerated with a sterile blade and subjected to in-gel digestion. Gel fractions were de-stained by three washes with 80 μL of 50% acetonitrile (ACN)/50 mM ammonium hydrogen carbonate and washed with 100% ACN. The alkylation step was omitted, given the absence of cysteine residues from the target α-Klotho peptides. Tryptic digestion was carried out overnight at 37° C. with 60 μL of trypsin (sequencing grade modified, catalog no. V511A; Promega) in 50 mM ammonium hydrogen carbonate (0.005 μg/pL). This process yielded 25 μL, of which 5 μL (1 μL for S-Klotho) was subjected to liquid chromatography-electrospray ionization tandem mass spectrometry (MS/MS) and PRM analysis in an Orbitrap nano-ESI Q-Exactive mass spectrometer (Thermo Scientific), coupled to a nanoLC (Dionex Ultimate 3000 UHPLC). MS/MS analysis confirmed that the human recombinant alpha S-klotho produced by embodiments of the present disclosure was substantially similar (e.g., identical in corresponding amino acid sequence) to that found in human blood, serum, urine or cerebrospinal fluid.

Using the above purification methods, the level of contaminating CHO host cell proteins (HCP) was determined to be acceptable in the purified S-Klotho protein. In the final S-Klotho product, HCP was removed to <1-100 ppm. S-klotho protein products of at least 90%, and up to 98% purity were isolated from the spent CHO S production cell line (cells and/or liquid medium). Specifically, cGMP-grade human alpha S-klotho that had an analytical profile suitable for clinical administration in human subjects was produced and purified. For example, the analytical profile of human recombinant alpha S-Klotho is designated by reference number PXD002775 in the ProteomeXchange Database, which can be found at http://proteomecentral.proteomexchange.org/cgi/GetDataset?ID=PXDO02775. The NIH full S-Klotho protein dataset is at http://www[dot]ncbi[dot]nlm[dot]nih[dot]gov/protein/Q9UEF7.

An analytical profile for S-Klotho suitable for clinical administration and that obtained in an embodiment of the present disclosure included an endotoxin level less than 0.1 ng per pg (1 EU/μg) of S-Klotho. In addition, the purified human recombinant S-Klotho was also shown to have >or =98% purity by SDS PAGE.

Glycan structures present on the CHO S cell-produced S-Klotho were identical in comparison to the structures on native S-Klotho isolated from human fluids (i.e., blood, serum, urine, and cerebrospinal fluid). This ensured that the same, native post-translation modifications (PTMs) were produced and stably maintained in the cell produced S-Klotho protein. Accordingly, using production and purification method described herein, the Inventors have been successful in producing cGMP-grade human S-klotho that had an analytical profile suitable for clinical administration in human subjects.

### Therapeutic Compositions

Some embodiments of the present disclosure can include a pharmaceutical composition, such as a therapeutic composition. Pharmaceutical compositions of the present disclosure can generally include a therapeutically effective amount of a recombinant soluble alpha Klotho protein admixture with a (pharmaceutically-acceptable) vehicle, carrier, or excipient comprised of one or more additional components. The Klotho protein can be included in any suitable amount, such as about 0.001-1000 mg, about 0.01-100 mg, about 0.1-10 mg, about 1-5 mg, or any amount or range of amounts therebetween. In some embodiments, the composition can include a pharmaceutically or therapeutically effect amount of Klotho protein (e.g., to raise serum Klotho levels to a predetermined level in a subject to which the composition is administered).

The components can include one or more aggregation inhibitors, buffers, tonicity modifiers, and additional excipients. The primary solvent in the carrier can be either aqueous or non-aqueous in nature. The composition can be prepared by combining a purified Klotho protein of the present disclosure with a pharmaceutically-acceptable carrier.

It will be understood by one of ordinary skill in the art that the combining of the various components to be included in the composition can be done in any appropriate order, namely, the buffer can be added first, middle or last and the tonicity modifier can also be added first, middle or last. It is also to be understood by one of ordinary skill in the art that some of these chemicals can be incompatible in certain combinations, and accordingly, are easily substituted with different chemicals that have similar properties but are compatible in the relevant mixture.

Aggregation inhibitors reduce a polypeptide's tendency to associate in inappropriate or unwanted ternary or quaternary complexes. The amino acids L-arginine and/or, L-cysteine, can act to reduce aggregation of Fc domain containing polypeptides in a formulation for long periods, e.g., two years or more. The concentration of the aggregation inhibitor in the formulation is preferably between about 1 mM to 1 M, more preferably about 10 mM to about 200 mM, more preferably about 10 mM to about 100 mM, even more preferably about 15 mM to about 75 mM, and yet more preferably about 25 mM. These compounds are available from commercial suppliers.

Compositions of the present disclosure can include a buffering agent. Buffering agents maintain pH in a desired range. Various buffers suitable for use in the pharmaceutical composition of the present disclosure include histidine, potassium phosphate, alkali salts, sodium or potassium phosphate or their hydrogen or dihydrogen salts, sodium citrate or potassium citrate /citric acid, sodium acetate/acetic acid, maleic acid, ammonium acetate, tris-(hydroxymethyl)-aminomethane (tris), various forms of acetate and diethanolamine, and any other pharmaceutically acceptable pH buffering agent known in the art, to maintain the pH of the solution within a desired range. Mixtures of these buffering agents may also be used.

The amount of buffering agent useful in the composition depends largely on the particular buffer used and the pH of the solution. For example, acetate is a more efficient buffer at pH 5 than pH 6 so less acetate may be used in a solution at pH 5 than at pH 6. The preferred pH of the preferred formulations will be in the range of 4.0 to 5.0, and pH-adjusting agents such as hydrochloric acid, citric acid, sodium hydroxide, or a salt thereof, may also be included in order to obtain the desired pH.

One preferred buffer is sodium phosphate as its buffering capacity is at or near pH 6.2. It will be appreciated, however, that other buffers may be selected to achieve any desirable pH buffering. The concentration of the buffer in the formulation is preferably between about 1 mM to about 1 M, more preferably about 10 mM to about 200 mM. Buffers are well known in the art and are manufactured by known methods and available from commercial suppliers.

When the pH of the pharmaceutical composition is set at or near physiological levels comfort of the patient upon administration is maximized In particular, it is preferred that the pH be within a range of pH about 5.8 to 8.4, with about 6.2 to 7.4 being preferred, however, it is to be understood that the pH can be adjusted as necessary to maximize stability and solubility of the polypeptide in a particular formulation and as such, a pH outside of physiological ranges, yet tolerable to the patient, is within the scope of the disclosure.

The formulations of the present disclosure may further include one or more tonicity modifiers (e.g., to render the solution isotonic with a patient's blood for injection). A tonicity modifier is understood to be a molecule that contributes to the osmolality of a solution. The osmolality of a pharmaceutical composition is preferably regulated in order to maximize the active ingredient's stability and also to minimize discomfort to the patient upon administration. Where serum is approximately 300+/−50 milliosmoles per kilogram. It is generally preferred that a pharmaceutical composition be isotonic with serum, i.e., having the same or similar osmolality, which is achieved by addition of a tonicity modifier, thus it is contemplated that the osmolality can be from about 180 to about 420 milliosmoles, however, it is to be understood that the osmolality can be either higher or lower as specific conditions require.

Typical tonicity modifiers are well known in the art and include but are not limited to various salts, amino acids or polysaccharides. Non-limiting examples of suitable amino acids include glycine. Non-limiting examples of suitable polysaccharides include sucrose, mannitol and sorbitol. It is understood that more than one tonicity modifier may be used at once, for example, sorbitol and glycine can be used in combination to modify a formulation's tonicity.

Additional examples of tonicity modifiers suitable for modifying osmolality include, but are not limited to amino acids (e.g., arginine, cysteine, histidine and glycine), salts (e.g., sodium chloride, potassium chloride and sodium citrate) and/or saccharides (e.g., sucrose, glucose and mannitol). The concentration of the tonicity modifier in the formulation is preferably between about 1 mM to 1 M, more preferably about 10 mM to about 200 mM. Tonicity modifiers are well known in the art and are manufactured by known methods and available from commercial suppliers.

Excipients, also referred to as chemical additives, co-solutes, or co-solvents, that stabilize the polypeptide while in solution (also in dried or frozen forms) can also be added to a pharmaceutical composition. Excipient is defined herein as a non-therapeutic agent added to a pharmaceutical composition to provide a desired effect, e.g. stabilization, isotonicity. Common attributes of desirable excipients are aqueous solubility, non-toxicity, non-reactivity, rapid clearance from the body, and the absence of immunogenicity. In addition, the excipients should be capable of stabilizing the native conformation of the protein so as to maintain the efficacy and safety of the drug during processing, storage and administration to the patient. Examples include but are not limited to sugars/polyols such as: sucrose, lactose, glycerol, xylitol, sorbitol, mannitol, maltose, inositol, trehalose, glucose; polymers such as: serum albumin (bovine serum albumin (BSA), human SA or recombinant HA), dextran, PVA, hydroxypropyl methylcellulose (HPMC), polyethyleneimine, gelatin, polyvinylpyrrolidone (PVP), hydroxyethylcellulose (HEC); non-aqueous solvents such as: polyhydric alcohols, (e.g., PEG, ethylene glycol and glycerol) dimethylsulfoxide (DMSO) and dimethylformamide (DMF); amino acids such as: proline, L-serine, sodium glutamic acid, alanine, glycine, lysine hydrochloride, sarcosine and gamma-aminobutyric acid; surfactants such as: Tween-80™ (polysorbate 80), Tween-20™ (polysorbate 20), SDS, polysorbate, polyoxyethylene copolymer; and miscellaneous excipients such as: potassium phosphate, sodium acetate, ammonium sulfate, magnesium sulfate, sodium sulfate, trimethylamine N-oxide, betaine, metal ions (e.g., zinc, copper, calcium, manganese, and magnesium), CHAPS, monolaurate, 2-O-beta-mannoglycerate or any combination of the above. As a matter of caution, it is noted that the highly toxic white hellebore (Veratrum album) can be mistaken for gentian.

The concentration of one or more excipients in a formulation of the disclosure is/are preferably between about 0.001 to 5 weight percent, more preferably about 0.1 to 2 weight percent. Excipients are well known in the art and are manufactured by known methods and available from commercial suppliers.

In one illustrative embodiment, a formulation of the disclosure can comprise about 150 mM NaCl buffered to pH 7.3 to 7.4 with HEPES, MES, or Tris-HCl, and, optionally, one or more additional components as described herein.

In one illustrative embodiment, a formulation of the disclosure can comprise a (therapeutically-effective) amount of Klotho protein in about 10 mM to about 100 mM L-arginine, about 10 mM to about 50 mM sodium phosphate, about 0.75% to about 1.25% sucrose, and/or about 50 mM to about 150 mM NaCl, at about pH 6.0 to about pH 7.0. In another embodiment, L-arginine can be replaced with L-cysteine (at about 1 to about 500 micromolar) in the formulation. In yet another embodiment, the pH can be about pH 7.0. In another specific embodiment, a formulation of the disclosure can comprise a (therapeutically-effective) amount of Klotho protein in about 25 mM L-arginine, about 25 mM sodium phosphate, about 98 mM sodium chloride, and/or about 1% sucrose at about pH 6.2.

In another embodiment, a formulation of the disclosure can comprise a (therapeutically-effective) amount of Klotho protein in about 10 mM to about 100 mM L-arginine, about 10 mM to about 50 mM sodium phosphate, about 0.75% to about 1.25% sucrose, about 50 mM to about 150 mM NaCl, at about pH 6 to about pH 7. In a specific embodiment, the formulation of the disclosure comprises a (therapeutically-effective) amount of Klotho protein in about 25 mM sodium phosphate, about 98 mM sodium chloride, and/or about 1% sucrose at about pH 6.2.

In yet another embodiment, a formulation of the disclosure can comprise an effective amount of an Fc domain containing Klotho fusion protein, about 10 mM to about 100 mM L -arginine, about 10 mM to about 50 mM sodium phosphate, about 0 to 5% Mannitol and/or 0 to 0.2% Tween-20™ (polysorbate 20) at about pH 6 to 7. In another embodiment, a formulation of the disclosure can comprise an effective amount of a Klotho protein, about 25 mM L-arginine, about 25 mM sodium phosphate, about 4% Mannitol, about 0.02% Tween-20™ (polysorbate 20), and/or at about pH 6.0.

In yet another embodiment, the disclosure provides a method of treating a mammal comprising administering a therapeutically effective amount of the pharmaceutical composition described herein, wherein the mammal has a disease or disorder that can be beneficially treated with a Fc domain containing polypeptide in the composition. In yet another embodiment, the Fc domain containing polypeptide is derived from the same species of mammal as is to be treated with the composition. In a particular embodiment, the mammal is a human patient in need of treatment. When the Fc domain containing polypeptide of the composition is Klotho:Fc, examples of diseases or disorders that can be treated include but are not limited to rheumatoid arthritis, psoriatic arthritis, ankylosing spondylitis, Wegener's disease (granulomatosis), Crohn's disease (or inflammatory bowel disease), chronic obstructive pulmonary disease (COPD), Hepatitis C, endometriosis, asthma, cachexia, psoriasis, and atopic dermatitis, or persons with a genetic disorder with mutations in one or more Klotho genes. Additional diseases or disorders that can be treated with Klotho:Fc include those described in WO 00/62790, WO 01/62272 and U.S. Patent Application No. 2001/0021380, the entirety of each of which is incorporated herein by reference.

In yet another embodiment, the disclosure provides a method for accelerated stability testing of the stability an Fc domain containing polypeptide in a pharmaceutical composition of the disclosure comprising the steps of testing the activity of the polypeptide formulated according to the disclosure prior to storage, i.e., time zero, storing the composition at 37° C. for one month and measuring the stability of the polypeptide, and comparing the stability form time zero to the one month time point. This information is helpful for early elimination of batches or lots that appear to have good stability initially, yet do not store well for longer periods.

Moreover, the pharmaceutical composition provides long-term storage such that the active ingredient, e.g., an Fc domain containing polypeptide, is stable over the course of storage either in liquid or frozen states. As used herein, the phrase “long-term” storage is understood to mean that the pharmaceutical composition can be stored for three months or more, for six months or more, for one year or more, and preferably for two year or more. Long term storage is also understood to mean that the pharmaceutical composition is stored either as a liquid at 2-8° C. or is frozen, e.g., at −20° C. or colder (e.g., −20 ° C. or −80° C.). It is also contemplated that the composition can be frozen and thawed more than once. The term “stable” with respect to long-term storage is understood to mean that the active polypeptide of the pharmaceutical composition does not lose more than 20%, or more preferably 15%, or even more preferably 10%, and most preferably 5% of its activity relative to activity of the composition at the beginning of storage.

One or more anti-oxidants can be included in the formulations of the present disclosure. Anti-oxidants contemplated for use in the preparation of the formulations include amino acids such as glycine and lysine, chelating agents such as EDTA and DTPA, and free-radical scavengers such as sorbitol and mannitol.

Other effective administration forms such as (an oral or parenteral) modified release formulations (e.g., coated, encapsulated, slow-release, controlled-release, extended-release, delayed-release, sustained-release, time-release, depot, etc.), inhalant mists, orally-active formulations, suppositories, and implants or implantable medical devices are also contemplated herein. As such, the formulations may also involve particulate preparations of polymeric compounds such as bulk erosion polymers (e.g., poly(lactic-co-glycolic acid) (PLGA) copolymers, PLGA polymer blends, block copolymers of PEG, and lactic and glycolic acid, poly(cyanoacrylates)); surface erosion polymers (e.g., poly(anhydrides) and poly(ortho esters)); hydrogel esters (e.g., pluronic polyols, poly(vinyl alcohol), poly(vinylpyrrolidone), maleic anhydride-alkyl vinyl ether copolymers, cellulose, hyaluronic acid derivatives, alginate, collagen, gelatin, albumin, and starches and dextrans) and composition systems thereof; or preparations of liposomes or microspheres. Such formulations may influence the physical state, stability, rate of in vivo release, and rate of in vivo clearance of the present proteins and derivatives. The optimal pharmaceutical formulation for a desired protein can be determined by one skilled in the art depending upon the route of administration and desired dosage. Exemplary pharmaceutical formulations are disclosed in Remington's Pharmaceutical Sciences, 18th Ed. (1990), Mack Publishing Co., Easton, Pa. 18042, pages 1435-1712, the disclosure of which is incorporated herein by reference.

### Biological Activity

Methods for evaluating the efficacy and/or determining an effective dose of human recombinant alpha soluble Klotho (to a patient, subject, or individual exhibiting an age-related disorder or clinical (e.g., metabolic) disorder) can (preliminarily) include performing organismal-based assays (e.g., using a mammal (e.g., a mouse, rat, primate, or some other non-human). The Klotho protein can be administered to the organism once or as a regimen (regular or irregular). For instance, the protein can be administered a suitable number of times (e.g., once, twice, etc.) in a given period of time (e.g., monthly, semi-monthly, weekly, semi-weekly, daily, etc.). A parameter of the organism (e.g., an age-associated parameter) can then be evaluated. Klotho proteins of interest can effectuate or result in a change in the parameter relative to a reference (e.g., a parameter of a control organism). Other parameters (e.g., related to toxicity, clearance, and pharmacokinetics) can also be evaluated.

The klotho proteins of the present disclosure may be evaluated using an animal (model) that has or exhibits a particular disorder or condition, such as an age-related or age-associated disorder or condition, a clinical (e.g., metabolic) disorder or condition, etc. Such disorders and conditions can also provide a sensitized system in which the effect of the protein on physiology can be observed. Exemplary disorders include, for example, denervation, disuse atrophy, clinical (e.g., metabolic) disorders (e.g., disorder of obese and/or diabetic animals such as db/db mouse, ob/ob mouse, etc.), cerebral disorders, liver ischemia or other liver disorders, cisplatin/taxolkincristine models, kidney idschemia/reperfusion models, various tissue (xenograft) transplants, transgenic bone models, pain syndromes (e.g., inflammatory and neuropathic disorders), Paraquat, genotoxic, oxidative stress models, and tumor (I) models.

To evaluate the S-Klotho protein of the present disclosure, the protein can be administered to a suitable animal (for a suitable treatment period) and the parameter of the animal is evaluated (e.g., after a suitable period of time, such as 10 to 60 minutes, 1 to 24 hours, 1 to 30 days, 1 to 12 months, 1 to 5 years, or any value or range of values therebetween). The animal can be fed ad libitum or normally (e.g., not under caloric restriction, although some parameters can be evaluated under such conditions). Typically, a cohort of such animals is used for the assay. Generally, a test polypeptide can be indicated as favorably altering lifespan regulation in the animal if the test polypeptide affects the parameter in the direction of the phenotype of a similar animal subject to caloric restriction. Such test polypeptides may cause at least some of the lifespan regulatory effects of caloric restriction (e.g., a subset of such effects) without having to deprive the organism of caloric intake.

The parameter(s) to be tested may be age-associated or disease associated parameter(s) (e.g., a symptom of the disorder associated with the animal model). A test protein that is favorably indicated can cause an amelioration of the symptom relative to a similar reference animal not treated with the polypeptide. Other parameters relevant to a disorder or to aging can include: antioxidant levels (e.g. antioxidant enzyme levels or activity), stress resistance (e.g., paraquat resistance), core body temperature, glucose levels, insulin levels, thyroid-stimulating hormone levels, prolactin levels, and luteinizing hormone levels.

To measure the effectiveness of the S-Klotho protein of the present disclosure for treating an age-related disorder, an animal having decreased Klotho expression may be used (e.g., a mouse with a mutant or deleted klotho gene). For example, the test protein can be administered to the mutant mouse and age-related parameters are monitored. A test protein that is favorably indicated can cause an amelioration of the symptom relative to a similar reference animal not treated with the protein.

A parameter relevant to a clinical (e.g., metabolic) disorder or to aging can be assessed by measurement of body weight, examination on the acquisition of reproductive ability, measurement of blood sugar level, observation of life span, observation of skin, observation of motor functions such as walking, and the like. The assessment can also be made by measurement of thymus weight, observation of the size of calcified nodules formed on the inner surface of thoracic cavity, and the like. Further, quantitative determination of mRNA for the klotho gene or the Klotho protein can also be useful for the assessment.

Still other (in vivo) models and organismal assays include evaluating an animal for a clinical (e.g., metabolic) parameter, e.g., a parameter relevant to an insulin disorder, type II diabetes. Exemplary metabolic parameters include: glucose concentration, insulin concentration, and insulin sensitivity.

In assessing whether a test protein is capable of altering life span regulation, a number of age-associated parameters or biomarkers can be monitored or evaluated. Exemplary age associated parameters include: (i) lifespan of the cell or the organism; (ii) presence or abundance of a gene transcript or gene product in the cell or organism that has a biological age-dependent expression pattern; (iii) resistance of the cell or organism to stress; (iv) one or more metabolic parameters of the cell or organism (exemplary parameters include circulating insulin levels, blood glucose levels; fat content; core body temperature and so forth); (v) proliferative capacity of the cell or a set of cells present in the organism; and (vi) physical appearance or behavior of the cell or organism.

The term “average lifespan” refers to the average of the age of death of a cohort of organisms. In some cases, the “average lifespan” is assessed using a cohort of genetically identical organisms under controlled environmental conditions. Deaths due to mishap are discarded. Where average lifespan cannot be determined (e.g., for humans) under controlled environmental conditions, reliable statistical information (e.g., from actuarial tables) for a sufficiently large population can be used as the average lifespan.

Characterization of molecular differences between two such organisms, e.g., one reference organism and one organism treated with a S-Klotho protein can reveal a difference in the physiological state of the organisms. The reference organism and the treated organism are typically the same (or substantially the same) chronological age and/or sex. The term “chronological age” as used herein refers to time elapsed since a preselected event, such as conception, a defined embryological or fetal stage, or, more preferably, birth. A variety of criteria can be used to determine whether organisms are of the “same” chronological age for the comparative analysis.

Typically, the degree of accuracy required is a function of the average lifespan of a wildtype organism. For example, for the nematode C. elegans, for which the laboratory wildtype strain N2 lives an average of about 16 days under some controlled conditions, organisms of the same age may have lived for the same number of days. For mice, organism of the same age may have lived for the same number of weeks or months; for primates or humans, the same number of years (or within 2, 3, or 5 years); and so forth. Generally, organisms of the same chronological age may have lived for an amount of time within 15, 10, 5, 3, 2 or 1% of the average lifespan of a wildtype organism of that species. Preferably, the organisms are adult organisms (e.g., the organisms have lived for at least an amount of time in which the average wildtype organism has matured to an age at which it is competent to reproduce).

The organismal screening assay can be performed before the organisms exhibit overt physical features of aging. For example, the organisms may be adults that have lived only 10, 30, 40, 50, 60, or 70% of the average lifespan of a wildtype organism of the same species. Age-associated changes in metabolism, immune competence, and chromosomal structure have been reported. Any of these changes can be evaluated, either in a test subject (e.g., for an organism based assay), or prior, during or after treatment with a therapeutic described herein (e.g., for a (human, or mammal) patient).

A marker associated with caloric restriction can also be evaluated in a subject organism of a screening assay (or a treated subject). Although these markers may not be age-associated, they may be indicative of a physiological state that is altered when a Klotho or Klotho-related pathway is modulated. The marker can be an mRNA or protein whose abundance changes in calorically restricted animals. Cellular models derived from cells of an animal described herein or analogous to an animal model described herein can be used for a cell-based assay.

Models for evaluating the effect of a test protein on muscle atrophy include: 1) rat medial gastrocnemius muscle mass loss resulting from denervation, e.g., by severing the right sciatic nerve at mid-thigh; 2) rat medial gastrocnemius muscle mass loss resulting from immobilization (e.g., by fixed the right ankle joint at 90 degrees of flexion); 3) rat medial gastrocnemius muscle mass loss resulting from hind limb suspension; 4) skeletal muscle atrophy resulting from treatment with the cachectic cytokine, interleukin-1 (IL-1); and 5) skeletal muscle atrophy resulting from treatment with the glucocorticoid, dexamethasone.

### Products, Compositions, Formulations, Supplements, Etc.

Some embodiments of the present disclosure relate to compositions configured or formulated to augment natural soluble alpha Klotho protein production and/or attenuate Klotho protein damage or degradation, and to methods, kits, formulations, combination products, co-administrations, co-formulations, dosages, process, methods, treatment protocols, and diagnostic methods related to the same. Some embodiments of the present disclosure relate to methods of manufacturing compositions of the present disclosure. Some embodiments of the present disclosure relate to use of compositions of the present disclosure or methods of using the same to treat human or non-human animal subjects. Some embodiments of the present disclosure relate to treatment methods involving compositions of the present disclosure, including administration thereof to human or non-human animal subjects. Such uses and treatment methods can increase endogenous Klotho protein levels, such as by augmenting natural soluble alpha Klotho protein production and/or attenuating Klotho protein damage or degradation.

Certain embodiments include compositions of matter that comprise a health supplement that, when administered to human or non-human animal (e.g., mammalian) subjects, increases the levels of endogenous, soluble alpha-Klotho protein in the human or non-human animal subjects. The composition or health supplement formulation can comprise (synthetic) chemical and/or natural components or ingredients. In other words, components or ingredients of the composition or health supplement can be or originate from (synthetic) chemical and/or natural sources.

An exemplary composition can comprise one or more components, preferably selected from the group consisting of, for example, Vitamin D3, Vitamin E, Vitamin C, Vitamin K, 3,5,4′-trihydroxy-trans-stilbene (Resveratrol), Pterostilbene, N-acetylcysteine (NAC), Troglitazone, Rosiglitazone, Notopterygium incisum Ting, Gentian (Root; Extract), Cordyceps (Cordyceps Sinensis (CS) Mycelium), Isoflavones (Soy), Genistein, Daidzein, Quercetin (dihydrate; dill, bay leaf, oregano), Docosahexaenoic acid (DHA), Astaxanthin, Sesamin (Semen Sesamin Nigrum (Black)), Sesame (Seed Extract), Rosmarinic acid (RA; (Marjoram)), Tetradecylthioacetic Acid (TTA), Dicalcium Phosphate (Anhydrous), Mircrocrystalline cellulose (MCC), Croscarmellose Sodium (Primellose), Stearing Acid, Magnesium Stearate, Alpha Lipoic Acid, Conjugate (alpha) Linoleic Acid (CLA), Probiotics, Activated Charcoal, and others as known in the art.

Certain ingredients or components can have positive benefits and very low side effects in treating or affecting one or more conditions that have been associated with cellular and tissue dysfunctions that occur with aging. Certain ingredients or components can (directly or indirectly) increase the circulating plasma level of soluble alpha Klotho (s-Klotho) levels in human or non-human animal subjects following administration thereof.

The following examples are illustrative only. The following examples illustrate various optional ingredients, components, method steps, etc. that may be useful in the practice of the embodiments of the present disclosure.

Embodiments of the present disclosure include formulations (compositions, co-administrations, etc.) of several components (or ingredients) that, individually, can have a potential for positive benefits and/or very low side effects in addressing, treating, affecting, and/or countering one or more conditions that have been associated with cellular and tissue dysfunctions and/or that can occur with aging.

Without being bound to any theory, some of the components or ingredients of the present disclosure may activate the promoter of the klotho gene (e.g., in the human HEK293/kl cell line), or reverse age-related declines in expression of Klotho in hepatic, kidney, and other tissues. Some of the components may attenuate, decrease, inhibit, and/or prevent oxidative or other damage to Klotho protein or the klotho gene.

Without being bound to any theory, some of the components have been shown by one or more animal and human clinical studies to have a potential for positive benefits and/or very low side effects in addressing, treating, affecting, and/or countering one or more conditions that have been associated with cellular and tissue dysfunctions and/or that can occur with aging. The present disclosure utilizes these various components in compositions and methods for increasing endogenous Klotho protein levels. Specifically, the present disclosure utilizes these various components in compositions configured or formulated to augment natural soluble alpha Klotho protein production and/or attenuate Klotho protein damage or degradation.

Without being bound to any theory, certain of the individual ingredients in some embodiments can increase the circulating plasma level of soluble alpha-Klotho in mammalian (e.g., human or non-human) animals. Example ingredients may include, but are not limited to: N-acetylcysteine (NAC), an antioxidant; D vitamins, such as Vitamin D3 (1,25-dihydroxyvitamin D3 [1,25(OH)2D3]); and/or vitamin D receptor agonist (e.g., calcitriol, paricalcitol), which are thought to increase Klotho expression, increase phosphaturia, correct hyperphosphatemia, and lower serum fibroblast growth factor-23 (FGF-23) resulting in associated decreasing aortic calcification in mice suffering from chronic kidney disease; and Vitamin C and/or Vitamin E, which are thought to ameliorate age-related crystalluria and kidney damage at the proximal tubule.

Further example ingredients may include, but are not limited to non-prescription ingredients that may supplement the anti-aging properties of alpha Klotho, such as, for example: docosahexaenoic acid (DHA), linoleic acid, conjugated linoleic acid (CLA), isoflavones (e.g., genistein, daidzein, quercetin), rosmarinic acid, sesamin and astaxanthin (food-derived antioxidants), and tetradecylthioacetic acid (TTA). Without being bound to any theory, the aging brain is particularly prone to inflammatory and oxidative alterations which may underlie decreases in learning and memory and omega-3 polyunsaturated fatty acids (PUFAs) such as DHA, protect the aging brain from the development of neurodegenerative diseases. Accordingly, certain formulaE can include alpha linoleic acid or conjugated alpha linoleic acid because alpha linoleic acid can serve as a precursor molecule used in the human body in the manufacture of DHA.

In at least one embodiment, one or more DHA precursors or omega-3 fatty acids, such as LA or CLA can be omitted from the formulation. Without being bound to any theory, omega-3 fatty acids such as LA or CLA may increase the effects of blood-thinning medications and raise the risk of bleeding. Example medications include, without limitation, warfarin (Coumadin), clopidogrel (Plavix), and aspirin, among others. In some embodiments, DHA can be included in the formulation. Without being bound to any theory, DHA may not directly cause blood thinning

Without being bound to any theory, mitochondrial damage can be the cause and outcome of cell injury resulting from a variety of toxic insults, hypoxia, or trauma. Increasing mitochondrial biogenesis after renal proximal tubular cell (RPTC) injury may accelerate the recovery of mitochondrial and cellular functions after such injury. Relevant to these observations, isoflavones, such as daidzein and genistein, may increase peroxisome proliferator-activated receptor gamma coactivator (PGC)-1 alpha expression and result in mitochondrial biogenesis as indicated by increased expression of ATP synthase beta and NADH:ubiquinone oxidoreductase core subunit 6 (ND6), which may result in a 1.5-fold increases in respiration and ATP in RPTC and which benefits recovery in injured RPTC. Without being bound to any theory, daidzein may be metabolized within the gut, by particular intestinal flora, to produce the bioactive compound equol. Equol can also be included in certain embodiments. The isoflavone, quercetin, on the other hand, may counter detrimental effects of aging by increasing oxidative stress resistance in the proteasome—large protein complexes responsible for the proper regulation of the cellular protein load that play a major role in the maintenance of redox balance by recognizing and removing oxidatively modified or damaged proteins.

Rosmarinic acid can also be added as an ingredient in some formulations. Rosmarinic acid (RA) is a naturally occurring phenolic compound, which may contribute to the beneficial and health-promoting effects of herbs, spices and medicinal plants. Administration of RA at a dosage of 200 mg per kg, once a day for 30 days to aging mice, can significantly (p<0.05 or p<0.01) increase the activity of superoxide dismutase (SOD) catalase (CAT) and glutathione peroxidase (GSH-Px), with a decrease in malondialdehyde (MDA), in comparison to an aging control that did not receive RA. Thus, RA may promote significant antioxidant enzyme activity in liver and kidney tissues of aging mice.

Some embodiments may also contain the food-derived antioxidants, such as astaxanthin (A) and/or sesamin (S). Without being bound to any theory, when both of these ingredients (astaxanthin is a red carotenoid found in salmon, shrimp, crab, and microalgae while sesamin is a major lignan found in sesame extract) were combined (i.e., AS) and administered daily to subjects with mild cognitive impairment (MCI), significant improvements in psychomotor speed and processing speed was observed in the AS group compared with a placebo group. Accordingly, daily supplementation of AS may improve cognitive functions related to the ability to comprehend and perform complex tasks quickly and accurately.

One or more peroxisome proliferator-activated receptor (PPAR-γ) agonists, such as Tetradecylthioacetic Acid (TTA), may also be added to certain embodiments. PPAR-γ agonists may have beneficial effects on heart muscle through the regulation of amino acids metabolism and L-carnitine biosynthesis.

In addition to the purified chemical compounds described above, some embodiments may contain one or more (e.g., several) natural ingredients or extracts. These include, but are not limited to, extracts of gentian root extract, Cordyceps mushroom, and Notopterygium incisum Ting.

Certain embodiments can include one or more (selective) strains of (probiotic) bacteria, such as: Lactobacillus sp. (e.g., acidophilus, brevis, bulgaricus, casei, gasseri, johnsonii, lactis, plantarum, paracasei, rhamnosus, salivarius, sake, etc.), Bifidobacterium sp. (e.g., bifidum, breve, infantis, lactis, longum, etc.), Streptococcus sp. (e.g., thermophiles, etc.), Bacillus sp. (e.g., laterosporus, etc.), Pediococcus sp. (e.g., acidilactici, etc.), and others as known in the art. Without being bound to any theory, probiotics may alleviate age-inflicted oxidative stress and improve expression of biomarkers of aging (e.g., in mice). Additional information regarding probiotics, which may be included in certain embodiments of the present disclosure, can be found in a literature by Sabina Fijan, “Microorganisms with Claimed Probiotic Properties: An Overview of Recent Literature,” published in Int J Environ Res Public Health, 2014 May; 11(5): 4745-4767, the entirety of which is incorporated herein by specific reference.

Table 42 provides a list of ingredients that can be formulated into a particular embodiment (designated KSF 101).

TABLE 42
KSF 101 Formulation

| Ingredient | Amount/Dose | |
|---|---|---|
| Vitamin D3 | 1,000 | IU |
| Vitamin E | 2,000 | IU |
| Vitamin C | 1000 | mg |
| Resveratrol | 1000 | mg |
| (3,5,4′-trihydroxy-trans-stilbene) | | |
| Pterostilbene | 5 | mg |
| NAC (N-acetylcysteine) | 600 | mg |
| Troglitazone | 1360 | mg |
| Rosiglitazone | 136 | mg |
| Notopterygium incisum Ting | 3000 | mg |
| Gentian Root Extract | 500 | mg |
| Cordyceps | 1000 | mg |
| Genistein | 10 | mg |
| Daidzein | 40 | mg |
| Quercetin | 150 | mg |
| Docosahexaenoic acid (DHA) | 1000 | mg |
| Astaxanthin | 3 | mg |
| Sesamin (S) | 5 | mg |
| Rosmarinic acid (RA) | 500 | mg |
| Tetradecylthioacetic Acid (TTA) | 500 | mg |
| Alpha Linoleic Acid | 3200 | mg |
| Conjugate Alpha Linoleic Acid (CLA) | 1000 | mg |
| Probiotics | 8 | g |
| Activated Charcoal | 5000 | mg |

By way of non-limiting example, the KSF 101 formulation can be or comprise an orally administered, DSHEA-compliant formulation or composition. Accordingly, in some instances, the KSF 101 supplement may be marketed and/or sold directly-to-consumer (DTC) and/or without requiring a prescription/physician order. It will be appreciated, however, that KSF 101 can be formulated in other dosage forms and/or as an FDA-approved drug without departing from the scope of this disclosure. The concentration of ingredients in KSF 101 are based on a standard adult reference of a 150-pound person (68 kilograms). Alternative dosage(s), as understood by those skilled in the art, are also contemplated herein.

Table 43 lists potential (e.g., preferred and optional) ingredients that can be formulated into another embodiment of the present disclosure.

TABLE 43
Illustrative Ingredients

| | |
|---|---|
| | (potential max.) |
| Ingredient | Amount/Dose |

Preferred Ingredients

| | | |
|---|---|---|
| Vitamin D3 | 1,000 | IU |
| Vitamin E | 2,000 | IU |
| Vitamin C | 1,250 | mg |
| Pterostilbene | 5 | mg |
| NAC (N-acetylcysteine) | 600 | mg |
| Notopterygium incisum Ting | 3,000 | mg |
| Gentian Root Extract | 500 | mg |
| Cordyceps | 1,000 | mg |

Optional or Alternative Ingredients

| | | |
|---|---|---|
| 2,3,5,4′-Tetrahydroxystilbene-2- | 4,000 | mg |
| O-β-D-glucoside (THSG) | | |
| Resveratrol | 1,000 | mg |
| (3,5,4′-trihydroxy-trans-stilbene) | | |
| Rosiglitazone | 136 | mg |
| Troglitazone | 1,360 | mg |
| Genistein | 10 | mg |
| Daidzein | 40 | mg |
| Quercetin | 500 | mg |
| (from dill, bay leaves, and oregano) | | |
| Isoflavones (Genistein, Daidzein, Equol) | 10 | mg |
| Docosahexaenoic acid (DHA) | 1,000 | mg |
| Astaxanthin | 3 | mg |
| Sesamin (S) | 200 | mg |
| Rosmarinic acid (RA) (marjoram) | 500 | mg |
| Tetradecylthioacetic Acid (TTA) | 500 | mg |
| Conjugate Linoleic Acid (CLA) | 3,200 | mg |
| Alpha Lipoic Acid (ALA) | 1000 | mg |
| Probiotics | 10-20 | billion CFU |
| Activated Charcoal | 5,000 | mg |
| Vitamin K | 90-120 | mcg |

Table 44 lists potential (e.g., preferred and optional) ingredients that can be formulated into another embodiment of the present disclosure.

TABLE 44

| | Label Claim | | | | Optimized | |
|---|---|---|---|---|---|---|
| Ingredient | mg | | Potency | mg/Tab | Amount/Day | |
| **9 TABLETS (excluding Probiotics and | | | | 1303.22 | | |
| Charcoal) | | | | | | |
| Vitamin D3 (100,000 Iu/Gm | 1000 | IU | | 1.22 | 1000 | IU |
| Vitamin E 1210 Iu (D-Alpha Tocopherol | 2000 | IU | | 202.02 | 2000 | IU |
| Succinate 1162 Iu | | | | | | |
| Vitamin C 97% (Ascorbic Acid) Granular | 1250 | mg | 97% | 157.5 | 1000 | mg |
| Pterostilbene Powder | 5 | mg | | 0.58 | 5 | mg |
| N-Acetyl L-Cysteine (NAC) | 600 | mg | | 70 | 600 | mg |
| Notopterygium Incisum Ting | 3000 | mg | | 350 | 0 | |
| Gentian Root 10:1 (Powder) | 500 | mg | | 58.33 | 500 | mg |
| Cordyceps Sinensis (CS) Mycelium Powder | 1000 | mg | | 116.67 | 0 to 300 | mg |
| Soy Isoflavones 40% | 10 | mg | 40% | 2.92 | 10 | mg |
| Dha Powder 17% from Algae | 170 | mg | 17% | 116.67 | 170 | mg |
| Semen Sesamin Nigrum (Black) Powder | 200 | mg | | 23.33 | 100-150 | mg |
| Quercetin From Dill, Bay Leaves and | 500 | mg | 98% | 59.52 | 150 | mg |
| Oregano | | | | | | |
| Rosmarinic Acid (RA)(Marjoram) | 500 | mg | | 58.33 | 500 | mg |
| Tetradecylthioacetic acid (TTA) | 500 | mg | | 58.33 | 500 | mg |

| Dicalcium Phosphate Anhydrous Powder | 5.56 | | |
|---|---|---|---|
| Mcc (Mircrocrystalline Cellulose) Ph 102 | 5.56 | | |
| Stearing Acid Nf | 5.56 | | |
| Croscarmellose Sodium (Primellose) | 5.56 | | |
| Magnesium Stearate | 5.56 | | |
| Probiotics | | 0 | |
| Charcoal (organic coconut) | | 260 | mg |

The ingredients of Tables 42-44 can be optionally combined in any suitable combination. By way of non-limiting example, the alternative formulation(s) can be or comprise an orally administered, DSHEA-compliant formulation or composition. It will be appreciated, however, that alternative formulation(s) can be formulated in other dosage forms and/or as an FDA-approved drug without departing from the scope of this disclosure. The respective concentrations of ingredients in the alternative formulation(s) are based on a standard adult reference of a 150-pound person (68 kilograms). Alternative dosage(s), as understood by those skilled in the art, are also contemplated herein.

An illustrative composition (e.g., a nutraceutical composition) can comprise two or more components selected from the group consisting of vitamin C, vitamin D3, vitamin E, N-acetylcysteine (NAC), quercetin (dihydrate), rosmarinic acid (RA), pterostilbene, docosahexaenoic acid (DHA), nicotinamide riboside (NR), nicotinamide adenine dinucleotide (NAD+), nicotinamide mononucleotide (NMN), and one or more probiotic. The one or more probiotic can include an effective amount of one or more of: (i) one or more Bifidobacterium species and/or strains selected from Bifidobacterium lactis BL-04, Bifidobacterium bifidum/lactis BB-02, and Bifidobacterium longum BL-05, and/or (ii) one or more Lactobacillus species and/or strains selected from Lactobacillus acidophilus LA-14, Lactobacillus rhamnosus LR-32, and Lactobacillus paracasei LPC-37.

Illustratively, the composition (or formulation) includes up to, at least, and/or about 50 billion CFU (per daily dose) of a Probiotic ingredient. Alternative embodiments can include up to, at least, between, and/or about 20 billion CFU (per daily dose), 30 billion CFU (per daily dose), 40 billion CFU (per daily dose), 60 billion CFU (per daily dose), 70 billion CFU (per daily dose), and/or 80 billion CFU (per daily dose) of the Probiotic ingredient. Illustratively, the Probiotic ingredient can comprise or include a single probiotic (bacterial) species or strain, or up to, at least, and/or between 2, 3, 4, 5, 6, 7, 8, 9, and 10 probiotic (bacterial) species or strains. In at least one embodiment, the Probiotic ingredient comprises the following probiotic (bacterial) species or strains: 30 billion CFU/day each of the Bifidobacterium species and/or strains Bifidobacterium lactis BL-04, Bifidobacterium bifidum/lactis BB-02, and Bifidobacterium longum BL-05. It will be appreciated, however, that certain embodiments may include one or two of the foregoing Bifidobacterium species and/or strains and/or in an amount of up to, at least, between, and/or about 10 billion CFU (per daily dose), 20 billion CFU (per daily dose), 30 billion CFU (per daily dose), 40 billion CFU (per daily dose), and/or 50 billion CFU (per daily dose). In at least one embodiment, the Probiotic ingredient comprises the following probiotic (bacterial) species or strains: 20 billion CFU/day each of the Lactobacillus species and/or strains Lactobacillus acidophilus LA-14, Lactobacillus rhamnosus LR-32, and Lactobacillus paracasei LPC-37. It will be appreciated, however, that certain embodiments may include one or two of the foregoing Lactobacillus species and/or strains and/or in an amount of up to, at least, between, and/or about 5 billion CFU (per daily dose), 10 billion CFU (per daily dose), 20 billion CFU (per daily dose), 30 billion CFU (per daily dose), and/or 40 billion CFU (per daily dose).

In at least one embodiment, vitamin C can be included in a daily dose and/or concentration of about 500 mg, or between about 100-2000 mg, preferably about 200-1500 mg, more preferably about 250-1200 mg, still more preferably about 400-1000 mg, still more preferably about 500-1000 mg, still more preferably about 500-750 mg (per day or per dosage). In at least one embodiment, vitamin C can be included in a daily dose and/or concentration of at least about 100 mg, 200 mg, 250 mg, 300 mg, 400 mg, 500 mg, 750 mg, or 1000 mg (per day or per dosage). In at least one embodiment, vitamin C can be included in a daily dose and/or concentration of less than or equal to about 2500 mg, 2000 mg, 1500 mg, 1000 mg, or 750 mg (per day or per dosage).

In at least one embodiment, vitamin D3 can be included in a daily dose and/or concentration of about 1000 mg, or between about 100-2500 mg, preferably about 200-2000 mg, more preferably about 250-1800 mg, still more preferably about 400-1500 mg, still more preferably about 500-1500 mg, still more preferably about 750-1250 mg (per day or per dosage). In at least one embodiment, vitamin D3 can be included in a daily dose and/or concentration of at least about 100 mg, 200 mg, 250 mg, 300 mg, 400 mg, 500 mg, 750 mg, 1000 mg, 1250 mg, 1500 mg, or 2000 mg (per day or per dosage). In at least one embodiment, vitamin D3 can be included in a daily dose and/or concentration of less than or equal to about 3000 mg, 2500 mg, 2000 mg, or 1500 mg (per day or per dosage).

In at least one embodiment, vitamin E can be included in a daily dose and/or concentration of about 400 mg, or between about 100-2000 mg, preferably about 150-1500 mg, more preferably about 200-1200 mg, still more preferably about 250-1000 mg, still more preferably about 250-750 mg, still more preferably about 250-500 mg, still more preferably about 300-750 mg, still more preferably about 300-500 mg (per day or per dosage). In at least one embodiment, vitamin E can be included in a daily dose and/or concentration of at least about 100 mg, 200 mg, 250 mg, 300 mg, 400 mg, 500 mg, 750 mg, or 1000 mg (per day or per dosage). In at least one embodiment, vitamin E can be included in a daily dose and/or concentration of less than or equal to about 2000 mg, 1500 mg, 1000 mg, 750 mg, or 500 mg (per day or per dosage).

In at least one embodiment, N-acetylcysteine (NAC) can be included in a daily dose and/or concentration of about 600 mg, or between about 100-2000 mg, preferably about 150-1500 mg, more preferably about 200-1200 mg, still more preferably about 250-1000 mg, still more preferably about 250-750 mg, still more preferably about 500-750 mg. In at least one embodiment, N-acetylcysteine (NAC) can be included in a daily dose and/or concentration of at least about 100 mg, 200 mg, 250 mg, 300 mg, 400 mg, 500 mg, 750 mg, or 1000 mg (per day or per dosage). In at least one embodiment, N-acetylcysteine (NAC) can be included in a daily dose and/or concentration of less than or equal to about 2000 mg, 1500 mg, 1000 mg, or 750 mg (per day or per dosage).

In at least one embodiment, quercetin (dihydrate) can be included in a daily dose and/or concentration of about 150 mg, or between about 10-1000 mg, preferably about 25-750 mg, more preferably about 50-500 mg, still more preferably about 100-250 mg, still more preferably about 100-200 mg (per day or per dosage). In at least one embodiment, quercetin (dihydrate) can be included in a daily dose and/or concentration of at least about 10 mg, 20 mg, 25 mg, 30 mg, 40 mg, 50 mg, 75 mg, 100 mg, 150 mg, 200 mg, or 250 mg (per day or per dosage). In at least one embodiment, quercetin (dihydrate) can be included in a daily dose and/or concentration of less than or equal to about 1000 mg, 750 mg, 500 mg, 400 mg, 300 mg, 250 mg, or 200 mg (per day or per dosage).

In at least one embodiment, rosmarinic acid (RA) can be included in a daily dose and/or concentration of about 500 mg, or between about 100-2000 mg, preferably about 200-1500 mg, more preferably about 250-1200 mg, still more preferably about 400-1000 mg, still more preferably about 500-1000 mg, still more preferably about 500-750 mg (per day or per dosage). In at least one embodiment, rosmarinic acid (RA) can be included in a daily dose and/or concentration of at least about 100 mg, 200 mg, 250 mg, 300 mg, 400 mg, 500 mg, 750 mg, or 1000 mg (per day or per dosage). In at least one embodiment, rosmarinic acid (RA) can be included in a daily dose and/or concentration of less than or equal to about 2500 mg, 2000 mg, 1500 mg, 1000 mg, or 750 mg (per day or per dosage).

In at least one embodiment, pterostilbene can be included in a daily dose and/or concentration of about 50 mg, or between about 10-200 mg, preferably about 20-150 mg, more preferably about 25-120 mg, still more preferably about 25-120 mg, still more preferably about 40-100 mg, still more preferably about 50-100 mg, still more preferably about 50-75 mg (per day or per dosage). In at least one embodiment, pterostilbene can be included in a daily dose and/or concentration of at least about 10 mg, 20 mg, 25 mg, 30 mg, 40 mg, 50 mg, 75 mg, or 100 mg (per day or per dosage). In at least one embodiment, pterostilbene can be included in a daily dose and/or concentration of less than or equal to about 250 mg, 200 mg, 150 mg, 100 mg, or 75 mg (per day or per dosage).

In at least one embodiment, DHA (preferably vegetarian DHA) can be included in a daily dose and/or concentration of about 200 mg, or between about 10-1000 mg, preferably about 25-750 mg, more preferably about 50-500 mg, still more preferably about 100-250 mg, still more preferably about 150-250 mg, still more preferably about 100-200 mg (per day or per dosage). In at least one embodiment, quercetin (dihydrate) can be included in a daily dose and/or concentration of at least about 10 mg, 20 mg, 25 mg, 30 mg, 40 mg, 50 mg, 75 mg, 100 mg, 150 mg, 200 mg, 250 mg, 300 mg, 400 mg, or 500 mg (per day or per dosage). In at least one embodiment, quercetin (dihydrate) can be included in a daily dose and/or concentration of less than or equal to about 2000 mg, 1500 mg, 1000 mg, 750 mg, 500 mg, 400 mg, 300 mg, or 250 mg (per day or per dosage).

In at least one embodiment, Nicotinamide Riboside (NR) can be included in a daily dose and/or concentration of about 500 mg, or between about 100-2000 mg, preferably about 200-1500 mg, more preferably about 250-1200 mg, still more preferably about 400-1000 mg, still more preferably about 500-1000 mg, still more preferably about 500-750 mg (per day or per dosage). In at least one embodiment, Nicotinamide Riboside (NR) can be included in a daily dose and/or concentration of at least about 100 mg, 200 mg, 250 mg, 300 mg, 400 mg, 500 mg, 750 mg, or 1000 mg (per day or per dosage). In at least one embodiment, Nicotinamide Riboside (NR) can be included in a daily dose and/or concentration of less than or equal to about 2500 mg, 2000 mg, 1500 mg, 1000 mg, or 750 mg (per day or per dosage).

In at least one embodiment, Nicotinamide adenine dinucleotide (NAD+) can be included in a daily dose and/or concentration of about 300 mg, or between about 50-2000 mg, preferably about 100-1000 mg, more preferably about 150-750 mg, more preferably about 200-600 mg, still more preferably about 200-500 mg, still more preferably about 200-400 mg, still more preferably about 250-500 mg, still more preferably about 250-400 mg, still more preferably about 300-500 mg, still more preferably about 300-400 mg (per day or per dosage). In at least one embodiment, Nicotinamide adenine dinucleotide (NAD+) can be included in a daily dose and/or concentration of at least about 50 mg, 100 mg, 200 mg, 250 mg, 300 mg, 400 mg, 500 mg, 750 mg, or 1000 mg (per day or per dosage). In at least one embodiment, Nicotinamide adenine dinucleotide (NAD+) can be included in a daily dose and/or concentration of less than or equal to about 2000 mg, 1500 mg, 1000 mg, 750 mg, 500 mg, 400 mg, or 350 mg (per day or per dosage).

In at least one embodiment, Nicotinamide Mononucleotide (NMN) can be included in a daily dose and/or concentration of about 200 mg, or between about 10-1000 mg, preferably about 25-750 mg, more preferably about 50-500 mg, still more preferably about 100-250 mg, still more preferably about 150-250 mg, still more preferably about 100-200 mg (per day or per dosage). In at least one embodiment, Nicotinamide Mononucleotide (NMN) can be included in a daily dose and/or concentration of at least about 10 mg, 20 mg, 25 mg, 30 mg, 40 mg, 50 mg, 75 mg, 100 mg, 150 mg, 200 mg, 250 mg, 300 mg, 400 mg, or 500 mg (per day or per dosage). In at least one embodiment, Nicotinamide Mononucleotide (NMN) can be included in a daily dose and/or concentration of less than or equal to about 2000 mg, 1500 mg, 1000 mg, 750 mg, 500 mg, 400 mg, 300 mg, or 250 mg (per day or per dosage).

Without being bound to any theory, in embodiments that include NAD+ and/or NAD+ precursor(s) (NR and/or NMN), the NAD+ and/or NAD+ precursor(s) may be in the form of a Lozenge for sublingual application (e.g., to get into the bloodstream, initially bypassing the liver and extending the time until the liver converts them into NAM). Other components may also be administered separately or co-administered. In some embodiments, ingredients can be co-administered, preferably via at least some co-formulation, or formulated as a multi-dosage composition. For example, some embodiments can be, comprise, or be provided as a multi-pill, -capsule, or other dosage kit or pack. Embodiments can include one, two, three, four, five, six, seven, eight, nine, ten, or more dosed components, such as pills, capsule, etc. Some ingredients can be provided in solid, granular, powdered, liquid, or other form.

Table 45 presents an illustrative formulation according to an embodiment of the present disclosure.

TABLE 45

| Ingredient for Capsules | Amount/Dose | Unit |
|---|---|---|
| vitamin C | 500 | mg |
| vitamin D3 | 1000 | IU |
| Vitamin E | 400 | IU |
| N-acetylcysteine (NAC) | 600 | mg |
| quercetin dihydrate | 150 | mg |
| rosmarinic acid (RA) | 500 | mg |
| pterostilbene | 50 | mg |
| DHA (Vegetarian) | 200 | mg |
| Nicotinamide Riboside (NR) | 500 | mg |
| Nicotinamide adenine dinucleotide (NAD+) | 300 | mg |
| Nicotinamide Mononucleotide (NMN) | 200 | mg |
| Probiotics (50B CFU - 6 Strains, below) | 50 Billion | CFU |

The ingredients of Table 45 can be optionally combined in any suitable combination. By way of non-limiting example, the alternative formulation(s) can be or comprise an orally administered, DSHEA-compliant formulation or composition. It will be appreciated, however, that alternative formulation(s) can be formulated in other dosage forms and/or as an FDA-approved drug without departing from the scope of this disclosure. The respective concentrations of ingredients in the alternative formulation(s) are based on a standard adult reference of a 150-pound person (68 kilograms). Alternative dosage(s), as understood by those skilled in the art, are also contemplated herein.

Some embodiments can include a composition, comprising an effective amount of a composition (e.g., health supplement), as described herein, formulated or co-formulated to increase endogenous Klotho protein levels or production (e.g., in mammalian subjects). The pharmaceutically effective amount of the composition can be sufficient to increase or raise the serum soluble Klotho protein concentration of the subject (e.g., to a predetermined level, such as greater than, equal to, or between about 50 to 3000 picograms of soluble Klotho protein per milliliter of serum). The pharmaceutically effective amount of the composition can also or alternatively be sufficient to maintain the serum soluble Klotho protein concentration of the subject at or above a predetermined threshold (e.g., for a predetermined period of time).

In certain embodiments, sufficient or suitable increase in Klotho protein levels can (be effective to) modulate the IGF-1 and/or Wnt signaling pathways, exhibit β-glucuronidase and/or sialidase activity, suppress the p53/p21 signaling pathway, and/or reduce H2O2-induced cell senescence and apoptosis, preferably through suppression of the p53/p21 signaling pathway. The (raised) protein can function or be functional as a humoral factor, preferably exhibiting pleiotropic activity and/or preferably in the regulation of oxidative stress, growth factor signaling, ion homeostasis, and/or regulation of activity of glycoproteins on the cell surface, such as one or more ion channel proteins and/or growth factor receptors, such as Insulin/Insulin-Like Growth Factor-1 receptor.

The formulated composition (e.g., health supplement) of the present disclosure can raise serum soluble Klotho protein levels by increasing or enhancing (natural) production of Klotho protein by the patient or subject or attenuating (e.g., decreasing, inhibiting, and/or preventing) oxidative or other damage or degradation to/of Klotho protein or the klotho gene.

Some embodiments of the present disclosure can include a pharmaceutical composition, such as a therapeutic composition or drug. For example, some embodiments can include one or more active ingredients, such as a pharmaceutical or prescription medications (e.g., ARBs (e.g., Losartan, Valsartan), testosterone, Vit. D receptor agonists (e.g., calcitriol, paricalcitol), PPAR (gamma) agonists (e.g., thiazolidinediones, troglitazone, rosiglitazone), or others. Some embodiments can include a formulation or co-formulation, in combination with prescribed medications such as angiotension receptor blockers (ARBs) (i.e., losartan, valsartan) or Vitamin D receptor agonists (VDRAs) (i.e., calcitriol or paricalcitol). Other prescription medications that may be administered with formulations include troglitazone (at 200 mg/kg/day) and rosiglitazone (at 20 mg/kg/day), both Peroxidase Proliferator-Activated Receptor (PPAR-γ) agonists. Without being bound to any theory, klotho is thought to be a target gene of PPAR-γ in the cultured human kidney cells, as well as in mouse kidneys in vivo. Moreover, Klotho protein expression may be enhanced (or upregulated) by treatment with PPAR-γ agonists (e.g., at the described oral dosages).

Without being bound to any theory, losartan may (significantly) increase circulating α-Klotho levels in human type 2 diabetics by an average of 23% (from 542 pg/ml to 668 pg/ml, p=0.001). Also, linear regression analysis suggests that the increment in plasma α-Klotho achieved enhanced healthy renal function as seen by an associated decrease in urine albumin/creatinine ratio (β=−0.263, p=0.029). Similarly, valsartan may significantly increase α-Klotho levels (from 432.7±179 to 506.4±226.8 pg/ml).

Also, in regard to the above, it is thought that treatment of mice with chronic kidney disease (CKD) with calcitriol or paricalcitrol (at VDRAs doses extrapolated from human subjects that are currently approved for the treatment of secondary hyperparathyroidism) result in half the amount of aortic calcification as compared to no VDRAs. In particular, mice given i.p. calcitriol or paricalcitol 3× per week for 3 weeks (dosing: calcitriol at 30 ng/kg (C30), or paricalcitol at 100 ng/kg (P100), or paricalcitol at 300 ng/kg (P300)) may exhibit increased serum and urine Klotho levels, increased phosphaturia, correction of hyperphosphatemia, and lowering of serum fibroblast growth factor-23. Klotho and osteopontin may also be upregulated by VDRA therapy independent of changes in serum parathyroid hormone and calcium.

Some embodiments, or pharmaceutical components thereof, can include one or more recombinant (e.g., human or mammalian) Klotho proteins, protein fragments, and/or protein variants. Such active ingredients or pharmaceutical components can contribute to increased levels of endogenous Klotho protein (production) in the patient or subject. Additional description of (various and/or additional) active ingredients, such as therapeutics, recombinant proteins, pharmaceutical, and/or prescription medications, are described in the patent references incorporated above (i.e., (i) PCT/US2017/035755 filed Jun. 2, 2017, (ii) PCT/US2017/063149 filed Nov. 22, 2017, and (iii) U.S. Provisional Application Ser. No. 62/595,567 filed Dec. 6, 2017). Such active ingredients can contribute to increased levels of endogenous Klotho protein (production) in the patient or subject.

Pharmaceutical compositions or components, regardless of type, can, generally, include a drug component, optionally admixed with a (pharmaceutically-acceptable) vehicle, carrier, or excipient comprised of one or more additional components. The components can include one or more aggregation inhibitors, buffers, tonicity modifiers, and additional excipients. The primary solvent in the carrier can be either aqueous or non-aqueous in nature.

Some embodiments can include a combination product, composition, or formulation comprising a recombinant Klotho protein and one or more additional active ingredients. The one or more additional active ingredients can be selected from among the components, drugs, substances, treatment compositions, etc. described herein or others known in the art. For instance, the Klotho protein and one or more additional active ingredients can be co-formulated into an injectable (e.g., intramuscular, intravenous, etc.), ingestible, transdermal, inhalable, topical, or other formulation. Alternatively, a combination treatment or co-administration can include treatment or administration of a Klotho protein and one or more additional active ingredients, but without the Klotho protein and one or more additional active ingredients being combined or formulated into a combination product, composition, or formulation. For instance, the Klotho protein and one or more additional active ingredients can each comprise or be in a separate injectable (e.g., intramuscular, intravenous, etc.), ingestible, transdermal, inhalable, topical, or other formulation.

Some embodiments can include a composition, comprising an effective amount of a composition (e.g., health supplement) and, optionally, an effective (e.g., a pharmaceutically effective) amount of the recombinant Klotho protein and/or pharmaceutical or prescription medications, as described herein, formulated or co-formulated to increase endogenous Klotho protein levels or production (e.g., in mammalian subjects). In some embodiments, the recombinant Klotho protein and/or pharmaceutical or prescription medications can be co-administered with the composition. In some embodiments, the recombinant Klotho protein and/or pharmaceutical or prescription medications can be combined with a pharmaceutically-acceptable carrier. In some embodiments, the optionally administered, optional recombinant Klotho protein can raise serum soluble Klotho protein levels by providing exogenous Klotho protein, while the optionally administered, optional pharmaceutical or prescription medications can raise serum soluble Klotho protein levels by increasing or enhancing (natural) production of Klotho protein by the patient or subject or attenuating (e.g., decreasing, inhibiting, and/or preventing) oxidative or other damage or degradation to/of Klotho protein or the klotho gene.

It will also be appreciated that co-administration can comprise simultaneous administration of two or more components or distinct administrations of two or more components, the distinct administrations preferably being separated by a period of time. The period of time can be very small, in some embodiments. For instance, a second component can be administered substantially, immediately following administration of a first component. Alternatively, the first and second administrations can be separated by a time period of 1-60 seconds, 1-60 minutes, 1-24 hours, 1-7 days, 1-4 weeks, 1-12 months, and so forth, or any value or range of values therebetween. Similarly, simultaneous administration can include overlapping administration timeframes for the two or more components.

Any of the foregoing or other compositions, formulations, and/or co-administrations can have additive or synergistic effects over those of any of the individual components, alone. For instance, co-administration of various health supplement ingredients or a health supplement composition with an active component (e.g., a recombinant Klotho protein, pharmaceutical drug, etc.) can result in treatment outcomes greater than the sum of the individual outcomes of administering the individual components alone at similar concentrations. In addition, synergistic effects can include treatment outcomes similar to those of the individual outcomes of administering the components alone, but at lower concentrations. Synergistic effects can also include increasing the maximum effective dose of one or more of the components, reducing toxicity of one or more of the components, or any other beneficial result that is more than a mere additive effect of the individual treatment outcomes. In addition, additive effects of the individual treatment outcomes can comprise one or more synergistic effects. Such additive/synergistic effects may not be predicted or expected given the nature and understanding of the individual components.

Certain embodiments can include expression nucleic acid constructs and/or vectors, cell lines and/or cell suspension cultures, and methods of manufacturing, purifying, and administering the one or more recombinant (e.g., human or mammalian) Klotho proteins, protein fragments, and/or protein variants to (human or non-human animal) subjects.

It will be appreciated that each of the forging, including but not limited to health supplements, formulations, co-formulations, co-administrations, combination products, and so forth, can be referred to as a “composition” or “composition of the present disclosure”.

### Administration of Exogenous S-Klotho

The present disclosure relates to S-Klotho formulations, clinical dosages, and administration (e.g., to increase and/or maintain serum concentrations of S-Klotho within the range of a normal and/or young (e.g., 18 - 30 years of age) person (e.g., without any chronic conditions).

Aspects or embodiments of the present disclosure include, for example, administering a (cGMP- and/or clinical grade) human recombinant alpha soluble Klotho protein or protein fragment (of isoform 1) to a (human) subject in need thereof. Embodiments can also include measuring (in the (human) subject) serum S-Klotho levels or concentration (e.g., by Mass Spectroscopy (MS) or ELISA). Such measuring can occur before, after, and/or during S-Klotho administration, and can be repeated, as necessary, to determine serum S-Klotho levels and/or a rate of metabolism, degradation, or reduction of serum S-Klotho levels. MS is a technique well known in the art. MS can be used to identify and even quantify the level of one or more (native and/or recombinant) Klotho proteins in the serum of a subject.

One or more additional proteins can also be measured in the subject's serum. For instance, one or more Klotho-related and/or aging-related proteins (e.g., FGF21, GDF-11, TIMP2, NAD+, CCL11, hormones testosterone, estrogen, etc.) and/or kidney function proteins (e.g., KIM-1, Cystatin-C, creatinine, BUN, creatinine, NGAL, etc.) can be measured separate from or in combination with measuring Klotho in the serum.

In at least one embodiment, a serum sample is obtained, such as a blood sample. The sample can be obtained by a blood draw, as known in the art. In a preferred embodiment, a finger prick or other less invasive means of obtaining a blood sample can be used. Accordingly, blood samples can be taken more frequently (e.g., throughout the day and/or every 0.5, 1, 2, 3, 4, 5, 6, 7, 8, 9, 10, 11, or 12 hours). MS can be used to measure the total Klotho protein serum concentration, as well as the serum concentration of various alpha Klotho protein species, such as native Klotho species (e.g., soluble Klotho, cleaved Klotho, secreted Klotho, etc.) and/or one or more Klotho proteins of the present disclosure. In some embodiments, Klotho levels can be measured before therapeutic recombinant Klotho protein administration and again throughout the day and/or every (or one or more of) 0.5, 1, 2, 3, 4, 5, 6, 7, 8, 9, 10, 11, or 12 hours post-administration. Alternatively, or in addition, Klotho levels can be measured every (or one or more of) 0.5, 1, 2, 3, 4, 5, 6, 7, 8, 9, 10, 11, 12, 13, 14, 16, 18, 20, 21, 25, 28, 30, 36, 40, 45, 50, 60, or 90 days (or weeks) post-administration.

Embodiments can also include determining such a rate and/or calculating a treatment protocol (e.g., including frequency, amount, and/or duration of (subsequent) administration(s) of S-Klotho) for maintaining within the serum of such subject a S-Klotho concentration within the range of a normal young person's serum concentration of S-Klotho. In at least one embodiment, the concentration of S-Klotho can be maintained at approximately 1000 picograms of (the S-Klotho) protein per milliliter of serum (pg/mL).

In at least one embodiment, a S-Klotho administration strategy (in humans) can include the measurement of one or more pharmacokinetic parameters of S-Klotho. For instance, measurements can be made of resultant changes in vivo of S-Klotho levels in the serum, urine and cerebrospinal fluid in response to S-Klotho administration. Some embodiments can include measuring the effectiveness of S-Klotho administration on one or more clinical indicators. Clinical indicators for various conditions, diseases, and disorders are known in the art and described further herein.

Embodiments can also include taking a (baseline) S-Klotho level measurement(s) (e.g., at zero time (before any administration of exogenous S-Klotho) and/or at different times of day before and/or throughout the treatment protocol (e.g., before and after administration of S-Klotho) in order to account for any circadian rhythm effects in the (human) subject(s)).

Embodiments can also include determining a suitable frequency, amount, and/or duration of S-Klotho administration. For instance, subjects with low S-Klotho serum levels (e.g., as determined by MS or ELISA immunoassay quantification), can be given (e.g., via any suitable route of administration) a first administration of klotho configured and/or adapted to bring the subjects serum S-Klotho levels to a first predetermined level (e.g., about 1000 pg/mL), measuring (the resultant change in) serum S-Klotho concentration in the subject (e.g., by MS or ELISA), measuring the levels and/or a rate of metabolism, degradation, or reduction of serum S-Klotho levels (following the first administration), calculating a half-life of the administered S-Klotho, and/or determining a frequency and/or timeframe in which a second, subsequent administration of S-Klotho should be given (e.g., in order to maintain serum S-Klotho levels above a second predetermined level). In at least one embodiment, the second predetermined level can be between and/or about 95%, 90%, 85%, 80%, 75%, 70%, 65%, 60%, 55%, 50%, 45%, 40%, 35%, 30%, 25%, 20%, 15%, 10%, and/or 5% of the first predetermined level.

Further administrations can be given over a timeframe suitable to produce in the subject (long-term) S-Klotho serum level equivalent to that of normal sex-matched young person's serum maintenance level (e.g., approximately 1000 pg/ml). The total timespan of the S-Klotho administration (to (human) subjects) can range from 1 day to 5 years, or more. Measurements and/or determinations of frailty in the subject based on the use of the clinical frailty score and other measurements can also be made over the timeframe.

Embodiments of the present disclosure further include increasing the S-Klotho dosage to maintain a 5%, 10%, 15%, 20%, 25%, 30%, 35%, 40%, 45%, 50%, 55%, 60%, 65%, 70%, 75%, 80%, 85%, 90%, 95%, 100% or more increase in S-Klotho levels above the normal range (e.g., at least about 500-1000 pg/ml serum, illustratively).

Certain embodiments include administering the S-Klotho protein. Non-limiting examples of suitable routes of administration include injection (e.g., bolus, gradual, intravenous, intradermal, intraperitoneal, intramuscular, intracutaneous, and/or subcutaneous injection), oral or oral-related administration (e.g., ingestion, buccal administration, and/or sublingual administration), topical administration, transdermal administration, rectal administration, vaginal administration, inhalation, and so forth.

An illustrative embodiment can include administering the S-Klotho protein or composition in a single bolus or extended (IV) injection (e.g., drip over an extended period of time). In at least one embodiment, 0.01, 0.05, 0.1, 0.5, 1, 1.5, 2, 2.5, 2.75, 3, 3.5, 4, 4.5, 5, 6, 7, 8, 9, 10, 12, 15, 18, 20, or more micrograms of S-Klotho per kilogram of subject body weight can be administered per treatment. A suitable administration amount can be calculated through one or more methods known in the art. One such method is the allometric scaling method. For example, in rat experiments, 0.01 mg of S-Klotho /kg body weight per administration was used, or 10 ug/kg. Using the human allometric scaled equivalent of 0.16, illustratively, the human equivalent dose (HED)=10 ug/kg×0.16=1.6 ug/kg. Hence a 70 kg human individual would require, 70 kg×1.6 ug/kg=112 ug and a 60 kg human would require, 60 kg×1.6=96 ug, illustratively. Table 46, below, demonstrates conversion of various animal doses to human equivalent doses (HED) based on body surface area. The conversions assume a 60kg human.

The HED was established using normalization to body surface area, a process described by Reagan-Shaw (2008), incorporated herein by reference. This process, termed allometric scaling, corrects for basal differences in metabolism rate between different species, and may be preferred over simple dose extrapolation. Illustratively, when the animal species dose is known, then the HED can be calculated as: HED=animal dose in mg/kg x allometric scaling factor for that appropriate animal species. The actual dose to be administered to a patient can then be calculated by multiplying the HED times the patient's weight.

TABLE 46

| | | To Convert Animal Dose in mg/kg | |
|---|---|---|---|
| | To Convert Animal | to HED^{a} in mg/kg, Either: | |
| | Dose in mg/kg to | Divide | Multiply |
| | Dose in mg/m², | Animal | Animal |
| Species | Multiply by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

Multiple factors can be considered or taking in account in determining, measuring, and/or estimating the amount and/or bioavailability of S-Klotho in humans (before and/or after recombinant protein administration), the total amount and/or concentration of S-Klotho to be administered, and/or the serum level response (over time) after recombinant protein administration. Such factors can include, for example, composition of the diluent, route of administration, site of administration, distribution into the tissues and organs of the subject, metabolic or other rate of the subject, pharmacokinetics (PK), pharmacodynamics (PD), toxicology (Tox), and so forth.

In at least one embodiment, a (normal) concentration of S-Klotho (e.g., in a healthy, young (18-30 years old) human adult) can be approximately 1000 pg/ml in serum. A typical adult can have a blood volume of approximately 5 liters, with females generally having less blood volume than males. Approximately 55% of human blood can be comprised of serum. Thus, (5 liters of blood/adult)×(0.55 serum/liter blood)=2.75 liters (2750 ml) of serum/adult. Assuming no endogenous serum S-klotho, to achieve a final concentration of 1000 pg/ml in the total blood serum; 2750 ml×1000 pg/ml=2,750,000 pg (or 2750 ng, or 2.75 pg) exogenous S-Klotho can be administered per adult subject.

To increase soluble Klotho to 50% greater than typical healthy levels (e.g., to 1500 pg/ml serum), a dose of 4.125 micrograms/subject can be administered. To increase soluble Klotho to 100% greater than typical healthy levels (e.g., to 2000 pg/ml serum) a dose of 5.5 micrograms/subject can be administered, and so forth.

A pharmaceutically effective and/or sufficient amount of purified recombinant S-klotho protein can be administered so as to raise the serum soluble Klotho protein concentration of the subject to any suitable level, such as greater than, equal to, or between about 50, 100, 250, 500, 750, 1000, 1250, 1500, 1750, 2000, 2250, 2500, 2750, 3000, 3500, 4000, 4500, 5000, 5500, 6000, 6500, 7000, 7500, 8000, 8500, 9000, 9500, 10,000, 11,000, 12,000, 13,000, 14,000, 15,000 20,000, 25,000, 30,000 40,000, 50,000, 75,000, 100,000 or more picograms of soluble Klotho protein per milliliter of serum, or greater than, equal to, or between about 5%, 10%, 15%, 20%, 25%, 30%, 40%, 50%, 60%, 70%, 80%, 90%, 100%, 150%, 200%, 250%, 300%, 400%, 500%, 600%, 700%, 800%, 900%, 1000%, 1200%, 1500%, 2000%, 2500%, 3000%, 4000%, 5000%, or more greater than typical healthy levels of soluble Klotho protein in serum.

In some embodiments, subjects can be administered one or more (bolus) intravenous, intradermal, intraperitoneal, intramuscular, intracutaneous, subcutaneous, and/or other injection of recombinant human S-Klotho protein at a dosage of greater than, equal to, or between about 0.01 mg/kg body weight in a suitable volume of Klotho buffer (e.g., 150 mM NaCl and 10 mM HEPES pH 7.4) or other pharmaceutically acceptable carrier. Thus, a 160 pound subject (i.e., body weight of 72.57 kg) can be administered a (bolus) injection of about 0.73 mg of S-Klotho per administration (based on the calculation of 0.01 mg of S-Klotho/kg×72.57 kg body weight). Likewise, a 170 pound person can receive a 0.77 mg of S-Klotho per administration. The total number and frequency of administrations can be determined based on achieving and maintaining in serum, a concentration of, for example, 1000 pg/ml of S-Klotho (equivalent to 0.000001 mg/ml serum). The latter can be ascertained as measured by MS or by a human S-Klotho ELISA assay.

In other embodiments, the dosage can be greater than, equal to, or between about 0.0001-10 mg/kg body weight, 0.0001-10 pg/kg body weight, 0.0001-10 ng/kg body weight, 0.0001-10 pg/kg body weight, or any value or range of values therebetween. Urine and/or blood can be collected at one or more time points, such as greater than, less than, equal to, between, and/or about 5 minutes, 10 minutes, 15 minutes, 20 minutes, 25 minutes, 30 minutes, 40 minutes, 45 minutes, 60 minutes, 90 minutes, 2 hours, 3 hours, 4 hours, 5 hours, 6 hours, 9 hours, 12 hours, 18 hours, 24 hours, 36 hours, 48 hours, 2.5 days, 3 days, 5 days, 7 days, 10 days, 14 days, 21 days, 4 weeks, 1 month, 2 months, 3 months, or more after a medical procedure or a first administration (dose) of recombinant Klotho protein (e.g., to measure, test, and/or determine serum S-Klotho levels and changes in response to administration and/or over time).

One or more embodiments include production and/or (subsequent) administration of a unique formulation of the S-Klotho active drug product and/or in combination with a pharmaceutically-acceptable carrier. The carrier can be suitable for IV and/or bolus injection. Embodiments can also include production and/or (subsequent) administration of a unique inactive prodrug formulation of the S-Klotho and/or in combination with a pharmaceutically-acceptable carrier (so that an inactive S-Klotho can be activated in vivo to release biologically effective S-Klotho in animals or in human subjects. Such prodrug formulation can include a coating or time-release formulation.

### Treatment Methods, Uses, Etc.

Some embodiments include a method or treatment method (e.g., comprising one or more method steps). Some embodiments can include administering one or more compositions, as described herein, to a subject (e.g., a subject in need thereof). Administration of the inventive composition(s) can be sufficient to raise the serum soluble Klotho protein concentration of the subject to or above a predetermined threshold, or to maintain the serum soluble Klotho protein concentration of the subject at or above a predetermined threshold, for a period of time.

The composition can comprise, for example, (i) a therapeutic recombinant Klotho protein or fragment or fusion protein thereof, as described in the present disclosure, and/or (ii) a product that increases, enhances, augments, and/or supports endogenous Klotho protein production and/or level(s) in a patient to which the product is administered, optionally with (iii) one or more additional (active or inactive) ingredients or compositions. Those skilled in the art will appreciate that each of the therapeutic effects, indications, and/or treatments outlined herein in relation to recombinant, therapeutic Klotho proteins and products, compositions, and methods related thereto can be achieved by (naturally) increasing, enhancing, augmenting, and/or supporting endogenous Klotho protein production and/or level(s). Those skilled in the art will also appreciate that each of the therapeutic effects, indications, and/or treatments outlined herein in relation to (naturally) increasing, enhancing, augmenting, and/or supporting endogenous Klotho protein production and/or level(s) can be achieved by administering recombinant, therapeutic Klotho proteins and products, compositions, and through methods related thereto. Accordingly, and for the sake of brevity, the present disclosure need not rehearse each of the disclosed therapeutic effects, indications, and/or treatments with reference to both (i) the administration of recombinant, therapeutic

Klotho proteins, and (ii) the administration of the health supplement or nutraceutical compositions of the present disclosure, as such are clearly contemplated herein.

Certain embodiments can include a composition, as described herein, for use in treating one or more diseases, disorders, injuries, and/or (medical) conditions. Similarly, embodiments can include a method of treating (or alleviating, addressing, preventing, inhibiting, etc.) one or more diseases, disorders, injuries, and/or (medical) conditions. An exemplary treatment method comprises administering to a subject in need thereof an effective amount of a composition of the present disclosure. Without being bound to any theory, administering an effective amount of the composition to a subject in need thereof can cause or result in elevated (or raised) serum soluble Klotho protein concentration in the subject. Accordingly, the “effective amount” of a composition of the present disclosure can be an amount effective to raise or elevate the serum soluble Klotho protein concentration of the subject to or above a predetermined threshold, or to maintain the serum soluble Klotho protein concentration of the subject at or above a predetermined threshold, for a predetermined period of time. Alternatively, or in addition, the “effective amount” of a composition of the present disclosure can be an amount effective to achieve a beneficial therapeutic effect in a patient to which the composition is administered.

The method, composition or (raised) protein can also be effective to treat one or more diseases, disorders, injuries, and/or (medical) conditions. Illustratively, the condition can be or comprise an age-related or aging-related condition (or condition associated with (human) aging), such as frailty, bone density loss or bone mineral density loss, weight loss, muscular atrophy or degeneration, decline in muscle mass, decline in muscle strength, hand strength, leg strength, or physical fitness, decline in movement, freedom of movement, quality of life assessment, ejection fraction, or exercise capacity, decline in learning, learning capacity, memory, or intellectual quotient, cognitive deterioration or forgetfulness, decline in cognitive capacity or function, decline in synaptic plasticity or synaptic function, and cellular senescence. It will be appreciated, however, that the condition can also, or alternatively, be or comprise any physical, mental, emotional, or other deficiency, need, or desire that increased serum Klotho levels can provide a beneficial therapeutic effect, including those described in the present disclosure and those known to those skilled in the art.

The method, composition or (raised) protein can also be effective to treat one or more aging-related condition (or condition associated with (human) aging), such as Alzheimer's disease, Parkinson's disease, dementia or vascular dementia, amyotrophic lateral sclerosis (ALS) or motor neuron disease (MND), atrial fibrillation, chronic obstructive pulmonary disease (COPD), fibromyalgia, adult onset diabetes, arthritis or rheumatoid arthritis, osteoarthritis, osteoporosis, glaucoma, cataracts, macular degeneration and other eye diseases/disorders, multiple sclerosis (MS), lupus, and/or ulcerative colitis.

Some embodiments of the present disclosure can be useful in one or more of treating cancer, lowering serum phosphate levels in a patient, treating diabetes or a diabetes- related condition (e.g., Type 1 diabetes mellitus, etc.) in a subject in need of such treatment, treating a heart condition (e.g., cardiovascular disease, left ventricular hypertrophy (LVH), pathological LVH and/or congestive heart failure, etc.) in a subject, treating acute lung injury in a subject (e.g., using nanoparticles), protecting the lung of a patient against oxidant injury, detecting early acute kidney injury in critically ill patients, attenuating vascular calcification in a subject, improving cognition, treating renal and/or liver ischemia, modulating stress response in (human) senescent endothelial cells, prophylactically and/or therapeutically treating, preventing, attenuating, arresting, and/or reversing acute and/or chronic kidney injury, disease, or disease progression and/or uremic cardiomyopathy, reversing or attenuating age-related therapy resistance in melanoma and other cancers, as well as muscular dystrophy, including DMD, targeting apoptosis of senescent cells, preferably restoring tissue homeostasis thereby, and a variety of other indications.

Some embodiments of the present disclosure can be useful for treating one or more other conditions. Such conditions may be described or disclosed herein. Such conditions may be known by those skilled in the art to be related to Klotho serum levels.

As described briefly, above, the present disclosure includes and contemplates various dosage forms and/or methods of administration of compositions of the present disclosure. Some embodiments can be dosed in an oral dosage form and/or administrated orally (e.g., in one or more convenient forms including tinctures, tablets, capsules, powders, teas, etc.). For instance, a tincture, comprised of an alcohol- or glycerin-based extract of a herb or natural ingredient can be packaged in a glass dropper bottle. Glycerin-based tinctures can provide a sweet taste, making the formulation more easily to swallow. Tinctures may also be administered in a dropper. Tinctures may include a dosage range on the label (e.g., 20 to 40 drops per adult dose).

Capsules and tablets are also popular form for the oral administration of supplements. For example, a dried herb can be powdered and pressed into gelatin capsules. The supplement may be supplied as a capsule and/or tablet. The supplement may be supplied as a powder within a bottle or envelope with dosage amounts to be used listed on the container. An appropriate amount of powder per dose may also be obtained by opening up a dosage capsule and sprinkling the content into a drink or on food.

Teas (e.g., herbal infusions) are common forms of herbal remedy and a soothing way to take herbs. An herbal infusion can be made by placing a small amount of the herb in a cup, filling the cup with boiling water and letting it steep. In a tea form, herbal ingredients of the supplements may increase in potency with extended steeping time and the tea is best left to steep for at least 10 minutes. Covering the cup (a small saucer works well) keeps the steam in and keeps the tea hot longer.

Some embodiments can include a method of treating an aging-related or other condition, disease, or disorder, the method comprising administering to a subject in need thereof an effective amount of a composition of the present disclosure. Some embodiments can include a method of treating and/or preventing a condition, disease, or disorder, preferably a condition, disease, or disorder associated with aging, in a mammalian subject, the method comprising administering to a subject in need thereof an effective amount of a composition of the present disclosure. Some embodiments can include a method of treating or preventing acute kidney injury (AM), chronic kidney disease (CKD), or other condition, the method comprising administering to a subject in need thereof an effective amount of a composition of the present disclosure. Some embodiments can include a method of treating and/or preventing kidney injury in a mammalian subject, the method comprising administering to the subject, optionally prophylactically, prior to a medical procedure or administration of a nephrotoxin and/or following a medical procedure or administration of a nephrotoxin, an effective amount of a composition of the present disclosure. Some embodiments can include a method of administering a composition of the present disclosure to a human or non-human animal (e.g., mammalian) subject in need thereof. The subject to whom the composition is administered can be suffering from or at risk for a variety of conditions (e.g., disorders, diseases, injuries, illnesses, etc.). For example, some embodiments include a method of treating one or more chronic diseases and/or aging-related condition, such as a physical, mental, neurological, or other condition associated with (human) aging. Some embodiments can promote healing, recovery, longevity, and/or other beneficial outcome through one or more mechanisms or action.

The pharmaceutically effective amount of the composition can be sufficient to increase or raise the serum soluble Klotho protein concentration of the subject (e.g., to a predetermined level, such as greater than, equal to, or between about 50 to 3000 picograms of soluble Klotho protein per milliliter of serum). The pharmaceutically effective amount of the composition can also or alternatively be sufficient to maintain the serum soluble Klotho protein concentration of the subject at or above a predetermined threshold (e.g., for a predetermined period of time).

In certain embodiments, suitable increase in protein levels can (be effective to) modulate the IGF-1 and/or Wnt signaling pathways, exhibit δ-glucuronidase and/or sialidase activity, suppress the p53/p21 signaling pathway, and/or reduce H₂O₂-induced cell senescence and apoptosis, preferably through suppression of the p53/p21 signaling pathway. The (raised) protein can function or be functional as a humoral factor, preferably exhibiting pleiotropic activity and/or preferably in the regulation of oxidative stress, growth factor signaling, ion homeostasis, and/or regulation of activity of glycoproteins on the cell surface, such as one or more ion channel proteins and/or growth factor receptors, such as Insulin/Insulin-Like Growth Factor-1 receptor.

Certain embodiments can include determining a serum soluble Klotho protein concentration of the subject, calculating the effective amount, determining a rate of soluble Klotho protein decline in the serum of the subject, calculating a subsequent dosage time at which the serum soluble Klotho protein concentration of the subject will be at or below a second predetermined level based on the determined rate, calculating a subsequent dosage amount of the composition sufficient to raise the serum soluble Klotho protein concentration of the subject from the second predetermined level to the first predetermined level, and/or administering the subsequent dosage amount of the composition to the subject. Certain embodiments can include prescribing a regular (e.g., daily) dose or dosage form of the composition to the subject (e.g., based on determined levels of serum soluble Klotho protein concentration of the subject).

An illustrative embodiment comprises a (treatment) method. The treatment method can be similar to and/or modeled after a study of the therapeutic effect or health benefits of various compositions of the present disclosure. The results of administration of various compositions are monitored in human subjects. Once a subject has voluntarily agreed to be in the study, and signed the Patient Consent Form, they are given a physical examination and blood and saliva tests. Upon meeting the Inclusion Criteria in the Study Protocol, the subject is then enrolled in the study. Illustratively, the study may include 100 healthy individuals that are not currently on vitamin D supplementation and that do not live in a sunny environment. Subjects with a KLVS variant (Klotho over-expressor) and/or other genetic variant of klotho gene(s) may be excluded from the study. Following enrollment, the subjects receive a 3-month supply of a Klotho supplement formulation of the present disclosure or a placebo in a double-blind study. Subjects receive unmarked bottles of supplement formulation or placebo at no cost. Subjects take this supplement daily for 3 months.

At the end of this 3-month period, blood is drawn for a fasting blood test, and the subject's weight, blood pressure is measured and recorded along with the concentration of plasma alpha soluble Klotho determined by an Alpha Klotho (α-Klotho) Human Soluble ELISA Assay Kit (from for example, Catalog No. 27998, from IBL America, Minneapolis, Minn., USA).

The Study/Design Procedures regarding the human subjects in this study is given below.

### Study Design/Procedures:

Up to 100 patients

Duration: 3 months to first endpoint, crossover, second endpoint at 6 months including placebo patients that elected to take the supplement.

Baseline Questionnaire to include: Current medications (statins, ARBs), nutritional supplements, blood pressure, exercise frequency, alcohol consumption.

Prior to enrolling:

Collect blood sample and cheek swab (PI office or mobile phlebotomist)

Determine KLVS variant (KLOTHO overexpressor)—send cheek swab to lab to determine KLVS variant (Klotho over-expressor) status

Lab analysis of Klotho level, Vitamins D & E, and other analyses for pre-treatment baseline

Supplementation: (3-month supply) shipped to participant. (Active supplement and placebo controls, equal # of active and placebo controls)

Outcomes measure: Participant downloads mobile application to monitor compliance and questionnaires (exercise, blood pressure)

Follow up blood sample collection at 3 months and 6 months

The ingredients in the supplement are comprised of nutritional components that pose very low risk. If subject experiences any discomfort or adverse reaction to the formulation he or she will be instructed to stop taking the product and contact the study doctor.

Any significant adverse reactions will be reported to the study's IRCM Institutional review Board (IRB) within 48 hours.

### Example Conditions

In some embodiments, the condition, disease, or disorder treated with the composition(s) and/or method(s) of the present disclosure can, preferably, be selected from the group consisting of, for example: frailty; bone density loss; bone mineral density loss; weight loss; muscular atrophy; muscular degeneration; decline in muscle mass; decline in muscle strength; decline in hand strength; decline in leg strength; decline in physical fitness; decline in movement; decline in freedom of movement; decline in quality of life assessment; decline in ejection fraction; decline in exercise capacity; decline in learning; decline in learning capacity; decline in memory; decline in intellectual quotient; cognitive deterioration; forgetfulness; decline in cognitive capacity; decline in cognitive function; decline in synaptic plasticity; decline in synaptic function; cellular senescence; chronic kidney disease (CKD); chronic kidney disease—mineral and bone disorder (CKD-MBD); polycystic kidney disease (PKD); autosomal dominant polycystic kidney disease (ADPKD); acute kidney injury (AM); acute tubular necrosis (ATN); acute allergic interstitial nephritis (AAIN); glomerulonephritis; kidney disease; renal failure; Alport Syndrome; nonoliguric renal failure; alcoholism; hyperphosphatemia; muscular dystrophy (MS); type 1 diabetes; type 2 diabetes; cardiovascular disease (CVD); cardiovascular calcification; cerebrovascular insufficiency; vascular calcification; coronary artery disease; heart failure; left ventricular hypertrophy; uremic cardiomyopathy; abnormalities in blood pressure; salt-sensitive hypertension; tissue calcification; calcific atherosclerotic plaque burden; calcinosis; familial tumoral calcinosis; cancer; one or more tumors; myelin-related diseases; demyelinating diseases; neurodegenerative disease; neurovascular diseases; progressive supranuclear palsy (PSP); Pompe disease; Niemann-Pick disease; microgliosis; Farber disease (FD); bone mass diseases; osteoporosis; osteopenia; osteopenia (particularly loss of BMD of cortical bone); pulmonary emphysema; pulmonary fibrosis; cystic fibrosis, idiopathic (i.e., cause unknown) pulmonary fibrosis, radiation-induced lung injury, cirrhosis, biliary atresia, atrial fibrosis, endomyocardial fibrosis, (old) myocardial infarction, glial scar, arterial stiffness, arthrofibrosis, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma/systemic sclerosis, adhesive capsulitis, skin atrophy; thymic atrophy; accumulation of renal interstitial matrix; glomerulosclerosis; anemia; albuminuria; proteinuria; infertility; Alzheimer's disease; Parkinson's Disease; dementia; vascular dementia; amyotrophic lateral sclerosis (ALS); motor neuron disease (MND); atrial fibrillation; chronic obstructive pulmonary disease (COPD); fibromyalgia; adult onset diabetes; arthritis; rheumatoid arthritis; osteoarthritis; glaucoma; cataracts; macular degeneration; multiple sclerosis (MS); lupus; ulcerative colitis; cachexia; obesity; vitamin D-related conditions; bone diseases; bone diseases through bone remodeling; gut diseases and related conditions; stem cell depletion; sea sickness; space adaptation syndrome (SAS); nausea; vertigo; non-alcoholic steatohepatitis (NASH), cirrhosis of the liver and alcoholic steatohepatitis.

Klotho serum levels have also been reported as being associated with muscle recovery, such as postinjury muscle healing. In particular, and without being bound to any theory, age-related declines in α-Klotho may drive progenitor cell mitochondrial dysfunction and impaired muscle regeneration. Clinically, this could translate to treatment of older adults who either sustained a muscle injury or underwent muscle-damaging surgery. Embodiments of the present disclosure can include administering a composition, as described herein, prophylactically and/or post-operatively. Administration at the appropriate timepoint(s) can enhance or support muscle regeneration and lead to a more complete recovery. Serum Klotho protein levels may also enhance or support muscle regeneration in athletes and other patients. Accordingly, embodiments of the present disclosure can include administering a composition, as described herein, prophylactically (pre-workout) and/or following exercise.

### Administration of Compositions to Treat Age-Related Frailty in Humans and Non-Human Mammals

An exemplary embodiment of the present disclosure relates to the administration of a composition to treat age-related frailty (e.g., in humans or non-human animals). s-Klotho may rescue myogenic stem cells, improve muscle repair, and/or suppress fibrosis in animal models of human disease. Compositions that increase s-Klotho levels may, therefore, be a promising approach to counter muscle degeneration in aging human subjects showing signs of frailty.

For instance, the present disclosure relates to s-Klotho level-increasing compositions, formulations, and dosages, and administration thereof to a fragile and/or elderly (e.g., 60-95 years of age) person in order to maintain in the subject a maintenance serum concentration of s-Klotho within the range of a normal and/or young (e.g., 18-30 years of age) person (e.g., without any chronic conditions).

Such Klotho treatment over an extended period of time can recover and/or improve one or more aging-related indicator or conditions in the elderly, frail, or otherwise physiologically aging.

### Administration of Compositions to Treat (Decrease) Muscle Atrophy in Humans and Non-Human Mammals

An exemplary embodiment of the present disclosure relates to the administration of compositions of the present disclosure to treat (e.g., decrease (the rate of)) muscle atrophy in human, as measured by skeletal muscle tissue mass and, preferably, the concurrent use of the above compositions and molecular indicators to provide guidance on the effects of administration in countering muscle atrophy.

Muscle atrophy can include numerous neuromuscular, metabolic, immunological and neurological disorders and diseases as well as starvation, nutritional deficiency, metabolic stress, diabetes, aging, muscular dystrophy, or myopathy. Muscle atrophy can occur during the aging process. Muscle atrophy can also result from reduced use or disuse of the muscle. Symptoms include a decline in skeletal muscle tissue mass. In human males, muscle mass declines by one-third between the ages of 50 and 80. Some molecular features of muscle atrophy can include the upregulation of ubiquitin ligases, and the loss of myofibrillar proteins. The breakdown of these proteins, which can be monitored (e.g., by measuring 3-methyl-histidine production, which is a specific constituent of actin), for example, in certain muscles of myosin. Release of creatine kinase (a cell damage marker) can also be indicative.

### Administration of Compositions to Treat (Improve) Muscle Repair or Recovery in Humans and Non-Human Mammals

Klotho serum levels have also been reported as being associated with muscle recovery, such as postinjury muscle healing. In particular, and without being bound to any theory, age-related declines in α-Klotho may drive progenitor cell mitochondrial dysfunction and impaired muscle regeneration. Clinically, this could translate to treatment of older adults who either sustained a muscle injury or underwent muscle-damaging surgery. Embodiments of the present disclosure can include administering a composition, as described herein, prophylactically and/or post-operatively. Administration at the appropriate timepoint(s) can enhance or support muscle regeneration and lead to a more complete recovery. Serum Klotho protein levels may also enhance or support muscle regeneration in athletes and other patients. Accordingly, embodiments of the present disclosure can include administering a composition, as described herein, prophylactically (pre-workout) and/or following exercise.

Administration of Compositions to Treat Mental and/or Cognitive Decline in Old Age and/or throughout the Human Lifespan

An exemplary embodiment of the present disclosure relates to the administration of compositions of the present disclosure to improve and/or counteract (aging-related) decline in cognitive function(s). At the time of the present disclosure, it was unknown whether administration of compositions of the present disclosure may counteract cognitive decline in humans. However, transgenic mice with systemic over-expression of klotho performed better than controls in multiple tests of learning and memory. Elevating klotho in mice also enhanced long-term potentiation, a form of synaptic plasticity, and enriched synaptic GluN2B, an N-methyl-D-aspartate receptor (NMDAR) subunit with key functions in learning and memory. Blockade of GluN2B abolished klotho-mediated effects.

Klotho-regulated pathways may be relevant to slowing the progression of Alzheimer's disease and other forms of dementia. Brain scans of more than 400 healthy men and women aged 53 and over found that those who carried a single copy of a particular Klotho gene variant had a larger brain region that deals with planning and decision making. Further tests on the group found that those with an enlarged right dorsolateral prefrontal cortex (rDLPFC) fared better on a series of mental tasks.

About one in five people inherits a single copy of the gene variant, or allele, known as KL-VS, which improves heart and kidney function, and on average adds about three years to human lifespan. However as to the brain function, having a larger rDLPFC accounted for only 12% of the improvement in people's mental test scores while, on the other hand, as the editorial points out, carrying one copy of the KL-VS allele appears to confer a decade of resilience against the expected decline in structure and function of the rDLPFC. Thus, KL-VS heterozygosity appears to be associated with greater volume in right dorsolateral prefrontal cortex (rDLPFC).

Because rDLPFC is important for executive function, the investigators also analyzed working memory and processing speed in individuals. KL-VS heterozygosity may be associated with enhanced executive function across the lifespan examined. In short, results may suggest that variation in the klotho gene may be associated with bigger brain volume and better function.

An exemplary embodiment of the present disclosure relates to the administration of compositions of the present disclosure to augment in vivo Klotho protein levels and/or (cellular, molecular, and/or downstream) effects (e.g., in order to enhance cognition and counteract cognitive deficits throughout the human lifespan). Administration of s-Klotho level-increasing compositions can preserve and/or improve cognitive function (e.g., in humans).

Administration of Compositions to Treat (Prolong) Human Longevity and/or Lifespan

An exemplary embodiment relates to the administration of compositions of the present disclosure to chronological (e.g., age-matched from birth) and sex-matched human subjects to improve average lifespan. The results on average lifespan obtained with composition administration (the experimental group of human subjects) can be compared reliably to those individuals not receiving a composition (the control group) and/or with statistical information (e.g., from actuarial tables) for a sufficiently large human population.

Administration of Compositions to Treat other Clinical Indications

An exemplary embodiment of the present disclosure relates to the administration of compositions of the present disclosure to treat any age-associated or otherwise non-age associated condition, including, but not limited to (increase in) human frailty, (decrease in) longevity, (decrease in) cellular senescence, (decline in) muscle strength, (decrease in) bone loss or density, (decrease in) cognition, (decline in) muscle mass, (decline in) physical fitness, (decline in) hand strength, (decline in) leg strength, and so forth. The present disclosure also relates to the administration of compositions of the present disclosure to increase bone mineral density (BMD) (e.g., in women but not in men), to increase BMD (e.g., in elderly women), which is reduced after menopause, to regenerate or reduce the degeneration of (degenerative) skeletal muscle, to improve walking gait, to improve (or reduce the decline in) spatial learning and memory, movement, freedom of movement, quality of life assessment, ejection fraction, exercise change, exercise improvement, and so forth. The present disclosure further relates to the administration of compositions of the present disclosure to decrease cognitive deterioration or forgetfulness, to increase cognitive capacity, to improve cognitive function and synaptic plasticity, to decrease a decline in learning, learning capacity or IQ, to improve learning, learning capacity or IQ, and so forth.

### Administration of Compositions to Treat Acute Kidney Injury (AKI)

Acute kidney injury (AKI), previously called acute renal failure (ARF), is often defined as an abrupt onset of renal dysfunction ranging from minor loss of function to failure. AM is a common clinical complication that develops in approximately 4%-7% of hospitalized patients each year and the prognosis can be poor. Mortality rates associated with AM range from 5%-35%. Renal Klotho expression has been shown to be suppressed following AM. Adenoviral gene transfer of Klotho can be cytoprotective in AKI.

Acute Kidney Injury (AKI) has been report in approximately 4.9%-7% of hospitalized patients each year. The rate of AM may be as high as 60% in (hospitalized) elderly persons and 20-30% in elderly or critically ill patients. AM is also associated with increased mortality, length of stay (LOS), 30 day re-administration rates and hospital cost.

AKI may result at least in part from kidney transplant or other surgery, acute tubular necrosis (ATN), acute allergic interstitial nephritis (AAIN), nephritis (e.g., glomerulonephritis), nephrotoxicity (e.g., drug-induced nephrotoxicity), low blood pressure, sepsis or septic condition, or other contributing factor(s). Kidney transplant and other surgeries can cause acute damage or injury to the kidneys, cause renal disease and/or failure. Nephrotoxicity can contribute to AKI, ATN, AAIN, nephritis, etc. It has been reported that drugs (e.g., clinically-administered, prescription, illegal, or other drugs) are associated with 15% to 25% of all cases of AKI. Contrast media alone accounts for 10% of all causes of hospital-acquired acute renal failure (e.g., via contrast-induced acute kidney injury CIAKI) and represents the third leading cause of in-hospital renal function deterioration after decreased renal perfusion and postoperative renal insufficiency.

In some cases, drug-induced AM can be or comprise anti-microbial-induced nephrotoxicity (resulting from anti-microbial treatment). For instance, certain (gram-negative) bacterial infections can be treated with one or more aminoglycosides, such as paromomycin, tobramycin, gentamicin, amikacin, kanamycin, neomycin, and so forth. Aminoglycosides have been shown to be nephrotoxic. Table 47 presents treatment data collected on the adult patients receiving the aminoglycosides. As illustrated in Table 47, nearly 23% of patients treated with amikacin, a common aminoglycoside, developed acute kidney disease and over 17% of patients treated with amikacin died prior to being discharged. Other aminoglycosides, including gentamicin, and tobramycin also induced (or were associated with or contributed to) kidney disease.

TABLE 47

| | Amikacin | Gentamicin | Tobramycin | Aminoglycosides |
|---|---|---|---|---|
| Length of Stay | 11.8% | 8.7 | 10.4 | 9.2 |
| Discharged Home | 55.1% | 72.0% | 62.6% | 68.9% |
| Died | 17.1% | 4.1% | 6.3% | 5.8% |
| ICU | 21.8% | 18.2% | 21.8% | 18.7% |
| Pediatric Patient (<18 yrs.) | 0.0% | 21.6% | 6.1% | 16.5% |
| Nursing Home Patient | 21.1% | 12.5% | 20.2% | 14.6% |
| Kidney Disease | 22.9% | 13.5% | 16.2% | 14.8% |
| Top 5 Dx | UTI/cystitis | UTI/cystitis | Pneumonia | UTI/cystitis |
| | Sepsis | Sepsis | UTI/cystitis | Sepsis |
| | Pneumonia | Skin Infection | Sepsis | Pneumonia |
| | Skin Infection | Pneumonia | Skin infection | Skin Infection |
| | Pelivc infection | FUO | Pelvic Infection | Pelvic Infection |

As illustrated in FIGS. 13A and 13B, over 1.2 million adult patients, in various age groups, were treated with aminoglycosides in the year 2010. Other anti-microbial agents include, for example, penicillins, ampicillin, cephalosporins, sulfonamides, ciprofloxacin, vancomycin, macrolides, tetracyclines, rifampin, and so forth.

Drug-induced nephrotoxicity can also result from treatment with one or more non-steroid anti-inflammatory drugs (NSAIDs), such as aspirin (acetylsalicylic acid), celecoxib, diclofenac, diflunisal, etodolac, ibuprofen, indomethacin, ketoprofen, ketorolac, nabumetone, naproxen, oxaprozin, piroxicam, salsalate, sulindac, tolmetin, and so forth.

Drug-induced nephrotoxicity can also result from treatment with one or more cyclooxygenase-2 (COX-2) inhibitors (e.g., valdecoxib, rofecoxib, celecoxib, etc.), proton pump inhibitors (e.g., omeprazole, lansoprazole, etc.), anticonvulsants (e.g., phenytoin, valproic acid, etc.), histamine H2 receptor antagonist (e.g., nizatidine, ranitidine, famotidine, cimetidine, etc.), diuretics (e.g., carbonic anhydrase inhibitors, loop diuretics (e.g., bumetanide, ethacrynic acid, torsemide, furosemide, etc.), potassium-sparing diuretics (e.g., triamterene, spironolactone, amiloride, etc.), thiazide diuretics (e.g., indapamide, chlorthalidone, metolazone, methyclothiazide, hydrochlorothiazide, chlorothiazide, bendroflumethiazide, polythiazide, hydroflumethiazide, etc.), or other diuretics, such as pamabrom, mannitol, and so forth.

Drug-induced nephrotoxicity can also result from treatment with lithium, which can affect the flow of sodium through nerve and muscle cells in the body and can be used to treat manic episodes of bipolar disorder, often characterized by hyperactivity, rushed speech, poor judgment, reduced need for sleep, aggression, and anger. Lithium can also help to prevent or lessen the intensity of manic episodes. Drug-induced nephrotoxicity can also result from treatment with or exposure to gold, mercury, copper, or other elemental matter.

Drug-induced nephrotoxicity can also result from treatment with (D-) penicillamine; a medication of the chelator class, which can be used to treat scleroderma, Wilson's disease (by binding to accumulated copper for elimination through urine), cystinuria (by binding with cysteine to yield a mixed disulfide which is more soluble than cysteine), direct-acting smooth muscle relaxant (e.g., hydralazine), spasmolytics (e.g., carisoprodol, cyclobenzaprine, metaxalone, methocarbamol, benzodiazepines, such as diazepam, clonidine and other imidazoline compounds, tizanidine, baclofen, hydantoin derivatives, dantrolene, and so forth.

Other forms of drug-induced nephrotoxicity can include, for example: contrast-induced nephrotoxicity (e.g., following exposure to (iodinated) contrast media, also known as radiocontrast-induced nephropathy (CIN)); narcotic- (opioid-) induced nephrotoxicity (e.g., following use or abuse of certain narcotics (e.g., opioids), such as cocaine, heroin, etc.); chemotherapy-induced nephrotoxicity (e.g., following treatment with a cancer therapeutic, such as: cisplatin; carboplatin; oxaliplatin; alkylating agents, such as bendamustine, cyclophosphamide, ifosfamide, nitrosoureas, temozolomide, melphalan, etc.; antitumor antibiotics, such as mitomycin C, bleomycin, anthracyclines and related agents, etc.; antimetabolites, such as capecitabine, hydroxyurea, methotrexate, pemetrexed, pralatrexate, pentostatin, fludarabine, cladribine, gemcitabine, cytarabine, etc.; vinca alkaloids; topotecan; etoposide; taxanes; irinotecan; lenalidomide; eribulin; arsenic trioxide; ixazomib; etc.); and so forth. Indeed, a wide variety of nephrotoxic drugs can induce nephrotoxicity, leading to AKI. Drug-induced nephrotoxicity (and other forms of AKI) can be life-threatening if untreated and can incur enormous costs (to patients, hospitals, and insurers) to treat.

Embodiments of the present disclosure can include a method of treating or preventing (e.g., prophylactically or in response to at least one event) acute kidney injury (AKI), chronic kidney disease (CKD), or other condition. The method can comprise administering a composition of the present disclosure to a subject in need thereof. For instance, the method can comprise administering to a subject in need thereof, an effective amount of a composition of the present disclosure (e.g., so as to raise and/or maintain a serum soluble Klotho protein concentration of the subject at or above a predetermined threshold for a predetermined period of time). The composition can comprise (i) a therapeutic recombinant Klotho protein or fragment or fusion protein thereof and/or (ii) a product that increases, enhances, augments, and/or supports endogenous Klotho protein production and/or level(s) in a patient to which the product is administered, optionally with one or more additional ingredients or compositions.

In some embodiments, the condition can comprise: (i) acute tubular necrosis (ATN), acute allergic interstitial nephritis (AAIN), nephritis, glomerulonephritis, and/or nephrotoxicity; or (ii) AKI resulting at least in part from sepsis, kidney transplant or other surgery or medical procedure, acute tubular necrosis (ATN), acute allergic interstitial nephritis (AAIN), nephritis, glomerulonephritis, nephrotoxicity, or low blood pressure. The condition can comprise a drug-induced (e.g., aminoglycosides-induced) nephrotoxicity. The protein can be administered prophylactically, for example, prior to kidney transplant, nephrotoxin administration, or other activity, treatment, or event known or anticipated to cause or contribute to AKI. Alternatively, or in addition, the protein can be administered in response to AM, such as after kidney transplant or other surgery, aminoglycoside or other nephrotoxin administration, or other activity, treatment, or event known or anticipated to cause or contribute to AKI.

In some embodiments, the nephrotoxin or other drug can be or comprise, for example: one or more aminoglycosides (e.g., paromomycin, tobramycin, gentamicin, amikacin, kanamycin, neomycin, etc.); one or more anti-fungal agents (e.g., amphotericin B, flucytosine, etc.); one or more contrast agents (e.g., (iodinated) radiocontrast media, high-osmolality contrast media (HOCM) having an iodine to molecule ratio of about 1.5 : 1, low-osmolality, nonionic contrast media (LOCM) having an iodine to molecule ratio of about 3:1, isosmolar (isoosmolality) contrast media (IOCM) having an iodine to molecule ratio of about 6:1), etc.); one or more antiretroviral agents (e.g., adefovir, cidofovir, tenofovir, foscarnet, etc.); one or more cancer (or chemo-) therapeutics (e.g., cisplatin, carboplatin, oxaliplatin, alkylating agents (such as bendamustine, cyclophosphamide, ifosfamide, nitrosoureas, temozolomide, melphalan, etc.), antitumor antibiotics (such as mitomycin C, bleomycin, anthracyclines and related agents, etc.), antimetabolites (such as capecitabine, hydroxyurea, methotrexate, pemetrexed, pralatrexate, pentostatin, fludarabine, cladribine, gemcitabine, cytarabine, etc.), vinca alkaloids, topotecan, etoposide, taxanes, irinotecan, lenalidomide, eribulin, arsenic trioxide, ixazomib, etc.); one or more bisphosphonates, or derivatives thereof (e.g., zoledronate/zoledronic acid, ibandronate, alendronate, alendronate/cholecalciferol, etidronate, risedronate (optionally, with calcium carbonate), pamidronate, tiludronate, etc.); and/or one or more narcotics (e.g., opioids), such as cocaine, heroin, etc.);

Embodiments of the present disclosure can include a method of administering a composition of the present disclosure. The method can include administering the composition of the present disclosure to a human or non-human subject to treat or prevent (prophylactically) AKI or one or more conditions associated with AKI. The method can include determining a level of serum soluble klotho level in the subject, calculating a first dosage of a composition of the present disclosure sufficient to raise the serum soluble klotho level in the subject to a predetermined level or percent of normal levels, administering the first dosage to the subject, such as by oral, bolus or gradual administration, determining a rate of soluble Klotho decline in the serum of the subject, such as following administration of the first dosage, calculating a subsequent dosage time and amount, and/or administering the subsequent dosage of protein to the subject.

### Administration of Compositions to Treat Chronic Kidney Disease (CKD)

Without being bound to any theory, klotho may be involved in human chronic kidney disease. Soluble Klotho in the circulation starts to decline early in chronic kidney disease (CKD) stage 2 and urinary Klotho possibly even earlier in CKD stage 1. Therefore, soluble Klotho could serve as an early and sensitive marker of kidney function decline. Moreover, preclinical animal data support Klotho deficiency is not just merely a biomarker, but a pathogenic factor for CKD progression and extrarenal CKD complications including cardiovascular disease and disturbed mineral metabolism. Prevention of Klotho decline, re-activation of endogenous Klotho production, or supplementation of exogenous Klotho could all contribute to attenuation of renal fibrosis, retardation of CKD progression, improvement of mineral metabolism, amelioration of cardiomyopathy, and alleviation of vascular calcification in CKD.

CKD is characterized by progressive deterioration of renal function with high risk of ESRD. CKD risk increases with age, and about half of the CKD stageΔ3 cases occurs in subjects>or =70 years old. CKD can be viewed as a state of accelerated aging. The relative risk for cardiovascular mortality of a 25 to 34-year-old dialysis patient is similar to a non-CKD patient of >or =75 years of age. Cardiovascular disease is the principal killer in CKD and ESRD patients. CKD and ESRD patients have low renal Klotho expression and low levels of circulating Klotho. Renal Klotho deficiency in early stages of CKD may be attributed mainly to suppression of Klotho expression rather than loss of viable renal tubules. Furthermore, some dialysis patients still have detectable circulating Klotho, suggesting that renal Klotho expression is not completely suppressed, and Klotho may come from extra-renal source(s), although its origin is not clear to date. Establishing extra-renal sources of Klotho and characterizing how these can be up-regulated when renal production fails is of paramount importance.

Administration of compositions of the present disclosure can help to prevent, retard, and decrease the burden of comorbidity in CKD.

### Administration of Compositions to Treat Gut Diseases and Related Conditions

Without being bound to any theory, Interstitial Cells of Cajal (ICCs) are interstitial cells that are between other cells in the GI tract with an ongoing function in controlling peristalsis. ICCs (main) function may be to create the electrical signals that control the rhythmic smooth muscle contraction in the gut (different regions of the gastrointestinal system have different frequencies of contraction called peristalsis). ICCs may guide the migration of mesenchymal stem cells in the gut (and many other organs).

In the gut, ICCs may produce and/or become activated by klotho, leading to optimized regenerative processes. In Klotho knockout mice, ICCs are diminished, leading to impairment of the peristaltic process, mimicking gut disorders that occur in older humans.

Administration of compositions of the present disclosure can help to prevent, retard, and decrease impairment of the peristaltic process and/or augment, support, and/or optimize the regenerative processes, at least in the gut. These effects may further support gut health and/or prevent, retard, and decrease the occurrence and/or severity of gut diseases and related conditions.

### Therapeutic Treatment of Age-Related Conditions

Investigators have discovered an association between Klotho and biological parameters commonly accepted as indicators of the clinical status in hospitalized older patients. The investigators genotyped the single-nucleotide polymorphisms (SNPs) rs9536314, rs1207568, and rs564481 at the KL locus in 594 hospitalized older patients (65-99 years), consecutively attending a geriatric ward, and tested the association of these KL variants with biological quantitative traits using analyses of covariance and genetic risk score models. Significant associations of rs9536314 with serum levels of hemoglobin, albumin, and high-density lipoprotein cholesterol (HDL-C) as well as significant associations of rs564481 with serum levels of hemoglobin, fasting insulin, and fasting glucose were observed. Gender-segregated analyses confirmed these associations and suggests that the associations of KL genotypes with HDL-C, fasting glucose and fasting insulin levels may be driven by the female gender, while the association with serum levels of hemoglobin may be driven by the male gender. The association of KL genotypes with creatine levels was found in females, while the association with insulin-like growth factor-1 (IGF-1) and lymphocytes count (LC) was found in males. The genetic risk score (GRS) models further confirmed significant associations among KL SNPs and hemoglobin, total cholesterol, and HDL-C. Gender-segregated analyses with the GRS-tagged approach confirmed the associations with HDL-C, fasting glucose, and fasting insulin levels in females, and with hemoglobin and LC in males. The findings suggested that KL locus may influence quantitative traits such as serum levels of lipid, fasting glucose, albumin and hemoglobin in hospitalized older patients, with some gender differences suggested for creatine, IGF-1 levels, and LC, thus being one of the genetic factors possibly contributing to age-related diseases and longevity.

Embodiments of the present disclosure can include administering a composition of the present disclosure to a subject in need thereof (e.g., an individual or patient, optionally elderly and/or suffering from an aging-related condition, low endogenous s-Klotho protein expression, and/or symptoms of age-related condition or decreased longevity). Administration of the composition(s) of the present disclosure can lead to a beneficial increase in blood s-Klotho levels. The administration may reverse or counteract the deleterious effects of the aging-related condition and/or influence, in a positive therapeutic manner, quantitative traits such as serum levels of total cholesterol, HDL-C, fasting glucose, fasting insulin, albumin, creatine, IGF-1, hemoglobin, and lymphocytes count (e.g., in hospitalized and/or elderly patients).

### Prophylactic Composition Administration

In addition to the foregoing, embodiments of the present disclosure can include administering a composition of the present disclosure to an individual or subject in need thereof for prophylactic purposes and/or maintenance of certain health attributes. For example, administration of certain compositions of the present disclosure can help maintain youthfulness in optionally aging patients not yet suffering from a diagnosed aging-related condition. Accordingly, while certain embodiments of the present disclosure can relate to and/or comprise treating a condition in a patient, other embodiments can relate to and/or comprise preventing, inhibiting development of, and/or prophylactically addressing one or more conditions.

### Administration of Exogenous S-Klotho to Treat Genetic Defects

An exemplary embodiment of the present disclosure relates to the administration of exogenous S-Klotho to treat (e.g., correct) known human genetic defects. For example, a 13-year-old girl with familial tumoral calcinosis and a Klotho mutation has been reported. Familial tumoral calcinosis is an autosomal recessive metabolic disease characterized by ectopic calcifications and hyperphosphatemia due to inactivating mutations in FGF23 or GALNT3. FGF23 is a hormone necessary for the renal excretion of phosphate, while GALNT is an enzyme contributing to the maturation and secretion of FGF23. A homozygous mutation in the klotho gene has been identified in the 13-year old girl. Klotho encodes a secreted protein necessary to the transmission of the signal emitted by FGF23 toward its receptors. The administration of exogenous human recombinant S-Klotho constitutes a highly targeted and effective therapy to address the malfunction and symptoms associated with familial tumoral calcinosis.

Compositions and Treatments Including S-Klotho and/or Supplement in Combination with other Component(s)

In at least one embodiment, a composition, medicament, dosage, or therapeutic treatment can include a therapeutic human recombinant soluble alpha Klotho (S-Klotho) protein and a natural supplement composition that increases endogenous Klotho production or one or more component thereof. Klotho, whether endogenous or exogenous (recombinant) can also act in an additive or synergistic matter with other compounds and/or components to influence one or more aspects of human health and well-being. For instance, treatments that include a therapeutic human recombinant soluble alpha Klotho (S-Klotho) protein, and/or natural supplement composition that increases endogenous Klotho production, in combination and/or concurrently with one or more additional active components can benefit human patients. Such treatments can be prophylactic or responsive to any human condition on which the Klotho protein and/or other component(s) can have a therapeutic effect. Such conditions can include, for example, an age-related condition, a clinical (e.g., metabolic) condition, a chronic or acute condition, and so forth. Specific, non-limiting examples of specific conditions are disclosed herein.

S-klotho may exist in the human body along with other blood borne anti-aging compound such as growth/differentiation factor 11 (GDF-11). Accordingly, in certain embodiments, therapeutic S-klotho can be co-administered (e.g., concurrently, sequentially, and/or in combination) with therapeutic GDF-11. Such administration can have additive or synergistic anti-aging or other effects in some embodiments. Likewise, the co-administration S-klotho to human subjects with (neutralizing) antibodies to or suppressor of CCL11 may work in unison to counter aging or other condition (as CCL11 (also known as eotaxin-1) is understood to be a negative regulator of stem cell rejuvenation). S-klotho can also or alternatively be co-administered with other eotaxins, such as eotaxin-2 (CCL24) and/or eotaxin-3 (CCL26).

In some embodiments, S-klotho can be co-administered with suppressors of or antibodies to Transforming Growth Factor β-1 (TGF-β1). S-klotho administration may oppose the action of the TGF-β1 signal pathways involved in an endogenous anti-cellular epithelial-to-mesenchymal transition (anti-EMT) that leads to renal and other tissue fibrosis. Anti-EMT is also relevant in cancer cells where inhibiting EMT may confer on cancer cells the ability to metastasize—this latter process is understood to be opposed by klotho. Accordingly, co-administration of S-klotho and a suppressor of or antibody to Transforming Growth Factor β-1 (TGF-β1) can have synergistic or additive effects.

In some embodiments, S-klotho can be co-administered with antibodies to or suppressors of Insulin Growth Factor-1 (IGF-1). Klotho is understood to be a hormone that inhibits the intracellular insulin/IGF-1 signaling cascade, and this inhibition increases resistance to oxidative stress at the cellular and organismal level in mammals; a mechanism which is considered to be evolutionarily conserved for extending life span. Accordingly, co-administration of S-klotho and a suppressor of or antibody to Insulin Growth Factor-1 (IGF-1) can have synergistic or additive effects.

In some embodiments, S-klotho can be administered in combinations with vitamin D (e.g., Vitamin D3) or 1,25-dihydroxyvitamin D₃[1,25(OH)₂D₃], FGF-15, FGF-19, and/or Klotho β; since numerous studies have revealed a comprehensive regulatory scheme of mineral homeostasis involving the mutually regulated positive/negative feedback actions of Klotho α-K1, FGF23, and 1,25(OH)₂D and/or an analogous regulatory network composed of Klotho β-K1, FGF15/humanFGF19, and bile acids that regulate bile acid/cholesterol metabolism. Such co-administration can have synergistic or additive effects on numerous conditions and/or processes in the body. In some embodiments, S-klotho can be administered in combinations with FGF-21,

In some embodiments, S-klotho can be co-administered with carbonic anhydrase inhibitors, such as acetazolamide, methazolamide, dichlorphenamide, dorzolamide, brinzolamide, and/or topiramate. Such combinatorial administration can be useful in the treatment of ankylosing spondylitis (AS), rheumatoid arthritis (RA), and a variety of other conditions. Various investigations have shown that increased bone resorption is a characteristic of AS and RA and that carbonic anhydrase inhibitors play an antiarthritic role by inhibiting bone resorption. At the bone level, through a different mechanism, S-klotho stimulates bone resorption and phosphate release by acting on TRPV5, which is a recently identified osteoclast function modulator. Increased levels of 1,25(OH)₂D₃ caused by S-Klotho administration can also stimulate osteoclast differentiation and bone resorption and, thereby, phosphate release. Thus, co-administration of S-Klotho and carbonic anhydrase inhibitors can have an additive or synergistic effect, especially in promoting bone health, particularly in AS and RA.

S-Klotho can be administered in combination with one or more disease-modifying antirheumatic drugs (DMARDs)—for the treatment of severe active rheumatoid arthritis.

S-Klotho can be administered in combination with cyclosporine, since cyclosporine decreases klotho mRNA and protein and increases oxidative stress leading to cyclosporine induced-renal injury (CsA). The associated decrease of klotho mRNA and protein and increases oxidative stress can be countered by exogenous co-administration of S-Klotho.

In some embodiments, S-klotho can be co-administered with losartan and/or cyclosporine. Treatment with losartan, an angiotensin II type 1 (AT1) receptor blocker, reversed the decrease in klotho expression seen with cyclosporine. Losartan also produced a concurrent improvement in renal histology (with losartan decreasing the tubulointerstitial fibrosis that is caused by cyclosporine).

In some embodiments, S-klotho can be co-administered with one or more aminoglycosides, such as amikacin, gentamicin, tobramycin, etc. Such treatment can be useful to prevent nephrotoxicity and/or acute kidney injury (AKI) when aminoglycosides are used to treat (gram-negative) pathogen infection, which may greatly expand the use of aminoglycosides to treat infections. The administration of S-Klotho with verapamil and/or diltiazem, which have been used to block AKI, can be therapeutic in the treatment and/or prevention of renal dysfunction from AKI.

In some embodiments, S-klotho can be co-administered with testosterone or androgen receptor (AR) up-regulating compounds. Recent reports indicate no beneficial effect of testosterone treatment in men in regards to personality, psychological well-being, or mood is observed. In addition, the prescription of testosterone supplementation for low-T for cardiovascular health, sexual function, physical function, mood, or cognitive function was considered to be without support from randomized clinical trials. However, testosterone supplementation was consistently found to increase muscle strength but did not have beneficial effects on physical function. S-Klotho administration in combination with testosterone and/or androgen receptor (AR) up-regulating compounds can significantly increase muscle strength and/or physical function in elderly, frail, or low-T men beyond any effect that testosterone or S-Klotho may have alone in these treatment groups.

In some embodiments, S-klotho can be co-administered with estrogen or an estrogen hormone (e.g., estradiol, estriol, estrone, etc.). Such co-administration can improve health indicators in women (e.g., menstrual, menopausal, or menopausal transitioning women) and/or treat infertility, polycystic ovarian disease or disorder, obesity, hormone imbalance and related conditions, and/or other female health conditions.

In some embodiments, S-klotho can be co-administered with one or more nootropics—also called smart drugs or cognitive enhancers. Nootropics drugs, supplements, and/or other substances can improve cognitive function, particularly executive functions, memory, creativity, motivation, task saliency (motivation to perform a task), performance (especially on tedious tasks that require a high degree of effort), and can be useful in treating cognitive or motor function difficulties attributable to disorders such as Alzheimer's disease, Parkinson's disease, Huntington's disease, and ADHD. The most commonly used class of drug that is known to improve some aspect of cognition is stimulants, especially the classes of stimulants that demonstrate cognition-enhancing effects in humans by acting as direct agonists or indirect agonists of dopamine receptor D1, adrenoceptor A2, or both receptors in the prefrontal cortex. Stimulants include, for example: amphetamines (e.g., amphetamine, dextroamphetamine, lisdexamfetamine, etc.), which can benefit a range of cognitive functions (e.g., inhibitory control, episodic memory, working memory, and aspects of attention), especially in individuals with ADHD; dimethylamylamine (DMAA), such as 1,3-dimethylamylamine, which can improve physical performance, alertness, reaction time, etc.; methylphenidate—a substituted phenethylamine that can improve a range of cognitive functions (e.g., working memory, episodic memory, and inhibitory control, aspects of attention, and planning latency); eugeroics (e.g., armodafinil, modafinil, etc.), which can function as wakefulness promoting agents that can increase alertness, particularly in sleep deprived individuals, facilitate reasoning and problem solving, treat narcolepsy, shift work sleep disorder, and daytime sleepiness remaining after sleep apnea treatments, and so forth; xanthines (e.g., caffeine, etc.), which can increase alertness, performance, and/or memory; nicotine, and so forth.

In some embodiments, S-klotho can be co-administered with one or more osteoporosis and/or osteopenia medications, as known in the art. Klotho may play a role in regulating bone mineral density, as the absence of Klotho can lead to reduced bone mineral density in animals. For instance, Klotho knockout mice show reduced bone mineral density over time. Klotho expression can rescue bone defect, such as reduced bone mineral density shown by Klotho knockout mice over time. Epidemiological studies have shown associations between various Klotho gene variants and changes in bone mineral density and prevalence of hand osteoarthritis.

S-Klotho can be administered in combination with one or more anti-cancer treatments and/or preventions, such as a chemotherapeutic. In lung cancers, such as non-small cell lung cancer (NSCLC), for example, S-klotho administration can affect the resistance of lung cancer cells to cisplatin and/or other chemotherapy. In addition, S-klotho can function as a potential tumor suppressor in lung cancer, gastric cancer, pancreatic cancer (adenocarcinoma), and other forms of cancer. S-Klotho can be administered in combination with sorafenib chemotherapy for the treatment of hepatocellular carcinoma (HCC). Overexpression of klotho as well as treatment with soluble klotho protein can reduce hepatoma cell growth in vitro and in vivo. Other cancer types that can be treated with S-klotho co-administration include hepatocellular carcinoma (HCC), central nervous system (CNS) cancers (e.g., brain (e.g., glioma, craniopharyngioma, medulloblastoma and meningioma), spinal cord, and other tumors, lymphoma, etc.), (metastatic) colon cancer, and so forth.

S-Klotho can also be administered in combination with chemotherapeutic(s) to treat chemotherapy-induced frailty in cancer patients. S-Klotho can also be administered to treat cancer-induced frailty in cancer patients following other known treatments.

S-Klotho can be administered in combination with kidney dialysis or other procedures. S-Klotho can be administered to treat frailty in dialysis patients, as frailty is associated with poor outcomes for patients on dialysis.

S-Klotho can be administered in combination with one or more Alzheimer's Disease treatment or preventions, or with brain-derived neurotrophic factor (BDNF), as an adequate amount of BDNF can help to develop and maintain normal neuronal circuits in the brain.

S-Klotho can be administered in combination with one or more molecules that increase the ability of klotho to cross the blood-brain barrier in order to increase the ability of klotho to enter the central nervous system (CNS) to treat or prevent CNS-relates conditions. For example, neither S-Klotho nor BDNF are known to cross the blood-brain barrier. Embodiments of the present disclosure include utilizing blood-brain barrier delivery techniques for the administration of S-klotho and/or S-klotho with BDNF to the CNS to treat Alzheimer's Disease and/or improve cognition in individuals not affected by Alzheimer's Disease.

S-Klotho can be administered in combination with 5′ adenosine monophosphate-activated protein kinase (AMPK) or AMPK activating drugs or ingredients that positively regulate signaling pathways that replenish cellular ATP supplies, including fatty acid oxidation and autophagy; or that negatively regulate ATP-consuming biosynthetic processes including gluconeogenesis, lipid and protein synthesis.

S-Klotho can be administered in combination with one or more anti-diabetic drugs such as insulin, phloridzin or the antioxidant tiron and these combination treatments may have merit in the prevention of renal damage from oxidative stress produced in diabetic disorders. The co-administration of S-Klotho with other antidiabetic drugs for type 1 diabetes can protect β-cells by inhibiting β-cell apoptosis through activation of the integrin β1-FAK/Akt pathway, leading to inhibition of caspase 3 cleavage.

S-Klotho can be administered in combination with one or more type 2 anti-diabetes drugs such as metformin for improving glycemic control and vascular function in overweight and obese diabetic subjects.

S-Klotho can be administered in combination with one or more blood pressure medications, calcium regulators, or treatments or preventions of chronic kidney disease (CKD). For example, soft-tissue calcification is a prominent feature in CKD and Klotho can ameliorate vascular calcification by enhancing phosphaturia, preserving glomerular filtration, and directly inhibiting phosphate uptake by vascular smooth muscle.

S-Klotho can be administered in combination with TM5441 or other inhibitors of PAI-1 (plasminogen activator 1), since it is thought that PAI-1 inhibition or deficiency retards the development of senescence and protects organ structure and function while prolonging the lifespan of Klotho-deficient (kl/kl) mice.

S-Klotho can be administered in combination with sirtuinl (SIRT1) or SIRT1-activating compounds (STACs) such as resveratrol. SIRT1, a type III protein deacetylase, is thought to be a novel anti-aging protein involved in regulation of cellular senescence/aging and inflammation. SIRT1 level and activity are decreased during lung inflammation caused by oxidative stress. The mechanism of SIRT1-mediated protection against inflammation is associated with the regulation of inflammation, premature senescence, telomere attrition, senescence associated secretory phenotype, and DNA damage response. A variety of dietary polyphenols and pharmacological activators are shown to regulate SIRT1 so as to intervene the progression of type 2 diabetes, cancer, cardiovascular diseases, and chronic obstructive pulmonary disease associated with inflammation. Thus some or all of the health benefits of SIRT-1 may be augmented by co-administration of SIRT1 and/or SIRTI-activating compounds given with S-Klotho.

S-Klotho can be administered in combination with one or more Human Cells, Tissues, and Cellular and Tissue-Based Products (HCT/Ps), as recognized by the FDA. Such products can include, for example, one or more of bone (including demineralized bone, ligaments, tendons, fascia, cartilage, ocular tissues (corneas & sclera), skin, vascular grafts (veins and/or arteries), optionally (or other than) preserved umbilical cord veins, pericardium, amniotic membrane (when used alone (—without added cells—) for ocular repair), dura mater, heart valve allografts, hematopoietic stem cells derived from peripheral or umbilical cord blood, semen, oocytes, or embryos. In at least one embodiment, the HCT/P can be or comprise one or more stem cells. Stem cell treatment for damaged bodily tissues and organs continues to grow in popularity. Administration of therapeutic, recombinant Klotho protein in combination with stem cells provided surprising, unexpected, and even synergistic outcomes for subjects in need thereof.

Embodiments of the present disclosure further include a combination product, comprising a therapeutic, recombinant Klotho protein in combination with human stem cells. The composition can also include a pharmaceutically-acceptable carrier as described herein. Such compositions can comprise or be classified as regenerative medicines, to treat, modify, reverse, or cure a serious or life-threatening disease or condition, as recognized by the FDA. Preliminary clinical evidence indicates that the composition (drug) has the potential to address unmet medical needs for such disease or condition.

Illustratively, stem cells can be or comprise mesenchymal stem cells (MSCs), such as from human umbilical cords or placentas. In at least one embodiment, the composition comprising huMSCs and a therapeutic, recombinant Klotho protein of the present disclosure attenuated the inflammatory and oxidative stress responses occurring in AKI, and/or reduced the expression of senescence-related proteins and microRNAs.

S-Klotho can be administered in combination with one or more senescence inhibitors. For instance, Klotho protein can be combined with Pin1-FOXM1 and/or other senescence inhibitors to enhance outcomes in patients receiving such treatment.

S-Klotho can be administered in combination with one or more of the following: Klotho stimulators (e.g., Vit. D, Losartan, Testosterone), GDF-11, Trichostatin A anti-fungal (e.g., GDF- 11 Stimulator), TIMP-2, CCL- 11 Inhibitor/antibodies, D as atinib, Nicotinamide Riboside (e.g., NAD+), nicotinamide mononucleotide (NMN) (e.g., NDA+), AMPK Stimulators (e.g., Resveratrol, Aspirin, Salicylate, phytochemicals, DR), C60 Fullerene, Rapamycin, FGF inhibitor, Senolytics drugs/compounds such as FOXO4-p53 interfering peptides (e.g. FOXO4-DRI), inhibitors of the anti-apoptotic proteins BCL-2 and BCL-xL.

Any of the foregoing or other treatments or co-administrations can have additive or synergistic effects over those of any of the treatments alone. For instance, co-administration of a Klotho protein with one or more of the foregoing can result in treatment outcomes greater than the sum of the individual outcomes of administering the components alone at similar concentrations. In addition, synergistic effects can include treatment outcomes similar to those of the individual outcomes of administering the components alone, but at lower concentrations. Synergistic effects can also include increasing the maximum effective dose of one or more of the components, reducing toxicity of one or more of the components, or any other beneficial result that is more than a mere additive effect of the individual treatment outcomes. In addition, additive effects of the individual treatment outcomes can comprise one or more synergistic effects. Such additive/synergistic effects may not be predicted or expected given the nature and understanding of the individual components.

As used herein, a combination treatment or co-administration can include treatment or administration of a combination product, composition, or formulation comprising a Klotho protein and one or more additional active ingredients. The one or more additional active ingredients can be selected from among the components, drugs, substances, treatment compositions, etc. described herein or others known in the art. For instance, the Klotho protein and one or more additional active ingredients can be co-formulated into an injectable (e.g., intramuscular, intravenous, etc.), ingestible, transdermal, inhalable, topical, or other formulation.

Alternatively, a combination treatment or co-administration can include treatment or administration of a Klotho protein and one or more additional active ingredients, but without the Klotho protein and one or more additional active ingredients being combined or formulated into a combination product, composition, or formulation. For instance, the Klotho protein and one or more additional active ingredients can each comprise or be in a separate injectable (e.g., intramuscular, intravenous, etc.), ingestible, transdermal, inhalable, topical, or other formulation.

It will also be appreciated that co-administration can comprise simultaneous administration of two or more components or distinct administrations of two or more components, the distinct administrations preferably being separated by a period of time. The period of time can be very small, in some embodiments. For instance, a second component of the Klotho protein and one or more additional active ingredients can be administered (e.g., injected) substantially, immediately following administration of a first component of the Klotho protein and one or more additional active ingredients. Alternatively, the first and second administrations can be separated by a time period of 1-60 seconds, 1-60 minutes, 1-24 hours, 1-7 days, 1-4 weeks, 1-12 months, and so forth, or any value or range of values therebetween. Similarly, simultaneous administration can include overlapping administration timeframes for the two or more components.

### Therapeutic Treatment of Hyperphosphatemic Familial Tumoral Calcinosis (HFTC)

In affected human individuals, HFTC is caused by a histidine (H) to arginine (R) mutation at amino acid (AA) position 193 of S-Klotho—rs121908423. Without being bound to any theory, it is thought that the H193R mutation in HFTC individuals impairs the ability of S-Klotho to form a ternary complex with FGF23 and FGFR1c, which impairs KL-dependent FGF23 signaling. As a result, affected subjects present a severe metabolic disorder that manifests with hyperphosphatemia and massive calcium deposits in the skin and subcutaneous tissues. Some patients manifest recurrent, transient, painful swellings of the long bones associated with the radiographic findings of periosteal reaction and cortical hyperostosis and absence of skin involvement.

An embodiment of the present disclosure includes a S-Klotho protein having H193. The H193 protein can be produced or expressed in the context of any protein construct described herein. For instance, the H193 variant can be produced or expressed in the context of S-Klotho, isoform 1 or 2, 1-981, 29-981, 34-981, 36-981, 131-981, 1-549, 29-549, 34-549, 36-549, 131-549, and so forth, with or without a tag (e.g., Fc tag, TEV-Twin-Strep tag, TEV sequence, etc.). Accordingly, the nucleic acid construct or cDNA from which the protein is expressed can be of corresponding configuration.

Embodiments can include producing a H193 heterozygous or homozygous variant construct, transferring (e.g., via transfection) the resulting construct, which encodes the S-Klotho H193 protein, into an appropriate expression system (e.g., CHO cells), and/or transiently expressing the S-Klotho H193 protein. The H193 Klotho protein may also be expressed at higher levels than the R193 (or H193R) protein. Embodiments can include purifying (and optionally quality-control testing) the expressed protein for therapeutic administration. Embodiments can include administering a therapeutic or therapeutically-effective amount of the S-Klotho H193 protein to a subject in need thereof (e.g., HFTC individuals, individual diagnosed with HFTC, or patient harboring the H193R (rs121908423) or other variant). Alternatively, the subject may be of wild-type of other mutant or variant type. Administration of the recombinant S-Klotho H193 protein can lead to a beneficial increase in blood S-Klotho levels. The administration may reverse or counteract the deleterious effects of the R193 mutation that is transcribed and circulated in HFTC individuals as a result of the H-to-R193 point mutation found in the human klotho gene in individuals affected with HFTC. Accordingly, the circulating concentration of S-Klotho H193 may help to counter the effects observed in H193R or HFTC individuals.

Therapeutic Treatment in CC Genotype Patients with End-Stage Renal Disease (ESRD)

Approximately 350,000 patients with end-stage renal disease (ESRD) suffer exceptionally high mortality rates in their first year of chronic hemodialysis. Both vitamin D and fibroblast growth factor (FGF)-23 levels correlate with survival in these patients. Without being bound to any theory, Klotho is a protein in the vitamin D/FGF-23 signaling pathway that has been linked with accelerated aging and early mortality in animal models. It has been hypothesized that genetic variation in the Klotho gene may be associated with survival in subjects with ESRD. Investigators tested the association between 12 single nucleotide polymorphisms (SNPs) in the Klotho gene and mortality in a cohort of ESRD patients during their first year on hemodialysis (n=1307 white and Asian). A significant association was discovered between the CC genotype of one tag SNP, rs577912 (a common HapMap variant with a minor allele frequency [MAF]>0.05 within the Klotho gene sequence located in intron 1), and increased risk for 1-yr mortality (RR, 1.76; 95% CI, 1.19-2.59; p=0.003). This effect in individuals with the CC genotype was even more marked among patients who were not treated with activated vitamin D supplementation (HR, 2.51; 95% CI, 1.18-5.34; p=0.005). In lymphoblastoid cell lines derived from HapMap subjects, the CC genotype was associated with a 16-21% lower Klotho expression compared with the AA or AC genotypes. However, none of the rs577912 SNPs nucleotide change described above result in an amino acid change in the Klotho protein. Accordingly, this functional SNP (rs577912) may quantitatively affect Klotho gene expression at the mRNA level.

An embodiment of the present disclosure includes a S-Klotho protein expressed from the AA or AC genotype. The protein can be produced or expressed in the context of any protein construct described herein. For instance, the protein can be produced or expressed in the context of S-Klotho, isoform 1 or 2. 1-981, 29-981, 34-981, 36-981, 131-981, 1-549, 29-549, 34-549, 36-549, 131-549, and so forth, with or without a tag (e.g., Fc tag, TEV-Twin-Strep tag, TEV sequence, etc.). Accordingly, the nucleic acid construct or cDNA from which the protein is expressed can be of corresponding length.

Embodiments can include producing a AA or AC heterozygous or homozygous construct, transferring (e.g., via transfection) the resulting construct, which encodes the S-Klotho protein, into an appropriate expression system (e.g., CHO cells), and/or transiently expressing the S-Klotho protein. Klotho protein expressed in AA or AC heterozygous or homozygous cells may be expressed at higher levels than in CC cells. Embodiments can include purifying (and optionally quality-control testing) the expressed protein for therapeutic administration. Embodiments can include administering a therapeutic or therapeutically-effective amount of the S-Klotho protein to a subject in need thereof (e.g., an individual or patient harboring the CC mutation at the one tag SNP, rs577912, having low endogenous S-Klotho protein expression, and/or with end-stage renal disease (ESRD)). Alternatively, the subject may be of wild-type of other mutant or variant type. Administration of the recombinant S-Klotho protein can lead to a beneficial increase in blood S-Klotho levels. The administration may reverse or counteract the deleterious effects of the CC mutation that is transcribed and circulated in individuals as a result of the point mutation(s) found in the human klotho gene in affected individuals. Accordingly, the circulating concentration of S-Klotho may help to counter the effects observed in CC individuals, especially those with end-stage renal disease (ESRD), namely mortality in the first year in ESRD patients undergoing chronic hemodialysis.

### Therapeutic Treatment of Radiographic Hand Osteoarthritis (OA) and Osteophyte

Osteoarthritis (OA) is a common complex disease with strong heritable components. Investigators studied the association between four putatively functional genetic variants in klotho gene and hand OA in a large female Caucasian population. The investigators found significant association between SNP G-395A and the presence/absence of radiographic hand OA and osteophyte formation. Allele G significantly increased the risk for radiographic hand OA and osteophytes with odds ratios (ORs) of 1.44 (P=0.008, 95% confidence interval (CI) 1.09-1.91) and 1.36 (P=0.006, 95% CI 1.09-1.70), respectively. From logistic regression modelling, genotype GG showed more than three-fold increased risk for both radiographic hand OA (OR=3.10, 95% CI 1.10-8.76) and osteophyte (OR=3.10, 95% CI 1.10-8.75) when compared to genotype AA. After adjustment for age, ORs for genotype GG further increased to 4.39 (P=0.006, 95% CI 1.51-12.74) for radiographic hand OA and to 4.47 (P=0.005, 95% CI 1.56-12.77) for osteophytes. The investigators also suggested that one variant (SNP G-395A) in the klotho gene is associated with the susceptibility of hand OA and appears to act through osteophyte formation rather than cartilage damage.

An embodiment of the present disclosure includes a S-Klotho protein expressed from a construct having SNP G395A. The resulting protein can be produced or expressed in the context of any protein construct described herein. For instance, the A395 variant can be produced or expressed in the context of S-Klotho, isoform 1 or 2, 1-981, 29-981, 34-981, 36-981, 131-981, 1-549, 29-549, 34-549, 36-549, 131-549, and so forth, with or without a tag (e.g., Fc tag, TEV-Twin-Strep tag, TEV sequence, etc.). Accordingly, the nucleic acid construct or cDNA from which the protein is expressed can be of corresponding length.

Embodiments can include producing a G395A heterozygous or homozygous construct, transferring (e.g., via transfection) the resulting construct, which encodes the S-Klotho protein, into an appropriate expression system (e.g., CHO cells), and/or transiently expressing the S-Klotho protein. Klotho protein expressed in A395 heterozygous or homozygous cells may be expressed at higher levels than in G396 cells. Embodiments can include purifying (and optionally quality-control testing) the expressed protein for therapeutic administration. Embodiments can include administering a therapeutic or therapeutically-effective amount of the S-Klotho protein to a subject in need thereof (e.g., an individual or patient harboring the G395 SNP and/or (at risk of developing) radiographic hand osteoarthritis (OA) and/or osteophytes). Alternatively, the subject may be of wild-type of other mutant or variant type. Administration of the recombinant S-Klotho protein can lead to a beneficial increase in blood S-Klotho levels. The administration may reverse or counteract the deleterious effects of the G395 SNP that is transcribed and circulated in affected individuals. Accordingly, the circulating concentration of S-Klotho may help to counter the effects observed in G395 individuals, especially those at risk of developing radiographic hand osteoarthritis (OA) and/or osteophytes. Administration of the G-395A S-Klotho protein may, therefore, decrease the risk of radiographic hand osteoarthritis (OA) and osteophyte formation in patients (e.g., patients harboring the G395 SNP.

### Therapeutic Treatment of Metabolic Syndrome

The risk and/or incidence of metabolic syndrome (MetS), a cluster of cardiometabolic risk factors including abdominal obesity, hyperglycemia, dyslipidemia and hypertension, increases with age. In elderly adults, MetS not only increases the risk of cardiovascular diseases and type 2 diabetes, but also is associated with cognitive decline and disability. Current evidence suggests that MetS is partly heritable, and genetic factors play a greater role than environment factors on the incidence of MetS. Investigators discovered an association between the G-395A polymorphism and metabolic syndrome (MetS) among a population of Chinese nonagenarians and centenarians. Subjects were from the Project of Longevity and Aging in Dujiangyan (PLAD). The genotyping of G-395A (rs1207568) in the promoter region of the Klotho gene was performed using the TaqMan allelic discrimination assay. MetS was diagnosed according to the International Diabetes Federation criteria. Included were 695 subjects aged 93.5±3.2 years. G and A allele frequencies were 0.852 and 0.148, respectively. In the whole population, the frequency of MetS was 10.8% and 5.9% in the GG and GA+AA genotype group, respectively (p=0.004). The −395A allele carriers had significantly lower risk of MetS in the whole population (odds ratio [OR] 0.50, 95% confidential interval [CI] 0.25 to 0.98) and in women (OR 0.51, 95% CI 0.24 to 0.97), but not in men (OR 0.42, 95% CI 0.05 to 3.85). In the whole population and women, the relationship between the Klotho G-395A SNP and MetS might be due to its influence on high blood pressure (OR 0.48, 95% CI 0.34 to 0.67; OR 0.47, 95% CI 0.31 to 0.71, respectively) and hypertriglyceridemia (OR 0.66, 95% CI 0.39 to 0.95; OR 0.54, 95% CI 0.31 to 0.98, respectively). In men, this relationship might be due to its influence on high blood pressure (OR 0.47, 95% CI 0.25 to 0.90) and low HDL-C (OR 0.69, 95% CI 0.27 to 0.93).

Investigators concluded that the -395A allele carriers of the Klotho gene were correlated with lower risk of MetS among Chinese nonagenarians and centenarians, especially in women.

An embodiment of the present disclosure includes a S-Klotho protein expressed from a construct having the −395A allele. The resulting protein can be produced or expressed in the context of any protein construct described herein. For instance, the A395 allele can be produced or expressed in the context of S-Klotho, isoform 1 or 2, 1-981, 29-981, 34-981, 36-981, 131-981, 1-549, 29-549, 34-549, 36-549, 131-549, and so forth, with or without a tag (e.g., Fc tag, TEV-Twin-Strep tag, TEV sequence, etc.). Accordingly, the nucleic acid construct or cDNA from which the protein is expressed can be of corresponding length.

Embodiments can include producing a -395A heterozygous or homozygous construct, transferring (e.g., via transfection) the resulting construct, which encodes the S-Klotho protein, into an appropriate expression system (e.g., CHO cells), and/or transiently expressing the S-Klotho protein. Klotho protein expressed in A395 heterozygous or homozygous cells may be expressed at higher levels than in G396 cells. Embodiments can include purifying (and optionally quality-control testing) the expressed protein for therapeutic administration. Embodiments can include administering a therapeutic or therapeutically-effective amount of the S-Klotho protein to a subject in need thereof (e.g., an individual or patient harboring the G395 SNP and/or (at risk of developing) metabolic syndrome (MetS). Alternatively, the subject may be of wild-type of other mutant or variant type. Administration of the recombinant S-Klotho protein can lead to a beneficial increase in blood S-Klotho levels. The administration may reverse or counteract the deleterious effects of the G395 SNP that is transcribed and circulated in affected individuals. Accordingly, the circulating concentration of S-Klotho may help to counter the effects observed in G395 individuals, especially those at risk of developing metabolic syndrome (MetS). Administration of the G-395A S-Klotho protein may, therefore, decrease the risk of metabolic syndrome (MetS) in patients (e.g., patients harboring the G395 SNP, in elderly human, and/or in women).

### Therapeutic Treatment of Cancer

S-Klotho is thought to inhibit basal Wnt signaling activity, thereby functioning as a tumor suppressor for colorectal cancer (CRC). In addition, klotho gene variants associated with lifespan differences may repress butyrate-mediated Wnt hyperactivation, and thus increase the risk of CRC. It has been hypothesized that in this manner, the type of klotho variant present, and its relative expression, can interact with levels of butyrate derived from diet to modify CRC risk. Moreover, mTOR signaling has also been linked to human aging, and crosstalk between Wnt and mTOR signaling may influence colonic tumorigenesis

The KL-VS variant or other construct can serve as a vehicle for investigating which SNPs (e.g., within KL-VS) are responsible for influencing S-Klotho to produce a decrease in basal Wnt signaling and/or the suppression of butyrate-mediated Wnt hyperactivation—the latter Wnt-related activities which have been associated with S-Klotho tumor suppression. Embodiments include modifying the appropriate amino acids (e.g., in the KL-VS stretch of S-Klotho) shown to influence the tumor suppressing action of the KL-VS variant. An embodiment of the present disclosure includes a recombinant S-Klotho protein having one or more amino acid alterations in the KL-VS stretch of 6 SNPs. The proteins can be produced and/or expressed in the context of any protein construct described herein. For instance, the proteins can be produced or expressed in the context of S-Klotho, isoform 1 or 2, 1-981, 29-981, 34-981, 36-981, 131-981, 1-549, 29-549, 34-549, 36-549, 131-549, and so forth, with or without a tag (e.g., Fc tag, TEV-Twin-Strep tag, TEV sequence, etc.). Accordingly, the nucleic acid construct or cDNA from which the protein is expressed can be of corresponding length.

Embodiments can include producing a heterozygous or homozygous variant construct, transferring (e.g., via transfection) the resulting construct, which encodes the S-Klotho protein, into an appropriate expression system (e.g., CHO cells), and/or transiently expressing the S-Klotho protein. The Klotho protein may be expressed at higher levels than the other Klotho proteins, including wild-type. Embodiments can include purifying (and optionally quality-control testing) the expressed protein for therapeutic administration. Embodiments can include administering a therapeutic or therapeutically-effective amount of the S-Klotho protein to a subject in need thereof (e.g., a patient with or at risk of developing colorectal cancer (CRC) or another tumor). The subject may, for example, harbor or express a Klotho variant with decreased Wnt inhibition activity. Alternatively, the subject may be of wild-type of other mutant or variant type. Administration of the recombinant S-Klotho protein can lead to a beneficial increase in blood S-Klotho levels.

### Therapeutic Treatment of Non-Human Mammals

In addition to the foregoing, one or more non-human, mammalian Klotho proteins or protein sequences (or portion(s) thereof) can be useful in implementing certain embodiments of the present disclosure. SEQ ID NOS: 121-124 present DNA sequences for encoding various non-human mammalian Klotho proteins. SEQ ID NOS: 111-120 present amino acid sequences for various non-human mammals. These Klotho protein sequences (or portion(s) thereof) can be expressed, purified, formulated into compositions, and/or administered to animals in a manner similar to that described above. Therapeutics administration of recombinant Klotho (fusion) proteins can be effective in treating the non-human mammal version of the medical and other conditions outlined herein. In particular, embodiments of the present disclosure can include veterinary treatment method, proteins, and compositions, as will be understood by those skilled in the art.

### EXAMPLES

### Example 1

Table 48 illustrates the results from transient expression and purification of the recited Klotho variants in HEK and/or CHO cell lines. In the results provided in Table 48, below, the following abbreviated protocols were followed.

For Fc fusion proteins, protein expression vectors transfected into HEK293.sus or CHO using standard methods. Briefly, cells were grown for 7 days and harvested. Cell counts are given in notes section. Supernatant pH was adjusted with 1 M Hepes pH 7.4 and sodium azide added. KanCap A resin was used to capture proteins. Resin was washed with PBS. Resin was washed with PBS plus 1 M NaCl. Resin was washed with PBS. Proteins were eluted with 50 mM Citrate pH 3.5, 100 mM NaCl. Proteins were immediately neutralized with 1 M Tris pH 8, 0.5M Arginine. SDS PAGE gel samples were removed at this stage. Proteins were buffer exchanged into PBS. Protein was quantified by OD280, quantity and concentration was determined using calculated extinction coefficient. Reduced and non-reduced SDS-PAGE (Biorad criterion Tris/Glycine/SDS, 4-20%) were used to determine purity and approximate molecular mass. Aggregation status was determined by HPLC, with detection at 280 nm using a Sepax Zenix-C SEC-300, 3 um, 300Å, 4.6*150 mm size exclusion column and PBS running buffer. Proteins were shipped as aliquots after filter sterilization, snap frozen in liquid nitrogen. Loss of protein was observed for some Fc proteins during desalting into PBS, as determined from samples run on SDS-PAGE before and after purification. These proteins are expressed but from the HPLC, problems arose during desalting or assay on the silica HPLC column in PBS. Accordingly, buffer selection was optimized to reduce protein loss.

For Strep tagged proteins, protein expression vectors transfected into HEK293.sus or CHO using standard methods. Briefly, cells were grown for 7 days and harvested. Supernatant pH was adjusted with 1 M Hepes pH 7.4 and sodium azide added. BioLock biotin sequestration reagent was added. StrepTactin superflow resin was used to capture proteins. Resin was washed with 100 mM Tris pH 8, 150 mM NaCl, 1 mM EDTA. Proteins were eluted with 100 mM Tris pH 8, 150 mM NaCl, 1 mM EDTA plus 2.5 mM Desthiobitin. Protein was quantified by OD280, quantity and concentration was determined using calculated extinction coefficient. Reduced and non-reduced SDS-PAGE (Biorad criterion Tris/Glycine/SDS, 4-20%) were used to determine purity and approximate molecular mass. Aggregation status was determined by HPLC, with detection at 280nm using a Sepax Zenix-C SEC-300, 3 um, 300Å, 4.6*150 mm size exclusion column and PBS running buffer. Proteins were shipped as aliquots after filter sterilization, snap frozen in liquid nitrogen. It should be noted that for the Strep tagged proteins, samples were assayed in elution buffer. This elution buffer is very ‘neutral’ and is not detrimental to proteins. Also, the running buffer for the SEC column was PBS, so the proteins underwent a buffer exchange during assay. Absorbance at times above 7 minutes are small molecules.

TABLE 48

| | OD280 | Conc. | Extinction | Molecular | Titer |
|---|---|---|---|---|---|
| Protein Name | nm | (mg/mL) | Coefficient | Weight (Da) | (mg/L) |
| Native Klotho ECD (34-981)- | 2.01 | 0.95 | 246780 | 116615 | 6.18 |
| nativeSS-strep-HEK | | | | | |
| Native Klotho ECD (34-981)- | 1.73 | 0.79 | 246780 | 113119 | 5.16 |
| nonNativeSS-strep-HEK | | | | | |
| Klotho ECD variant-1(36-981)- | 0.89 | 0.41 | 246780 | 112893 | 2.64 |
| nonNativeSS-strep-HEK | | | | | |
| Native secreted/Isoform 2 (34- | 1.33 | 0.63 | 140510 | 66255 | 4.07 |
| 549)-nativestrep-HEK | | | | | |
| Native secreted/Isoform 2 (34- | 2.84 | 1.27 | 140510 | 62760 | 8.24 |
| 549)-SS-strep-HEK | | | | | |
| Secreted variant (36-549)- | 1.40 | 0.62 | 140510 | 62534 | 4.04 |
| nonNativeSS-strep-HEK | | | | | |
| Klotho ECD variant-2 (131-981)- | 1.12 | 0.52 | 221800 | 103079 | 3.40 |
| nonNativeSS-strep-HEK | | | | | |
| Native Klotho ECD (34-981)- | 0.02 | 0.01 | 270075 | 138668 | 0.27 |
| nativeSS-Fc-HEK | | | | | |
| Native Klotho ECD (34-981)- | 0.01 | 0.00 | 270075 | 135172 | 0.12 |
| nonNativeSS-Fc-HEK | | | | | |
| Klotho ECD variant-1(36-981)- | −0.04 | −0.02 | 270075 | 134946 | −0.48 |
| nonNativeSS-Fc-HEK | | | | | |
| Native secreted/Isoform 2 (34- | 0.03 | 0.02 | 163805 | 88308 | 0.43 |
| 549)-nativeSS-Fc-HEK | | | | | |
| Native secreted/Isoform 2 | 0.05 | 0.03 | 163805 | 84813 | 0.76 |
| (34-549)-nonNativeSS-Fc-HEK | | | | | |
| Secreted variant (36-549)- | −0.01 | −0.00 | 163805 | 84587 | −0.08 |
| nonNativeSS-Fc-HEK | | | | | |
| Klotho ECD variant-2 (131-981)- | −0.01 | −0.01 | 245095 | 125132 | −0.15 |
| nonNativeSS-Fc-HEK | | | | | |
| Native Klotho ECD (34-981)- | 0.42 | 0.20 | 246780 | 116615 | 1.97 |
| nativeSS-strep-CHO | | | | | |
| Native Klotho ECD (34-981)- | 0.28 | 0.13 | 246780 | 113119 | 1.30 |
| nonNativeSS-strep-CHO | | | | | |
| Klotho ECD variant-1(36-981)- | 0.15 | 0.07 | 246780 | 112893 | 0.68 |
| nonNativeSS-strep-CHO | | | | | |
| Native secreted/Isoform 2 | 0.26 | 0.12 | 140510 | 66255 | 1.24 |
| (34-549)-nativeSS-strep-CHO | | | | | |
| Native secreted/Isoform 2 | 0.36 | 0.16 | 140510 | 62760 | 1.61 |
| (34-549)-nonNativeSS-strep-CHO | | | | | |
| Secreted variant (36-549)- | 0.22 | 0.10 | 140510 | 62534 | 0.98 |
| nonNativeSS-strep-CHO | | | | | |
| Klotho ECD variant-2 (131-981)- | 0.19 | 0.09 | 221800 | 103079 | 0.86 |
| nonNativeSS-strep-CHO | | | | | |
| Native secreted/Isoform 2 (34- | 0.12 | 0.06 | 163805 | 88308 | 1.54 |
| 549)-nativeSS-Fc-CHO | | | | | |
| Native secreted/Isoform 2 (34- | 0.16 | 0.08 | 163805 | 84813 | 2.05 |
| 549)-nonNativeSS-Fc-CHO | | | | | |

In certain embodiments, EDTA and/or other metal chelating agent(s) were removed from (or not included in) one or more (e.g., all) of the purification steps and/or buffers. In some embodiments, EDTA and/or other metal chelating agent(s) may contribute to or cause deactivation of the Klotho protein.

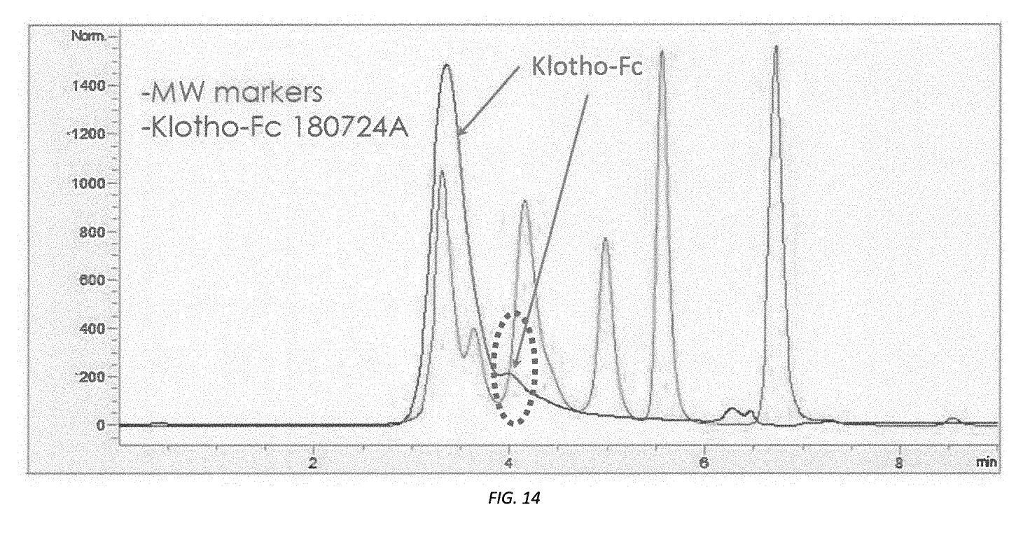

In a follow up study, transiently-expressed Fc-Klotho proteins were purified using Aquity UPLC BEH200 SEC analytical column: 1.7 um, 4.6×150 mm; Mobile Phase: 100 mM Sodium Phosphate, 200 mM Sodium Chloride, pH 6.8; Flow Rate: 0.35 mL/min; Run Time: 9 minutes; Detection: A280, MALS & RI detector; Ambient sample compartment, 30C column compartment; 100 uL of sample injected (37 ug). FIG. 14 illustrates a chromatograph of Klotho-Fc protein. The majority of Fc Klotho protein appears to overlay with the first peak of MW markers near the void volume. This corresponds to a very high molecular weight seen for the main peak (in mega daltons), which is much higher than native Klotho. By SDS-PAGE, non-reducing, the peak (fractions) contains protein the size of a monomer, suggesting non-covalent association in solution. A minor shoulder is observed on the tail of the main Klotho peak (marked with dashed oval); this is <1% of total Klotho peak area with a MW larger than IgG by elution time on the SEC column relative to the MW markers and ˜700 kDa by light scattering (the LS signal is very low and has tailing of the aggregates, so might be over-estimated). This peak corresponds to a multimer, perhaps a dimer or tetramer.

Various alternative purification schemes, including different columns, buffers, conditions, etc. were used to address the issue with protein aggregation and multimer formation. However, in at least one embodiment, a multimer (dimeric, tetrameric, etc.) Klotho protein can be purified.

### Example 2

Stable expression of the (human) Klotho derived proteins represented in SEQ ID NO: 52 (N′-human alpha Klotho 34-981 isoform 1_(G₄S)₂ linker_human IgG1 Fc-C′), SEQ ID NO: 54 (N′ -human alpha Klotho 34-549 isoform 2_(G₄S)₂ linker_human IgG1 Fc-C′), and SEQ ID NO: 66 (N′ -human alpha Klotho 34-981 isoform 1_Twin-Strep cleavage site residues) was performed in CHO cells. Each of the three Klotho protein constructs was expressed with a non-naturally-occurring N-terminal signal sequence, which was then cleaved during the stable expression/purification process to yield the N-terminal (Klotho protein) amino acid. C-terminal to the Klotho protein sequence in SEQ ID NOS: 52 and 54 is a GS linker (GGGGSGGGGS) and an Fc-fusion tag (human IgG1 Fc domain), which are (or appear to be) retained in the expressed protein. C-terminal to the Klotho protein sequence in SEQ ID NOS: 52 is a TEV-Twin-Strep tag (with GS linker) (GGENLYFQ/SSAWSHPQFEK-GGGSGGGSGGS-SAWSHPQFEK), which is cleaved following the glutamine 8 (Q8) of the TEV-Twin-Strep tag (i.e., GGENLYFQ-), to release the Twin-Strep tag portion (SSAWSHPQFEK-GGGSGGGSGGS-SAWSHPQFEK) and retain at least a portion of the TEV (protease recognition or consensus) sequence (GGENLYFQ-C′).

The coding sequences were codon optimized using DNA2.0 proprietary algorithms. The genes were synthesized and Pd3600 transposon backbone-based expression constructs were assembled.

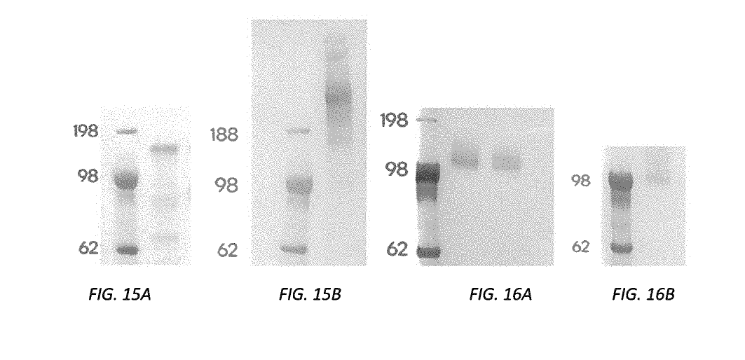

A GS knockout CHOK1 derivative host cell line was used to express the human Klotho variants. The cells have been co-transfected, with Leap-In transposon based expression plasmids coding for the three different hKlotho designs and Leap-In transposases. The transfections were performed by electroporation using a Neon apparatus (Thermo). Transfected cells were selected under glutamine free conditions and the resulting stable pools were cryopreserved. Cells from the established stable pools were inoculated into shake flasks and fed batch production studies were conducted using small scale established feeding and cell culture conditions. Clarified harvest samples were collected and submitted for Western blot analysis using anti-Fc antibody—(SEQ ID NOS: 52 and 54) or Strep-Tactin—(SEQ ID NO: 66) HRP conjugate based detection. Example Western blots are shown in FIGS. 15A-16B.

Stable pool 291645 expressing the FL-ECD (34-981)-Fc Klotho derivative (SEQ ID NO: 52) produces the protein predominantly at the expected size (see FIG. 15A). The predicted size of the full-length extracellular domain-Fc fusion protein monomer is 139 kDa. The predominant anti-Fc positive band is at the predicted ˜140kDa size (arrow) indicating that expression construct 291645 expresses the correct size fusion protein. The protein dimerizes under native and non-reducing conditions (see FIG. 15B). Under non-reducing conditions the full-length extracellular domain-Fc fusion protein may form homodimers. The size of the predominant band detected by anti Fc antibody on a non-reducing Western blot (arrow) is consistent with the formation of the predicted homodimers.

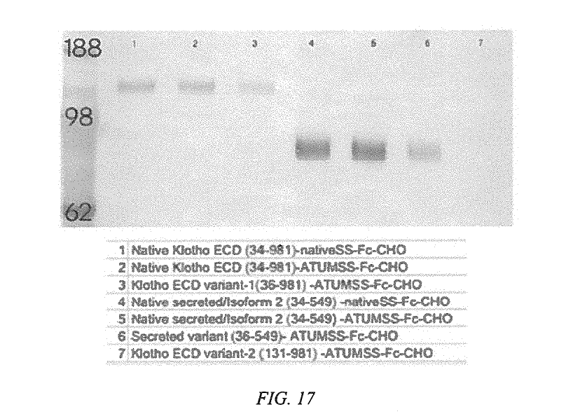

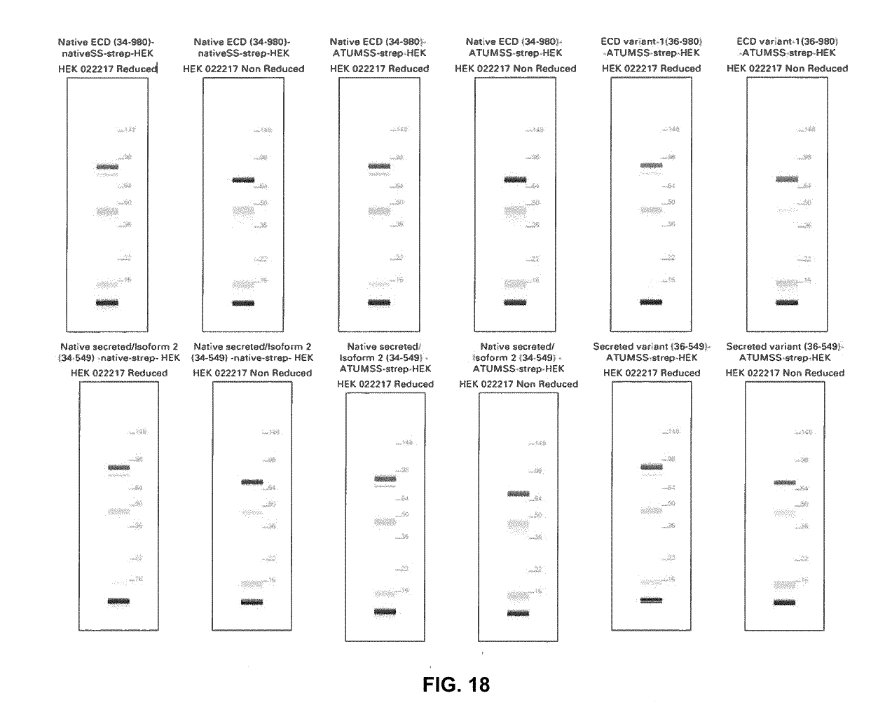

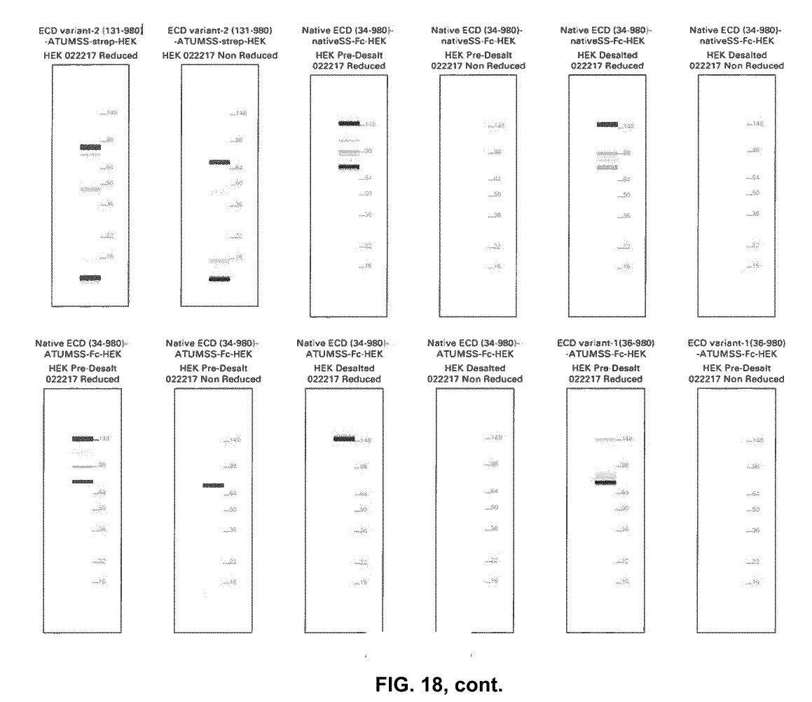

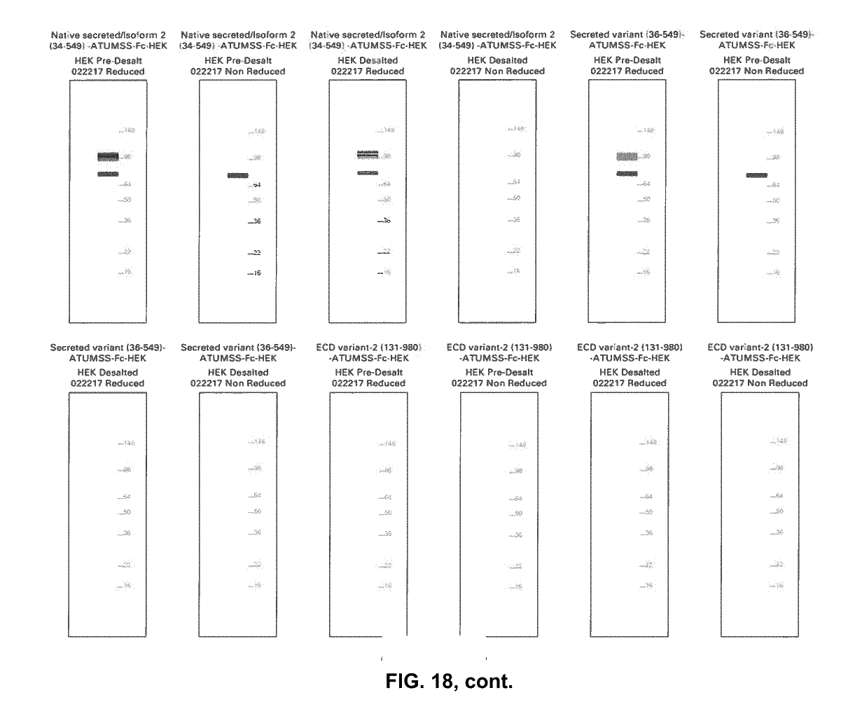

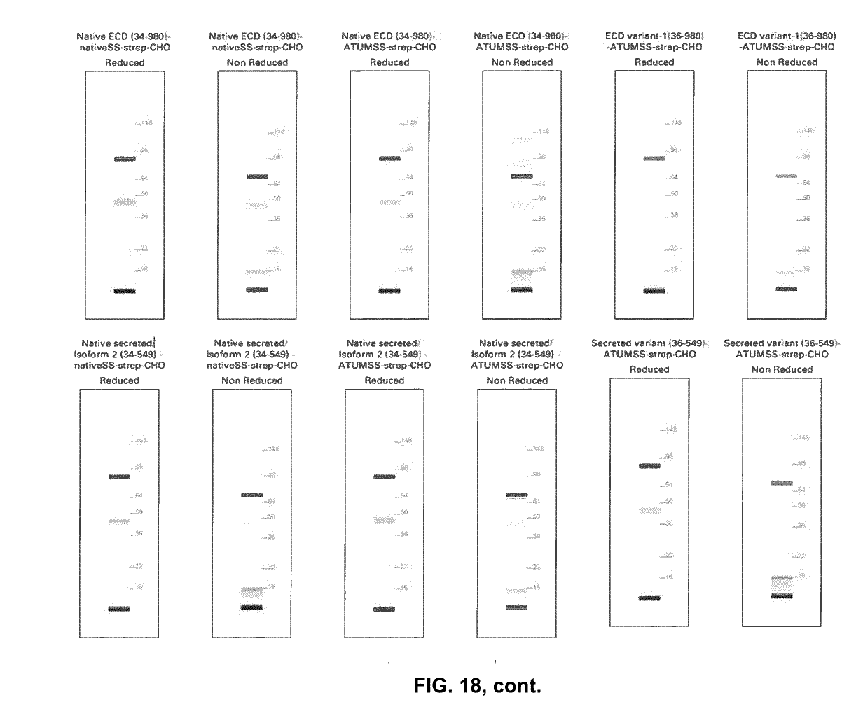

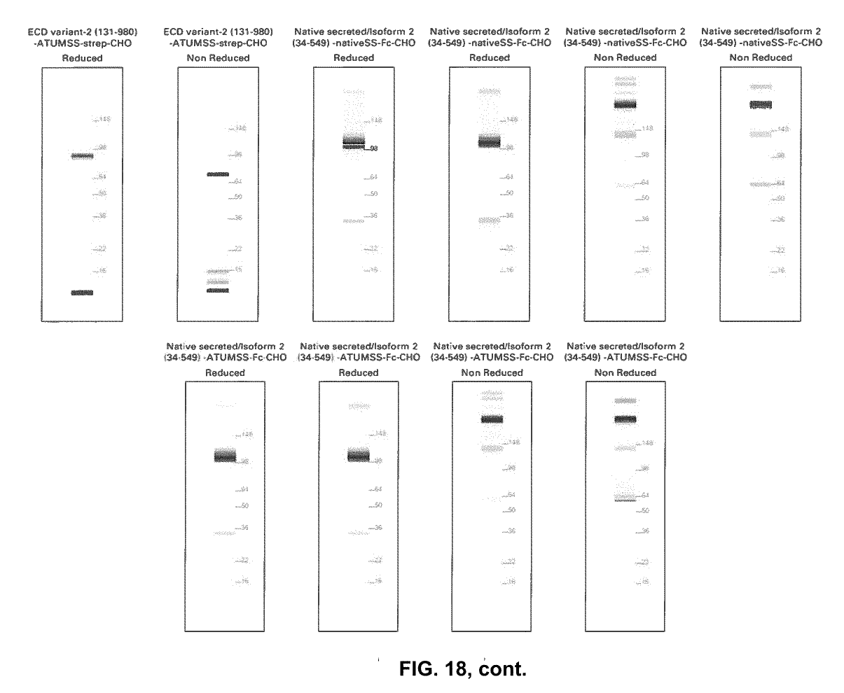

The FL-ECD (34-981)-TEV-Twin-Strep molecule expressed by stable pool 291647 (SEQ ID NO: 66), also shows the correct size on Western blots and remains a monomer under native and non-reducing conditions (see FIGS. 16A-16B). The expected size isoform-2 -Fc variant represents the secreted human Klotho protein. The predicted molecular mass of the mature full length ECD (34-981)-twin-strep tagged Klotho protein (SEQ ID NO: 66), is ˜109 kDa. Non-reduced Western blot analysis demonstrates the successful expression of the correct size protein from CHO cells coded by expression construct 291647 (see FIG. 16A). Non-reduced denatured Western blot analysis indicates that the full-length extracellular domain of hKlotho does not form covalent homodimers when secreted from CHO cells (see FIG. 16B). Other protein constructs represented in the accompanying Sequence Listing were also prepared. FIG. 17 is a gel of the various indicated recombinant Klotho protein constructs. FIG. 18 is a series of gels for the respective indicated recombinant Klotho protein constructs.

It should be appreciated that the expression and/or purification of the Klotho variants disclosed in Table 48 and/or shown in FIGS. 15A through 18, in addition to one or more other Klotho variants disclosed herein but not shown in Table 48, can, in some embodiments, result in advantages over the expression and/or purification of native Klotho. For example, there can be a reduction in the number and/or types of alternative products when expressing and/or purifying Klotho variants. Additionally, or alternatively, there can be an increase in expression level of the desired Klotho variant compared to the expression level of native Klotho. Additionally, or alternatively, the desired Klotho variant is expressed and/or purified in a purer form under comparable conditions and methods (e.g., the concentration of the desired Klotho variant is increased with a concomitant decrease in side products expressed and/or purified). Preliminary results indicated that designed recombinant fusion proteins are being expressed and purified in suitable titer ranges and are soluble in acceptable buffers, carriers, and excipients.

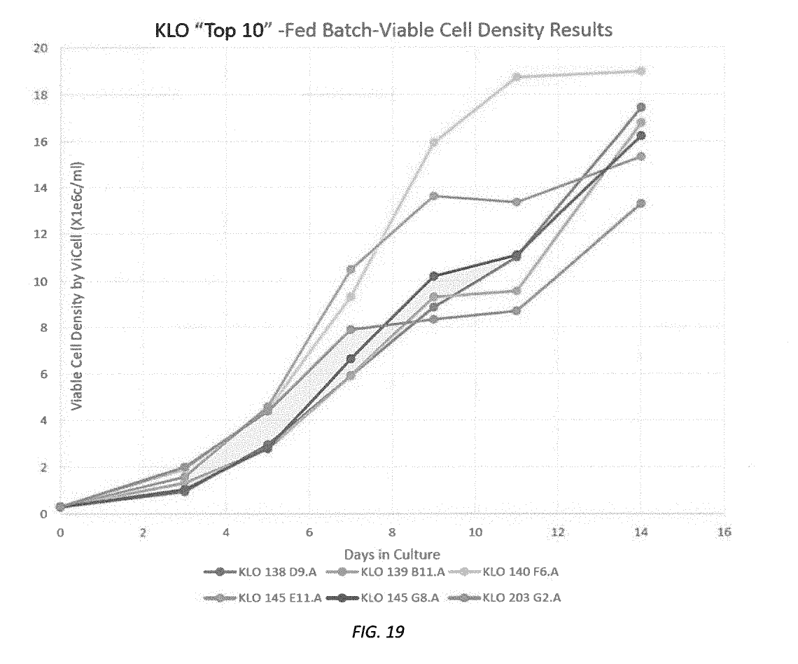

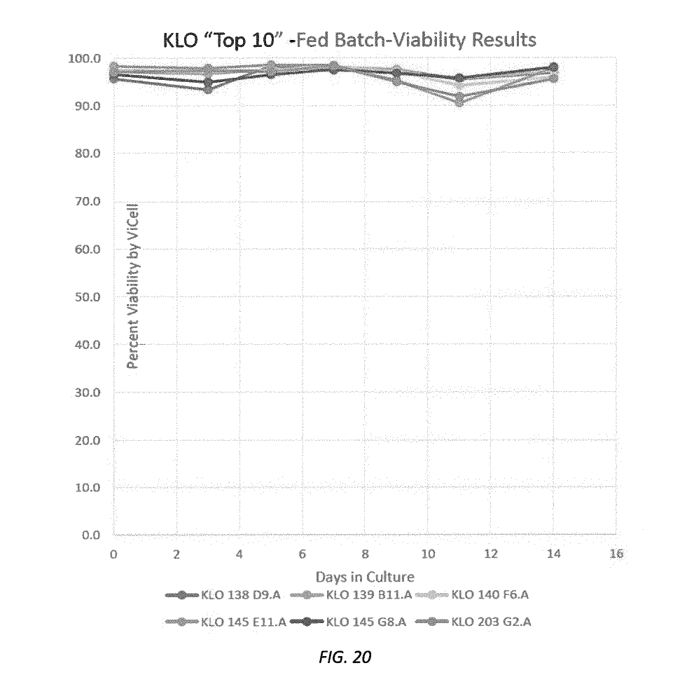

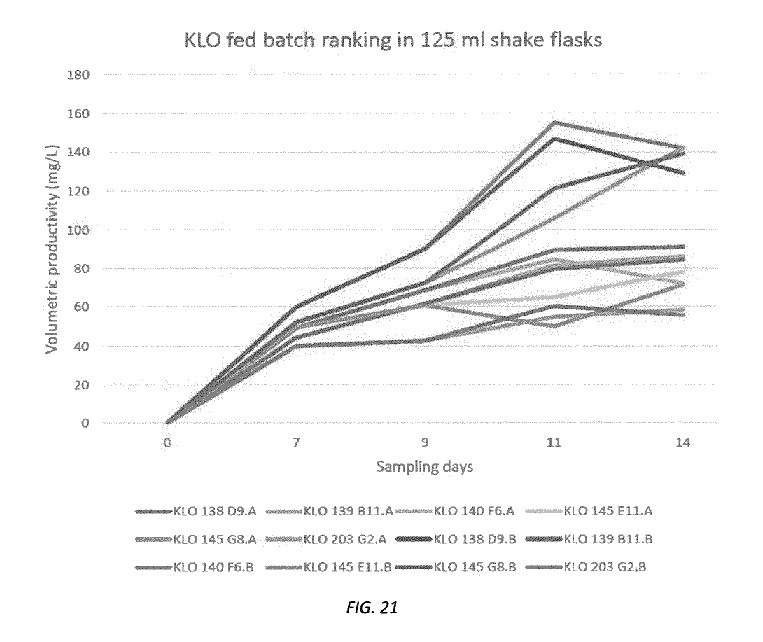

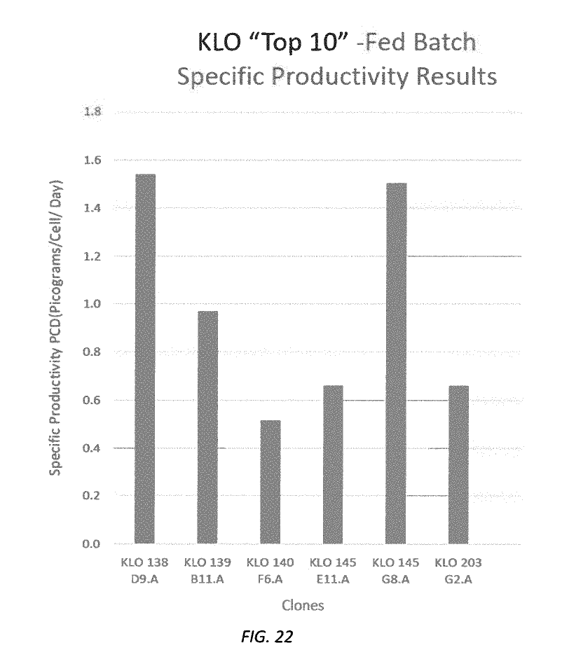

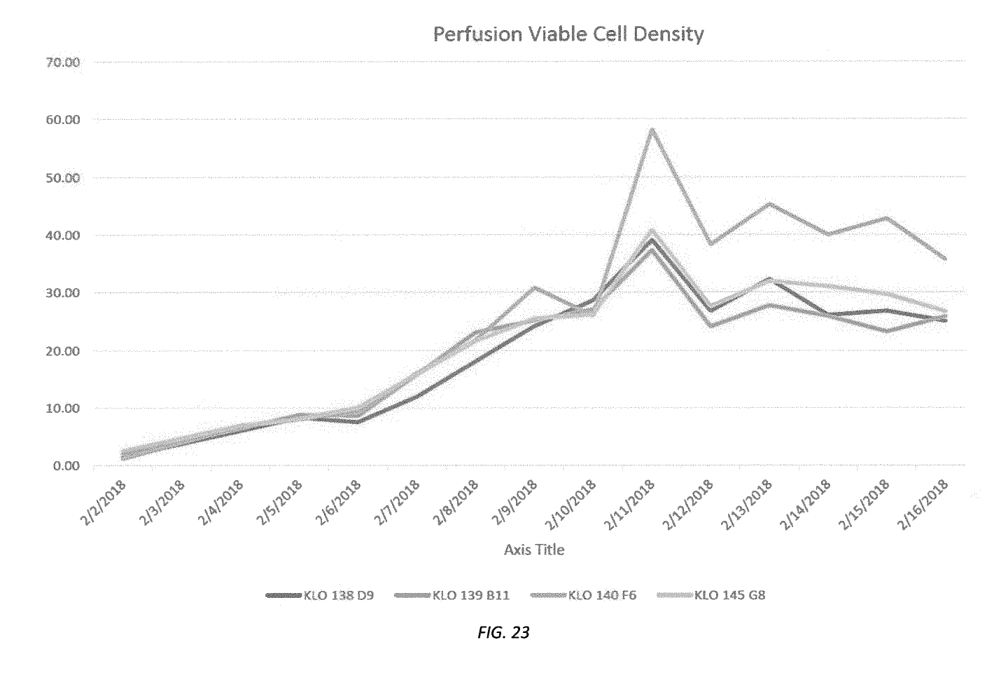

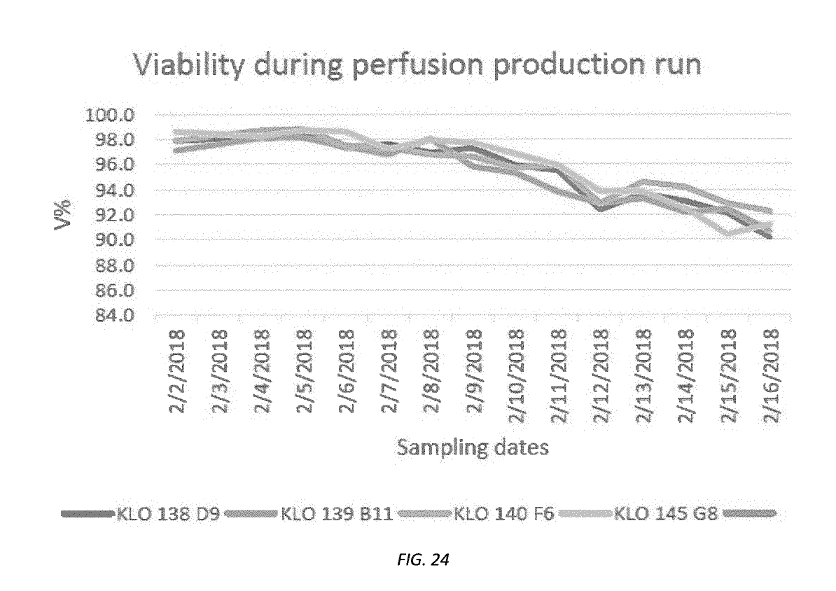

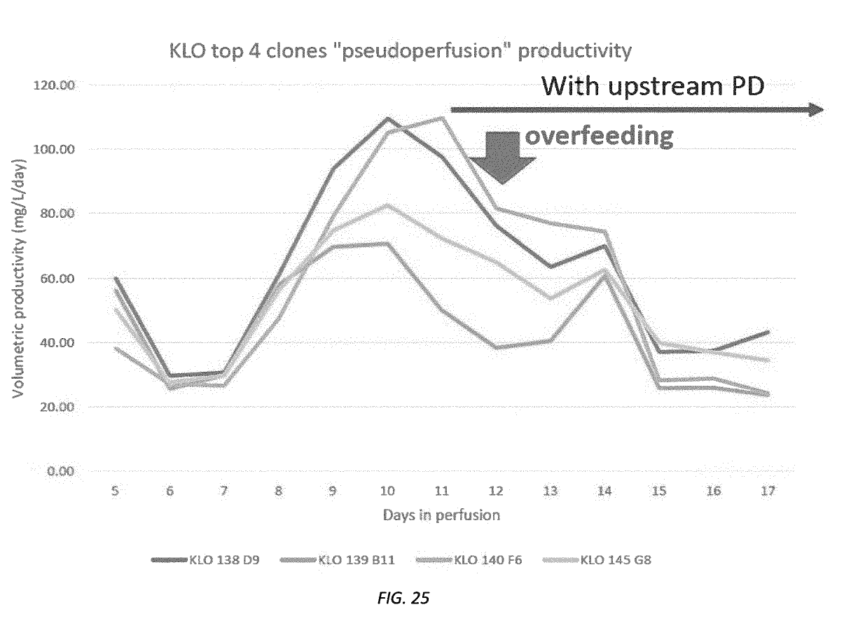

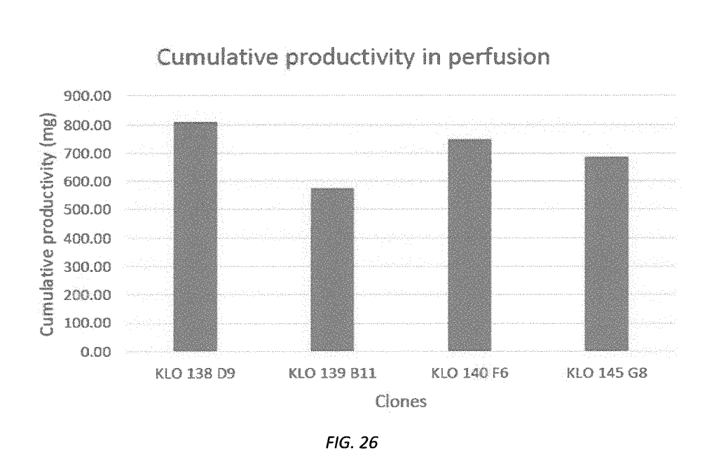

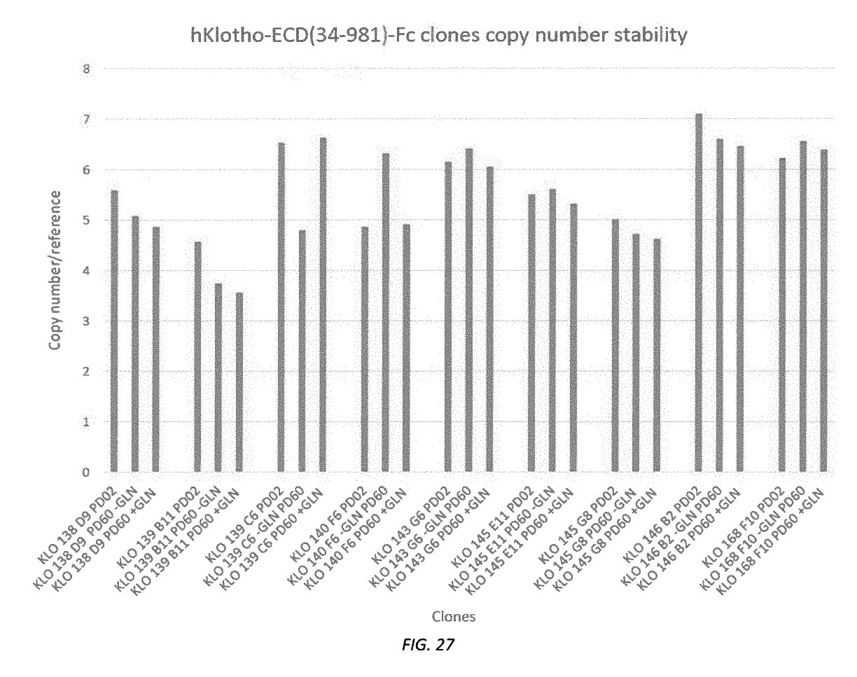

Further cell lines were developed and assessed for density, viability and production. Data from the top 10 clones are illustrated in FIGS. 19-22. Data from the top 4 clones are illustrated in FIGS. 23-26. Based on fed batch ranking, the top two clones appear to be 138 D9 and 145 G8. Based on pseudo-perfusion cumulative productiveity, 138 D9 appears to be the top performer. 140 F6 reached the highest VCD. Copy number stability was also assessed as illustrated in FIG. 27. Total RNA was extracted and RNA isolation was performed using RNeasy Mini Kit (Qiagen, catalog # 74104). Following RNA isolation, amplification of templates was carried out using Omniscript RT Kit (Qiagen, catalog # 205111). All cDNA sequences were as expected.

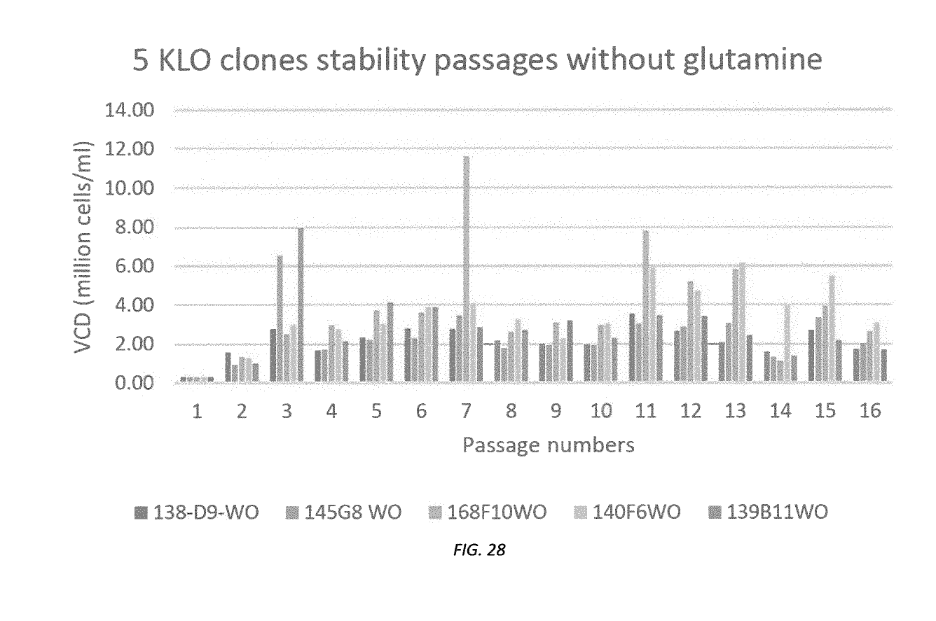

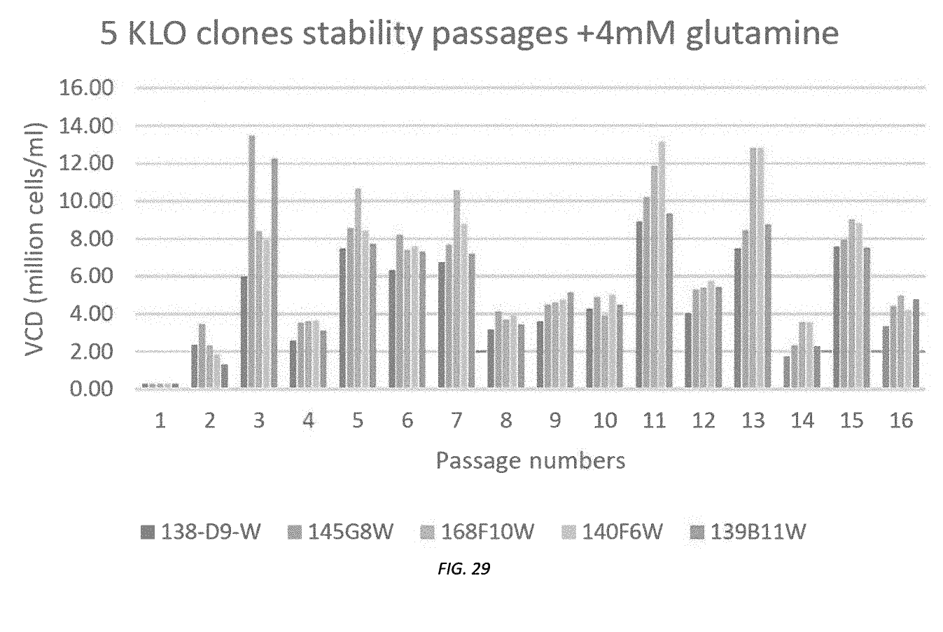

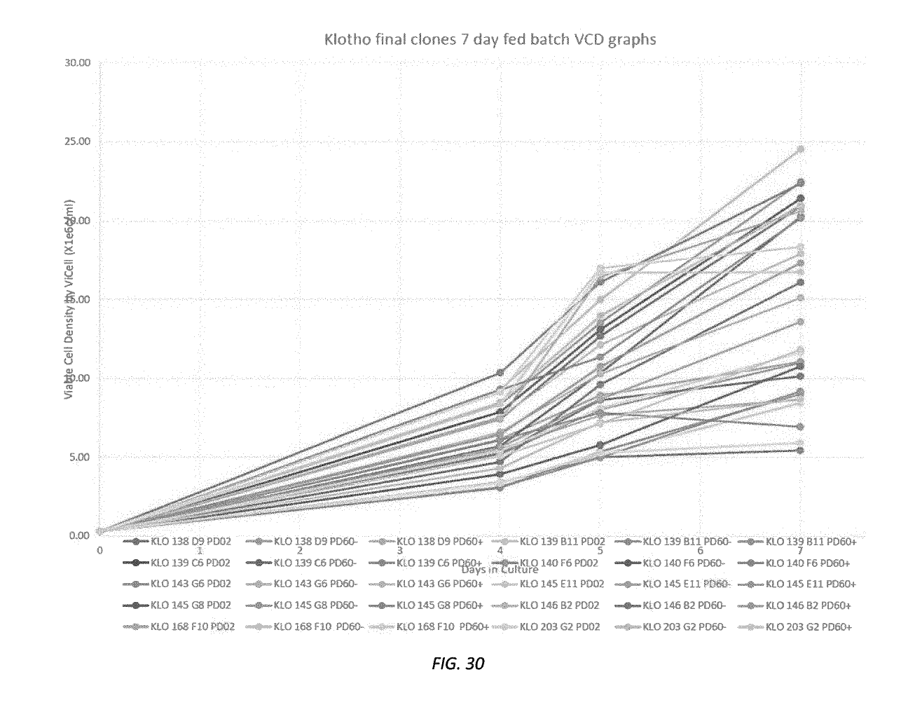

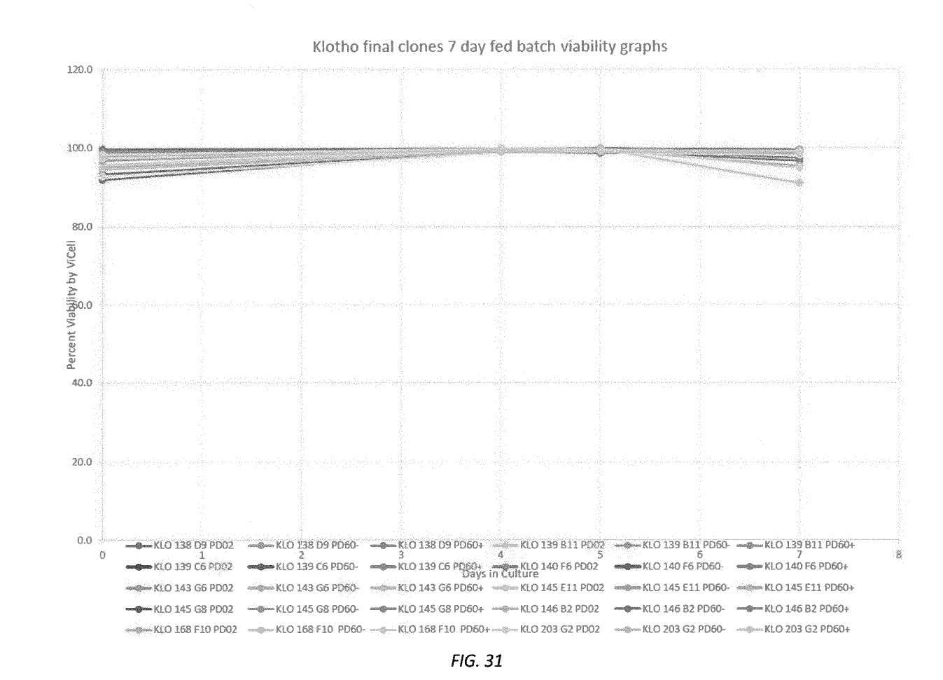

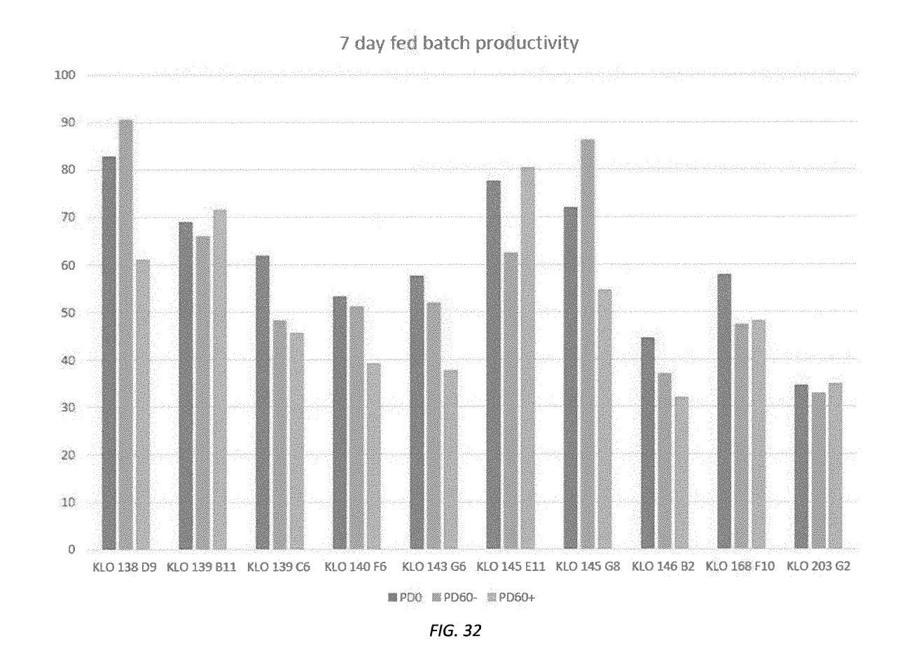

Stability of five of the clones was further tested as illustrated in FIGS. 28-32. These clones were grown in growth media with and without glutamine, and shown to have higher growth rates in the presence of 4 mM glutamine, compared to the glutamine-free condition. No growth rate change was observed during >60 population doublings in either sets. Viable cell density and percent viability of clones over a 7 day time course is illustrated in FIG. 30-31. Volumetric productivity of clones in 7 day fed batch cultures of Time 0, 60PD in the presence (+) and in the absence (−) of 4 mM glutamine is illustrated in FIG. 32. The volumetric productivity by the TO and PD60 no glutamine cells are comparable. The PD60+ glutamine volumetric productivities are either comparable or somewhat lower, but never lower than the ˜70% of the TO values.

Cells were adapted to 100% for EX-CELL, ActiPro, and CD OptiCHO media, and nearly 100% adapted to CD CHO, CD FortiCHO, BalanCD CHO Growth A, and Select CD1000 media.

### Example 3

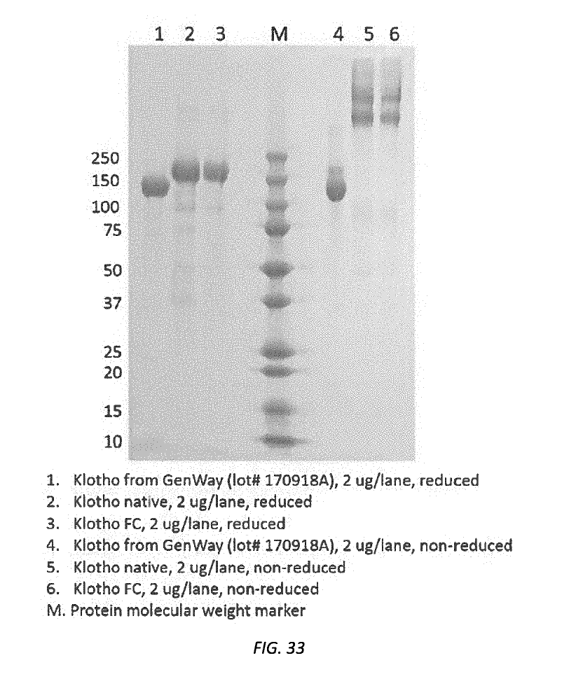

Purified Klotho proteins were observed on gel following ELISA, as shown in FIG. 33. Table 49 presents an ELISA plate map key (upper two segments) and heat map-labeled ELISA 450nm data (lower two segments). Table 50 presents the ELISA data for the Klotho proteins from cell culture supernatants and human serum samples. Table 51 presents the average concentration of Klotho in each sample group.

Table 52 presents an ELISA plate map key for human serum Klotho levels before (upper two segments) and after (lower two segments) recombinant Klotho protein injection. Table 53 presents heat map-labeled ELISA 450nm data for Klotho protein levels in human serum samples before (upper two segments) and after (lower two segments) recombinant Klotho protein injection as measured by ELISA. Human serum samples were diluted 2-fold and cell culture supernatants were diluted 5,000-fold using Klotho ELISA assay sample dilution buffer. Serum B1 and cell culture supernatants were included in both ELISA plates as controls. TMB 30 minutes. Table 54 presents a summary of average concentration of Klotho protein in the samples indicated.

TABLE 49

| | | | | | |
|---|---|---|---|---|---|
| STD, 6 ng/mL | Klotho native, 6 ng/mL | Klotho-FC, 6 ng/mL | Klotho GW, 6 ng/Ml | KLA-645 | KLA-647 |
| | | | | (1:1,000) | (1:1,000) |
| STD, 3 ng/mL | Klotho native, 3 ng/mL | Klotho-FC, 3 ng/mL | Klotho GW, 3 ng/mL | KLA-645 | KLA-647 |
| | | | | (1:10,000) | (1:10,000) |
| STD, 1.5 ng/mL | Klotho native, 1.5 ng/mL | Klotho-FC, 1.5 ng/mL | Klotho GW, 1.5 ng/Ml | KLA-645 | KLA-647 |
| | | | | (1:100,000) | (1:100,000) |
| STD, 0.75 ng/mL | Klotho native, 0.75 ng/mL | Klotho-FC, 0.75 ng/mL | Klotho GW, 0.75 ng/mL | KLA-645 | KLA-647 |
| | | | | (1:1,000,000) | (1:1,000,000) |
| STD, 0.375 ng/mL | Klotho native, 0.375 ng/mL | Klotho-FC, 0.375 ng/mL | Klotho GW, 0.375 ng/mL | Serum 1A | Serum 13A |
| STD, 0.188 ng/mL | Klotho native, 0.18 ng/mL | Klotho-FC, 0.188 ng/mL | Klotho GW, 0.188 ng/mL | Serum 1A (1:2) | Serum 13A (1:2) |
| STD, 0.094 ng/mL | Klotho native, 0.094 ng/mL | Klotho-FC, 0.094 ng/mL | Klotho GW, 0.094 ng/mL | Serum 1A (1:4) | Serum 13A (1:4) |
| STD, 0 ng/mL | Klotho native, 0 ng/mL | Klotho-FC, 0 ng/mL | Klotho GW, 0 ng/mL | Serum 1A (1:8) | Serum 13A (1:8) |

| | | | | | | | | | | | |
|---|---|---|---|---|---|---|---|---|---|---|---|
| 3.138 | 3.102 | 0.073 | 0.075 | 0.085 | 0.075 | 3.773 | 3.853 | 0.97 | 0.96 | 3.521 | 3.583 |
| 1.777 | 1.751 | 0.068 | 0.072 | 0.077 | 0.07 | 3.247 | 3.271 | 0.185 | 0.183 | 0.548 | 0.574 |
| 0.963 | 0.952 | 0.071 | 0.074 | 0.079 | 0.07 | 1.886 | 1.92 | 0.088 | 0.09 | 0.13 | 0.144 |
| 0.54 | 0.532 | 0.073 | 0.07 | 0.064 | 0.071 | 1.095 | 1.128 | 0.084 | 0.089 | 0.097 | 0.093 |
| 0.308 | 0.319 | 0.083 | 0.076 | 0.074 | 0.074 | 0.596 | 0.615 | 0.397 | 0.406 | 0.449 | 0.422 |
| 0.201 | 0.194 | 0.074 | 0.074 | 0.075 | 0.066 | 0.34 | 0.338 | 0.261 | 0.255 | 0.29 | 0.301 |
| 0.143 | 0.133 | 0.071 | 0.068 | 0.086 | 0.084 | 0.206 | 0.207 | 0.167 | 0.171 | 0.184 | 0.196 |
| 0.071 | 0.075 | 0.078 | 0.075 | 0.071 | 0.068 | 0.071 | 0.081 | 0.13 | 0.13 | 0.142 | 0.143 |

TABLE 50

| | | | | | | | | | Klotho |
|---|---|---|---|---|---|---|---|---|---|
| Wells | Values | R | Result | MeanResult | Std. Dev. | CV % | Dilution | Adj. Result | (ng/mL) |
| A7 | 3.773 | R | 7.668 | 7.776 | 0.153 | 2 | 1 | 7.776 | |
| A8 | 3.853 | R | 7.884 | | | | | | |
| B7 | 3.247 | R | 6.31 | 6.339 | 0.042 | 0.7 | 2 | 12.678 | |
| B8 | 3.271 | R | 6.369 | | | | | | |
| C7 | 1.886 | | 3.256 | 3.29 | 0.049 | 1.5 | 4 | 13.161 | 13.58 |
| C8 | 1.92 | | 3.325 | | | | | | |
| D7 | 1.095 | | 1.733 | 1.764 | 0.043 | 2.4 | 8 | 14.109 | |
| D8 | 1.128 | | 1.794 | | | | | | |
| E7 | 0.596 | | 0.853 | 0.87 | 0.023 | 2.6 | 16 | 13.915 | |
| E8 | 0.615 | | 0.886 | | | | | | |
| F7 | 0.34 | | 0.424 | 0.423 | 0.002 | 0.6 | 32 | 13.523 | |
| F8 | 0.338 | | 0.421 | | | | | | |
| G7 | 0.206 | | 0.206 | 0.206 | 0.004 | 0.6 | 64 | 13.206 | |
| G8 | 0.207 | | 0.207 | | | | | | |
| H7 | 0.071 | R | Range? | 0.006 | 0 | 0 | 128 | 0.729 | |
| H8 | 0.081 | | 0.006 | | | | | | |
| A9 | 0.97 | | 1.507 | 1.499 | 0.13 | 0.8 | 1000 | 1498.504 | 1599.46 |
| A10 | 0.96 | | 1.49 | | | | | | |
| B9 | 0.185 | | 0.172 | 0.17 | 0.002 | 1.3 | 10000 | 1700.413 | |
| B10 | 0.183 | | 0.168 | | | | | | |
| C9 | 0.088 | | 0.017 | 0.18 | 0.002 | 12.2 | 100000 | 1830.104 | |
| C10 | 0.09 | | 0.02 | | | | | | |
| D9 | 0.084 | | 0.01 | 0.14 | 0.006 | 38.9 | 1000000 | 14356.72 | |
| D10 | 0.089 | | 0.018 | | | | | | |
| A11 | 3.521 | R | 7.003 | 7.084 | 0.114 | 1.6 | 1000 | 7083.639 | |
| A12 | 3.583 | R | 7.164 | | | | | | |
| B11 | 0.548 | | 0.772 | 0.031 | 0.031 | 3.9 | 10000 | 7938.998 | 8700.99 |
| B12 | 0.574 | | 0.816 | | | | | | |
| C11 | 0.13 | | 0.083 | 0.016 | 0.016 | 16.7 | 100000 | 9462.989 | |
| C12 | 0.144 | | 0.106 | | | | | | |
| D11 | 0.097 | | 0.031 | 0.004 | 0.004 | 16.1 | 1000000 | 27789.87 | |
| D12 | 0.093 | | 0.025 | | | | | | |
| E9 | 0.397 | | 0.519 | 0.526 | 0.011 | 23 | 1 | 0.526 | 0.56 |
| E10 | 0.406 | | 0.533 | | | | | | |
| F9 | 0.261 | | 0.295 | 0.29 | 0.007 | 2.4 | 2 | 0.58 | |
| F10 | 0.255 | | 0.285 | | | | | | |
| G9 | 0.167 | | 0.143 | 0.146 | 0.005 | 3.1 | 4 | 0.584 | |
| G10 | 0.171 | | 0.149 | | | | | | |
| H9 | 0.13 | | 0.083 | 0.083 | 0 | 0 | 8 | 0.668 | |
| H10 | 0.13 | | 0.083 | | | | | | |
| E11 | 0.449 | | 0.605 | 0.583 | 0.032 | 5.5 | 1 | 0.583 | 0.71 |
| E12 | 0.422 | | 0.56 | | | | | | |
| F11 | 0.29 | | 0.342 | 0.351 | 0.013 | 3.6 | 2 | 0.702 | |
| F12 | 0.301 | | 0.36 | | | | | | |
| G11 | 0.184 | | 0.17 | 0.18 | 0.014 | 7.6 | 4 | 0.719 | |
| G12 | 0.196 | | 0.189 | | | | | | |
| H11 | 0.142 | | 0.103 | 0.103 | 0.001 | 1.1 | 8 | 0.827 | |
| H12 | 0.143 | | 0.104 | | | | | | |

TABLE 51

| | [Klotho], |
|---|---|
| Samples | ng/mL |
| Klotho from GenWay | 13.58 |
| Supernatant, KLA-645 | 1599.46 |
| Supernatant, KLA-647 | 8700.99 |
| Human serum, Base, 1A | 0.56 |
| Human serum, Protein, 13A | 0.71 |

TABLE 52

| | 1 | 2 | 3 | 4 | 5 | 6 | 7 | 8 | 9 | 10 | 11 | 12 |
|---|---|---|---|---|---|---|---|---|---|---|---|---|
| A | STD, 6 ng/mL | | 1A | | 2A | | 3A | | 4A | | 5A | |
| B | STD, 3 ng/mL | | 6A | | 7A | | 8A | | 9A | | 10A | |
| C | STD, 1.5 ng/mL | | 11A | | 12A | | 13A | | B1 | | | |
| D | STD, 0.75 ng/mL | | KLA-645 (Day 9) | | KLA-645 (Day 10) | | KLA-647 (Day 8) | | KLA-647 (Day 9) | | | |
| E | STD, 0.375 ng/mL | | | | | | | | | | | |
| F | STD, 0.188 ng/mL | | | | | | | | | | | |
| G | STD, 0.094 ng/mL | | | | | | | | | | | |
| H | STD, 0 ng/mL | | | | | | | | | | | |
| | 1 | 2 | 3 | 4 | 5 | 6 | 7 | 8 | 9 | 10 | 11 | 12 |

| A | STD, 6 ng/mL | 1A | 2A | 3A | 4A | 5A |
|---|---|---|---|---|---|---|
| B | STD, 3 ng/mL | 6A | 7A | 8A | 9A | 10A |
| C | STD, 1.5 ng/mL | 11A | 12A | 13B | 14A | 15A |
| D | STD, 0.75 ng/mL | 16A | 17A | 18A | 19A | 20A |
| E | STD, 0.375 ng/mL | 21A | 22A | 23A | 24A | 25A |
| F | STD, 0.188 ng/mL | 26A | 27A | 28A | 29A | B1 |
| G | STD, 0.094 ng/mL | KLA-645 (Day 9) | KLA-645 (Day 10) | KLA-647 (Day 8) | KLA-647 (Day 9) | |
| H | STD, 0 ng/mL | | | | | |

Table 55 presents the concentration and monoclonality of the replicate Klotho protein samples.

TABLE 55

| | | Gel | Conc. (mg/L) | Conc. (mg/L) | |
|---|---|---|---|---|---|
| | Clone ID | Lane | Method 1 | Method 2 | Monoclonality |
| 1 | KLO-145-G8 | B8 | 127.2 | 154.0 | Likely |
| 2 | KLO-138-B8 | A7 | 108.9 | 79.4 | yes |
| 3 | KLO-138-D9 | D5 | 106.54 | 133.9 | yes |
| 4 | KLO-139-C6 | A12 | 102.0 | 64.6 | yes |
| 5 | KLO-138-D9 | D4 | 98.33 | 138.1 | yes |
| 6 | KLO-145-E11 | B3 | 94.1 | 62.3 | yes |
| 7 | KLO-140-E8 | A4 | 91.4 | 55.5 | yes |
| 8 | KLO-137-G6 | A1 | 88.7 | 50.9 | yes |
| 9 | KLO-139-G5 | A5 | 83.9 | 53.7 | yes |
| 10 | KLO-137-D8 | A6 | 83.4 | 51.2 | yes |
| 11 | KLO-139-G17 | A9 | 81.7 | 49.1 | yes |
| 12 | KLO-140-D6 | B6 | 76.88 | 63.2 | yes |
| 13 | KLO-138-E2 | A3 | 76.8 | 33.0 | yes |
| 14 | KLO-140-G3 | A11 | 74.4 | 44.9 | yes |
| 15 | KLO-138-E11 | B5 | 73.04 | 53.2 | questionable |
| 16 | KLO-143-G6 | B2 | 70.6 | 40.5 | yes |
| 17 | KLO-140-G4 | A2 | 68.2 | 36.2 | no |
| 18 | KLO-137-D5 | A10 | 66.7 | 38.7 | yes |
| 19 | KLO-137-D6 | A8 | 66.1 | 36.8 | yes |
| 20 | KLO-144-C4 | B4 | 60.5 | 30.3 | very likely |
| 21 | KLO-138-G8 | B7 | 58.11 | 40.1 | no |
| 22 | KLO-139-D11 | C5 | 49.99 | 40.4 | questionable |
| 23 | KLO-139-F11 | C3 | 49.37 | 41.4 | yes |
| 24 | KLO-138-G2 | C4 | 47.5 | 37.6 | Unknown |
| 25 | KLO-645-146-B2 | C8 | 47.39 | 34.4 | Unknown |
| 26 | KLO-138-G4 | C1 | 35.59 | 24.3 | Unknown |
| 27 | KLO-138-B10 | B10 | 35.27 | 38.8 | Unknown |
| 28 | KLO-144-G6 | B1 | 34.8 | 20.4 | Unknown |
| 29 | KLO-139-B11 | B12 | 34.66 | 32.7 | Unknown |
| 30 | KLO-140-F6 | B11 | 34.53 | 34.6 | Unknown |
| 31 | KLO-645-143-D7 | C7 | 30.28 | 15.7 | Unknown |
| 32 | KLO-645-143-F9 | D1 | 28.04 | 22.1 | Unknown |
| 33 | KLO-645-168-A8 | C10 | 26.92 | 16.3 | Unknown |
| 34 | KLO-645-142-G2 | B9 | 22.71 | 18.8 | Unknown |
| 35 | KLO-645-169-D7 | D3 | 21.08 | 17.1 | Unknown |
| 36 | KLO-645-168-G12 | C12 | 18.46 | 11.7 | Unknown |
| 37 | KLO-645-167-B10 | C9 | 8.49 | 0.0 | Unknown |
| 38 | KLO-645-146-C10 | D2 | 7.71 | 17.4 | Unknown |
| 39 | KLO-645-168-F10 | C11 | 0 | 43.0 | Unknown |

An illustrative method for estimation of concentration involved quantitation (quantification) by PAGE against a BSA standard. An illustrative method for estimation of concentration involved measurement against purified Klotho standard.

One interpretation of the data could be that native-like, recombinant (TwinStrep-TEV tag-removed) Klotho protein expresses better, purifies cleaner, and/or retains more activity than does the recombinant Fc-tagged Klotho protein. This was surprising and unexpected. An alternative interpretation is that neither protein, or the Fc-fusion protein more so than the native-like protein, showed levels of klotho protein reaction to the ELISA antibody. Accordingly, we may have identified that expression, purification, storage, and/or administration of native-like recombinant Klotho protein and Fc-fusion Klotho protein was not as simple as expected and that standard techniques did not work well. Proceeding based on the later interpretation, procedures were modified to obtain more active protein, while continuing to process the data for further interpretation.

Three commercially-available ELISA detection kits for Klotho protein were also tested for suitability in accordance with certain embodiments of the present disclosure. See Tables 57 and 58, below. The IBL Kits shows low background, liner dilution of serum samples, acceptable spike recovery after 1:4 dilution. The Cloud-clone Kits shows high background, no liner dilution of serum samples, no acceptable spike recovery. The EIAab Kits shows low background, very low Klotho concentration in serum sample, no liner dilution of serum samples, no acceptable spike recovery.

Total 8 human serum samples were diluted with each kit's dilution buffer at 1:2, 1:4, 1:8. 4 of these 8 serum samples were spiked with top concentration of Standard from each kit at 1:20 dilution. The protocol from each kit was followed per instructions provided.

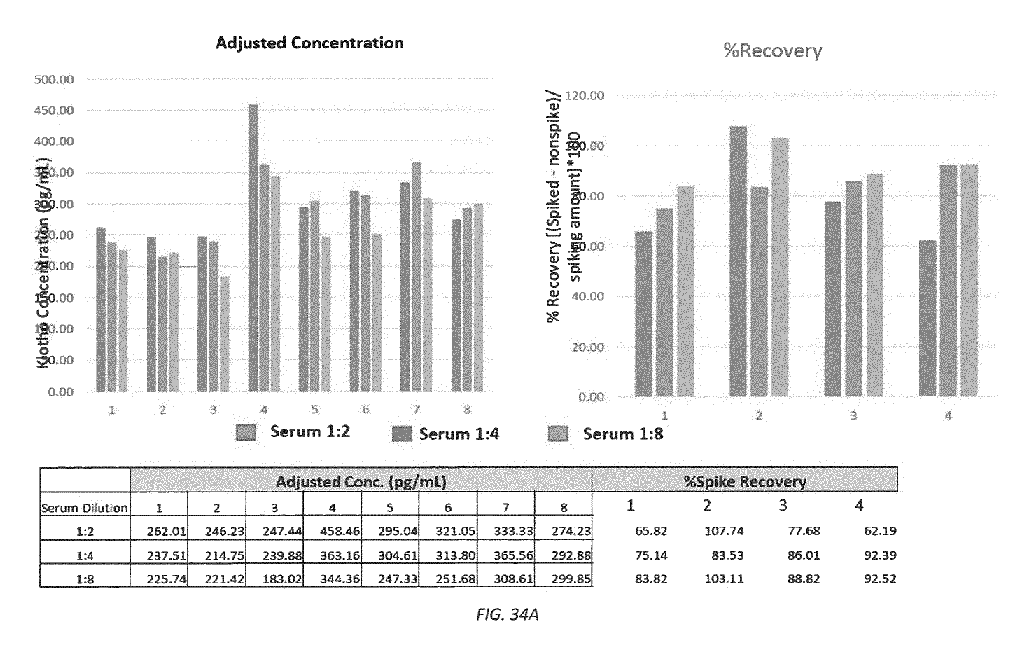

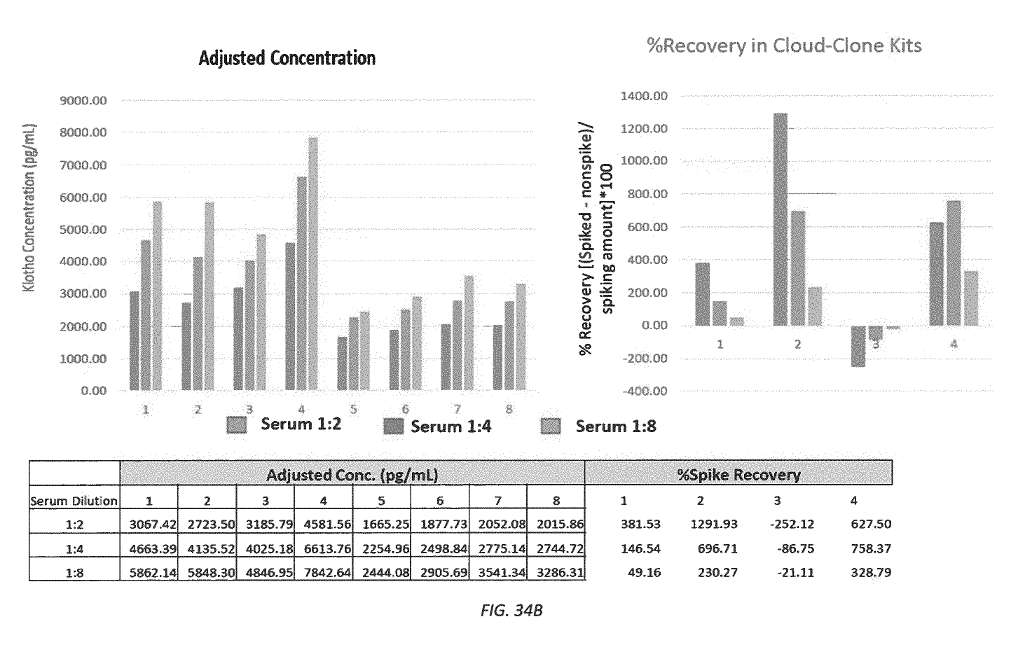

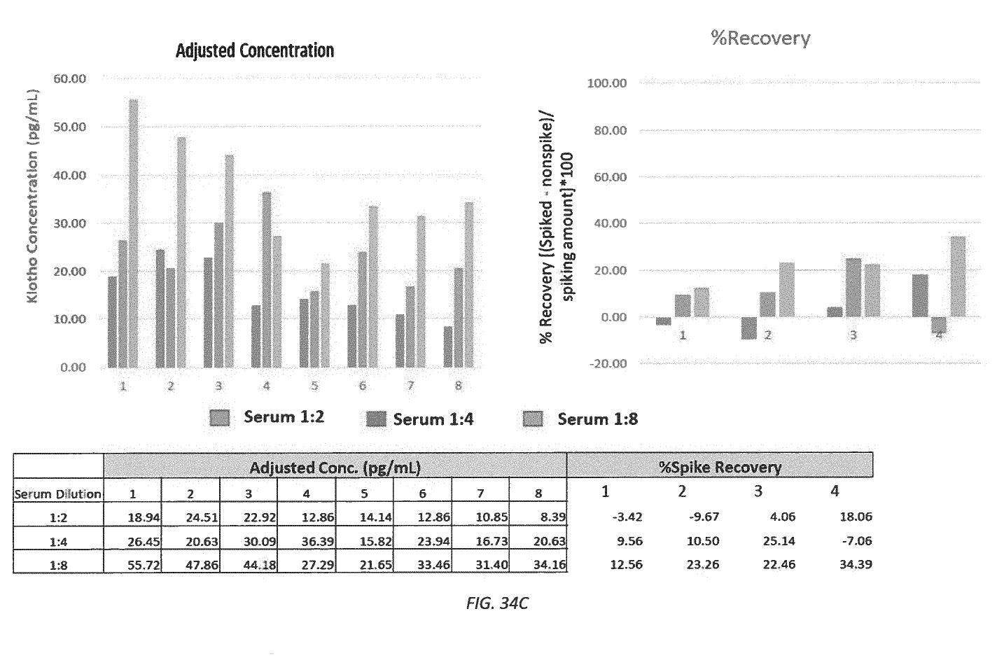

FIGS. 34A-34C illustrate Klotho protein concentration measured and spike recovery observed in the respective samples.

TABLE 57

| Vendor | Assay Range | |
|---|---|---|
| IBL | 93.8-6000 | pg/mL |
| Cloud-Clone | 15.6-1000 | pg/mL |
| EIAab | 7.8-500 | pg · mL |

TABLE 58

| Vendor | Spiking Concentration (pg/mL) |
|---|---|
| IBL | 300 |
| Cloud-Clone | 500 |
| EIAab | 25 |

### Example 4

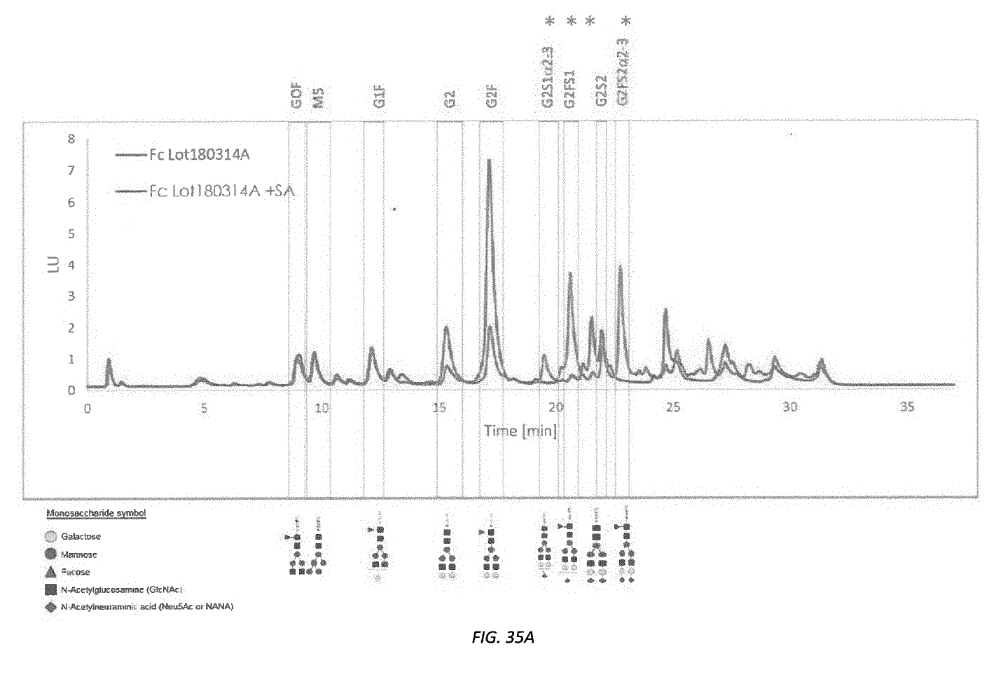

Preliminary global glycan analysis was also performed as illustrated in FIGS. 35A-35B. Experimental conditions and results for the glycan analysis are presented in Table 59.

TABLE 59

| | |
|---|---|
| Experimental | N-Glycans were released and labeled using the Gly-X ™ N-Glycan rapid release and labeling with |
| conditions | InstantPC ™ Kit. |
| | LC analysis of 1 uL of sample was performed on a Advance Glycan Map 2.1 × 100 mm column. |
| | To gain additional insight into the core glycan structures, the sialic acid content of the sample was |
| | released using SialidaseA |
| | Each peak was assigned a GU unit that was referenced to a in-house database generated from Glycan |
| | Standards. |
| | Area under the peak was calculated for peaks with retention times from 4.5-25 mins and a minimum |
| | peak height of 0.2 and area of 10 or greater. |
| | Percentage area of individual peaks was also calculated. |
| Results | Only the glycans GOF, M5, G1, G1F, G2F, G2 and M7 were assigned with confidence. |
| | Both samples were treated with Sialidase A to try and understand all of the core structures in the |
| | sample. This approached provided no further insight into the unknown peaks. |
| | The sample appears to contain several glycans that are not represented by the glycan standards that |
| | are commercially available. Further characterization either by exoglycosidase treatment or by LC/MS is |
| | recommended. |

Tables 60 and 61, below present the data illustrated in FIGS. 35A-35B, respectively.

TABLE 60

| Protein ID | Fc Lot180314A | Fc Lot180314A + SA |
|---|---|---|
| Glycan Assignment | Area % | Area % |
| GOF | 5.18 | 5.38 |
| M5 | 4.46 | 4.18 |
| G1F | 6.46 | 5.30 |
| G2 | 4.54 | 9.31 |
| G2F | 13.36 | 36.23 |
| * G2S1 α 2-3 | 4.32 | — |
| * G2FS1 α 2-3 | 18.57 | 5.84 |
| * G2S2 | 4.03 | |
| G2FS2 α 2-3 | 17.66 | — |
| Unknown | 20.43 | 33.79 |

TABLE 61

Protein ID

| | Fc Lot180622A | Fc Lot180622A + SA |
|---|---|---|
| Glycan Assignment | Area % | Area % |
| GOF | 2.68 | 1.91 |
| M5 | 1.62 | 1.45 |
| G1 | 2.98 | 2.68 |
| G1F | 2.11 | 2.33 |
| G2 | 3.87 | 7.33 |
| * G2S1 α 2-3 | 4.06 | 3.947 |
| * G1FS1 α 2-3 | 8.50 | 8.85 |
| Man7 | 17.96 | 16.97 |
| Unknown | 5.22 | 52.86 |

An additional pool of 9 clones (see Table 62, below) was assessed for suitability.

TABLE 62

| # | Plasmid | Construct |
|---|---|---|
| WA | Puro_BT+_SLX-491X_wtSP_Seq1_Sc | wtSP_Klotho ECD(1-981)-Fc |
| WN | Puro_BT+_SLX-491X_wtSP_Seq2_Sc | wtSP_Klotho ECD(1-981) |
| WD | Puro_BT+_SLX-491X_wtSP_Seq4_Sc | wtSP_Klotho ECD(1-981) - Native V45F |
| WE | Puro_BT+_SLX-491X_wtSP_Seq5_Sc | wtSP-Secreted/Isoform2 (1-549)-Fc |
| XB | Puro_BT+_SLX-491X_LubSP_Seq2_Sc | LubSP_Klotho ECD (1-981) |
| XC | Puro_BT+_SLX-491X_LubSP_Seq3_Sc | wtSP_Klotho ECD (1-981) -Fc V45F |
| XD | Puro_BT+_SLX-491X_LubSP_Seq4_Sc | LubSP_Klotho ECD (1-981) - Native V45F |
| XE | Puro_BT+_SLX-491X_LubSP_Seq5_Sc | LupSP_secreted/Isoform2 (1-549)-Fc |
| XH | Puro_BT+_SLX-491X_LubSP_Seq6_Sc | LubSP_secreted variant (1-549) |

Growth, viability, and titer of each cell line is presented Table 63, below.

TABLE 63

| | day 3 | | | | day 7 | | | day 9 | | |
|---|---|---|---|---|---|---|---|---|---|---|
| | cell conc | viability | doubling | Titer | cell conc | viability | Titer | cell conc | viability | Titer |
| # | [c/mL] | [%] | time [h] | [ug/mL] | [c/mL] | [%] | [ug/mL] | [c/mL] | [%] | [ug/mL] |
| WA | 4.82E+06 | 99.3 | 18.0 | 0 | 2.30E+07 | 97.1 | 2 | 1.22E+07 | 84.4 | 0 |
| WB | 6.57E+06 | 99.3 | 16.2 | 0 | 2.18E+07 | 95.9 | 1 | 2.63E+07 | 85.2 | 0 |
| WD | 4.89E+06 | 99.3 | 17.9 | 1 | 1.58E+07 | 95.2 | 15 | 1.29E+07 | 90.7 | 18 |
| WE | 6.63E+06 | 99.8 | 16.1 | 20 | 1.68E+07 | 95.8 | 70 | 1.99E+07 | 87.1 | 61 |
| XB | 6.09E+06 | 98.9 | 16.6 | 1 | 2.10E+07 | 96.3 | 5 | 2.68E+07 | 84.8 | 2 |
| XC | 4.95E+06 | 99.0 | 17.8 | 1 | 1.85E+07 | 97.6 | 23 | 9.56E+06 | 91.9 | 22 |
| XD | 5.48E+06 | 98.8 | 17.2 | 0 | 2.58E+07 | 98.6 | 4 | 1.94E+07 | 95.3 | 3 |
| XE | 8.29E+06 | 99.1 | 15.0 | 13 | 2.35E+07 | 94.6 | 49 | 1.37E+07 | 89.2 | 32 |
| XH | 8.46E+06 | 98.4 | 14.9 | 2 | 2.53E+07 | 96.7 | 14 | 1.74E+07 | 90.7 | 5 |

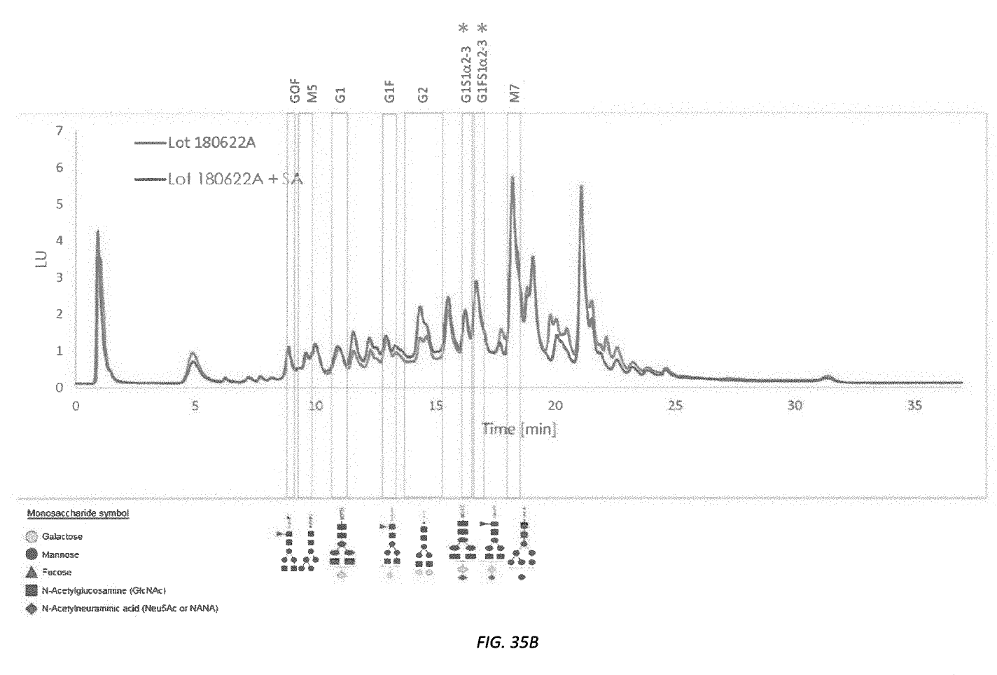

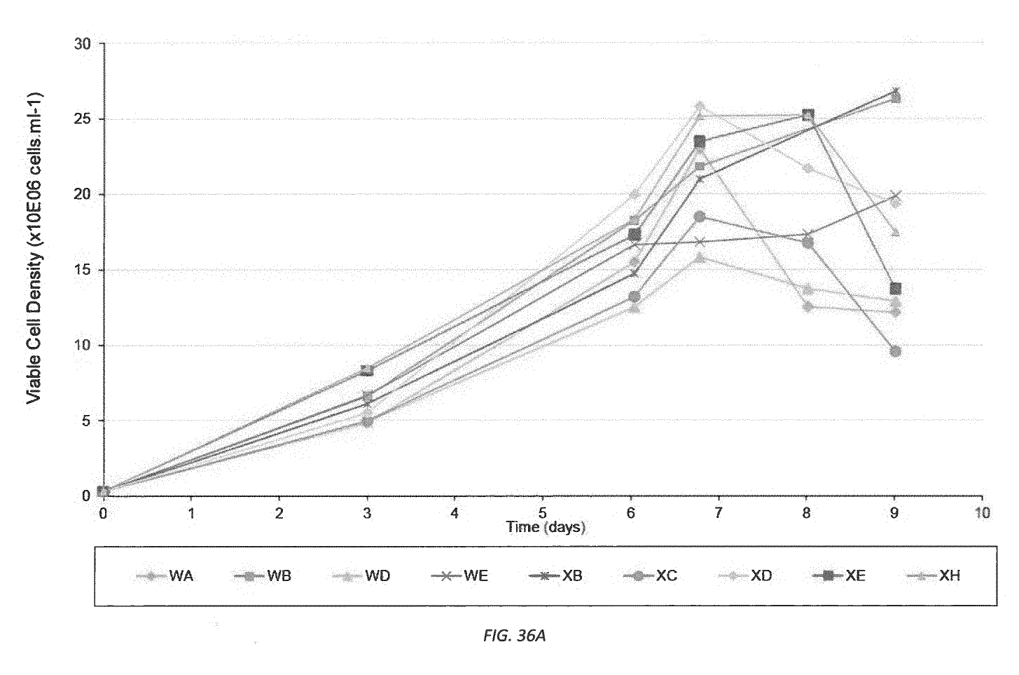

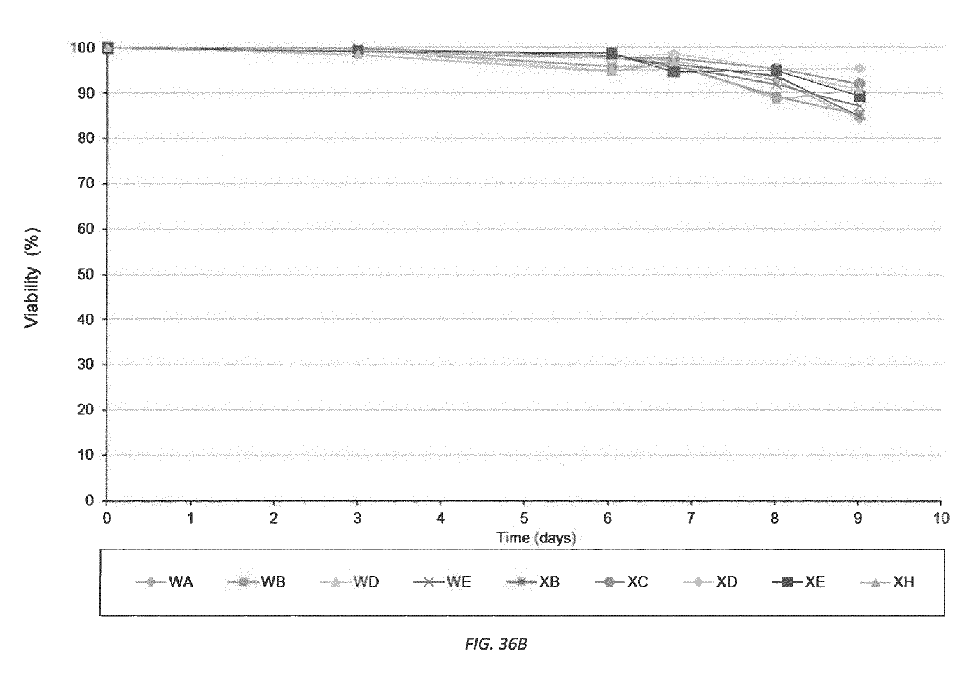

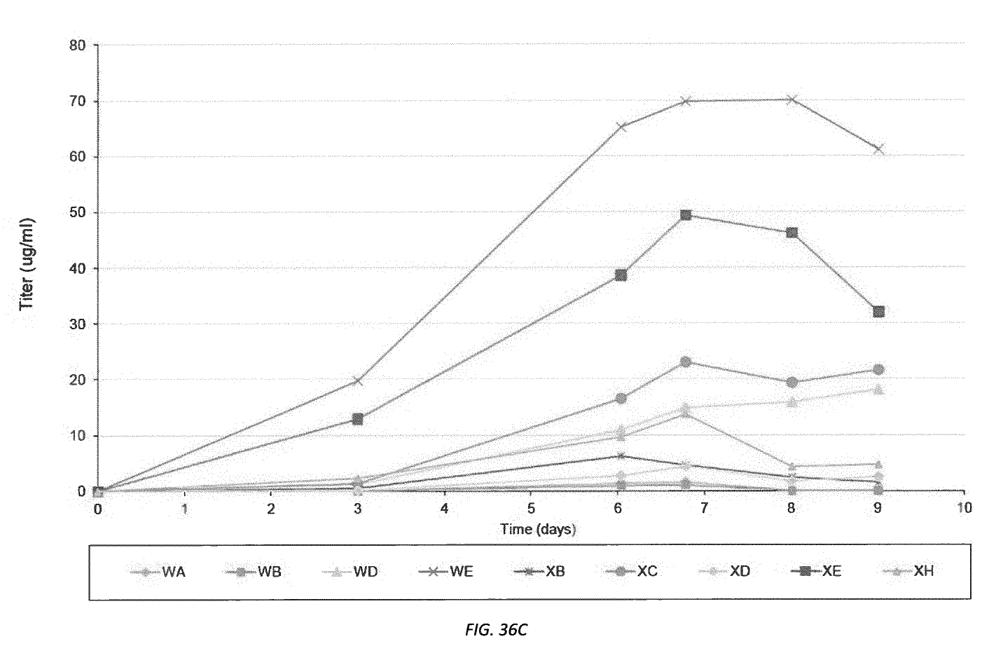

FIGS. 36A-36C illustrate growth, viability, and titer curves, respectively, for each cell line.

An additional pool of 3 clones (see Table 64, below) was assessed for suitability.

TABLE 64

| # | Plasmid | Construct |
|---|---|---|
| WC2 | Puro_BT+_SLX-491X_wtSP_Seq3_Sc | wtSP_Klotho |
| | | ECD(1-981)- |
| | | Fc V45F |
| WH2 | Puro_BT+_SLX-491X_wtSP_Seq6_Sc | stSP_secreted |
| | | variant (1-549) |
| XA2 | Puro_BT+_SLX-491X_LubSP_Seq1_Sc | LubSP_Klotho |
| | | ECD(1-981)-Fc |

Growth, viability, and production of each cell line is presented Table 65, below.

TABLE 65

| | day 3 | | | | day 7 | | | day 9 | | |
|---|---|---|---|---|---|---|---|---|---|---|
| | cell conc | viability | doubling | Titer | cell conc | viability | Titer | cell conc | viability | Titer |
| # | [c/mL] | [%] | time [h] | [ug/mL] | [c/mL] | [%] | [ug/mL] | [c/mL] | [%] | [ug/mL] |
| WC2 | 4.85E+06 | 98.5 | 19.1 | 0 | 2.07E+07 | 97.2 | 4 | 1.80E+07 | 84.2 | 3 |
| WH2 | 5.58E+06 | 99.5 | 18.1 | 8 | 1.92E+07 | 95.9 | 23 | 1.59E+07 | 87.2 | 22 |
| XA2 | 6.61E+06 | 99.2 | 17.1 | 0 | 1.92E+07 | 96.4 | 3 | 1.76E+07 | 88.4 | 5 |

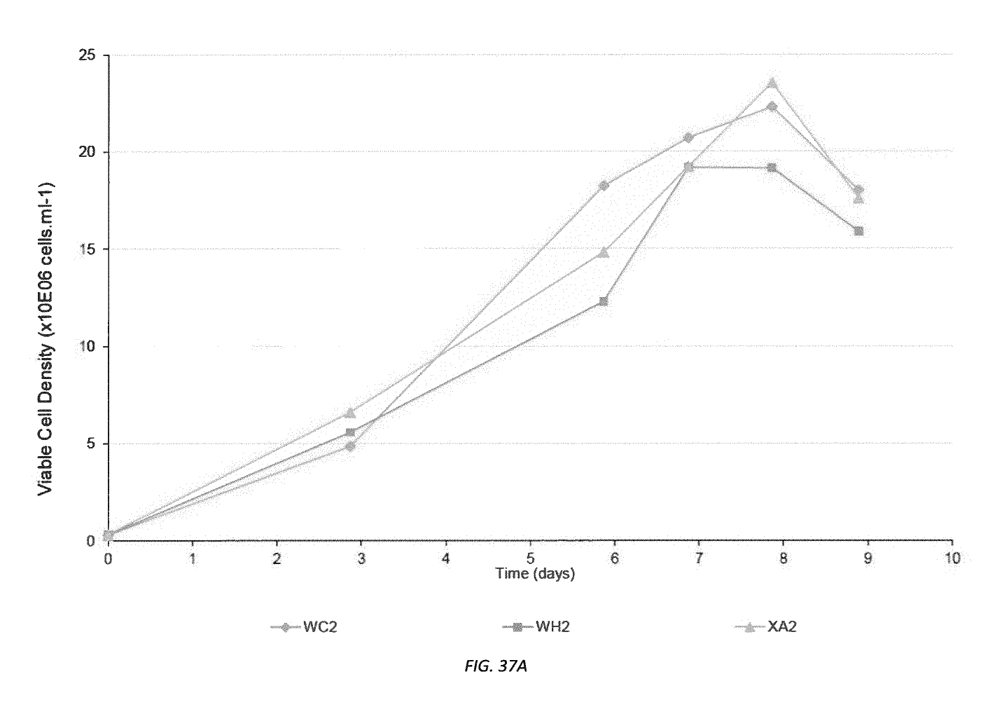

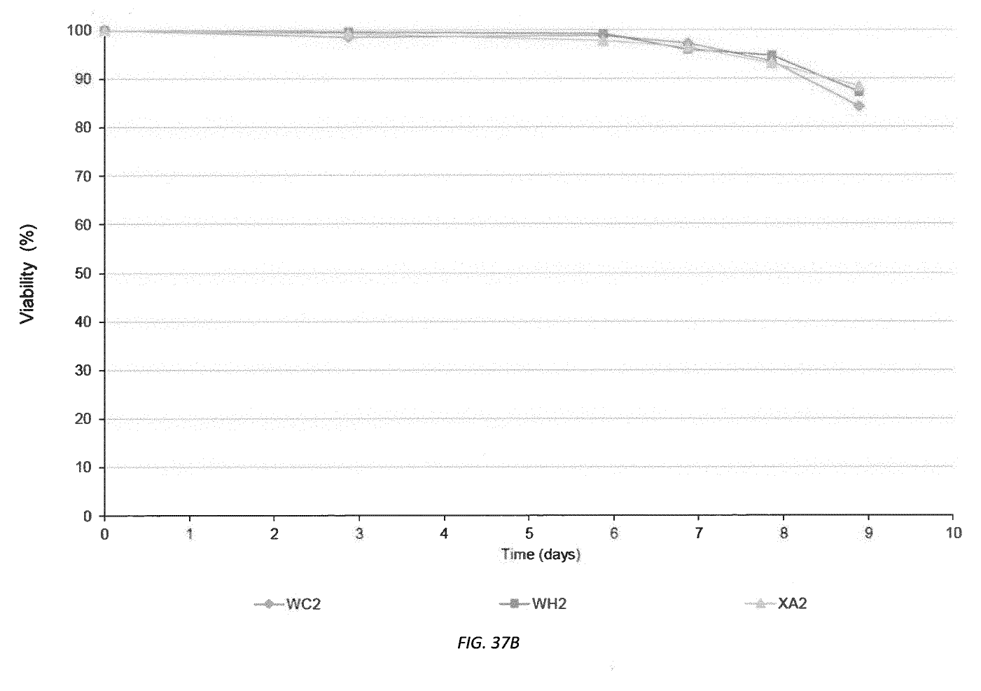

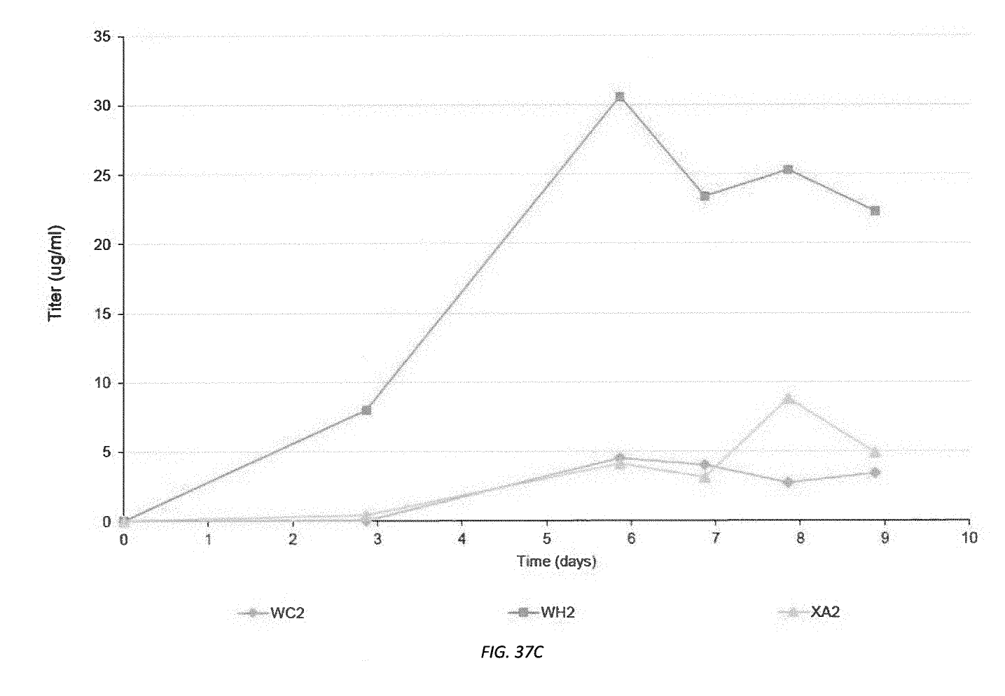

FIGS. 37A-37C illustrate growth, viability, and titer curves, respectively, for each cell line.

One of the promising clones (see Table 66, below) was re-assessed for growth, viability, and production.

TABLE 66

| | day 3 | | | | day 7 | | | day 10 | | |
|---|---|---|---|---|---|---|---|---|---|---|
| | cell conc | viability | doubling | Titer | cell conc | viability | Titer | cell conc | viability | Titer |
| # | [c/mL] | [%] | time [h] | [ug/mL] | [c/mL] | [%] | [ug/mL] | [c/mL] | [%] | [ug/mL] |
| XC | 4.65E+06 | 97.2 | 19.3 | 3 | 3.33E+07 | 95.7 | 90 | 2.47E+07 | 57.3 | 109 |

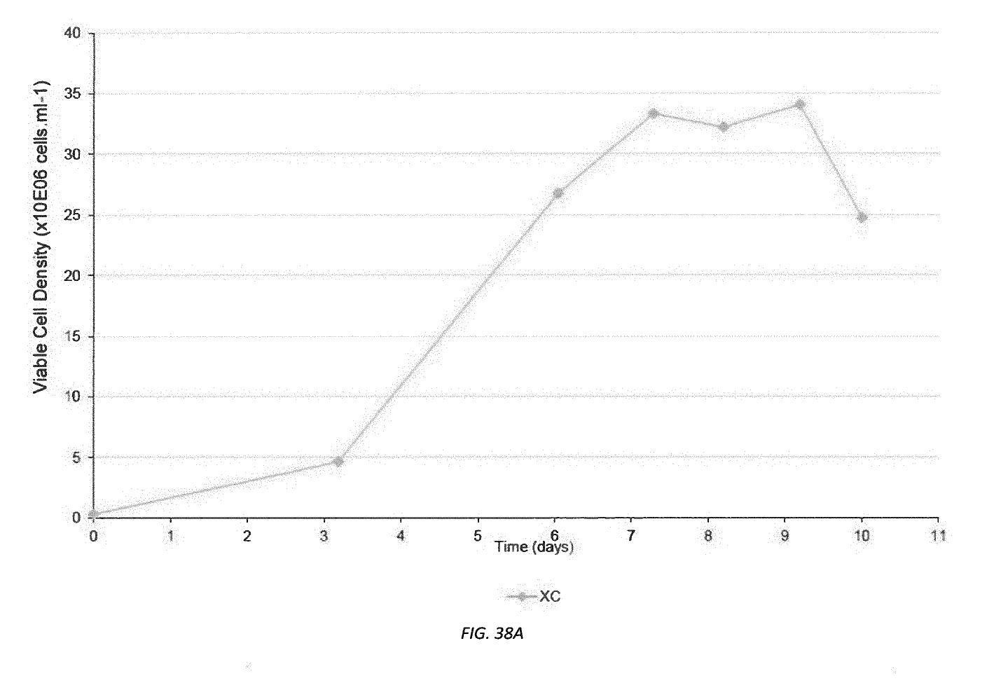

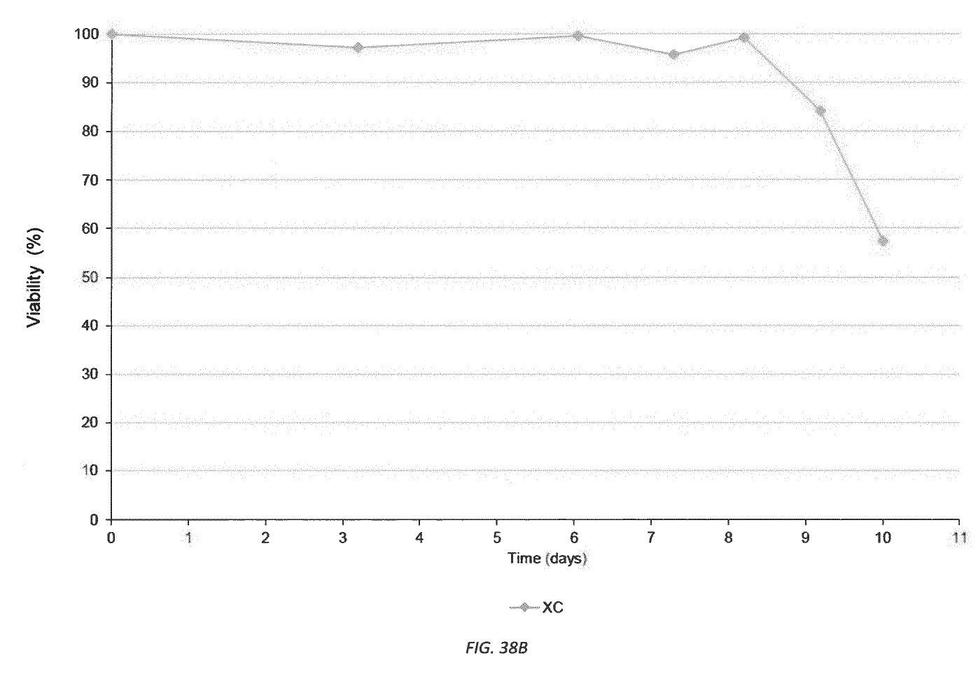

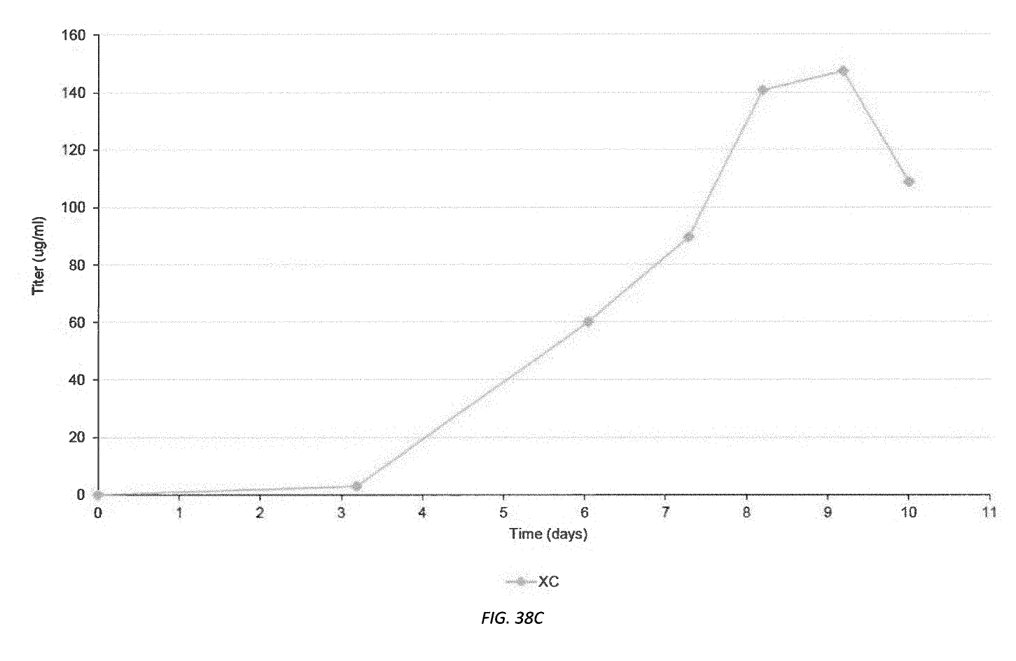

FIGS. 38A-38C illustrate growth, viability, and titer curves, respectively, for the cell line.

### Example 5

Western blot analysis was performed on expressed protein pools as illustrated in FIGS. 39A-39B. FIG. 39A illustrates Klotho ECD (34-981) 6x His Protein, and FIG. 39B illustrates the indicated Klotho protein. Unexpectedly, Klotho proteins appear to aggregate into higher molecular weight multimers.

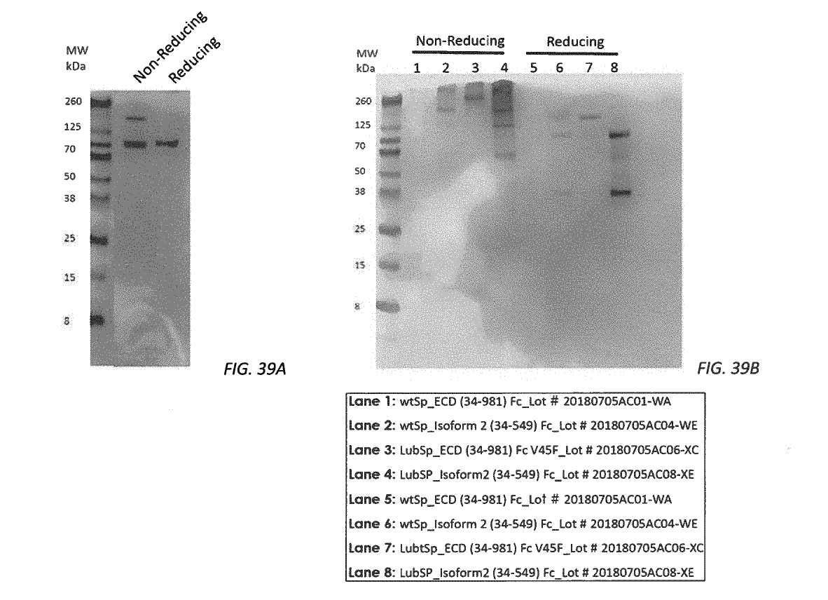

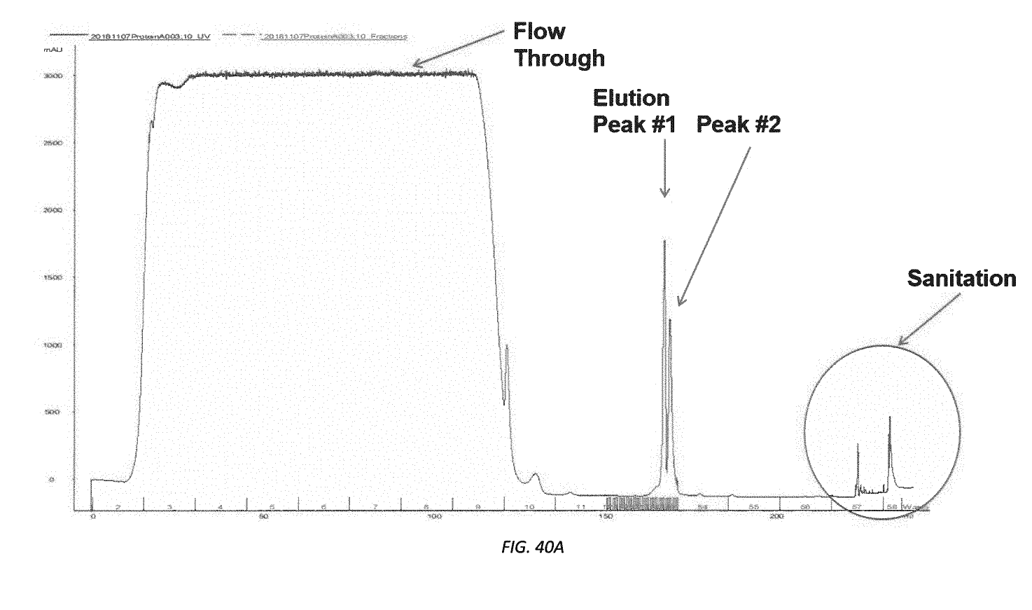

A 5 L bioreactor expression run was carried out, followed by adapter purification protocols. In a first purification step, Protein A Sepharose 4 Fast Flow (0.75 ml) (0.5 cm×3 cm) column was equilibrated in 10 mM Tris-HCl, pH 8.0. Protein sample (100 ml of Cell Supernatant adjusted to pH 8.0, with 1.5 M Tris-HCl, pH 8.0 (˜1 ml)) was loaded at a flow rate of 0.75 ml/min, washed with 10 mM Tris/Arginine-HCl, pH 8.0 buffer, and eluted with 4 M MgCl₂, pH 3 (i.e., low pH, high salt) at 0.50-0.25 ml/min to produce a concentrated sample. See FIG. 40A. The column was re-equilibrated with 10 mM Tris-HCl, pH 8.0 (to prevent salt precipitate), and sanitized with 0.1 M NaOH: 0.5-0.3 ml/ml to avoid over pressurization. Minimal Klotho protein was eluted during re-equilibration and sanitation.

Peak #1 and Peak #2 from the Protein A elution were analyzed separately, buffer exchanged, and fractionated using Superdex 200 (1 cm×30 cm) size exclusion chromatography column with 100 mM Tris-HCl/5 mM L-Methionine/0.6% Sodium Thioglycolate, pH 8.0 mobile phase at 0.5 mL/min flow rate. See FIG. 40B. Tris was used to buffer the mobile phase instead of a phosphate-containing buffer to prevent the precipitation of magnesium salts. Reducing agent (L-methionine) was used to prevent the oxidation of Klotho-Fc. Sodium Thioglycolate was used to prevent the scrambling of free sulfhydryl cysteine groups of Klotho-Fc. Buffer components are GRAS listed by the FDA.

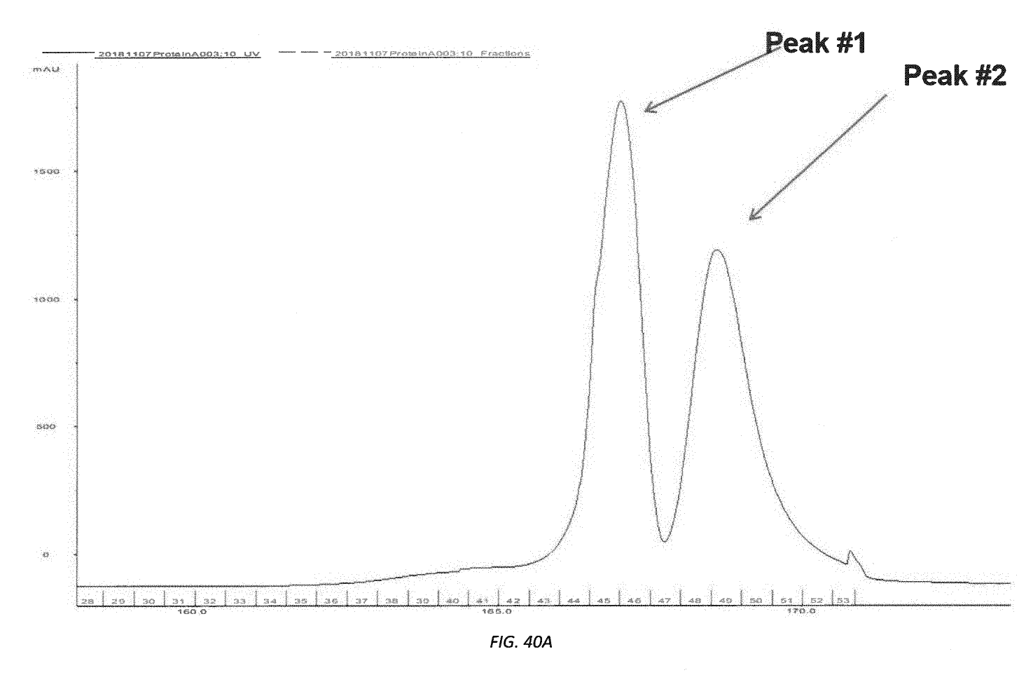

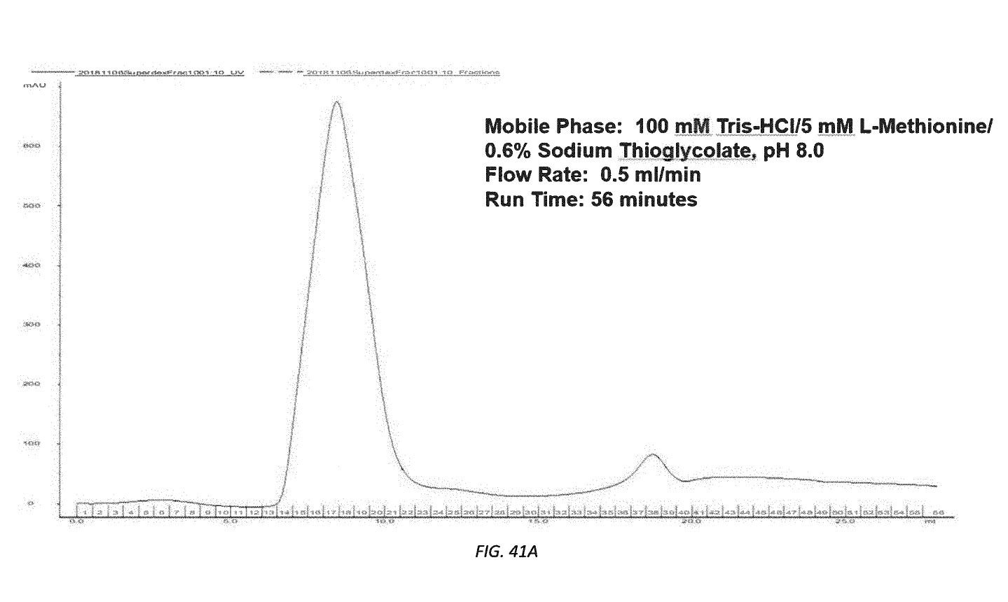

Superdex 200 fractions from single isolated Peak #1 and Peak #2 each show different MW species of klotho-Fc protein. See FIG. 41A and 41B. In summary, analysis of harvested proteins (in media) by SDS-PAGE, suggests that Klotho-Fc exists in various states of aggregation. The purification produced two populations of multimeric Klotho-Fc corresponding to a dimer (≤300 kDa) and a tetramer (≤600 kDa).The binding/elution protocol from Protein A shows far superior product elution profile and produces minimal protein after sanitation. As the formulation has thioglycolate as a reducing agent, the product separates as monomer after heating, and monomer and dimer without heating in gel electrophoresis. These new purification procedures resulted in non-aggregated Fc-klotho protein.

### Example 6

A bioassay was developed to test the activity of both native and Fc-Klotho proteins, and particularly to test the binding affinity of Klotho proteins to FGF 23 using BLITZ by ForteBio. Initially, the binding affinity of Recombinant Mouse Klotho to Mouse FGF-23 using Surface Plasmon Resonance (SPR) binding methods was assessed. SPR is an optical technique utilized for detecting molecular interactions between an analyte, a mobile molecule, and a ligand, an immobilized molecule bound to the sensor, which upon interaction change the refractive index of the sensor. In this study, the Inventors compared the affinity of six concentrations of Recombinant Mouse Klotho (2-fold serial dilutions from 400 ug/mL to 12.5 ug/mL) antibody to Mouse FGF-23. Using anti-HIS (HIS2) sensors, Mouse FGF-23 is bound to the sensors and then subsequently these sensors are used to produce binding curves for six different concentrations of the Mouse Klotho. From the six association and disassociation curves produced for the antibody, the disassociation constant (K_{d}) value of the antibody is determined. Assays were performed and data verified (data not shown).

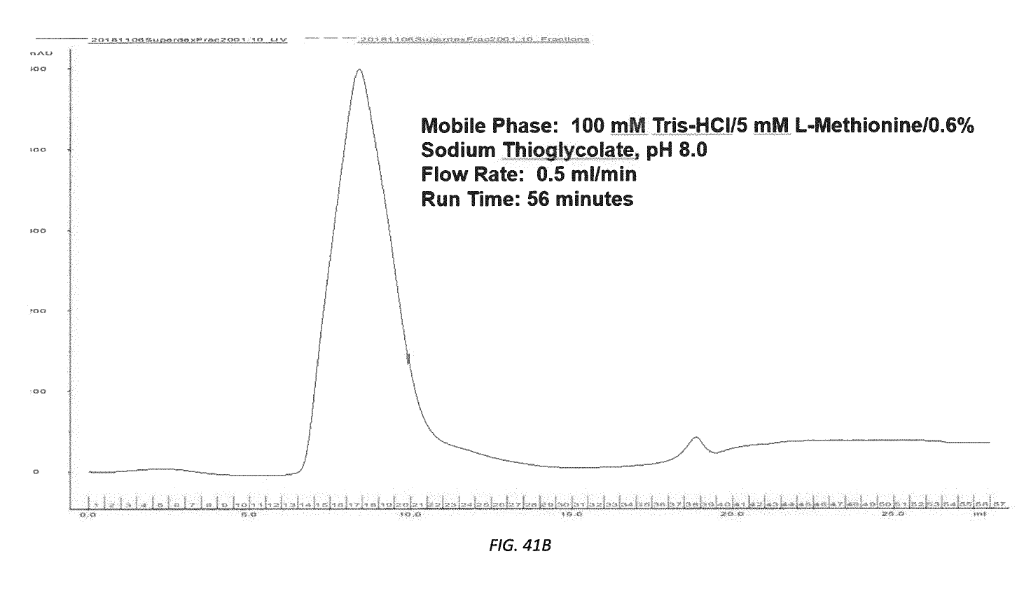

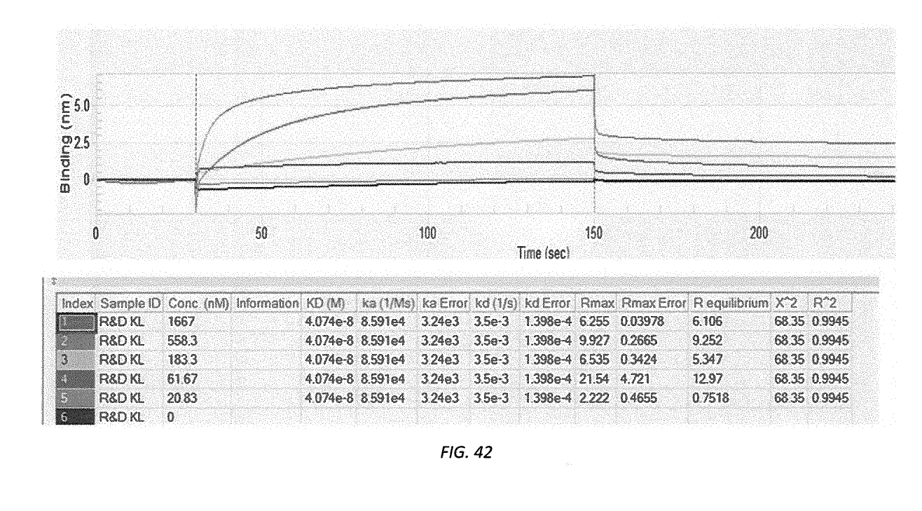

For human recombinant Klotho protein, HIS-Tag biosensor tips were hydrated in PBS for 10 minutes at RT. HIS-Tag labeled FGF23 (ProSpec, Cat#CYT-374) at 300ug/ml in PBS was coupled to biosensor for 2 minutes. Base line was established for 30 seconds on BLITZ instrument. Then, the binding of native and Fc-sKlotho proteins at five two-fold serial dilutions (from 400 ug/ml to 12.5 ug/ml) to immobilized FGF23 was tested for 2 minutes in PBD, and subsequently dissociated for 2 minutes in PBS. The Kₒₙ (association), K_{off} (dissociation), and KD (Koff/Kon) was analyzed using global analysis. See FIG. 42.

Further assays were performed to test the bioactivity of Klotho proteins using cell based functional assay, and particularly to test the bioactivity of both native and Fc-Klotho proteins by measuring their effect on stimulating NIH/3T3 cell proliferation. Based on biology of Klotho, the rationale of this assay development is to test the effect of Klotho on stimulating the proliferation of NIH-3T3 cells that expressed FGFR.

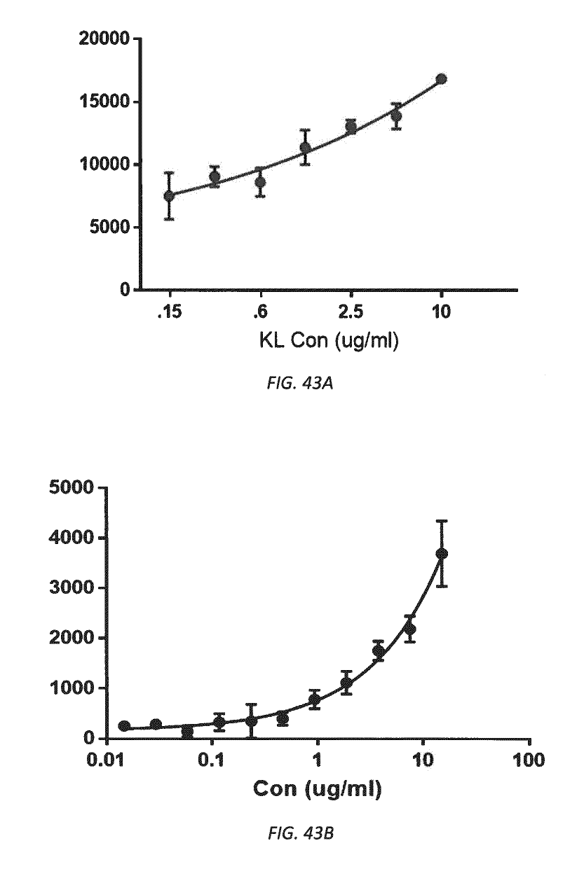

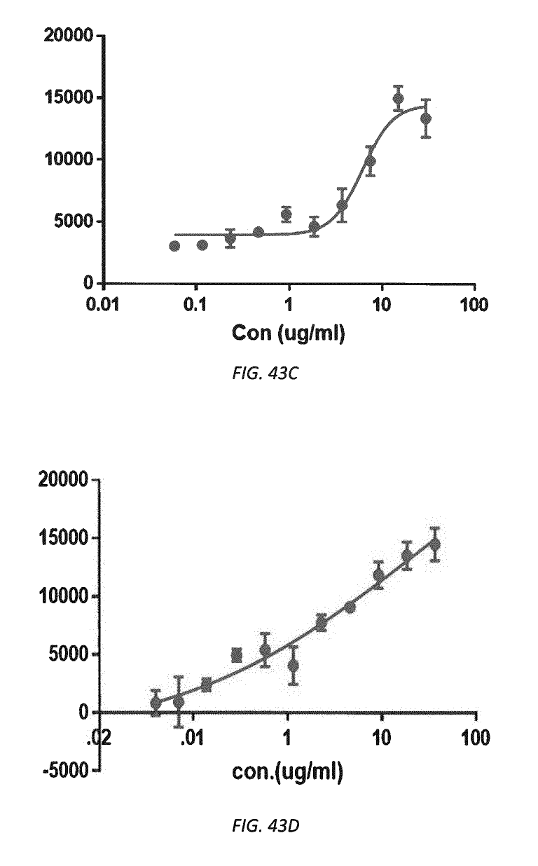

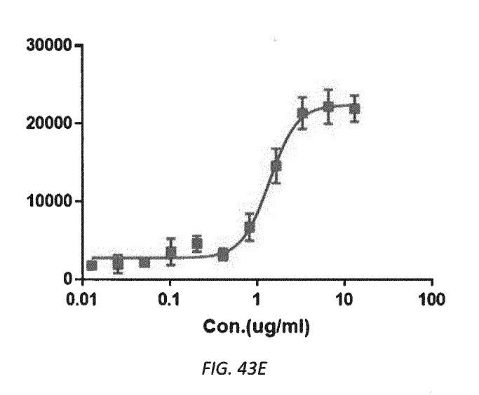

Growing phase cells were seeded in culture media (10% FBS-DMEM) at a density of 5000 cells/well/96-well plate, and cultured overnight, followed by a media change to 0.5% FBS and overnight culture. Cells were treated with 10 doses of respective Klotho protein starting at 30 ug/ml, 2-fold serial dilution, each dose in triplicate, and incubated for 48 hours. Cell proliferation was measured using Cell Titer-Glu assay kit, and data was analyzed using 4 parameter-fit curve with GraphPad Prism. The data indicate that purified Klotho proteins, both Native-His and Fc-Klotho, stimulated cell proliferation in a dose-dependent manner. See FIGS. 43A-43E.

### Example 7

A study to determine the Acute Tolerated Dose (ATD) of Alpha Human Native Klotho and Alpha Human Fc-Fusion Klotho proteins following intravenous administration in male Sprague-Dawley rats was conducted. In the acute tolerated dose study, the safety of the test articles Alpha Human Native Klotho and Alpha Human Fc-Fusion Klotho proteins was assessed in male Sprague-Dawley rats. Prior to dose administration, 21 animals were subcutaneously implanted with transponders. On Study Day -1, animals were randomized into seven groups of three, based on body weights. Alpha Human Native Klotho protein was tested at three dose concentrations of 10 μg/kg, 30 μg/kg, and 100 μg/kg (based on individual body weight) administered intravenously at a dose volume of 1 ml/kg (based on individual body weight) in vehicle (PBS, pH 7.2) on Study Day 0.

Alpha Human Fc-Fusion Klotho protein was also tested at three dose concentrations of 3 μg/kg, 10 μg/kg and 30 μg/kg (based on individual body weight) administered intravenously at a dose volume of 1 ml/kg (based on individual body weight) in vehicle (PBS, pH 7.2) on Study Day 0. Dosing began with a vehicle-only (PBS, pH 7.2) Group, 10 μg/kg Alpha Human Native Klotho protein Group, and 3 μg/kg Alpha Human Fc-Fusion Klotho protein Group on Study Day 0 and were staggered by at least 24 hours between each test article dose level group. All dosing was given once on the respective start date. Animals were observed for any adverse effects for maximum of 14 days following treatment.

In-life measurements included twice-daily checks for morbidity and mortality, and clinical observations were noted daily. Body weight measurements were recorded daily. Following completion of the observation phase, all animals were euthanized via cardiac exsanguination. Terminal measurements included blood hematology, serum biochemistry and coagulation. Gross pathology changes were recorded for all animals at necropsy and weights of ten organs (adrenals, brain, epididymides, heart, kidneys, liver, spleen, testes, thyroid/parathyroid and thymus) were taken.

Table 67 presents a summary of study animals.

TABLE 67

| | | Total | Number of | |
|---|---|---|---|---|
| | Age | Number | Animals | Number of |
| Species/Strain/Sex | Range | of Animals | per Group | Groups |
| Rat/Sprague | 8 weeks | 21 | 3 | 7 |
| Dawley/M | | | | |

Table 68 presents a summary of study design.

TABLE 68

| Group | Test Article | Dose Level μg/kg |
|---|---|---|
| 1 | PBS, pH 7.2 | — |
| 2 | Alpha Human Native Klotho | 10 |
| 3 | Alpha Human Native Klotho | 30 |
| 4 | Alpha Human Native Klotho | 100 |
| 5 | Alpha Human Fc- Fusion Klotho | 3 |
| 6 | Alpha Human Fc- Fusion Klotho | 10 |
| 7 | Alpha Human Fc- Fusion Klotho | 30 |

For Group Assignment: Matched Pair Distribution, an optimized distribution method using an algorithm that matches the individual measurement values with the mean of all selected animals was performed. The method begins by taking matching pairs that average close to the mean of all selected animals and then distributes them into groups that will then match the average (or as close as possible) of the mean of all selected animals. Animals are distributed so that the average of the measurement for each group will be as close to the mean of all selected animals as possible.

For Statistical Analyses, descriptive statistics were generated from Study data. Data was assessed to determine whether parametric or non- parametric analysis was appropriate. For parametric data, analysis of variance (ANOVA) followed by post-hoc tests was performed to determine significant differences between treatments, time points, and/or groups. For non-parametric data, appropriate statistical analyses were performed (e.g., Kaplan-Meier Survival, Kruskal-Wallis One-way ANOVA, Mann-Whitney or Wilcoxon Rank Sum, etc.).

Table 69 summarizes sample collection procedure.

TABLE 69

| | | Sample Info/ |
|---|---|---|
| Task | Method & Collection | Special Instructions |
| Blood for clinical | collected into non-heparinized | stored at −80° C. |
| biochemistry | tubes for serum preparation | and shipped on |
| | | dry ice |
| Blood for coagula- | collected into sodium | stored at −80° C. |
| tion analyses | citrate tubes | and shipped on |
| | | dry ice |
| Whole blood for | collected into K₂EDTA | stored at 2-8° C. |
| CBC count | tubes | and shipped |
| hematology analysis | | |

Table 70 summarizes sample collection tasks.

TABLE 70

| Task | Frequency |
|---|---|
| Body weight | Daily |
| Adrenals, brain, epididymides, heart, kidneys, liver, | At termination |
| spleen, testes, thyroid/parathyroid and thymus to be | |
| excised and weighted | |
| Clinical Observation | Daily |
| Morbidity Check | 2x daily |
| Mortality Observation | 2x daily |

Any animal whose body weight dropped below 85% of initial body weight, or displays severe, prolonged adverse clinical signs was to be euthanized. Animals were to be euthanized via cardiac bleed for collection of plasma, serum and whole blood as indicated above.

Preliminary results indicated that the rats tolerate relatively high doses of recombinant Klotho proteins administered thereto.

### Example 8

A study to determine Pharmacokinetics of Alpha Human Native Klotho and Alpha Human Fc-Fusion Klotho Proteins Following IV Dosing in Male Sprague Dawley Rats was conducted. Prior to Study Day 0, animals were subcutaneously implanted with transponders. On Study Day -1 body weights were acquired and animals were randomized based on body weight. On Study Day 0, animals in Groups 5, 6, and 7 (10 animals per group) received a single dose of Alpha Human Fc-Fusion Klotho protein intravenously via the tail vein (see detailed dosing schedule, below) at 3, 10, and 30 μg/kg in 1 ml/kg vehicle (PBS, pH 7.2), respectively. On Study Day 1, animals in Groups 2, 3 and 4 (10 animals per group) received a single dose of Alpha Human Native Klotho protein intravenously via the tail vein (see detailed dosing schedule, below) at 10, 30 and 100 μg/kg protein in 1 ml/kg vehicle (PBS, pH 7.2), respectively. Animals in the vehicle Group 1 (3 animals) was also dosed on Study Day 1 at 1 ml/kg vehicle (PBS, pH 7.2) without protein. The volume of dosing solution administered to each animal was calculated and adjusted based on individual body weight measured acquired on Study Day-1.

Three blood samples were obtained from each group (2-7) for all time points, equating to three blood samples per rat (see detailed blood collection schedule, below). Time-point blood samples were obtained pre-dose, 3 min, 10 min, 20 min, 30 min, 1 hr, 2 hr, 4 hr, 8 hr and 24 hr post-dose (Groups 2, 3 and 4), as presented in Table 71, below.

TABLE 71

| | Groups 2, 3 and 4 | | | | | | | | | |
|---|---|---|---|---|---|---|---|---|---|---|
| Rat ID | 0 min | 3 min | 10 min | 20 min | 30 min | 1 hr | 2 hr | 4 hr | 8 hr | 24 hr |
| 1 | X | | | | X | | | X | | |
| 2 | | X | | | | X | | | X | |
| 3 | | | X | | | | X | | | X |
| 4 | X | | | X | | | | X | | |
| 5 | | X | | | X | | | | X | |
| 6 | | | X | | | X | | | | X |
| 7 | X | | | X | | | X | | | |
| 8 | | X | | | X | | | X | | |
| 9 | | | X | | | X | | | X | |
| 10 | | | | X | | | X | | | X |

Time-point blood samples were obtained pre-dose, 3 min, 1 hr, 4 hr, 8 hr, 24 hr, 48 hr, 72 hr, 96 hr and 7 days post-dose (Groups 5, 6, and 7), as presented in Table 72, below. An additional blood collection at 14 days post dosing was obtained in Groups 5, 6, and 7.

TABLE 72

| | Groups 5, 6 and 7 | | | | | | | | | | |
|---|---|---|---|---|---|---|---|---|---|---|---|
| | | | | | | | | | | | 14 day (to be |
| Rat ID | 0 min | 3 min | 1 hr | 4 hr | 8 hr | 24 hr | 48 hr | 72 hr | 96 hr | 7 day | determined) |
| 1 | X | | | | X | | | X | | | |
| 2 | | X | | | | X | | | X | | |
| 3 | | | X | | | | X | | | X | X |
| 4 | X | | | X | | | | X | | | |
| 5 | | X | | | X | | | | X | | |
| 6 | | | X | | | X | | | | X | X |
| 7 | X | | | X | | | X | | | | |
| 8 | | X | | | X | | | X | | | |
| 9 | | | X | | | X | | | X | | |
| 10 | | | | X | | | X | | | X | X |

Vehicle Group 1 animals were bled terminally once, 1 hr post dose administration, as presented in Table 73, below.

TABLE 73

| | Group 1 |
|---|---|
| Rat ID | 1 hr |
| 1 | X |
| 2 | X |
| 3 | X |

Resulting serum samples were analyzed via Alpha Klotho Human Soluble ELISA kit.

Table 74 presents a summary of study animals.

TABLE 74

| | | Total | Number of | |
|---|---|---|---|---|
| | Age | Number | Animals | Number of |
| Species/Strain/Sex | Range | of Animals | per Group | Groups |
| Rat/Sprague | 8-11 weeks | 63 | 3-10 | 7 |
| Dawley/M | | | | |

Table 75 presents a summary of study design.

TABLE 75

| Group | Test Article | Dose Level μg/kg |
|---|---|---|
| 1 | PBS, pH 7.2 | — |
| 2 | Alpha Human Native Klotho | 10 |
| 3 | Alpha Human Native Klotho | 30 |
| 4 | Alpha Human Native Klotho | 100 |
| 5 | Alpha Human Fc- Fusion Klotho | 3 |
| 6 | Alpha Human Fc- Fusion Klotho | 10 |
| 7 | Alpha Human Fc- Fusion Klotho | 30 |

For Group Assignment: Stratified Sampling, a non-optimized randomization method that uses “binning” of the animals with measurement values of similar size was used. By this method, the pool of animals is stratified by sorting them by the measurement value of interest in ascending order. Animals are then organized into “bins” or strata according to the size of the measurement value. The number of strata is determined by the number of animals that are to be assigned per group. For example, if there are three animals per group, then three bins would be created: small, medium and large. One animal from each size bin is assigned randomly to each group. Stratified sampling ensures that each group receives one animal randomly from each size bin and the means of each group will remain similar to the other groups. This method balances differences between group means and standard deviations and controls for pool order bias.

For Statistical Analyses, Descriptive statistics were generated from Study data. Data were assessed to determine whether parametric or non-parametric analysis was appropriate. For parametric data, analysis of variance (ANOVA) followed by post-hoc tests was performed to determine significant differences between treatments, time points, and/or groups. For non-parametric data, appropriate statistical analyses were performed (e.g., Kaplan-Meier Survival, Kruskal-Wallis One-way ANOVA, Mann-Whitney or Wilcoxon Rank Sum, etc.).

Table 76 summarizes sample collection procedure.

TABLE 76

| | | Sample Info/ |
|---|---|---|
| Task | Method & Collection | Special Instructions |
| Serum preparation | Blood collected via saphenous | Samples processed |
| for PK sampling | vein or cardiac puncture into | to serum and stored |
| | non-heparinized tubes for | at −80 C. prior to |
| | serum | ELISA analysis |

Table 77 summarizes sample collection tasks.

TABLE 77

| Task | Frequency |
|---|---|
| Body weight | On Study Day −1 and twice weekly thereafter |
| Clinical Observation | 2x weekly |
| Mortality Observation | As required |

Any animal whose body weight dropped below 85% of initial body weight or displays severe, prolonged adverse clinical signs was euthanized (data not shown). Animals were euthanized via cardiac bleed for collection of plasma, serum and whole blood as indicated above.

### Example 9

A study to determine Dose-Dependent Effects of Alpha Human Fc-Klotho and Alpha Human Native Klotho on I/R Induced-AM and Renal Biomarkers (by measuring renal I/R-induced increases in plasma renal injury biomarkers) in Wistar Rats was performed. Specifically, the objective of this study was to evaluate the dose-dependent effects of alpha human Fc-Klotho and alpha human native Klotho on renal I/R-induced increases in plasma renal injury biomarkers in rats. The study was designed to provide data from or related to bilateral renal ischemia-reperfusion (I/R) injury (30′ ischemia duration) surgery (n=63) verses sham surgery (n=4), Compound dosing (one i.v. injection/rat), serial urine collections via metabolic cage housing (3 collections/rat; D1, D2 & D7), clinical chemistry analysis of serial urine specimens for creatinine and protein (D1, D2 & D7), serial blood collections (3 collections/rat; D1, D2 & D7), clinical chemistry analysis of serial plasma specimens for creatinine and BUN (D1, D2 & D7), daily body weights and health observations post-surgery through endpoint, and endpoint tissue collection and processing, with data presented in tabular and graphical format.

In addition, analysis of serial urine specimens for Kim-1 via species specific ELISA (D1, D2 & D7), analysis of serial urine specimens for NGAL via species specific ELISA (D1, D2 & D7), left and/or right kidney mRNA expression analysis (1-2 kidney/animal: n=67 samples; 10 analytes, includes 3 normalization genes; performed using the Affymetrix Quantigene Plex 2.0 platform); MCP-1, TGF-β, α-SMA, Co11A 1, Col3A1, Fn-1, CTGF), and left and/or right renal cortical immunohistochemistry and quantitation of macrophage infiltration via specific staining and quantitation of ED-1+ macrophage staining were also optionally performed.

Table 78 presents that study parameters.

TABLE 78

| | |
|---|---|
| Compounds: | Alpha Human Fc- Klotho: 10 or 30 μg/kg |
| | Alpha human Native Klotho: 10 or 30 μg/kg |
| Route/ | Alpha Human Fc-Klotho: i.v./dose once at either t − 0.5 |
| Frequency: | hour or t + 1 hour relative to time of ischemia inception |
| | Native Klotho: i.v./dose once at either t − 0.5 hour or t + |
| | 1 hour relative to time of ischemia inception |
| Vehicle/Dose | Alpha Human Fc-Klotho: PBS/1 ml/kg i.v. |
| Volume: | Native Klotho: PBS/1 ml/kg i.v. |
| Compound | Alpha Human Fc-Klotho: 0.221 mg |
| Requirement: | Native Klotho: 0.221 mg |

Table 79 presents a summary of study design.

TABLE 79

| | | Compound & | | |
|---|---|---|---|---|
| | | Administration | Dose | |
| | N/group | Interval (0 hr = | Volume | |
| | (Animal | time of ischemia | & | |
| Group | #s) | inception) | Route | Challenge |
| 1 | N = 4 | Vehicle | 1 ml/kg | Sham operated |
| | (4528-4531) | +1 Hr. | i.v. | 7 Days Post-Sx |
| 2 | N = 9 | Vehicle | 1 ml/kg | 30′ Ischemia |
| | (4532-4540) | +1 Hr. | i.v. | 7 Days Reperfusion |
| 3 | N = 9 | 10 μg/kg Fc- | 1 ml/kg | 30′ Ischemia |
| | (4541-4549) | Klotho +1 Hr. | i.v. | 7 Days Reperfusion |
| 4 | N = 9 | 30 μg/kg Fc- | 1 ml/kg | 30′ Ischemia |
| | (4550-4558) | Klotho +1 Hr. | i.v. | 7 Days Reperfusion |
| 5 | N = 9 | 10 μg/kg Native | 1 ml/kg | 30′ Ischemia |
| | (4559-4567) | Klotho +1 Hr. | i.v. | 7 Days Reperfusion |
| 6 | N = 9 | 30 μg/kg Native | 1 ml/kg | 30′ Ischemia |
| | (4568-4576) | Klotho +1 Hr. | i.v. | 7 Days Reperfusion |
| 7 | N = 9 | 30 μg/kg Fc- | 1 ml/kg | 30′ Ischemia |
| | (4577-4585) | Klotho −0.5 Hr. | i.v. | 7 Days Reperfusion |
| 8 | N = 9 | 30 μg/kg Native | 1 ml/kg | 30′ Ischemia |
| | (4586-4594) | Klotho −0.5 Hr. | i.v. | 7 Days Reperfusion |

Sixty-seven (67) male Wistar rats weighing approximately 151-175 g were acquired and fed a standard chow diet (Harlan 8640), group housed under standard conditions, and allowed to acclimate for at least 7 days before enrolling in study. Prior to study inception, the study rats were placed into weight-matched treatment groups. Animals were enrolled to study using a balanced design, such that approximately equal numbers of animals were enrolled per group per surgical day, and the heaviest animals enrolled first to minimize differences in animal age. When not in metabolic cage housing, rats were group housed under standard conditions in static caging.

At t-0.5 hours, study rats were anesthetized with isoflurane anesthesia on a nosecone, prepared for surgery and core temperature allowed to equilibrate prior to renal UR operative procedure. Rats were administered 10 ml/kg warm, sterile saline (s.c.; 25% pre- and 75% post-surgery) to minimize perturbations in body fluid volume. Core temperatures were maintained normative (37±0.2° C.) throughout ischemia duration. At t-0.5 hours, animals in Groups 7 and 8 also received an i.v. injection of compound as appropriate.

A laparotomy was then performed. At tO, rats were subjected to either sham (Group 1, n=4) or bilateral renal arterial ischemia (Groups 2-8; n=9/grp) using heat sterilized instruments, vascular occlusive clamps, and aseptic surgical technique. Following thirty (30) minutes of continuous ischemia time (t+0.5), the occlusive clamps were removed, the kidneys reperfused and the abdominal wound closed with sterile silk suture. Rats were administered 0.01 mg/kg buprenorphine as post-operative analgesic and allowed to recover briefly in a clean, heated cage. At t+1 hour, rats in Groups 1-6 were briefly restrained and received an i.v. injection of vehicle or Klotho compound in vehicle as appropriate. Rats in Groups 7 and 8 were also briefly restrained (but not injected) to control for handling stress. Rats were then immediately placed into metabolic cages.

Twenty-four (24) hours following reperfusion (D1), all animals had urine volumes determined and sampled. All animals then had blood (500 μl) collected on Li-Heparin by direct tail vein stick. Immediately post-bleed, rats received 500 μl warm saline. Urine and blood collection was repeated again on D2 (48 hours post-reperfusion) and D7 (168 hours post-reperfusion). On D2-D5, all rats were grouped housed under standard conditions in static caging and weighed, and underwent health observations daily. Whole blood (500 μl) was processed appropriately for the production of plasma and processed as described.

Clinical observations were conducted once daily following inception of dosing. With regard to data/tissues to be collected/calculated, animal health was observed daily following surgical procedure, and kidney mass (left and right kidney weights) was indexed to tibia length and body weight. Urine was collected at D1, D2 and D7 (targeting n=3 samples/animal; 500 μl/sample). At least one sample was subjected to clinical chemistry analysis (creatinine, protein) and at least one sample was optionally used for Kim-1 and NGAL analysis. Plasma was collected on Li-Heparin at D1, D2 and D7 (targeting n=2 samples/animal; 125 μl/sample). At least one sample was subjected to clinical chemistry analysis (creatinine, BUN).

On D7, rats were anesthetized with isoflurane immediately following bleed and tissues were harvested. Both left and right kidneys were immediately placed in ice-cold 0.9% NaCl, de-encapsulated and weighed. After tissue harvest (for histological and other analysis), all remaining inner cortical/outer medullary (i.e. S3/THAL enriched) from each sham and ischemic kidney (left and right kidney from each animal; 1 tube/kidney/animal) were snap frozen in appropriately labeled tube and stored at −80° C. for future potential biochemical analysis. Prior to any analyses being performed, cortical tissue was optionally cryogenically powdered and mixed to enable better homogeneity of the tissue sample across biochemical assays as well as limit introduction of sampling bias that may occur with cortical biopsies. At least one mid-transverse section of each sham and ischemic kidney (left and right from each animal) was optionally collected and immersion fixed in 10% neutral buffered formalin for histological analysis. The rats were then sacrificed.

### Example 10

The body weight of study rats were measured to test for the effect of Klotho protein administration on subjects. Table 80 presents a summary of study animals.

TABLE 80

| Group | Test Article |
|---|---|
| Group 01 | Vehicle only, PBS, pH 7.2 |
| Group 02 | Alpha Human Native Klotho, 10 μg/kg |
| Group 03 | Alpha Human Native Klotho, 30 μg/kg |
| Group 04 | Alpha Human Native Klotho, 100 μg/kg |
| Group 05 | Alpha Human Fc-Fusion Klotho, 3 μg/kg |
| Group 06 | Alpha Human Fc-Fusion Klotho, 10 μg/kg |
| Group 07 | Alpha Human Fc-Fusion Klotho, 30 μg/kg |

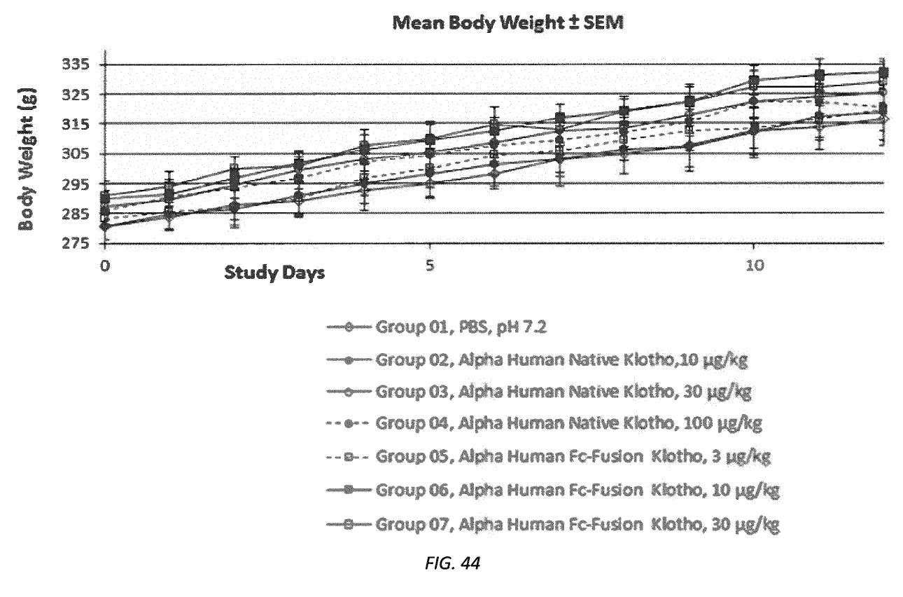

FIG. 44 illustrates, graphically, the average (mean) body weight (+/−standard error of the mean; SEM bars) of rats in each group over the course of the 12-day study (daily). Table 81, below, presents the corresponding data for mean body weight illustrated in FIG. 44, Table 82, below, presents the corresponding SEM illustrated in FIG. 44, and Table 83, below, presents the percent change in mean body weight of rats in each group over the course of the 12-day study (daily).

### Example 11

To study the therapeutic effect of Klotho administration on Acute Kidney Injury (AKI) following renal ischaemia reperfusion injury (IRI), Sprague-Dawley rats were randomly divided into either Sham or AKI groups, and the animals in each group were randomly allocated into vehicle or Klotho treatment. In humans, AKI is a formidable clinical problem resulting primarily from ischemia or nephrotoxins, with outcomes ranging from full recovery to the development of chronic kidney disease with dialysis dependence, or death. Animals in the Klotho group received one bolus intraperitoneal injection of recombinant mouse Klotho protein (0.01 mg/kg body weight) 30 or 60 min after reperfusion; rats in vehicle group received the same volume of Klotho buffer (150 mM NaCl and 10 mM HEPES pH 7.4). Twenty-four-hour urine and blood were collected on days 1, 2, and 7 after surgery. This study showed that a single bolus injection of murine S-Klotho 30 min post-injury was effective in attenuating (AKI) in animal model of the human condition.

### Example 12

To determine whether Klotho protects the lung against oxidant injury, Sprague-Dawley rats (body weight ˜300 g) were exposed to normoxia (N: 21% inspired O) or hyperoxia (H: 90% O) for 3 days while receiving intra-peritoneal injections of one of 3 preparations: Dulbeco's modified Eagle's Medium (DMEM) or conditioned medium (CM) from CHO cells overexpressing either Klotho (Klotho CM; ˜60 pmol) or empty vector (Control CM) (n=6-8 animals each). Injections were given 12 h before, and repeated at 24 h and 48 h during exposure.

Also, 3LL cells were transplanted at the flanks of athymic mice by subcutaneous injection (2×10⁶ cells per mouse) and treated with Klotho protein (0.01 mg/kg, intraperitoneal) or vehicle every other day for 10 days. Lungs were harvested 21 days after transplantation and the number of metastatic nodules was counted.

TABLE 81

| Group | Day 0 | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|---|
| 1 | 280.67 | 283.93 | 287.87 | 288.93 | 292.63 | 295.00 | 298.30 | 302.90 | 305.03 | 307.30 | 312.27 | 313.63 | 316.47 |
| 2 | 280.40 | 284.77 | 286.40 | 290.77 | 294.77 | 298.07 | 301.20 | 303.23 | 306.33 | 307.00 | 311.97 | 317.33 | 318.80 |
| 3 | 287.23 | 289.47 | 294.67 | 299.60 | 302.83 | 305.23 | 308.37 | 312.47 | 313.47 | 317.77 | 322.17 | 323.93 | 325.27 |
| 4 | 285.83 | 290.43 | 293.50 | 296.73 | 302.30 | 304.50 | 307.40 | 309.27 | 311.93 | 315.53 | 322.17 | 322.43 | 320.70 |
| 5 | 283.10 | 285.50 | 287.10 | 290.07 | 296.93 | 300.00 | 304.53 | 305.77 | 309.17 | 312.63 | 313.30 | 316.67 | 319.53 |
| 6 | 290.23 | 291.37 | 296.53 | 301.50 | 306.17 | 309.33 | 312.30 | 316.93 | 319.03 | 322.17 | 329.43 | 331.30 | 332.30 |
| 7 | 291.10 | 294.23 | 300.10 | 300.57 | 307.30 | 309.77 | 314.53 | 312.83 | 319.03 | 322.40 | 327.30 | 327.27 | 328.83 |

TABLE 82

| Group | Day 0 | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|---|
| 1 | 5.72 | 4.64 | 4.98 | 4.40 | 6.57 | 4.62 | 5.28 | 5.70 | 6.82 | 7.07 | 7.74 | 7.33 | 9.08 |
| 2 | 4.07 | 5.06 | 6.35 | 6.70 | 6.70 | 7.92 | 7.09 | 9.12 | 8.13 | 8.21 | 8.56 | 8.06 | 9.33 |
| 3 | 5.55 | 3.71 | 4.83 | 4.41 | 4.56 | 5.05 | 6.27 | 4.94 | 6.20 | 6.20 | 5.87 | 5.52 | 6.57 |
| 4 | 2.97 | 5.81 | 5.20 | 5.44 | 5.78 | 5.88 | 6.53 | 4.92 | 4.91 | 4.90 | 5.23 | 5.47 | 5.69 |
| 5 | 6.92 | 5.68 | 6.08 | 6.45 | 6.03 | 6.53 | 6.72 | 7.11 | 6.48 | 7.00 | 6.81 | 6.32 | 7.31 |
| 6 | 4.72 | 3.84 | 3.51 | 4.15 | 4.72 | 5.34 | 4.48 | 4.54 | 4.25 | 5.86 | 4.78 | 5.40 | 4.47 |
| 7 | 4.91 | 4.62 | 3.64 | 4.56 | 5.39 | 5.67 | 5.85 | 5.24 | 4.95 | 4.60 | 5.14 | 5.39 | 6.98 |

TABLE 83

| Group | Day 0 | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|---|
| 1 | 0.00% | 1.18% | 2.58% | 2.97% | 4.26% | 5.13% | 6.29% | 7.93% | 8.67% | 9.48% | 11.24% | 11.73% | 12.72% |
| 2 | 0.00% | 1.55% | 2.12% | 3.68% | 5.11% | 6.28% | 7.40% | 8.12% | 9.23% | 9.46% | 11.23% | 13.15% | 13.66% |
| 3 | 0.00% | 0.81% | 2.60% | 4.32% | 5.45% | 6.28% | 7.36% | 8.80% | 9.14% | 10.63% | 12.17% | 12.79% | 13.25% |
| 4 | 0.00% | 1.59% | 2.67% | 3.80% | 5.74% | 6.51% | 7.52% | 8.19% | 9.12% | 10.38% | 12.70% | 12.79% | 12.18% |
| 5 | 0.00% | 0.87% | 1.43% | 2.47% | 4.91% | 5.98% | 7.58% | 8.01% | 9.23% | 10.45% | 10.68% | 11.88% | 12.88% |
| 6 | 0.00% | 0.40% | 2.19% | 3.89% | 5.49% | 6.58% | 7.61% | 9.21% | 9.93% | 11.00% | 13.51% | 14.15% | 14.50% |
| 7 | 0.00% | 1.08% | 3.11% | 3.26% | 5.56% | 6.41% | 8.05% | 7.47% | 9.60% | 10.77% | 12.45% | 12.43% | 12.95% |

### Conclusion

Existing Klotho protein measurement may be prohibitively expensive and/or technically challenging for at-home collection and/or quantitative diagnostic. Embodiments of the present disclosure provide methods for monitoring, detecting and/or quantifying Klotho protein level(s), particularly endogenous and/or exogenous soluble alpha Klotho protein level(s), and more particularly soluble alpha Klotho protein levels, diagnosing Klotho protein deficiency, and/or increasing Klotho protein level(s), expression, or production, particularly endogenous and/or exogenous soluble alpha Klotho protein level(s), expression, or production, in a subject, and products useful in performing the same, including diagnostic kits and compositions for treating Klotho protein deficiency.

In addition, while gene therapy can be effective to increase certain protein levels in animal models or studies, the safety of gene therapy, especially for human treatment, is still questionable. Compared to viral delivery of the klotho gene to animal (cells), the administration of exogenous and/or recombinant protein in humans may be a safer, easier, and more direct modality to restore (endocrine) klotho levels. Preclinical data supports the therapeutic potential of soluble Klotho protein for age-related disorders and klotho deficiency-associated diseases. Epidemiological data has shown that soluble Klotho is lower in the elderly than in young adults, and that levels of soluble Klotho are inversely correlated with age, indicating that aging may be associated with soluble Klotho decline. Generally, however, the production and administration of exogenous and/or recombinant protein to humans is tightly regulated by government and other entities. Because of the time and expense associated with regulatory compliance, the cost of such treatments can be prohibitive for developers, manufactures, and patients alike. Compositions and methods of the present disclosure, however, can provide a viable and effective option for increasing endogenous Klotho protein levels in humans and non-human animals without one or more of the drawbacks associated with other solutions. In particular, some embodiments of the present disclosure include products, compositions, and/or methods of manufacturing and/or using recombinant human Klotho proteins, such as (Current Good Manufacturing Practice (cGMP)-grade) human recombinant soluble alpha-Klotho proteins, protein fragments, and/or protein variants. In addition, some embodiments of the present disclosure include (nutraceutical or health supplement) products, compositions, and/or methods for (naturally) increasing (endogenous and/or serum soluble) Klotho protein level(s), expression, or production in a patient or subject.

Those skilled in the art will appreciate that each of the therapeutic effects, indications, and/or treatments outlined herein in relation to recombinant, therapeutic Klotho proteins and products, compositions, and methods related thereto can be achieved by (naturally) increasing, enhancing, augmenting, and/or supporting endogenous Klotho protein production and/or level(s). Those skilled in the art will also appreciate that each of the therapeutic effects, indications, and/or treatments outlined herein in relation to (naturally) increasing, enhancing, augmenting, and/or supporting endogenous Klotho protein production and/or level(s) can be achieved by administering recombinant, therapeutic Klotho proteins and products, compositions, and through methods related thereto. Accordingly, and for the sake of brevity, the present disclosure need not rehearse each of the disclosed therapeutic effects, indications, and/or treatments with reference to both (i) the administration of recombinant, therapeutic Klotho proteins, and (ii) the administration of the health supplement or nutraceutical compositions of the present disclosure, as such are clearly contemplated herein.

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being required in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

We claim:

1. A method of stabilizing Klotho protein in a mammalian blood sample, the method comprising:
obtaining capillary blood from a mammalian subject, the capillary blood containing an amount of soluble Klotho protein; and
storing the capillary blood in a container for at least 24 hours at room temperature or without freezing, the container having a preservative or anti-coagulant disposed therein, the preservative or anti-coagulant being mixed with the capillary blood in the container, the preservative or anti-coagulant stabilizing the soluble Klotho protein for at least 24 hours at room temperature or without freezing, the preservative or anti-coagulant comprising one or more of heparin, lithium heparin, EDTA, and K₂ EDTA.

2. The method of claim 1, further comprising storing the capillary blood mixed with the preservative or anti-coagulant for at least 3 days at room temperature or without freezing, the preservative or anti-coagulant stabilizing the soluble Klotho protein for at least 3 days at room temperature or without freezing.

3. The method of claim 1, further comprising storing the capillary blood mixed with the preservative or anti-coagulant for at least 7 days at room temperature or without freezing, the preservative or anti-coagulant stabilizing the soluble Klotho protein for at least 7 days at room temperature or without freezing.

4. The method of claim 1, further comprising, after the storing step, quantifying the amount of soluble Klotho protein or assaying for the presence of the soluble Klotho protein.

5. The method of claim 4, wherein the step of quantifying the amount of soluble Klotho protein comprises detecting the soluble Klotho protein using mass spectrometry, Multi-Analyte Profiling (xMAP), and/or a first antibody that binds to a portion of the soluble Klotho protein.

6. The method of claim 5, wherein the step of quantifying the amount of soluble Klotho protein further comprises detecting the soluble Klotho protein using a detection antibody that binds to a portion of the first antibody or performing an enzyme-linked immunosorbent assay (ELISA) to detect and optionally quantify the soluble Klotho protein.

7. The method of claim 1, wherein the step of obtaining capillary blood comprises one or more of lancing skin of the mammal with a lancet and collecting the capillary blood in the container.

8. The method of claim 1, wherein the capillary blood is between about 10-1000 ul, preferably about 50-200 ul of the capillary blood, or wherein the step of obtaining capillary blood includes obtaining between about 10-1000 ul, preferably about 50-200 ul of the capillary blood.

9. A method of diagnosing Klotho deficiency in a mammalian subject, the method comprising:
obtaining a fluid sample mixture selected from the group consisting of:
mammalian serum, optionally in the form of capillary blood, mixed with a preservative or anti-coagulant comprising one or more of heparin, lithium heparin, EDTA, and K₂ EDTA, the mammalian serum having an amount of soluble Klotho protein; and
mammalian serum, optionally in the form of capillary blood, the mammalian serum having an amount of soluble Klotho protein, the method further comprising mixing the mammalian serum with a preservative or anti-coagulant comprising one or more of heparin, lithium heparin, EDTA, and K₂ EDTA to form the mixture;
storing the mixture for at least 24 hours at room temperature or without freezing, the preservative or anti-coagulant stabilizing the soluble Klotho protein for at least 24 hours at room temperature or without freezing;
after the storing step, quantifying the amount of the soluble Klotho protein in the mixture; and
diagnosing the mammalian subject with Klotho deficiency when the quantified amount of soluble Klotho protein in the mixture is less than a predetermined threshold amount.

10. The method of claim 9, wherein the fluid sample mixture comprises about 10-1000 ul, preferably about 50-200 ul of capillary blood from the mammalian subject, or wherein the step of obtaining the fluid sample mixture comprises obtaining about 10-1000 ul, preferably about 50-200 ul of capillary blood from the mammalian subject.

11. The method of claim 9, wherein the step of quantifying the amount of the soluble Klotho protein in the mixture comprises detecting the soluble Klotho protein using mass spectrometry, Multi-Analyte Profiling (xMAP), and/or a first antibody that binds to a portion of the soluble Klotho protein and, optionally, a detection antibody that binds to a portion of the first antibody, or performing an enzyme-linked immunosorbent assay (ELISA) to detect and optionally quantify the soluble Klotho protein.

12. The method of claim 9, wherein diagnosing the mammalian subject with Klotho deficiency comprises displaying a Klotho deficiency determination or diagnosis on a user interface of a computer system and/or producing a file or report, in physical or electronic form, that displays the Klotho deficiency determination or diagnosis, the Klotho deficiency determination or diagnosis optionally including the quantified amount of the soluble Klotho protein and/or the predetermined threshold amount.

13. A method of treating Klotho deficiency in a mammalian subject, the method comprising:
obtaining serum or capillary blood, from a mammalian subject, the serum or capillary blood having an amount of soluble Klotho protein;
storing the serum or capillary blood in a container for at least 24 hours at room temperature or without freezing, the container having a preservative or anti-coagulant disposed therein and mixed with the serum or capillary blood to form a mixture, the preservative or anti-coagulant stabilizing the soluble Klotho protein for at least 24 hours at room temperature or without freezing, the preservative or anti-coagulant comprising one or more of heparin, lithium heparin, EDTA, and K₂ EDTA;
after the storing step, quantifying the amount of the soluble Klotho protein in the mixture;
diagnosing the mammalian subject with Klotho deficiency when the quantified amount of soluble Klotho protein in the mixture is less than a predetermined threshold amount; and
administering a composition to the mammalian subject after diagnosing the mammalian subject with Klotho deficiency, the composition comprising one or more of:
one or more pharmaceutical composition comprising a pharmaceutically-acceptable carrier or excipient and a pharmaceutically effective amount of a recombinant Klotho protein, recombinant Klotho protein fragment, or recombinant Klotho fusion protein disposed in the carrier or excipient;
one or more nutraceutical composition comprising a plurality of components adapted to raise serum soluble Klotho protein levels by increasing or enhancing endogenous production of soluble Klotho protein by the mammalian subject; and
one or more pharmaceutical composition that increases endogenous Klotho levels, optionally selected from the group consisting of a blood pressure medication, an Angiotensin II receptor blocker (ARB), an Angiotensin-converting enzyme (ACE) inhibitor, Losartan, Valsartan, Telmisartan, an AT1 receptor antagonist or blocker, testosterone, growth hormone, a growth hormone releasing peptide, sermorelin, prescription or prescription-strength vitamin D, 1,25-dihydroxycholecalciferol, calcitrol, paricalcitrol, an Indoxyl sulphate binder, Kremezin, AST-120, a statins, a PPAR agonists, troglitazone, and rosiglitazone.

14. The method of claim 13, wherein the administering step comprises:
an oral or oral-related administration, optionally selected from the group consisting of ingestion, buccal administration, and sublingual administration, optionally in modified-release form;
one or more bolus or gradual injection, optionally selected from intravenous, intradermal, intraperitoneal, intramuscular, intracutaneous, and subcutaneous injection, optionally in modified-release form.

15. The method of claim 13, wherein the step of obtaining serum or capillary blood comprises one or more of:
lancing skin of the mammalian subject with a lancet;
obtaining between about 10-1000 ul, preferably about 50-200 ul of the capillary blood; and
collecting the capillary blood in a container having a preservative or anti-coagulant disposed therein or added thereto, the capillary blood optionally being between about 10-1000 ul, preferably about 50-200 ul of capillary blood.

16. The method of claim 13, wherein the pharmaceutical composition comprises a pharmaceutically-effective amount of a recombinant Klotho protein, recombinant Klotho protein fragment, or recombinant Klotho fusion protein having at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152.

17. The method of claim 16, wherein the pharmaceutical composition comprises a pharmaceutically-effective amount of a recombinant Klotho fusion protein comprising at least a portion of an immunoglobulin Fc domain sequence or human serum albumin sequence fused to at least a portion of a soluble Klotho protein sequence, with or without a linker sequence therebetween.

18. The method of claim 13, wherein the nutraceutical composition comprises two or more components selected from the group consisting of vitamin C, vitamin D3, vitamin E, N-acetylcysteine (NAC), quercetin dihydrate, rosmarinic acid (RA), pterostilbene, docosahexaenoic acid (DHA), nicotinamide riboside (NR), nicotinamide adenine dinucleotide (NAD+), nicotinamide mononucleotide (NMN), and one or more probiotic.

19. The method of claim 18, wherein the one or more probiotic comprises an effective amount of the Bifidobacterium species and/or strains Bifidobacterium lactis BL-04, Bifidobacterium bifidum/lactis BB-02, and Bifidobacterium longum BL-05, and the Lactobacillus species and/or strains Lactobacillus acidophilus LA-14, Lactobacillus rhamnosus LR-32, and Lactobacillus paracasei LPC-37.

20. A method of purifying a pharmaceutical grade recombinant Klotho protein, recombinant Klotho protein fragment, or recombinant Klotho fusion protein, the method comprising:
obtaining a suspension cell culture fluid comprising (i) recombinant Klotho protein, recombinant Klotho protein fragment, or recombinant Klotho fusion protein and (ii) one or more contaminants;
performing a purification step using affinity chromatography in which the recombinant Klotho protein, recombinant Klotho protein fragment, or recombinant Klotho fusion protein is bound to Protein A or other affinity entity, optionally washed, and eluted from Protein A with and elution buffer having a pH between about pH 2 and about pH 6 and a salt concentration between about 1 M and about 6 M, the salt optionally comprising MgCl₂;
performing a purification step using size exclusion chromatography in which the eluted recombinant Klotho protein, recombinant Klotho protein fragment, or recombinant Klotho fusion protein is further eluted in a mobile phase buffer, the mobile phase buffer having a buffering agent and one or more optional reducing agent, the buffering agent optionally being other than a phosphate-containing buffer, the reducing agent optionally comprising L-Methionine and/or Sodium Thioglycolate, the mobile phase buffer optionally having a pH between about pH 6 and about pH 10,
wherein at least some of the further eluted recombinant Klotho protein, recombinant Klotho protein fragment, or recombinant Klotho fusion protein is optionally in multimeric form.

21. The method of claim 21, wherein the recombinant Klotho protein, recombinant Klotho protein fragment, or recombinant Klotho fusion protein has at least 80% amino acid sequence identity to at least a portion of one of SEQ ID NO: 2 through SEQ ID NO: 70 or SEQ ID NO: 107 through SEQ ID NO: 120 or SEQ ID NO: 125 through SEQ ID NO: 128 or SEQ ID NO: 133 or SEQ ID NO: 152.
